(19) United States
(12) Patent Application Publication
(10) Pub. No.: US 2024/0226382 A1
(43) Pub. Date: Jul. 11, 2024

(54) DRUG-RELEASE IMPLANT FOR LAPAROSCOPIC SURGERY

(71) Applicant: Innocoll Pharmaceuticals Limited, Athlone (IE)

(72) Inventors: Gwendolyn NIEBLER, Chester Sorings, PA (US); Alexandra DIETRICH, Regensburg (DE); Louis PASCARELLA, Basking Ridge, NJ (US)

(73) Assignee: Innocoll Pharmaceuticals Limited, Athlone (IE)

(21) Appl. No.: 18/555,134

(22) PCT Filed: Apr. 12, 2021
(86) PCT No.: PCT/EP2022/059810

§ 371 Date: Oct. 12, 2023

Related U.S. Application Data
(60) Provisional application No. 63/173,909, filed on Apr. 12, 2021.
(60) Provisional application No. 63/177,928, filed on Apr. 21, 2021.
(60) Provisional application No. 63/234,206, filed on Aug. 17, 2021.

(51) Int. Cl. A61L 27/54; A61K 31/445; A61L 27/24; A61L 27/56; A61M 31/00; A61M 37/00

(52) U.S. Cl. CPC A61L 27/54; A61K 31/445; A61L 27/24; A61L 27/56; A61M 31/002; A61M 37/0069; A61L 2300/204; A61L 2300/402

(57) ABSTRACT

A method for implanting a drug delivery device by a laparoscopic procedure may include inserting the drug delivery device through a proximal end of a trocar, moving the drug delivery device through the trocar to an implantation site at a distal end of the trocar. The drug delivery device is then positioned at the implantation site using a manipulator through the trocar.

### CROSS-REFERENCE TO RELATED APPLICATIONS

The present application is an International Application which claims priority to U.S. Provisional Application Nos. 63/173,909 filed Apr. 12, 2021, 63/177,928 filed Apr. 21, 2021, and 63/234,206 filed Aug. 17, 2021, each of which applications incorporated herein in its entirety.

### FIELD

The present disclosure relates generally to the implants for controlled drug release, and in particular, to implants for controlled drug release that are adapted for implantation by laparoscopic surgery.

### BACKGROUND

It is widely accepted that patients typically experience less postoperative pain with minimally invasive (laparoscopic) surgery, which in turn contributes to faster patient recovery, reduced hospital stay, and lower hospital costs compared to the corresponding open procedure. These advantages mean that laparoscopic techniques are being developed and applied for an increasing variety of surgical procedures, enabling progressively more operations to be conducted on an ambulatory basis. However, there remains little doubt that many patients undergoing ambulatory surgery still suffer significant postoperative pain, with 30% reporting moderate to severe pain after 24 hours. Indeed, despite the need for smaller incisions compared to open procedures, the degree of visceral trauma is similar or even more extensive with laparoscopic access. Therefore, the management of early postoperative pain (i.e., for at least the first 24-48 hours) is arguably just as important for patients who are promptly discharged after ambulatory surgery as for those who remain under hospital care for longer with potent analgesics readily at hand if needed.

In recent years, laparoscopy, i.e., minimally invasive techniques, has been applied for a variety of surgeries and, due to its various benefits, laparoscopy has become state of the art for the therapy of diverse indications. Compared to traditional “open” procedures, laparoscopy is a low-risk method that requires only small incisions in the abdominal wall. At each incision a tubular instrument known as trocar is inserted. Through this trocar the surgeon can install materials and instruments, e.g., graspers, or drugs, as well as abstract tissue. Due to the minimally invasive nature of laparoscopy, the patients profit from a shorter recovery as well as less scarring. Furthermore, laparoscopic surgery provides a basis for robotic-assisted surgery, which has started to replace classical surgery.

### SUMMARY

The disclosure provides a method of implanting a drug delivery device for controlled, sustained release of a drug substance at an implantation site, the method comprising: inserting the drug delivery device or a segment thereof into a trocar; moving the drug delivery device or segment thereof through the trocar; and placing the drug delivery device or segment thereof at the implantation site. In some embodiments, the drug delivery device comprises a fibrillar collagen matrix and at least one drug substance selected from an amino amide anesthetic, an amino ester anesthetic, and mixtures thereof. In some embodiments, the drug substance is selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix. In some embodiments, the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm. In some embodiments, the method further comprising removing the drug delivery device from a blister pack enclosure, the drug delivery device comprising a first side which is the blister side, and a second side which is the side opposite to the blister side, wherein the blister side has an increased elasticity compared to the side opposite to the blister side. In some embodiments, the drug delivery device comprises a first side and a second side, wherein the first side has an increased elasticity compared to the second side. In some embodiments, the drug delivery device comprises a first side and a second side, wherein the first side is more stretchable compared to the second side. In some embodiments, the drug delivery device comprises a first side and a second side, wherein the first side comprises one or more beveled surfaces or edges. In some embodiments, the drug delivery device comprises a first side and a second side, wherein the first side is convex, and the second side is concave. In some embodiments, the drug delivery device comprises a first side and a second side, wherein the first side is more flexible compared to the second side. In some embodiments, the method further comprising compressing the drug delivery device prior to inserting into a trocar. In some embodiments, the compressing comprises compressing the drug delivery device between two substantially parallel compression surfaces, wherein when fully closed the compression surfaces define a gap of about 0.5 mm to about 1.6 mm, or about 0.6 mm to about 1.2 mm, or about 0.8 mm to about 1 mm. In some embodiments, the compressing comprises compressing the drug delivery device to a thickness of between about 10% to about 35% of the uncompressed drug delivery device. In some embodiments, after compressing, the drug delivery device re-expands to a thickness of between about 30% to about 70% of an uncompressed drug delivery device. In some embodiments, after compressing, the drug delivery device re-expands to a thickness of between about 2 mm to about 3 mm. In some embodiments, the method further comprising partitioning the drug delivery device into two or more segments, wherein each segment is placed at the implantation site independently. In some embodiments, the method further comprising folding or rolling the drug delivery device or segment thereof prior to insertion into the trocar. In some embodiments, the method further comprising folding or rolling the drug delivery device or drug delivery device segment prior to insertion into the trocar, wherein the first side is substantially at the exterior of the folded or rolled drug delivery device or drug delivery device segment. In some embodiments, the trocar has an internal diameter in a range from about 5 mm to about 16 mm. In some embodiments, the trocar has an internal diameter of about 5 mm, about 8 mm, about 10 mm, or about 12 mm. In some embodiments, the inserting of the drug delivery device or a segment thereof into a trocar is performed with a grasper. In some embodiments, the moving of the drug delivery device or segment thereof through the trocar is performed with a grasper. In some embodiments, the placing of the drug delivery device or segment thereof at the implantation site is performed with a grasper. In some embodiments, the method further comprises unrolling or unfolding the drug delivery device or segment thereof after placement at the implantation site. In some embodiments, the release dissolution profile of a sum of drug delivery device segments is substantially similar to the release dissolution profile of the unpartitioned drug delivery device, wherein the total dose of drug substance in the sum of drug delivery device segments and total dose of drug substance in the unpartitioned drug delivery device is substantially similar. In some embodiments, the release dissolution profile of the sum of drug delivery device segments is within about 1% and about 20% at any point in time substantially similar to the release dissolution profile of the unpartitioned drug delivery device, wherein the total dose of drug substance in the sum of drug delivery device segments and total dose of drug substance in the unpartitioned drug delivery device is substantially similar. In some embodiments, the total dose is about 100 mg, about 200 mg, or about 300 mg.

The disclosure also provide for the use of a drug delivery device or segments thereof for controlled, sustained release of a drug substance at an implantation site, wherein the drug delivery device or segments thereof are configured for inserting into a trocar, moving through the trocar, and placing at the implantation site. In some embodiments, the drug delivery device comprises a fibrillar collagen matrix and at least one drug substance selected from an amino amide anesthetic, an amino ester anesthetic, and mixtures thereof, or wherein the drug substance is selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix. In some embodiments, the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm. In some embodiments, the drug delivery device being removable from a blister pack enclosure, the drug delivery device comprising a first side which is the blister side, and a second side which is the side opposite to the blister side, wherein the blister side has an increased elasticity compared to the side opposite to the blister side. In some embodiments, the drug delivery device or segment thereof comprises a first side and a second side, wherein the first side has an increased elasticity compared to the second side, and/or wherein the first side is more stretchable compared to the second side, and/or wherein the first side comprises one or more beveled surfaces or edges, and/or wherein the first side is convex, and the second side is concave, and/or wherein the first side is more flexible compared to the second side. In some embodiments, the drug delivery device or segment thereof is further configured to be compressed between two substantially parallel compression surfaces, wherein when fully closed the compression surfaces define a gap of about 0.5 mm to about 1.6 mm, or about 0.6 mm to about 1.2 mm, or about 0.8 mm to about 1 mm, and/or wherein the drug delivery device is configured to be compressed to a thickness of between about 10% to about 35% of the uncompressed drug delivery device. In some embodiments, the drug delivery device is configured to re-expand after compressing to a thickness of between about 30% to about 70% of an uncompressed drug delivery device, and/or to a thickness of between about 2 mm to about 3 mm. In some embodiments, the drug delivery device is configured to be partitioned into two or more segments, wherein each segment is configured to be placed at the implantation site independently. In some embodiments, the drug delivery device or segment thereof is configured to be folded or rolled. In some embodiments, the drug delivery device or segment thereof is configured to be folded or rolled, wherein the first side is substantially at the exterior of the folded or rolled drug delivery device or drug delivery device segment. In some embodiments, the drug delivery device or segment thereof is configured for delivery through a trocar having an internal diameter in a range from about 5 mm to about 16 mm, and/or an internal diameter of about 5 mm, about 8 mm, about 10 mm, or about 12 mm. In some embodiments, the drug delivery device or segment thereof is configured to be inserted into a trocar with a grasper, and/or to be moved through the trocar with a grasper, and/or placed at the implantation site with a grasper. In some embodiments, the drug delivery device or segment thereof is configured to be unfolded or unrolled after placement at the implantation site. In some embodiments, the release dissolution profile of a sum of drug delivery device segments is substantially similar to the release dissolution profile of the unpartitioned drug delivery device, wherein the total dose of drug substance in the sum of drug delivery device segments and total dose of drug substance in the unpartitioned drug delivery device is substantially similar, or wherein the release dissolution profile of the sum of drug delivery device segments is within about 1% and about 20% at any point in time substantially similar to the release dissolution profile of the unpartitioned drug delivery device, wherein the total dose of drug substance in the sum of drug delivery device segments and total dose of drug substance in the unpartitioned drug delivery device is substantially similar. In some embodiments, the total dose is about 100 mg, about 200 mg, or about 300 mg.

The disclosure also provides a kit for implanting a drug delivery device or a segment thereof during laparoscopic surgery, the kit comprising: a drug delivery device for controlled, sustained release of a drug substance at an implantation site, and a compression device for compressing the drug delivery device. In some embodiments, the kit further comprising instructions for a clinician for implanting the drug delivery device at an implantation site during a laparoscopic procedure. In some embodiments, the compression device comprises a top plate and a bottom plate. In some embodiments, the instructions comprise the steps for: placing a drug delivery device between the plates of the compression device; compressing the drug delivery device between the plates of the compression device; optionally cutting the compressed drug delivery device into a desired shape; and folding or rolling the cut compressed drug delivery device in a shape amenable to be inserted through a trocar. In some embodiments, the drug delivery device comprises a fibrillar collagen matrix and at least one drug substance selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix. In some embodiments, the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm. In some embodiments, the drug delivery device is removable from a blister pack enclosure, the drug delivery device comprising a first side which is the blister side, and a second side which is the side opposite to the blister side, wherein the blister side has an increased elasticity compared to the side opposite to the blister side, and/or wherein the blister side is more stretchable compared to the side opposite to the blister side, and/or wherein the blister side comprises one or more beveled surfaces or edges, and/or wherein the blister side is more stretchable compared to the side opposite to the blister side, and/or wherein the blister side is convex, and the side opposite to the blister side is concave, and/or wherein the blister side is more flexible compared to the side opposite to the blister side. In some embodiments, the folding or rolling the cut compressed drug delivery device step further comprises folding or rolling the drug delivery device or segment thereof with the first side substantially at the exterior of the folded or rolled drug delivery device or drug delivery device segment, and wherein the instructions further comprise the step of unrolling or unfolding the drug delivery device or segment thereof after placement at the implantation site.

### BRIEF DESCRIPTION OF THE DRAWINGS

Various features of illustrative embodiments of the present disclosure are described below with reference to the drawings. The illustrated embodiments are intended to illustrate, but not to limit, the present disclosure. The drawings contain the following figures:

FIG. 1 shows a picture of XaraColl, a bupivacaine implant.

FIGS. 2A-2F schematically illustrate the cuts (red lines) and holds (blue arrow) used for conforming the clinical XaraColl implant for inserting via a trocar during a laparoscopic procedure in accordance with some embodiments.

FIGS. 3A-3F are representative images of implant segments being held by different types of graspers for insertion through a trocar during a laparoscopic procedure in accordance with some embodiments.

FIGS. 4A-4D are representative images of trocars used for laparoscopic implantation of segments drug-release implant in accordance with some embodiments.

FIGS. 5A and 5B are representative images of the process of inserting an implant segment through a proximal end of a trocar and the implant segment exiting the trocar into a simulated body cavity in accordance with some embodiments.

FIGS. 6A-6E are images and schematic representations of various embodiments of molding and handling of implant and/or implant segments by grasper for inserting through the trocar in accordance with some embodiments.

FIG. 7A schematically illustrates a custom tool with long grasper jaws for handling of implant or implant segments for inserting through the trocar in accordance with some embodiments.

FIG. 7B schematically illustrates a custom tool forming a trocar funnel to guide and form an implant or a segment thereof through the trocar, and potentially hold the trocar valves open in accordance with some embodiments.

FIG. 8A is a representative image of the process of wetting an underside of the implant or implant segment by dipping it in water prior to inserting it through the trocar in accordance with some embodiments.

FIG. 8B is a representative image of the process of wetting the mouth of the trocar prior to inserting the implant or implant segment through the trocar in accordance with some embodiments.

FIG. 8C is a representative image of the process of wetting an underside of the implant or implant segment by spraying the implant with water prior to inserting it through the trocar in accordance with some embodiments.

FIG. 9A shows embodiments for cylindrical/half-cylindrical drug delivery device implants, and FIG. 9B shows embodiments for cylindrical/half-cylindrical drug delivery device implants enclosed in a similarly shaped canister for delivery through a trocar.

FIG. 10 shows the release profile of different shapes and sizes of reformulated implant in accordance with some embodiments.

FIG. 11 shows representative images of the process of inserting cylindrical/half-cylindrical drug delivery device implants through a trocar.

FIG. 12 shows a chart for possible options for reformulating the implant to allow insertion into an enclosure device or a canister for insertion through a trocar for implantation during a laparoscopic procedure in accordance with some embodiments.

FIG. 13 shows the compression of XaraColl using a compression device followed by cutting of XaraColl for insertion into a trocar.

FIG. 14 shows folding of cut XaraColl, wherein the cut XaraColl is folded twice and the folded XaraColl is placed into a grasper.

FIG. 15 shows insertion of the grasper, holding the folded XaraColl, into a trocar.

FIG. 16 shows a close up of the compression device used to compress XaraColl.

FIG. 17 shows compression of a XaraColl implant utilizing a compression tool.

FIG. 18 shows compression tools designed for this study. Left: compression frame, Middle: compression tool generation 1.0, Right: compression tool generation 1.1 (stainless steel).

FIG. 19 shows a scheme for compression (left side) and compression with compression tool (right side).

FIG. 20 shows that a compressed XaraColl with 2 mm thickness can be easily cut with scissors.

FIG. 21 shows the blister (top left) and Tyvek (top right) side of XaraColl and SEM images (200× zoom) of the blister (bottom left) and Tyvek side (bottom right).

FIG. 22 shows the first folding step (left side) and side view after the second folding step (right side).

FIG. 23 shows a scheme for half XaraColl folding (left side) and a XaraColl folding detailed view (right side).

FIG. 24 shows different grasper types with different forceps. Above: pre-selection of graspers, from left to right, straight grasping forceps, straight grasping forceps-fenestrated, rat tooth grasping forceps, duckbill grasping forceps, babcock grasping forceps, clinch grasping forceps, intestinal grasping forceps, Maryland grasping forceps. Below: graspers studied in detail, 1) single action intestinal grasper, 2) double action intestinal grasper, 3) double action babcock grasper, 4) double action Maryland grasper.

FIG. 25 shows XaraColl trocar insertion steps.

FIG. 26 shows Different trocar seals (first seals), left: MOLNLYCKE Health Care, right: ETHICON Endo-Surgery.

FIG. 27 shows 12- and 8-mm Trocars from ETHICON.

FIG. 28 is a chart of the XaraColl test groups investigated with dissolution testing.

FIG. 29 is a measurement flow chart for thickness evaluation.

FIG. 30 is a flow chart for folding evaluation.

FIG. 31 shows different types of damage that may occur during folding.

FIG. 32 contains graphs of thickness data for 3 seconds compression and 0.8 mm compression gap.

FIG. 33 contains graphs of thickness data for 1, 15, and 120 seconds compression with 0.8 mm compression gap.

FIG. 34 shows a swelling comparison, compressed vs. regular.

FIG. 35 is a chart showing the evaluation of graspers.

FIG. 36 provides detailed views of the Babcock grasper utilized in this study.

FIG. 37 shows correct grasper handling: “long grip” results in successful trocar insertion and release of the XaraColl implant without damage.

FIG. 38 shows incorrect grasper handling: “short grip” results damage of the XaraColl implant.

FIG. 39 is a chart comparing two different trocar seals.

FIG. 40 depicts a matrix moved through a 12 mm tube (left half) compared to a matrix moved through an 8 mm tube (right half).

FIGS. 41A-41B depict the dissolution testing of test groups 1-5. FIG. 41A is a chart of average (dark green) and single (light green) dissolution results of XaraColl (group 1). FIG. 41B is a chart of average (dark green) and single (light green) dissolution results of compressed XaraColl (group 2). FIG. 41C is a chart of average (dark blue) and single (light blue) dissolution results of XaraColl, half (group 3). FIG. 41D is a chart of average (dark blue) and single (light blue) dissolution results of XaraColl, trocar (group 4). FIG. 41E is a chart of average (orange) and single (light orange) dissolution results of XaraColl, max compression, half (group 5).

FIG. 42 is a chart overlaying the average dissolution results with standard deviation of XaraColl (group 1, dark green); XaraColl, compressed (group 2, light green); XaraColl, half (group 3, dark blue); XaraColl, trocar (group 4, light blue) and XaraColl, max. compression, half (group 5, orange).

FIG. 43 depicts the sample preparation technique for the laparoscopic application of XaraColl.

FIGS. 44A-44B are charts showing the dimensions and design features of different generations of compression tools. FIG. 44A is a chart of compression tool generations 1-3. FIG. 44B is a chart of compression tool generations 4-5.

FIGS. 45A-45Q depict the dissolution testing of groups 0-5 in Table 25.

FIG. 46 shows resistance to bending measurements using Zwicki 500N. Implants were placed in such a way that the dotted line is oriented in a 90° angle relative to the bending tool.

FIGS. 47A-47B show individual steps for the laparoscopic placement of XaraColl implants. FIG. 47A shows steps 1-8. FIG. 47B shows steps 9-16.

FIG. 48 is a classification of implant damage upon rolling: A) minor damage (<50%), B) major damage (>50%), C) complete break into two parts.

FIG. 49 shows different grasper types used within this study: 35 mm atraumatic bowel grasper (left), 40 mm atraumatic bowel grasper (middle), and 30 mm Babcock grasper (right).

FIG. 50 shows Ethicon (left) and Applied (right) trocar, A) trocar head with segmented seal, B) trocar tube with slit valve, C) trocar with head removed, D) complete trocar, E) trocar head with elastic ring seal, F) trocar head with cross slit valve, G) trocar with head removed, H) complete trocar.

FIG. 51 is a table of dissolution test groups investigated in Example 10.

FIG. 52 shows the design of the final compression tool generation 6.6; A) compression tool assembly with base and top part, B) base part, C) top part, D) side view from the short side, D) side view from the long side.

FIG. 53 provides technical drawings of the compression tool, A) top part: top view, B) top part: side view from the short side, C) top part: side view from the long side, D) base part: tow view, E) base part: side view from the short side, F) base part: side view from the long side.

FIG. 54 is chart of wet tensile strength for compressed and uncompressed matrices.

FIG. 55 contains charts showing the resistance to bending, resistance to pressure, and deformation at break of uncompressed and partially compressed matrices as well as an SEM image of an uncompressed matrix.

FIG. 56 provides data comparing the wet thickness of compressed and uncompressed matrices.

FIG. 57 shows SEM surface images of uncompressed (left) and compressed) implants (right).

FIG. 58 shows SEM cross section images of uncompressed (left) and compressed implants (right).

FIG. 59 shows SEM cross section images uncompressed (left), compressed (center), and pressed in FILK implants (right).

FIG. 60 shows different grasper types loaded with XaraColl implant: 35 mm atraumatic bowel grasper (left), 40 mm atraumatic bowel grasper (middle) and 30 mm Babcock grasper (right).

FIG. 61 shows XaraColl implants after passage through a trocar using different trocar-grasper type combinations.

FIG. 62 demonstrates the unrolling of implants after trocar passage using a wet sponge to mimic the abdominal wall. A) the implant is released from the grasper after trocar passage and partially unfolds, B) the ending of the implant is grabbed with a grasper, C) the implant is placed on the wet sponge, D) the implant is unrolled with the help of two graspers, E) six unrolled implants, which equates the dosage for inguinal hernia repair.

FIG. 63 is a graph of the average dissolution profiles of batches 21011207 (yellow: laparoscopy, orange: open surgery) and 21010507 (light green: laparoscopy, dark green: open surgery) with standard deviations.

FIG. 64 provides SEM cross section images of compressed and uncompressed XaraColl implants.

FIGS. 65A-65B depicts a sample preparation technique for the laparoscopic application of XaraColl.

FIG. 66 provides charts of the resistance to pressure and resistance to bending data for Xaracoll batch 21011207 and 21020507.

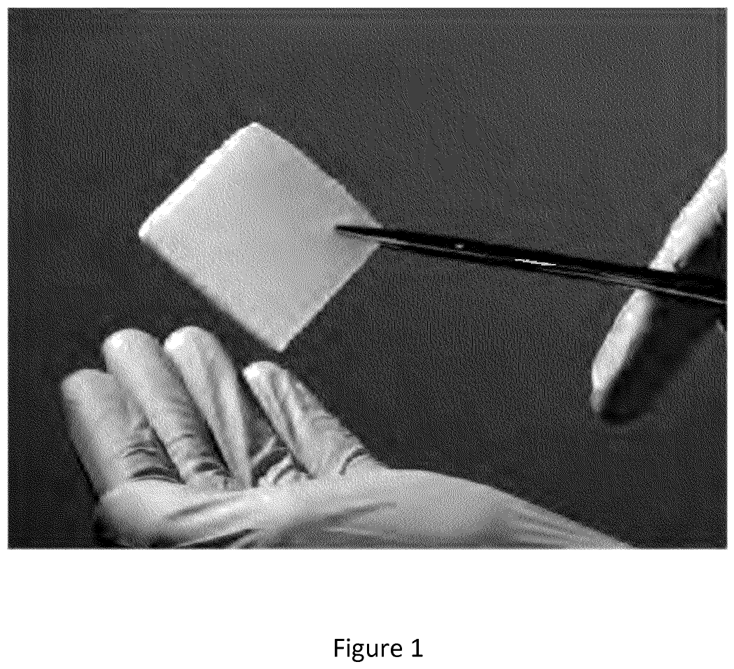

### DETAILED DESCRIPTION

The embodiments disclosed herein stem from the realization that while laparoscopic surgery offers certain advantages in terms of smaller incisions compared to open procedures, laparoscopic surgical procedures do not necessarily reduce the visceral trauma, and thus, management of post-operative pain continues to be of importance. Advantageously, the devices, methods and systems described herein provide implantable devices for controlled and sustained release of pain medication, that can be implanted via a laparoscopic procedure, such as, for example, via a trocar.

In accordance with some embodiments of the present disclosure, a method of implanting a drug delivery device includes inserting the drug delivery device into a trocar, moving the drug delivery through the trocar to an implantation site; and placing the drug delivery device at the implantation site through the trocar.

In some embodiments, the method of implanting a drug delivery device includes wetting the drug delivery device with a predetermined amount of water prior to inserting the drug delivery into the trocar. In some embodiments, the method may include cutting the drug delivery device in a shape suitable for insertion into the trocar prior to inserting the drug delivery device into a trocar.

In accordance with some embodiments of the present disclosure, a kit for implanting a drug delivery device includes a sterile trocar, a vial comprising a predetermined amount of sterile water, a sterile petri-dish, and the drug delivery device cut into a predetermined shape.

In accordance with some embodiments of the present disclosure, a kit for implanting a drug delivery device includes a sterile trocar, and an enclosure device shaped and sized to enable a clinician to insert the enclosure device through a proximal end of the sterile trocar. The enclosure device includes a drug delivery device enclosed therein, the drug delivery device. The enclosure device is structured to enable the clinician, upon moving the enclosure device through a distal end of the sterile trocar and to an implantation site within a body cavity of a living subject, to open the enclosure device and remove the drug delivery device therefrom.

In accordance with some embodiments of the present disclosure, a drug delivery device comprises an implant including a collagen matrix and a drug, and an enclosure enclosing the implant therein. The enclosure is shaped and sized to enable insertion of the enclosure through a proximal end of a sterile trocar. The enclosure is structured to enable the clinician, upon moving the enclosure device through a distal end of the sterile trocar and to an implantation site within a body cavity of a living subject, to open the enclosure device and remove the drug delivery device therefrom.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of breast reconstruction or augmentation, such embodiments can be used with various devices and implants. Furthermore, various applications of such embodiments and modifications thereto, which may occur to those who are skilled in the art, are also encompassed by the general concepts described herein.

As used herein, a “compression tool” is a medical device that is used to compress XaraColl in a controlled way. The compression tool consists of two parts, a bottom part and a top part. XaraColl is placed on the bottom part and is compressed to a defined gap thickness by using the top part.

As used herein, a “grasper” is a medical device that is used during a variety of surgeries. For the intended laparoscopic use of XaraColl, the grasper is used to hold the folded XaraColl and pass it through a trocar.

As used herein, a “sinker” is a customized stainless steel cage which prevents the samples from floating. Sinkers are defined in the USP.

As used herein, a “trocar” is a medical device that is made up of an awl (which may be a metal or a plastic sharpened or a non-bladed tip), a cannula (essentially a hollow tube), and a seal. Trocars are placed through the abdomen during laparoscopic surgery. The trocar functions as a portal for the subsequent placement of materials for wound treatment or other instruments, such as graspers, scissors, staplers, etc.

As used herein, a “vessel” is a small reactor that contains a defined amount of dissolution medium as well as the sample. Eight vessels are placed in a water bath for external temperature control. Each of the vessels has its own certificate of conformance.

Hernia repair is the most common general surgical procedure performed in the United States, and it has recently been estimated that 42% of males will develop an inguinal hernia in their lifetime. From studies comparing laparoscopic versus open repair of inguinal hernias, it is generally reported that laparoscopy offers the advantages of less postoperative pain and faster patient recovery, but at the expense of a more complex and longer operation, and perhaps with increased risk of serious complications. However, other authors have argued that laparoscopic repair can be performed efficiently without major complications. Laparoscopic repair of umbilical and ventral hernias have demonstrated similar benefits over open surgery.

Although laparoscopic procedures are widely considered less painful than the corresponding open procedure, well-controlled studies have shown this difference to be relatively modest. For example, in a large randomized trial that compared laparoscopic versus open inguinal hernia repair involving more than 2000 patients at 14 centers, the difference in mean pain intensity at rest using a 150 mm visual analog scale was only 10.2 mm on the day of surgery and 6.2 mm after 2 weeks. Similarly small differences were also reported during normal activities and patient perception of their worst pain. In another prospective study comparing laparoscopic and open repair of ventral hernias, patients undergoing laparoscopic surgery had significantly lower pain scores at 72 hours postoperatively, but not at 24 or 48 hours.

Since early hospital discharge is a primary goal of laparoscopic surgery, the importance of controlling immediate postoperative pain has been recognized and multimodal analgesia recommended. Infiltration of abdominal wounds and/or instillation of the extraperitoneal space with bupivacaine have been investigated as possible methods for reducing postoperative pain following laparoscopic inguinal hernia repair in a number of randomized controlled trials. However, the results are contradictory, with some researchers reporting a significant benefit and others concluding the opposite. Similarly mixed results have been reported with use of intraperitoneal bupivacaine in laparoscopic cholecystectomy, with some but by no means all studies demonstrating an analgesic effect. Another study concluded a detectable, albeit subtle and transient benefit. A recent systematic review and meta-analysis of randomized controlled trials acknowledged this high level of clinical heterogeneity, but still concluded there was evidence to support intraperitoneal administration of local anesthetics in laparoscopic cholecystectomy.

The characteristics of pain following laparoscopy are thought to differ considerably from those after laparotomy, with visceral pain predominating after laparoscopic cholecystectomy. Nevertheless, the reason for the inconsistent analgesic outcome with use of intraperitoneal bupivacaine following laparoscopic surgery is not obvious. One possible explanation is differences related to administration technique, since studies in patients undergoing laparoscopic inguinal hernia repair have shown that the timing of the instillation can influence its effectiveness. In another authoritative review of surgical wound infiltration, the importance of using controlled and meticulous techniques has been strongly emphasized. Other studies have suggested that a surgical hemostat comprised of oxidized regenerated cellulose and soaked with bupivacaine can control the visceral pain when placed in the gallbladder bed after laparoscopic cholecystectomy.

The safety and efficacy of, a bupivacaine-collagen implant, for postoperative analgesia in patients undergoing open inguinal hernioplasty and open gynecological surgery have recently been reported. The results indicate that the bupivacaine-collagen implant implanted intraoperatively was able to target and control visceral pain more efficiently than continuous perfusion of the surgical wound with bupivacaine for 72 hours postoperatively. Hence, the bupivacaine-collagen implant may be of particular benefit for reducing the postoperative pain normally experienced by patients undergoing laparoscopic surgery.

Accordingly, embodiments of the present disclosure provide a method for implanting drug delivery devices such as, for example, an implant for delivering an analgesic for postoperative pain management, during a laparoscopic surgical procedure. In some embodiments, the drug delivery device may be delivery drugs such as, for example, Bupivacaine, Levobupivacaine, Lidocaine, Mepivacaine, Prilocaine, Ropivacaine, Articaine, Trimecaine and their salts and prodrugs. In some embodiments, the implant may be a bupivacaine-collagen implant. The methods, devices and kits disclosed herein can be suitably modified for delivery of other drugs are contemplated within the scope of this disclosure.

The following disclosure describes the methods, systems, devices and kits for implanting a bupivacaine-collagen implant. However, those of skill in the art, upon understanding of the present disclosure, will be able to suitably modify the methods, systems, devices and kits disclosed herein for implanting other types of implants designed for controlled release of drugs upon implantation.

In an embodiment, the collagen used in the bupivacaine-collagen implant comprises dehumidified mature lyophilized milled collagen (LMC). In an embodiment, the dehumidified mature LMC is formed from isolated collagen that is lyophilized and matured in a permeable pouch in an environment of about 40° C. and about 65% relative humidity (RH). In an embodiment, the lyophilized collagen is maintained in the heated environment with controlled humidity until the dehydrated collagen reaches an LOD (loss on drying) of about 18%, forming mature LMC.

In an embodiment, the mature LMC is dehumidified in a permeable pouch in an environment of about 25° C. and a RH of about 15%. In an embodiment, the mature LMC is dehumidified until an LOD of about 10% is reached, forming dehumidified mature LMC.

Examples of bupivacaine-collagen implants are described in U.S. Pat. No. 8,034,368, which is incorporated herein by reference in its entirety for all purposes. Formulations for and methods of obtaining collagen that can be used in bupivacaine-collagen implants are described in U.S. Pat. No. 10,487,134, which is incorporated herein by reference in its entirety for all purposes. Other examples of drug delivery implants for controlled, sustained drug delivery are described in International Patent Application Publication Nos. WO 2019/071243, WO 2019/071245, WO 2019071246, WO 2019/136490, WO 2019/221853, WO 2020/047013, and WO 2020/046973; US Patent Application Publication Nos. US 2020/0246255, US 2021/0069101, and US 2020/0368398; and Chinese Patent Application Publication Nos. CN 111432807, CN 111655303, and CN 112367980, each of which is incorporated herein by reference in its entirety for all purposes.

The bupivacaine-collagen implants described in U.S. Pat. No. 8,034,368 is typically used for incisional anesthesia in herniotomy surgery. In some embodiments, the bupivacaine-collagen implant may be XaraColl. The typical size of a clinically used bupivacaine-collagen implant, such as XaraColl, is 5 cm×5 cm (×0.5 cm thick). In a typical clinical procedure, patients received three 5 cm×5 cm (×0.5 cm thick) sponges; one sponge divided between areas adjacent the surgical site (in this case, adjacent the location of the now-removed uterus), one sponge divided and placed across the incision in the wall of the body cavity (in this case, the peritoneum) and the final sponge divided and placed between the sheath and skin around the incision. Each sponge contained 50 mg of bupivacaine hydrochloride, giving a total dose of 150 mg per patient.

In some embodiments, the bupivacaine-collagen implant may be in the form of a depot for the treatment of postoperative pain via sustained, controlled release of bupivacaine. The depot may include a therapeutic region comprising bupivacaine. A control region of the depot comprises a bioresorbable polymer and a releasing agent mixed with the polymer. The releasing agent is configured to dissolve when the depot is placed in vivo to form diffusion openings in the control region. The depot is configured to be implanted at a treatment site in vivo and, while implanted, release bupivacaine at the treatment site for no less than 7 days. In some embodiments, bupivacaine in the therapeutic region comprises at least 50% of the total weight of the depot.

In some embodiments, the depot is configured to release the analgesic at the treatment site for no less than 14 days. In some embodiments, about 20% to about 50% of bupivacaine is released in the first about 3 to about 5 days of the 14 days, and at least 80% of the remaining bupivacaine is released in the last 11 days of the 14 days. In some embodiments, about 20% to about 40% of bupivacaine is released in the first 3 days of the 14 days, and at least 80% of the remaining bupivacaine is released in the last 11 days of the 14 days.

In some embodiments, at least 90% of the remaining bupivacaine is released in the last 11 days of the 14 days. In some embodiments, no more than 15% of the amount of bupivacaine is released in the first 2 days of the 14 days.

In some embodiments, the depot is configured to release bupivacaine at a first rate for a first period of time and at a second rate for a second period of time. The first rate may be greater than the second rate. The depot may be configured to release at least 90% of the analgesic in the therapeutic region within 14 days.

In some embodiments, the depot is configured to release about 100 mg to about 500 mg of bupivacaine to the treatment site per day.

In some embodiments, depot for the treatment of postoperative pain via sustained, controlled release of bupivacaine includes a therapeutic region comprising bupivacaine. A control region of the depot comprises a bioresorbable polymer and a releasing agent mixed with the polymer. The releasing agent is configured to dissolve when the depot is placed in vivo to form diffusion openings in the control region. The depot is configured to be implanted at a treatment site in vivo and, while implanted, release bupivacaine at the treatment site for no less than 14 days. About 20% to about 40% of bupivacaine is released in the first 3 days of the 14 days, and wherein at least 80% of the remaining bupivacaine is released in the last 11 days of the 14 days.

In some embodiments, a depot for the treatment of postoperative pain via sustained, controlled release of bupivacaine includes a therapeutic region comprising bupivacaine. A control region of the depot comprises a bioresorbable polymer and a releasing agent mixed with the polymer. The releasing agent is configured to dissolve when the depot is placed in vivo to form diffusion openings in the control region. The depot is configured to be implanted at a treatment site in vivo and, while implanted, release bupivacaine at the treatment site for no less than 3 days. The control region does not include bupivacaine at least prior to implantation of the depot at the treatment site.

In some embodiments, the implant may further include an antibiotic, an antifungal, and/or an antimicrobial. The antibiotic, the antifungal, and/or the antimicrobial is selected from at least one of amoxicillin, amoxicillin/clavulanate, cephalexin, ciprofloxacin, clindamycin, metronidazole, azithromycin, levofloxacin, sulfamethoxazole/trimethoprim, tetracycline(s), minocycline, tigecycline, doxycycline, rifampin, triclosan, chlorhexidine, penicillin(s), aminoglycides, quinolones, fluoroquinolones, vancomycin, gentamycin, cephalosporin(s), carbapenems, imipenem, ertapenem, antimicrobial peptides, cecropin-mellitin, magainin, dermaseptin, cathelicidin, α-defensins, and α-protegrins, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, and amphotericin B.

In some embodiments, the implant may further includes an anti-inflammatory agent selected from at least one of steroids, prednisone, betamethasone, cortisone, dexamethasone, hydrocortisone and methylprednisolone, non-steroidal anti-inflammatory drugs (NSAIDs), aspirin, Ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, and COX-2 inhibitors.

In some embodiments, the implant further includes at least one of: epinephrine, clonidine, transexamic acid.

In some embodiments, the releasing agent is a non-ionic surfactant. In some embodiments, the releasing agent has hydrophilic properties. In some embodiments, the releasing agent is a polysorbate. In some embodiments, the releasing agent is Tween 20. In some embodiments, the releasing agent is Tween 80. In some embodiments, the releasing agent is non-polymeric. In some embodiments, the releasing agent is not a plasticizer.

In some embodiments, the polymer is configured to degrade only after substantially all of bupivacaine has been released from the depot.

In some embodiments, the polymer is a copolymer. In some embodiments, the polymer is a terpolymer.

In some embodiments, the polymer includes at least one of polyglycolide (PGA), polycaprolactone (PCL), poly(DL-lactic acid) (PLA), poly(alpha-hydroxy acids), poly(lactide-co-glycolide) (PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hex-ane-co-sebacic acid, polyphosphazene, ethyl glycinate polyphosphazene, polycaprolactone co-butylacrylate, a copolymer of polyhydroxybutyrate, a copolymer of maleic anhydride, a copoly-mer of poly(trimethylene carbonate), polyethylene glycol (PEG), hydroxypropylmethylcellulose and cellulose derivatives, polysaccharides (such as hyaluronic acid, chitosan and starch), pro-teins (such as gelatin and collagen) or PEG derivatives, polyaspirins, polyphosphagenes, colla-gen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate)hydroxypropyl cellulose, hydroxypropyl methylcellulose, hydroxyethyl methylcellulose, carboxymethylcellulose or salts thereof, Carbo-pol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvi-nyl alcohols, propylene glycol, and poly(DL-lactide-co-glycolide-co-caprolactone).

In some embodiments, the polymer is one of poly(DL-lactide-co-glycolide-co-caprolactone) and poly(DL-lactide-co-glycolide)(PLGA).

In some embodiments, the polymer is poly(DL-lactide-co-glycolide-co-caprolactone) in a molar ratio of 60:30:10. In some embodiments, the polymer is poly(DL-lactide-co-glycolide)(PLGA) in a molar ratio of 50:50.

In some embodiments, the polymer is ester-terminated. In some embodiments, the polymer is a terpolymer that includes three polymers selected from the following: polyglycolide (PGA), polycaprolactone (PCL), poly(L-lactic acid) (PLA), poly(DL-lactic acid) (PLA), poly(trimethylene carbonate) (PTMC), poly-dioxanone (PDO), poly(4-hydroxy butyrate) (PHB), polyhydroxyalkanoates (PHA), poly(phosphazene), and polyethylene glycol.

In some embodiments, the polymer is a first polymer, and the therapeutic region includes a second polymer mixed with bupivacaine. In some embodiments, the first polymer and the second polymer are the same. In some embodiments, the first polymer and the second polymer are different.

In some embodiments, the first polymer and/or the second polymer include at least one of polyglycolide (PGA), polycaprolactone (PCL), poly(DL-lactic ac-id) (PLA), poly(alpha-hydroxy acids), poly(lactide-co-glycolide)(PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate es-ter), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphos-phazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic ac-id, chitosan and starch), proteins (such as gelatin and collagen) or PEG derivatives, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha to-copheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAB (sucrose acetate isobutyr-ate)hydroxypropyl cellulose, hydroxypropyl methylcellulose, hydroxyethyl methylcellulose, car-boxymethylcellulose or salts thereof, Carbopolpoly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, poly(DL-lactide-co-glycolide-co-caprolactone).

In some embodiments, the first polymer and/or the second polymer selected from the following: poly(DL-lactide-co-glycolide-co-caprolactone) and poly(DL-lactide-co-glycolide)(PLGA). In some embodiments, the first polymer and/or the second polymer is poly(DL-lactide-co-glycolide-co-caprolactone) and has a molar ratio of 60:30:10. In some embodiments, the first polymer and/or the second polymer is poly(DL-lactide-co-glycolide) and has a molar ratio of 50:50.

In some embodiments, the first polymer and/or the second polymer is ester-terminated. In some embodiments, the first polymer and/or the second polymer is a terpolymer that includes three polymers selected from the following: polyglycolide (PGA), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), and polyethylene glycol.

In some embodiments, the ratio of the releasing agent to the polymer in the control region is at least 1:1.

In some embodiments, the releasing agent is configured to dissolve when the depot is placed in contact with phosphate buffered saline to form diffusion openings.

In some embodiments, the releasing agent dissolves at a first rate and the polymer degrades at a second rate, wherein the first rate is greater than the second rate.

In some embodiments, the releasing agent dissolves in response to contact between the control region and the physiologic fluids at the treatment site. In some embodiments, diffusion openings in the control region are created via the dissolution of the releasing agent in response to physiologic fluids at the treatment site.

In some embodiments, the releasing agent is a first releasing agent and the therapeutic region includes a second releasing agent. Microchannels are created in the therapeutic region and the control region via dissolution of the first and/or second releasing agents. In some embodiments, at least some of the microchannels penetrate both the therapeutic region and the control region.

In some embodiments, the therapeutic region comprises a plurality of microlayers, and wherein at least some of the microchannels extend through consecutive microlayers.

In some embodiments, the control region comprises a first plurality of microlayers and the therapeutic region comprises a second plurality of microlayers, and wherein at least some of the microchannels extend through the first and second plurality of microlayers.

In some embodiments, a porosity of the depot is increased via dissolution of the releasing agent.

In some embodiments, bupivacaine is released one or more times in substantially discrete doses after implantation.

In accordance with some embodiments of the present disclosure, a method of implanting a drug delivery device for controlled, sustained release of an analgesic at an implantation site. The implant may be any implant disclosed herein. The method includes inserting the implant or a segment thereof into a trocar, moving the inserted implant or segment thereof through the trocar to the implantation site and placing the inserted implant or segment thereof at the implantation site.

In some embodiments, the implant may have a length of about 50 mm, a width of about 50 mm, and a thickness of about 5 mm. In some embodiments, the implant may have any shape, in particular, cylindrical, semi-cylindrical, corrugated, cuboid, hexahedral, or any other shape that will enable the implant to pass through an inner bore of a trocar. The method may further include cutting the implant into segments having a shape or a size suitable for insertion through the trocar. For example, a sheet-like implant may be cut into one or more segments having a shape selected from a square, a rectangle, a right triangle, and an elongate triangle. The segments may be ½, ⅓ or ¼ in size relative to the implant.

In some embodiments, the implant or a segment thereof is wetted prior to insertion into the trocar. The implant or the segment thereof may be wetted using water or saline which is preferably sterilized. The implant or the segment thereof may be wetted by dipping the implant or the segment thereof in a container such as a petri-dish having water or saline therein. In some embodiments, an edge or an edge of an underside of the implant or the segment thereof is dipped in the container. In some embodiments, the implant or the segment thereof may be wetted by dispensing water or saline on the implant or the segment thereof using a syringe or a pipette or a similar dispensing device. In some embodiments, the implant or the segment thereof may be wetted by spraying water or saline on the implant or segment thereof using a spray bottle or a similar spraying device.

In another embodiment, the implant or a segment thereof is compressed before insertion into the trocar. In an embodiment, the implant comprises one independent segment having dimensions of about 5 cm×5 cm×0.5 cm that is compressed to dimensions of about 5 cm×5 cm×0.2 cm. In an embodiment, the compressed implant is cut into two independent segments having dimensions of about 5 cm×2.5 cm×0.2 cm. In an embodiment, the compressed implant comprises about 2.5 mg/cm³ to about 7.5 mg/cm³ collagen. In an embodiment, the compressed implant comprises about 15 mg/cm³ to about 25 mg/cm³ bupivacaine, or a salt thereof. In one embodiment, the compressed implant comprises about 5 mg/cm³ collagen and about 20 mg/cm³ bupivacaine, or a salt thereof.

In an embodiment wherein the compressed implant is cut into two independent segments, each independent segment is folded in half a first time such that a top side, comprising one or more beveled edges, is on the inside of the fold and a bottom side, lacking beveled edges, is on the outside of the fold. In an embodiment, each folded segment has dimensions of about 2.5 cm×2.5 cm×0.4 cm. In an embodiment, the segment does not crack or break when folded. In an embodiment, the bottom side of the segment on the outside of the fold does not crack or break. In an embodiment, each folded segment is folded in half a second time, forming two twice folded segments each having dimensions of about 1.25 cm×2.5 cm×0.8 cm. In an embodiment, the segment does not crack or break when folded the second time. In an embodiment, the bottom side of the segment on the outside of the fold does not crack or break when the segment is folded the second time.

In an embodiment, one twice folded segment is placed into prongs of a grasper, wherein the prongs are configured for opening after insertion of the grasper through a trocar. In an embodiment, the grasper is inserted through the trocar such that the prongs of the grasper extend beyond the end of the trocar.

In some embodiments, the implant or the segment thereof may be inserted into an enclosure or a canister prior and the enclosure/canister may be inserted into the trocar. In such embodiments, placing the implant or the segment thereof at the implantation site may include opening. after the enclosure/canister has passed through the distal end of the trocar, the enclosure/canister and removing the implant or the segment thereof from the enclosure/canister. The implant or the segment thereof is then placed at the implantation site, and the enclosure/canister is removed through the trocar.

Embodiments of the present disclosure further include a kit for implanting a drug delivery device by laparoscopic surgery. The kit may include a trocar, an implant or a segment thereof described herein, and instructions for a clinician for implanting the implant or the segment thereof at an implantation site during a laparoscopic procedure.

The kit may further include a container comprising a predetermined amount of water or saline which is preferably sterile. In some embodiments, the kit may include a cutting device, such as a scissor, for cutting the implant into a segment thereof having a shape suitable for insertion through a trocar. In some embodiments, the kit may also include a trocar suitable for insertion of the implant or the segment thereof to the implantation during a laparoscopic procedure.

The instructions, in some embodiments, may instruct the clinician on how to wet the implant, how to insert the implant into a trocar, and/or how to move the implant through the trocar. The instructions may further include instructions relating to the cutting the implant into suitably sized and shaped segments for insertion through the trocar.

In some embodiments, the implant may have markings thereon for indicating lines along which to cut the implant. In addition, the implant may have markings thereon indicating preferred lines along which to hold/grasp the implant segment for successful insertion through the trocar.

In another aspect, the present disclosure provides a method of compressing a drug delivery device, the method comprising: placing a drug delivery device on a bottom plate of a compression device; placing a top plate of the compression device directly on top of the bottom plate; firmly pressing on the top plate of the compression device for several seconds; and removing the top plate of the compression device to provide a compressed drug delivery device, wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

The top and bottom plate of the compression device can be made of any solid material known to a person of skill in the art. In an embodiment, the solid material used for the top and bottom plates does not break or deform under pressure from a human hand. In an embodiment, the top and bottom plates are 3D printed using any material suitable for 3D printing. In an embodiment, the top and bottom plates each comprise a recessed portion large enough that the bottom surface of the drug delivery device (i.e. the implant) can fit into the recessed portion of the bottom plate and the top surface of the implant can fit into the recessed portion of the top plate. In an embodiment, the recessed portions have a depth such that when the top plate is placed on top of the bottom plate without the implant present, a gap of about 0.75 cm is formed between the recessed portion of the top plate and the recessed portion of the bottom plate.

In an embodiment, the drug delivery device compressed by the above method is described elsewhere herein. In an embodiment, the drug delivery device is described elsewhere herein as an implant.

In an embodiment, the method provides a compressed drug delivery device (i.e., a compressed implant) having dimensions of about 5 cm×5 cm×0.2 cm. In an embodiment, the method provides a compressed drug delivery device comprising about 2.5 mg/cm³ to about 7.5 mg/cm³ collagen. In an embodiment, the method provides a compressed drug delivery device comprising about 15 mg/cm³ to about 25 mg/cm³ bupivacaine, or a salt thereof. In an embodiment, the method provides a compressed drug delivery device comprising about 5 mg/cm³ collagen and about 20 mg/cm³ bupivacaine, or a salt thereof. In an embodiment, the method provides a compressed drug delivery device comprising about 5 mg/cm³ collagen and about 20 mg/cm³ bupivacaine hydrochloride.

In another aspect, the present disclosure relates to a method of preparing a compressed drug delivery device for insertion into a trocar, the method comprising: cutting the compressed drug delivery device into two or more independent segments, each segment having a top side comprising one or more beveled edges and a bottom side lacking beveled edges; folding each segment in half a first time such that the top side is on the inside of the fold and the bottom side is on the outside of the fold; and folding each segment in half a second time; wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

In an embodiment, the method further comprises placing one twice folded segment into prongs of a grasper, wherein the prongs are configured for opening after insertion of the grasper through a trocar. In an embodiment, the method further comprises inserting the grasper through the trocar such that the prongs of the grasper extend beyond the end of the trocar.

The compressed drug delivery device is described elsewhere herein. In an embodiment, the compressed drug delivery device is cut into two independent segments. In an embodiment, each segment has dimensions of about 5 cm×2.5 cm×0.2 cm. In an embodiment, each segment folded one time has dimensions of about 2.5 cm×2.5 cm×0.4 cm. In an embodiment, each segment folded two times has dimensions of about 2.5 cm×1.25 cm×0.8 cm. In an embodiment, each segment does not crack or break when folded. In an embodiment, the bottom side of each segment does not crack or break when folded.

The following clauses describe certain embodiments.

Clause 1. A method of implanting a drug delivery device for controlled, sustained release of an analgesic at an implantation site, the method comprising: inserting the drug delivery device or a segment thereof into a trocar; moving the inserted drug delivery device or segment thereof through the trocar; and placing the inserted delivery device or segment thereof at the implantation site.

Clause 2. The method of clause 1, wherein the drug delivery device or the segment thereof has any shape adapted for insertion into the trocar.

Clause 3. The method of clause 1, further comprising partitioning the drug delivery device into segments with a predetermined size.

Clause 4. The method of clause 3, wherein partitioning the drug delivery device comprises cutting the drug delivery device.

Clause 5. The method of clause 3 or 4, wherein the drug delivery device is partitioned into two or more segments, wherein each segment is placed at the implantation site independently.

Clause 6. The method of any one of clauses 1 to 5, wherein the drug delivery device has a length of about 50 mm, a width of about 50 mm, and a thickness of about 5 mm.

Clause 7. The method of any one of clauses 3 to 5, wherein partitioning the drug delivery device comprises cutting the drug delivery device into a shape selected from: a square, a rectangle, a right triangle, and an elongate triangle.

Clause 8. The method of any one of clauses 3 to 7, wherein the segments are about ½, about ⅓, or about ¼ in size relative to the drug delivery device.

Clause 9. The method of any one of clauses 1 to 7, wherein the trocar has an internal diameter in a range from about 10 mm to about 12 mm.

Clause 10. The method of any one of clauses 1 to 7, wherein the trocar has an internal diameter of about 5 mm, about 8 mm, about 10 mm, or about 12 mm.

Clause 11. The method of any one of clauses 1 to 10, wherein the drug delivery device is a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof.

Clause 12. The method of clause 11, wherein the at least one drug substance is substantially homogeneously dispersed in the collagen matrix.

Clause 13. The method of clause 11, wherein the at least one drug substance is present in an amount sufficient to provide a duration of local analgesia, local anesthesia or nerve blockade which lasts for at least about one day after administration.

Clause 14. The method of clause 11 wherein the drug substance is selected from Bupivacaine, Levobupivacaine, Lidocaine, Mepivacaine, Prilocaine, Ropivacaine, Articaine, Trimecaine, and their salts and prodrugs.

Clause 15. The method of clause 11, wherein the fibrillar collagen matrix is a Type I collagen matrix.

Clause 16. The method of clause 15, wherein the fibrillar collagen matrix is a Type I collagen matrix and the at least one drug substance is an amino amide anesthetic selected from bupivacaine and salts and prodrugs thereof.

Clause 17. The method of any one of clauses 1 to 16, wherein the drug delivery device comprises one or more collagen sponges.

Clause 18. The method of clause 17, wherein a collagen sponge comprises about 1.0 to about 30.0 mg/cm³ bupivacaine hydrochloride.

Clause 19. The method of clause 17, wherein a collagen sponge comprises about 2.0 to about 22.0 mg/cm³ bupivacaine hydrochloride.

Clause 20. The method of clause 17, wherein a collagen sponge comprises about 4.0 to about 15.0 mg/cm³ bupivacaine hydrochloride.

Clause 21. The method of clause 17, wherein a collagen sponge comprises about 6.0 to about 12.0 mg/cm³ bupivacaine hydrochloride.

Clause 22. The method of clause 17, wherein a collagen sponge comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 23. The method of clause 17, wherein a collagen sponge comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride.

Clause 24. The method of any one of clauses 17 to 23, wherein a collagen sponge comprises about 1.0 to about 20.0 mg/cm³ type I collagen.

Clause 25. The method of any one of clauses 17 to 24, wherein a collagen sponge comprises about 5.6 mg/cm³ type I collagen and between about 4.0 and about 22 mg/cm³ bupivacaine hydrochloride.

Clause 26. The method of any one of clauses 1 to 25, further comprising wetting the drug delivery device or the segment thereof, or a surface of the trocar, prior to insertion into the trocar.

Clause 27. The method of clause 26, wherein the wetting comprises wetting by sterile water or saline.

Clause 28. The method of clause 26, wherein the wetting comprises dipping an edge of the delivery device or segment thereof into a predetermined amount of sterile water or saline.

Clause 29. The method of clause 26, wherein the wetting comprises spraying a predetermine amount of sterile water or saline on the drug delivery device or segment thereof, and/or the surface of the trocar.

Clause 30. The method of clause 26, wherein the wetting comprises disposing a predetermined amount of sterile water or saline on the drug delivery device or segment thereof.

Clause 31. The method of any one of clauses 1 to 30, wherein the release dissolution profile of the sum of drug delivery segments is substantially similar to the release dissolution profile of the unpartitioned drug delivery device.

Clause 32. The method of any one of clauses 1 to 31, wherein the release dissolution profile of the sum of drug delivery segments is within about 1% and about 15% at any point in time substantially similar to the release dissolution profile of the unpartitioned drug delivery device.

Clause 33. Use of a fibrillar collagen matrix and at least one drug substance selected from the group consisting of amino amide anesthetics, amino ester anesthetics and mixtures thereof, for the manufacture of a drug delivery device for providing local analgesia, local anesthesia or nerve blockade at a site in a human or animal in need thereof.

Clause 34. The use according to clause 33, wherein the at least one drug substance is substantially homogeneously dispersed in the collagen matrix.

Clause 35. The use according to clause 33, wherein the at least one drug substance is present in an amount sufficient to provide a duration of local analgesia, local anesthesia or nerve blockade which lasts for at least about one day after administration.

Clause 36. The use according to clause 33, wherein the drug substance is selected from Bupivacaine, Levobupivacaine, Lidocaine, Mepivacaine, Prilocaine, Ropivacaine, Articaine, Trimecaine, and their salts and prodrugs.

Clause 37. The use according to clause 33, wherein the fibrillar collagen matrix is a Type I collagen matrix.

Clause 38. The use according to clause 33, wherein the fibrillar collagen matrix is a Type I collagen matrix and the at least one drug substance is an amino amide anesthetic selected from bupivacaine and salts and prodrugs thereof.

Clause 39. The use according to any one of clauses 34 to 39, wherein the drug delivery device comprises one or more collagen sponges.

Clause 40. The use according to clause 39, wherein a collagen sponge comprises about 1.0 to about 30.0 mg/cm³ bupivacaine hydrochloride.

Clause 41. The use according to clause 39, wherein a collagen sponge comprises about 2.0 to about 22.0 mg/cm³ bupivacaine hydrochloride.

Clause 42. The use according to clause 39, wherein a collagen sponge comprises about 4.0 to about 15.0 mg/cm³ bupivacaine hydrochloride.

Clause 43. The according to clause 39, wherein a collagen sponge comprises about 6.0 to about 12.0 mg/cm³ bupivacaine hydrochloride.

Clause 44. The use according to clause 39, wherein a collagen sponge comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 45. The use according to clause 39, wherein a collagen sponge comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride.

Clause 46. The use according to any one of clauses 33 to 45, wherein a collagen sponge comprises about 1.0 to about 20.0 mg/cm³ type I collagen.

Clause 47. The use according to clause 39, wherein a collagen sponge comprises about 5.6 mg/cm3 type I collagen and between about 4.0 and about 22 mg/cm³ bupivacaine hydrochloride.

Clause 48. The use according to any one of clauses 33 to 47, wherein the drug delivery device or a segment thereof is configured for sustained release of an analgesic at an implantation site.

Clause 49. The use according to any one of clauses 33 to 48, wherein the drug delivery device or a segment thereof is configured for inserting into a trocar, for moving through a trocar, and/or for placement at an implantation site.

Clause 50. The use according to any one of clauses 33 to 49, wherein the drug delivery device or the segment thereof has any shape adapted for insertion into a trocar.

Clause 51. The use of any one of clauses 34 to 51, wherein the drug delivery device is configured for partitioning into segments with a predetermined size

Clause 52. The use according to clause 51, wherein partitioning the drug delivery device comprises cutting the drug delivery device.

Clause 53. The use according to any one of clauses 33 to 52, wherein the drug delivery device is configured for partitioning into two or more segments, wherein each segment is placeable at an implantation site independently.

Clause 54. The use according to any one of clauses 33 to 53, wherein the drug delivery device has a length of about 50 mm, a width of about 50 mm, and a thickness of about 5 mm.

Clause 55. The use according to any one of clauses 52 to 54, wherein the drug delivery device is configured for partitioning by cutting the drug delivery device into a shape selected from: a square, a rectangle, a right triangle, and an elongate triangle.

Clause 56. The use according to any one of clauses 52 to 55, wherein the segments are about ½, about ⅓, or about ¼ in size relative to the drug delivery device

Clause 57. The use according to any one of clauses 33 to 47, wherein the drug delivery device or a segment thereof has a cylindrical or half-cylindrical shape, with a length between about 30 mm and about 100 mm, and a radius between about 3 mm and about 8 mm.

Clause 58. The use according to any one of clauses 49 to 57, wherein the trocar has an internal diameter in a range from about 10 mm to about 12 mm

Clause 59. The use according to any one of clauses 49 to 57, wherein the trocar has an internal diameter of about 5 mm, about 8 mm, about 10 mm, or about 12 mm.

Clause 60. The use according to any one of clauses 33 to 59, wherein the drug delivery device or segment thereof is configured for wetting.

Clause 61. The use according to clause 60, wherein the wetting comprises wetting by sterile water or saline.

Clause 62. The use according to clause 60, wherein the wetting comprises dipping an edge of the delivery device or segment thereof into a predetermined amount of sterile water or saline.

Clause 63. The use according to clause 60, wherein the wetting comprises spraying a predetermine amount of sterile water or saline on the drug delivery device or segment thereof, and/or the surface of the trocar.

Clause 64. The use of according to clause 60, wherein the wetting comprises disposing a predetermined amount of sterile water or saline on the drug delivery device or segment thereof.

Clause 65. The use according to any one of clauses 33 to 64, wherein the release dissolution profile of the sum of drug delivery segments is substantially similar to the release dissolution profile of the unpartitioned drug delivery device.

Clause 66. The use according to any one of clauses 33 to 64, wherein the release dissolution profile of the sum of drug delivery segments is within about 1% and about 20% at any point in time substantially similar to the release dissolution profile of the unpartitioned drug delivery device.

Clause 67. The use according to any one of clauses 33 to 64, wherein the drug substance release profile of a sum of drug delivery devices is within about 1% and about 20% at any point in time comparative to a sum of control drug delivery devices, wherein the total dose of drug substance in the sum of drug delivery devices and total dose of drug substance in the sum of control drug delivery devices is substantially similar.

Clause 68. The use according to clause 67, wherein the total dose of drug substance is about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

Clause 69. A kit for implanting a drug delivery device or a segment thereof during laparoscopic surgery, comprising: a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration; and instructions for a clinician for implanting the drug delivery device at an implantation site during a laparoscopic procedure.

Clause 70. The kit of clause 69, wherein the drug delivery device has marking thereon for indicating lines along which to cut the drug delivery device.

Clause 71. A kit for implanting a drug delivery device by laparoscopic surgery, comprising: a sterile trocar; a spray-bottle comprising a predetermined amount of sterile water or saline; a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; and instructions for a clinician for implanting the drug delivery device at an implantation site during a laparoscopic procedure using the sterile trocar.

Clause 72. A kit for implanting a drug delivery device by laparoscopic surgery, comprising: a sterile trocar; an enclosure device shaped and sized to enable a clinician to insert the enclosure device through a proximal end of the sterile trocar, the enclosure device enclosing a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; and instructions for a clinician for implanting the drug delivery device at an implantation site during a laparoscopic procedure using the sterile trocar.

Clause 73. A drug delivery device comprising: an enclosure device shaped and sized to enable insertion of the enclosure through a proximal end of a trocar; and an implant enclosed in the enclosure device.

Clause 74. The drug delivery device of clause 73, wherein the implant comprises a depot for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

Clause 75. The drug delivery device of clause 74, wherein the analgesic is selected from one of Bupivacaine, Levobupivacaine, Lidocaine, Mepivacaine, Prilocaine, Ropivacaine, Articaine, Trimecaine and their salts and prodrugs.

Clause 76. The drug delivery device of clause 74, wherein the fibrillar collagen matrix is a Type I collagen matrix.

Clause 77. The drug delivery device of clause 76, wherein the fibrillar collagen matrix is a Type I collagen matrix and the at least one drug substance is an amino amide anesthetic selected from bupivacaine and salts and prodrugs thereof.

Clause 78. The drug delivery device of any one of clauses 73 to 77, wherein the enclosure is configured for opening after insertion through a trocar, and delivery of the implant at an implantation site.

Clause 79. A pharmaceutical composition comprising: a collagen matrix; and an analgesic drug included in the collagen matrix, the composition having a release profile for releasing the analgesic drug, wherein the release profile for a predetermined fraction of a certain mass of the composition is same as that of the mass.

Clause 80. The pharmaceutical composition of clause 79, wherein the predetermined fraction is ½, ⅓, ¼, or ⅕.

Clause 81. A drug delivery device for providing local analgesia, local anesthesia or nerve blockade at a site in a human or animal in need thereof, the drug delivery device comprising a fibrillar collagen matrix and at least one drug substance selected from an amino amide anesthetic, an amino ester anesthetic, and mixtures thereof, wherein the drug delivery device comprises one or more independent segments, wherein a segment is configured for inserting into a trocar, for moving through a trocar, and/or for independent placement at an implantation site.

Clause 82. The drug delivery device according to clause 81, wherein the drug delivery device or a segment thereof has a cylindrical or half-cylindrical shape, each independently having a length between about 30 mm and about 120 mm, and a radius between about 3 mm and about 8 mm.

Clause 83. The drug delivery device according to clause 81 or 82, wherein the drug delivery device or a segment thereof comprises one or more collagen sponges

Clause 84. The drug delivery device according to any one of clauses 81 to 83, wherein the at least one drug substance is substantially homogeneously dispersed in the collagen matrix.

Clause 85. The drug delivery device according to any one of clauses 81 to 84, wherein the drug substance is present in an amount sufficient to provide a duration of local analgesia, local anesthesia or nerve blockade which lasts for at least about one day after administration.

Clause 86. The drug delivery device according to any one of clauses 81 to 85, wherein the drug substance is selected from Bupivacaine, Levobupivacaine, Lidocaine, Mepivacaine, Prilocaine, Ropivacaine, Articaine, Trimecaine, and their salts and prodrugs.

Clause 87. The drug delivery device according to any one of clauses 81 to 86, wherein the fibrillar collagen matrix is a Type I collagen matrix.

Clause 88. The drug delivery device according to any one of clauses 81 to 87, wherein the fibrillar collagen matrix is a Type I collagen matrix and the at least one drug substance is an amino amide anesthetic selected from bupivacaine and salts and prodrugs thereof.

Clause 89. The drug delivery device according to any one of clauses 81 to 88, wherein the drug delivery device comprises about 1.0 to about 30.0 mg/cm³ bupivacaine hydrochloride.

Clause 90. The drug delivery device according to any one of clauses 81 to 88, wherein the drug delivery device comprises about 2.0 to about 22.0 mg/cm³ bupivacaine hydrochloride.

Clause 91. The drug delivery device according to any one of clauses 81 to 88, wherein the drug delivery device comprises about 4.0 to about 15.0 mg/cm³ bupivacaine hydrochloride.

Clause 92. The drug delivery device according to any one of clauses 81 to 88, wherein the drug delivery device comprises about 6.0 to about 12.0 mg/cm³ bupivacaine hydrochloride.

Clause 93. The drug delivery device according to any one of clauses 81 to 88, wherein the drug delivery device comprises about comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 94. The drug delivery device according to any one of clauses 81 to 88, wherein the drug delivery device comprises about comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride.

Clause 95. The drug delivery device according to any one of clauses 81 to 94, wherein the drug delivery device comprises about 1.0 to about 20.0 mg/cm³ type I collagen.

Clause 96. The drug delivery device according to any one of clauses 81 to 88, wherein the drug delivery device comprises about 5.6 mg/cm³ type I collagen and between about 4.0 and about 22 mg/cm³ bupivacaine hydrochloride.

Clause 97. The drug delivery device according to any one of clauses 81 to 96, wherein the drug substance release profile of the drug delivery device or a sum of segments thereof is within about 1% and about 20% at any point in time comparative to a control drug delivery device or a sum of segments thereof, wherein the total dose of drug substance in the drug delivery device or the sum segments thereof and the total dose of drug substance in the control drug delivery device or a sum of segments thereof is substantially similar.

Clause 98. The drug delivery device according to any one of clauses 81 to 96, wherein the drug substance release profile of the drug delivery device or a sum of segments thereof is substantially similar at any point in time comparative to a control drug delivery device or a sum of segments thereof, wherein the total dose of drug substance in the drug delivery device or the sum segments thereof and the total dose of drug substance in the control drug delivery device or a sum of segments thereof is substantially similar

Clause 99. The drug delivery device according to any one of clauses 81 to 96, having a drug substance release of about 40-60% at about 30 minutes, about 65-85% at about 120 minutes, and at least 80% at about 360 minutes.

Clause 100. The drug delivery device according to any one of clauses 81 to 97, wherein the total dose of drug substance in the drug delivery device or the sum of segments thereof is about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

Clause 101. The drug delivery device according to any one of clauses 81-100, wherein the drug delivery device comprises one independent segment having dimensions of about 5 cm×5 cm×0.5 cm.

Clause 102. The drug delivery device according to any one of clauses 81-100, wherein the drug delivery device comprises a compressed drug delivery device comprising one independent segment having dimensions of about 5 cm×5 cm×0.2 cm.

Clause 103. The drug delivery device according to any one of clauses 81-100, wherein the drug delivery device comprises a compressed drug delivery device comprising one independent segment having dimensions of about 5 cm×5 cm×(about 0.2-0.4) cm

Clause 104. The drug delivery device according to any one of clauses 81-100, wherein the drug delivery device comprises a compressed drug delivery device cut into two independent segments having dimensions of about 5 cm×2.5 cm×0.2 cm.

Clause 105. The drug delivery device according to any one of clauses 81-100, wherein the drug delivery device comprises a compressed drug delivery device cut into two independent segments having dimensions of about 5 cm×2.5 cm×(about 0.1-0.4) cm.

Clause 106. The drug delivery device according to any one of clauses 102-105, wherein the compressed drug delivery device comprises about 2.5 mg/cm³ to about 7.5 mg/cm³ collagen.

Clause 107. The drug delivery device according to any one of clauses 102-106, wherein the compressed drug delivery device comprises about 15 mg/cm³ to about 25 mg/cm³ bupivacaine, or a salt thereof.

Clause 108. The drug delivery device according to any one of clauses 102-107, wherein the compressed drug delivery device comprises about 5 mg/cm³ collagen and about 20 mg/cm³ bupivacaine, or a salt thereof.

Clause 109. The drug delivery device according to clause 104 or 105, wherein each independent segment is folded in half a first time such that a top side, comprising one or more beveled edges, is on the inside of the fold and a bottom side, lacking beveled edges, is on the outside of the fold.

Clause 110. The drug delivery device according to clause 109, wherein each folded segment has dimensions of about 2.5 cm×2.5 cm×0.4 cm.

Clause 111. The drug delivery device according to clause 109 or 110, wherein the bottom side on the outside of the fold does not crack or break.

Clause 112. The drug delivery device according to any one of clauses 109-111, wherein each folded segment is folded in half a second time, forming two twice folded segments each having dimensions of about 1.25 cm×2.5 cm×0.8 cm.

Clause 113. The drug delivery device according to clause 112, wherein the bottom side on the outside of the fold does not crack or break.

Clause 114. The drug delivery device according to clause 113, wherein one twice folded segment is placed into prongs of a grasper, wherein the prongs are configured for opening after insertion of the grasper through a trocar.

Clause 115. The drug delivery device according to clause 114, wherein the grasper is inserted through the trocar such that the prongs of the grasper extend beyond the end of the trocar.

Clause 116. A method of compressing a drug delivery device, the method comprising: placing a drug delivery device on a bottom plate of a compression device; placing a top plate of the compression device directly on top of the bottom plate; firmly pressing on the top plate of the compression device for several seconds; and removing the top plate of the compression device to provide a compressed drug delivery device, wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 117. The method according to clause 116, wherein the bottom plate and the top plate each comprise a recessed portion such that when the top plate is placed on top of the bottom plate without the drug delivery device, a gap of about 0.75 cm is formed between the recessed portion of the top plate and the recessed portion of the bottom plate.

Clause 118. The method according to clause 116 or 117, wherein the compressed drug delivery device has dimensions of about 5 cm×5 cm×0.2 cm.

Clause 119. The method according to clause 116 or 117, wherein the compressed drug delivery device has dimensions of about 5 cm×5 cm×(about 0.1-0.4) cm.

Clause 120. The method according to any one of clause 116-119, wherein the compressed drug delivery device comprises about 2.5 mg/cm³ to about 7.5 mg/cm³ collagen

Clause 121. The method according to any one of clauses 116-120, wherein the compressed drug delivery device comprises about 15 mg/cm³ to about 25 mg/cm³ bupivacaine, or a salt thereof.

Clause 122. The method according to any one of clauses 116-121, wherein the compressed drug delivery device comprises about 5 mg/cm³ collagen and about 20 mg/cm³ bupivacaine, or a salt thereof.

Clause 123. The method according to any one of clauses 116-121, wherein the compressed drug delivery device comprises about 5 mg/cm³ collagen and about 20 mg/cm³ bupivacaine hydrochloride.

Clause 124. A method of preparing a compressed drug delivery device for insertion into a trocar, the method comprising: cutting the compressed drug delivery device into two or more independent segments, each segment having a top side comprising one or more beveled edges and a bottom side lacking beveled edges; folding each segment in half a first time such that the top side is on the inside of the fold and the bottom side is on the outside of the fold; and folding each segment in half a second time; wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 125. The method according to clause 124, further comprising placing one twice folded segment into prongs of a grasper, wherein the prongs are configured for opening after insertion of the grasper through a trocar.

Clause 126. The method according to clause 125, further comprising inserting the grasper through the trocar such that the prongs of the grasper extend beyond the end of the trocar.

Clause 127a. The method according to any one of clauses 124-126, wherein the compressed drug delivery device has dimensions of about 5 cm×5 cm×0.2 cm. Clause 127b. The method according to any one of clauses 124-126, wherein the compressed drug delivery device has dimensions of about 5 cm×5 cm×0.18 cm. Clause 127c. The method according to any one of clauses 124-126, wherein the compressed drug delivery device has dimensions of about 5 cm×5 cm×0.19 cm. Clause 127d. The method according to any one of clauses 124-126, wherein the compressed drug delivery device has dimensions of about 5 cm×5 cm×0.21 cm. Clause 127e. The method according to any one of clauses 124-126, wherein the compressed drug delivery device has dimensions of about 5 cm×5 cm×0.22 cm. Clause 127f. The method according to any one of clauses 124-126, wherein the compressed drug delivery device has dimensions of about 5 cm×5 cm×0.23 cm. Clause 127g. The method according to any one of clauses 124-126, wherein the compressed drug delivery device has dimensions of about 5 cm×5 cm×0.24 cm. Clause 127h. The method according to any one of clauses 124-126, wherein the compressed drug delivery device has dimensions of about 5 cm×5 cm×0.25 cm. Clause 127i. The method according to any one of clauses 124-126, wherein the compressed drug delivery device has dimensions of about 5 cm×5 cm×0.26 cm. Clause 127j. The method according to any one of clauses 124-126, wherein the compressed drug delivery device has dimensions of about 5 cm×5 cm×0.27 cm. Clause 127k. The method according to any one of clauses 124-126, wherein the compressed drug delivery device has dimensions of about 5 cm×5 cm×0.28 cm. Clause 127l. The method according to any one of clauses 124-126, wherein the compressed drug delivery device has dimensions of about 5 cm×5 cm×0.29 cm. Clause 127m. The method according to any one of clauses 124-126, wherein the compressed drug delivery device has dimensions of about 5 cm×5 cm×0.3 cm. Clause 127n. The method according to any one of clauses 124-126, wherein the compressed drug delivery device has dimensions of about 5 cm×5 cm×0.31 cm. Clause 127o. The method according to any one of clauses 124-126, wherein the compressed drug delivery device has dimensions of about 5 cm×5 cm×0.32 cm.

Clause 128. The method according to any one of clauses 124-126, wherein the compressed drug delivery device has dimensions of about 5 cm×5 cm×(about 0.1-0.4) cm.

Clause 129. The method according to any one of clauses 124-128, wherein the compressed drug delivery device comprises about 2.5 mg/cm³ to about 7.5 mg/cm³ collagen.

Clause 130. The method according to any one of clauses 124-129, wherein the compressed drug delivery device comprises about 15 mg/cm³ to about 25 mg/cm³ bupivacaine, or a salt thereof.

Clause 131a. The method according to any one of clauses 124-130, wherein the compressed drug delivery device comprises about 5 mg/cm³ collagen and about 20 mg/cm³ bupivacaine, or a salt thereof. Clause 131b. The method according to any one of clauses 124-130, wherein the compressed drug delivery device comprises about 15 mg/cm³ bupivacaine, or a salt thereof. Clause 131c. The method according to any one of clauses 124-130, wherein the compressed drug delivery device comprises about 16 mg/cm³ bupivacaine, or a salt thereof. Clause 131d. The method according to any one of clauses 124-130, wherein the compressed drug delivery device comprises about 17 mg/cm³ bupivacaine, or a salt thereof. Clause 131e. The method according to any one of clauses 124-130, wherein the compressed drug delivery device comprises about 18 mg/cm³ bupivacaine, or a salt thereof. Clause 131f. The method according to any one of clauses 124-130, wherein the compressed drug delivery device comprises about 19 mg/cm³ bupivacaine, or a salt thereof. Clause 131g. The method according to any one of clauses 124-130, wherein the compressed drug delivery device comprises about 20 mg/cm³ bupivacaine, or a salt thereof. Clause 131h. The method according to any one of clauses 124-130, wherein the compressed drug delivery device comprises about 21 mg/cm³ bupivacaine, or a salt thereof. Clause 131i. The method according to any one of clauses 124-130, wherein the compressed drug delivery device comprises about 22 mg/cm³ bupivacaine, or a salt thereof. Clause 131j. The method according to any one of clauses 124-130, wherein the compressed drug delivery device comprises about 23 mg/cm³ bupivacaine, or a salt thereof. Clause 131k. The method according to any one of clauses 124-130, wherein the compressed drug delivery device comprises about 24 mg/cm³ bupivacaine, or a salt thereof. Clause 131l. The method according to any one of clauses 124-130, wherein the compressed drug delivery device comprises about 25 mg/cm³ bupivacaine, or a salt thereof.

Clause 132a. The method according to any one of clauses 124-131, wherein the compressed drug delivery device comprises about 5 mg/cm³ collagen and about 20 mg/cm³ bupivacaine hydrochloride. Clause 132b. The method according to any one of clauses 124-130, wherein the compressed drug delivery device comprises about 15 mg/cm³ bupivacaine hydrochloride. Clause 132c. The method according to any one of clauses 124-130, wherein the compressed drug delivery device comprises about 16 mg/cm³ bupivacaine hydrochloride. Clause 132d. The method according to any one of clauses 124-130, wherein the compressed drug delivery device comprises about 17 mg/cm³ bupivacaine hydrochloride. Clause 132e. The method according to any one of clauses 124-130, wherein the compressed drug delivery device comprises about 18 mg/cm³ bupivacaine hydrochloride. Clause 132f. The method according to any one of clauses 124-130, wherein the compressed drug delivery device comprises about 19 mg/cm³ bupivacaine hydrochloride. Clause 132g. The method according to any one of clauses 124-130, wherein the compressed drug delivery device comprises about 20 mg/cm³ bupivacaine hydrochloride. Clause 132h. The method according to any one of clauses 124-130, wherein the compressed drug delivery device comprises about 21 mg/cm³ bupivacaine hydrochloride. Clause 132i. The method according to any one of clauses 124-130, wherein the compressed drug delivery device comprises about 22 mg/cm³ bupivacaine hydrochloride. Clause 132j. The method according to any one of clauses 124-130, wherein the compressed drug delivery device comprises about 23 mg/cm³ bupivacaine hydrochloride. Clause 132k. The method according to any one of clauses 124-130, wherein the compressed drug delivery device comprises about 24 mg/cm³ bupivacaine hydrochloride. Clause 132l. The method according to any one of clauses 124-130, wherein the compressed drug delivery device comprises about 25 mg/cm³ bupivacaine hydrochloride.

Clause 133. The method according to any one of clauses 124-132, wherein the compressed drug delivery device is cut into two independent segments.

Clause 134. The method according to clause 133, wherein each segment has dimensions of about 5 cm×2.5 cm×0.2 cm.

Clause 135. The method according to clause 133, wherein each segment has dimensions of about 5 cm×2.5 cm×(about 0.1-0.4) cm

Clause 136. The method according to clause 134 or 135, wherein each segment folded one time has dimensions of about 2.5 cm×2.5 cm×0.4 cm.

Clause 137. The method according to clause 134 or 135, wherein each segment folded two times has dimensions of about 2.5 cm×1.25 cm×0.8 cm.

Clause 138. The method according to any one of clauses 124-137, wherein the bottom side of the compressed drug delivery device does not crack or break when folded.

Clause 139. A kit for implanting a drug delivery device or a segment thereof during laparoscopic surgery, comprising: a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration; a compression device for compressing the drug delivery device; and instructions for a clinician for implanting the drug delivery device at an implantation site during a laparoscopic procedure.

Clause 140. The kit of clause 139, wherein the drug delivery device has marking thereon for indicating lines along which to cut the drug delivery device.

Clause 141. The kit of clause 139, wherein the compression device comprises a top plate and a bottom plate

Clause 142. The kit of clause 141, wherein the top plate of the compression device has an indent shaped to conform the same of the drug delivery device.

Clause 143. The kit of clause of 141, wherein one or both of the top plate and the bottom plate are sterilized prior to inclusion in the kit.

Clause 144. The kit of clause 141, wherein one or both of the top plate and the bottom plate comprise a ceramic.

Clause 145. The kit of clause 139, further comprising a scissor.

Clause 146. The kit of clause 141, wherein the instructions include the steps for: placing a drug delivery device on a bottom plate of a compression device; placing a top plate of the compression device directly on top of the bottom plate; firmly pressing on the top plate of the compression device for several seconds; removing the top plate of the compression device to provide a compressed drug delivery device; cutting the compressed drug delivery device into a desired shape; and folding the cut compressed drug delivery device in a shape amenable to be inserted through the trocar.

Clause 147. A kit for implanting a drug delivery device by laparoscopic surgery, comprising: a sterile trocar; a spray-bottle comprising a predetermined amount of sterile water or saline; a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a tamper device configured to compress the drug delivery device; a scissor for cutting the drug delivery device; and instructions for a clinician for implanting the drug delivery device at an implantation site during a laparoscopic procedure using the sterile trocar.

Clause 148. The kit of clause 147, wherein the instructions include the steps of: compressing the drug delivery device using the tamper; cutting the compressed drug delivery device into a desired shape; and folding the cut compressed drug delivery device into a shape amenable to be inserted through the trocar.

Clause 149. A kit for implanting a drug delivery device by laparoscopic surgery, comprising: a sterile trocar; an enclosure device shaped and sized to enable a clinician to insert the enclosure device through a proximal end of the sterile trocar, the enclosure device enclosing a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; a vice configured to compress the drug delivery device; and instructions for a clinician for implanting the drug delivery device at an implantation site during a laparoscopic procedure using the sterile trocar.

Clause 150. The kit of clause 149, wherein the instructions include the steps of compressing the drug delivery device using the vice; cutting the compressed drug delivery device into a desired shape; and folding the cut compressed drug delivery device into a shape amenable to be inserted through the trocar.

Clause 1001. A method of implanting a drug delivery device for controlled, sustained release of an analgesic at an implantation site, the method comprising: inserting the drug delivery device or a segment thereof into a trocar; moving the inserted drug delivery device or segment thereof through the trocar; and placing the inserted delivery device or segment thereof at the implantation site.

Clause 1002. The method of clause 1001, wherein the drug delivery device or the segment thereof has any shape adapted for insertion into the trocar.

Clause 1003. The method of clause 1001, further comprising partitioning the drug delivery device into segments with a predetermined size.

Clause 1004. The method of clause 1003, wherein partitioning the drug delivery device comprises cutting the drug delivery device.

Clause 1005. The method of clause 1003 or 1004, wherein the drug delivery device is partitioned into two or more segments, wherein each segment is placed at the implantation site independently.

Clause 1006. The method of any one of clauses 1001 to 1005, wherein the drug delivery device has a length of about 50 mm, a width of about 50 mm, and a thickness of about 5 mm.

Clause 1007. The method of any one of clauses 1003 to 1005, wherein partitioning the drug delivery device comprises cutting the drug delivery device into a shape selected from: a square, a rectangle, a right triangle, and an elongate triangle.

Clause 1008. The method of any one of clauses 1003 to 1007, wherein the segments are about ½, about ⅓, or about ¼ in size relative to the drug delivery device.

Clause 1009. The method of any one of clauses 1001 to 1007, wherein the trocar has an internal diameter in a range from about 10 mm to about 12 mm.

Clause 1010. The method of any one of clauses 1001 to 1007, wherein the trocar has an internal diameter of about 5 mm, about 8 mm, about 10 mm, or about 12 mm.

Clause 1011. The method of any one of clauses 1001 to 1010, wherein the drug delivery device is a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof.

Clause 1012. The method of clause 1011, wherein the at least one drug substance is substantially homogeneously dispersed in the collagen matrix.

Clause 1013. The method of clause 1011, wherein the at least one drug substance is present in an amount sufficient to provide a duration of local analgesia, local anesthesia or nerve blockade which lasts for at least about one day after administration.

Clause 1014. The method of clause 1011 wherein the drug substance is selected from Bupivacaine, Levobupivacaine, Lidocaine, Mepivacaine, Prilocaine, Ropivacaine, Articaine, Trimecaine, and their salts and prodrugs.

Clause 1015. The method of clause 1011, wherein the fibrillar collagen matrix is a Type I collagen matrix.

Clause 1016. The method of clause 1015, wherein the fibrillar collagen matrix is a Type I collagen matrix and the at least one drug substance is an amino amide anesthetic selected from bupivacaine and salts and prodrugs thereof.

Clause 1017. The method of any one of clauses 1001 to 1016, wherein the drug delivery device comprises one or more collagen sponges.

Clause 1018. The method of clause 1017, wherein a collagen sponge comprises about 1.0 to about 30.0 mg/cm³ bupivacaine hydrochloride.

Clause 1019. The method of clause 1017, wherein a collagen sponge comprises about 2.0 to about 22.0 mg/cm³ bupivacaine hydrochloride.

Clause 1020. The method of clause 1017, wherein a collagen sponge comprises about 4.0 to about 15.0 mg/cm³ bupivacaine hydrochloride.

Clause 1021. The method of clause 1017, wherein a collagen sponge comprises about 6.0 to about 12.0 mg/cm³ bupivacaine hydrochloride.

Clause 1022. The method of clause 1017, wherein a collagen sponge comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 1023. The method of clause 1017, wherein a collagen sponge comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride bupivacaine hydrochloride.

Clause 1024. The method of any one of clauses 1017 to 1023, wherein a collagen sponge comprises about 1.0 to about 20.0 mg/cm³ type I collagen.

Clause 1025. The method of any one of clauses 1017 to 1024, wherein a collagen sponge comprises about 5.6 mg/cm³ type I collagen and between about 4.0 and about 22 mg/cm³ bupivacaine hydrochloride.

Clause 1026. The method of any one of clauses 1001 to 1025, further comprising wetting the drug delivery device or the segment thereof, or a surface of the trocar, prior to insertion into the trocar.

Clause 1027. The method of clause 1026, wherein the wetting comprises wetting by sterile water or saline.

Clause 1028. The method of clause 1026, wherein the wetting comprises dipping an edge of the delivery device or segment thereof into a predetermined amount of sterile water or saline.

Clause 1029. The method of clause 1026, wherein the wetting comprises spraying a predetermined amount of sterile water or saline on the drug delivery device or segment thereof, and/or the surface of the trocar.

Clause 1030. The method of clause 1026, wherein the wetting comprises disposing a predetermined amount of sterile water or saline on the drug delivery device or segment thereof.

Clause 1031. The method of any one of clauses 1001 to 1025, wherein the step of inserting the drug delivery device or a segment thereof into a trocar is preceded by the steps of compressing the drug delivery device and optionally partitioning the compressed drug delivery device into segments with a predetermined size.

Clause 1032. The method of clause 1031, wherein the drug delivery device is compressed by: placing the drug delivery device on a bottom plate of a compression device; placing a top plate of the compression device directly on top of the bottom plate; firmly pressing on the top plate of the compression device for several seconds; and removing the top plate of the compression device to provide the compressed drug delivery device.

Clause 1033. The method of clause 1032, wherein the compressed drug delivery device, while in the compression device, is compressed to a thickness of between about 15% to about 25% of an uncompressed drug delivery device.

Clause 1034. The method of clause 1032 or 1033, wherein the compressed drug delivery device, while in the compression device, is compressed to a thickness of about 0.8 mm to 1.2 mm.

Clause 1035a. The method of any one of clauses 1032 to 1034, wherein the compressed drug delivery device, when removed from the compression device, re-expands to a thickness of between about 60% to about 70% of an uncompressed drug delivery device. Clause 1035b. The method of any one of clauses 1032 to 1034, wherein the compressed drug delivery device, when removed from the compression device, re-expands to a thickness of between about 40% to about 60% of an uncompressed drug delivery device.

Clause 1036a. The method of any one of clauses 1032 to 1035, wherein the compressed drug delivery device, when removed from the compression device, re-expands to a thickness of about 2.8 to about 3.2 mm. Clause 1036b. The method of any one of clauses 1032 to 1035, wherein the compressed drug delivery device, when removed from the compression device, re-expands to a thickness of about 2 to about 2.8 mm.

Clause 1037. The method of any one of clauses 1031 to 1036, further comprising rolling the compressed drug delivery device or independently rolling each segment thereof prior to insertion into the trocar.

Clause 1038. The method of clause 1037, wherein the drug delivery device or segment thereof has a top side comprising one or more beveled edges and a bottom side lacking beveled edges and the compressed drug delivery device or segment thereof is rolled such that the top side is exposed on the outside of the roll.

Clause 1039. The method of clause 1037 or 1038, wherein the step of placing the inserted delivery device or segment thereof at the implantation site is followed by unrolling the inserted delivery device or segment thereof.

Clause 1040. The method of any one of clauses 1001 to 1039, wherein the release dissolution profile of the sum of drug delivery segments is substantially similar to the release dissolution profile of the unpartitioned drug delivery device.

Clause 1041. The method of any one of clauses 1001 to 1040, wherein the release dissolution profile of the sum of drug delivery segments is within about 1% and about 15% at any point in time substantially similar to the release dissolution profile of the unpartitioned drug delivery device.

Clause 1042. Use of a fibrillar collagen matrix and at least one drug substance selected from the group consisting of amino amide anesthetics, amino ester anesthetics and mixtures thereof, for the manufacture of a drug delivery device for providing local analgesia, local anesthesia or nerve blockade at a site in a human or animal in need thereof.

Clause 1043. The use according to clause 1042, wherein the at least one drug substance is substantially homogeneously dispersed in the collagen matrix.

Clause 1044. The use according to clause 1042 or 1043, wherein the at least one drug substance is present in an amount sufficient to provide a duration of local analgesia, local anesthesia or nerve blockade which lasts for at least about one day after administration.

Clause 1045. The use according to any one of clauses 1042 to 1044, wherein the drug substance is selected from Bupivacaine, Levobupivacaine, Lidocaine, Mepivacaine, Prilocaine, Ropivacaine, Articaine, Trimecaine, and their salts and prodrugs.

Clause 1046. The use according to any one of clauses 1042 to 1045, wherein the fibrillar collagen matrix is a Type I collagen matrix.

Clause 1047. The use according to any one of clauses 1042 to 1046, wherein the fibrillar collagen matrix is a Type I collagen matrix and the at least one drug substance is an amino amide anesthetic selected from bupivacaine and salts and prodrugs thereof.

Clause 1048. The use according to any one of clauses 1042 to 1047, wherein the drug delivery device comprises one or more collagen sponges.

Clause 1049. The use according to clause 1048, wherein a collagen sponge comprises about 1.0 to about 30.0 mg/cm³ bupivacaine hydrochloride.

Clause 1050. The use according to clause 1048, wherein a collagen sponge comprises about 2.0 to about 22.0 mg/cm³ bupivacaine hydrochloride.

Clause 1051. The use according to clause 1048, wherein a collagen sponge comprises about 4.0 to about 15.0 mg/cm³ bupivacaine hydrochloride.

Clause 1052. The according to clause 1048, wherein a collagen sponge comprises about 6.0 to about 12.0 mg/cm³ bupivacaine hydrochloride.

Clause 1053. The use according to clause 1048, wherein a collagen sponge comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 1054. The use according to clause 1048, wherein a collagen sponge comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride.

Clause 1055. The use according to any one of clauses 1042 to 1054, wherein a collagen sponge comprises about 1.0 to about 20.0 mg/cm³ type I collagen.

Clause 1056. The use according to clause 1048, wherein a collagen sponge comprises about 5.6 mg/cm³ type I collagen and between about 4.0 and about 22 mg/cm³ bupivacaine hydrochloride.

Clause 1057. The use according to any one of clauses 1042 to 1056, wherein the drug delivery device or a segment thereof is configured for sustained release of an analgesic at an implantation site.

Clause 1058. The use according to any one of clauses 1042 to 1057, wherein the drug delivery device or a segment thereof is configured for inserting into a trocar, for moving through a trocar, and/or for placement at an implantation site.

Clause 1059. The use according to any one of clauses 1042 to 1058, wherein the drug delivery device or the segment thereof has any shape adapted for insertion into a trocar.

Clause 1060. The use of any one of clauses 1042 to 1059, wherein the drug delivery device is configured for partitioning into segments with a predetermined size.

Clause 1061. The use according to clause 1060, wherein partitioning the drug delivery device comprises cutting the drug delivery device.

Clause 1062. The use according to any one of clauses 1042 to 1061, wherein the drug delivery device is configured for partitioning into two or more segments, wherein each segment is placeable at an implantation site independently.

Clause 1063. The use according to any one of clauses 1042 to 1062, wherein the drug delivery device has a length of about 50 mm, a width of about 50 mm, and a thickness of about 5 mm.

Clause 1064. The use according to any one of clauses 1060 to 1063, wherein the drug delivery device is configured for partitioning by cutting the drug delivery device into a shape selected from: a square, a rectangle, a right triangle, and an elongate triangle.

Clause 1065. The use according to any one of clauses 1060 to 1064, wherein the segments are about ½, about ⅓, or about ¼ in size relative to the drug delivery device.

Clause 1066. The use according to any one of clauses 1042 to 1056, wherein the drug delivery device or a segment thereof has a cylindrical or half-cylindrical shape, with a length between about 30 mm and about 100 mm, and a radius between about 3 mm and about 8 mm.

Clause 1067. The use according to any one of clauses 1058 to 1066, wherein the trocar has an internal diameter in a range from about 10 mm to about 12 mm.

Clause 1068. The use according to any one of clauses 1058 to 1066, wherein the trocar has an internal diameter of about 5 mm, about 8 mm, about 10 mm, or about 12 mm.

Clause 1069. The use according to any one of clauses 1042 to 1068, wherein the drug delivery device or segment thereof is configured for wetting.

Clause 1070. The use according to clause 1069, wherein the wetting comprises wetting by sterile water or saline.

Clause 1071. The use according to clause 1069, wherein the wetting comprises dipping an edge of the delivery device or segment thereof into a predetermined amount of sterile water or saline.

Clause 1072. The use according to clause 1069, wherein the wetting comprises spraying a predetermined amount of sterile water or saline on the drug delivery device or segment thereof, and/or the surface of the trocar.

Clause 1073. The use according to clause 1069, wherein the wetting comprises disposing a predetermined amount of sterile water or saline on the drug delivery device or segment thereof.

Clause 1074. The use according to any one of clauses 1042 to 1068, wherein the drug delivery device is compressed, and the compressed drug delivery device has been optionally partitioned into segments with a predetermined size.

Clause 1075. The use according to clause 1074, wherein the drug delivery device is compressed by: placing a drug delivery device on a bottom plate of a compression device; placing a top plate of the compression device directly on top of the bottom plate; firmly pressing on the top plate of the compression device for several seconds; and removing the top plate of the compression device to provide the compressed drug delivery device.

Clause 1076. The use according to clause 1075, wherein the compressed drug delivery device, while in the compression device, is compressed to a thickness of between about 15% to about 25% of an uncompressed drug delivery device.

Clause 1077. The use according to clause 1075 or 1076, wherein the compressed drug delivery device, while in the compression device, is compressed to a thickness of about 0.8 mm to 1.2 mm.

Clause 1078. The use according to any one of clauses 1075 to 1077, wherein the compressed drug delivery device, when removed from the compression device, re-expands to a thickness of between about 60% to about 70% of an uncompressed drug delivery device.

Clause 1079. The use according to any one of clauses 1075 to 1078, wherein the compressed drug delivery device, when removed from the compression device, re-expands to a thickness of about 2.8 to about 3.2 mm.

Clause 1080. The use according to any one of clauses 1074 to 1079, wherein the compressed drug delivery device or each independent segment thereof is rolled for insertion into a trocar and for moving through the trocar to an implantation site.

Clause 1081. The use according to clause 1080, wherein the drug delivery device or segment thereof has a top side comprising one or more beveled edges and a bottom side lacking beveled edges and the compressed drug delivery device or segment thereof is rolled such that the top side is exposed on the outside of the roll.

Clause 1082. The use according to clause 1080 or 1081, wherein the rolled compressed drug delivery device or segment thereof is unrolled at the implantation site.

Clause 1083. The use according to any one of clauses 1042 to 1082, wherein the release dissolution profile of the sum of drug delivery segments is substantially similar to the release dissolution profile of the unpartitioned drug delivery device.

Clause 1084. The use according to any one of clauses 1042 to 1082, wherein the release dissolution profile of the sum of drug delivery segments is within about 1% and about 20% at any point in time substantially similar to the release dissolution profile of the unpartitioned drug delivery device.

Clause 1085. The use according to any one of clauses 1042 to 1082, wherein the drug substance release profile of a sum of drug delivery devices is within about 1% and about 20% at any point in time comparative to a sum of control drug delivery devices, wherein the total dose of drug substance in the sum of drug delivery devices and total dose of drug substance in the sum of control drug delivery devices is substantially similar.

Clause 1086. The use according to clause 1085, wherein the total dose of drug substance is about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

Clause 1087. A kit for implanting a drug delivery device or a segment thereof during laparoscopic surgery, comprising: a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration; and instructions for a clinician for implanting the drug delivery device at an implantation site during a laparoscopic procedure.

Clause 1088. The kit of clause 1087, wherein the drug delivery device has a marking thereon for indicating lines along which to cut the drug delivery device.

Clause 1089. A kit for implanting a drug delivery device by laparoscopic surgery, comprising: a sterile trocar; a spray-bottle comprising a predetermined amount of sterile water or saline; a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; and instructions for a clinician for implanting the drug delivery device at an implantation site during a laparoscopic procedure using the sterile trocar.

Clause 1090. A kit for implanting a drug delivery device by laparoscopic surgery, comprising: a sterile trocar; an enclosure device shaped and sized to enable a clinician to insert the enclosure device through a proximal end of the sterile trocar, the enclosure device enclosing a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; and instructions for a clinician for implanting the drug delivery device at an implantation site during a laparoscopic procedure using the sterile trocar.

Clause 1091. A drug delivery device comprising: an enclosure device shaped and sized to enable insertion of the enclosure through a proximal end of a trocar; and an implant enclosed in the enclosure device.

Clause 1092. The drug delivery device of clause 1091, wherein the implant comprises a depot for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

Clause 1093. The drug delivery device of clause 1092, wherein the anesthetic is selected from one of Bupivacaine, Levobupivacaine, Lidocaine, Mepivacaine, Prilocaine, Ropivacaine, Articaine, Trimecaine and their salts and prodrugs.

Clause 1094. The drug delivery device of clause 1092 or 1093, wherein the fibrillar collagen matrix is a Type I collagen matrix.

Clause 1095. The drug delivery device of any one of clauses 1092 to 1094, wherein the fibrillar collagen matrix is a Type I collagen matrix and the at least one drug substance is an amino amide anesthetic selected from bupivacaine and salts and prodrugs thereof.

Clause 1096. The drug delivery device of any one of clauses 1092 to 1095, wherein the enclosure is configured for opening after insertion through a trocar, and delivery of the implant at an implantation site.

Clause 1097. A pharmaceutical composition comprising: a collagen matrix; and an analgesic drug included in the collagen matrix, the composition having a release profile for releasing the analgesic drug, wherein the release profile for a predetermined fraction of a certain mass of the composition is same as that of the mass.

Clause 1098. The pharmaceutical composition of clause 1097, wherein the predetermined fraction is ½, ⅓, ¼, or ⅕.

Clause 1099. A drug delivery device for providing local analgesia, local anesthesia or nerve blockade at a site in a human or animal in need thereof, the drug delivery device comprising a fibrillar collagen matrix and at least one drug substance selected from an amino amide anesthetic, an amino ester anesthetic, and mixtures thereof, wherein the drug delivery device comprises one or more independent segments, wherein a segment is configured for inserting into a trocar, for moving through a trocar, and/or for independent placement at an implantation site.

Clause 1100. The drug delivery device according to clause 1099, wherein the drug delivery device or a segment thereof has a cylindrical or half-cylindrical shape, each independently having a length between about 30 mm and about 120 mm, and a radius between about 3 mm and about 8 mm.

Clause 1101. The drug delivery device according to clause 1099 or 1100, wherein the drug delivery device or a segment thereof comprises one or more collagen sponges.

Clause 1102. The drug delivery device according to any one of clauses 1099 to 1101, wherein the at least one drug substance is substantially homogeneously dispersed in the collagen matrix.

Clause 1103. The drug delivery device according to any one of clauses 1099 to 1102, wherein the drug substance is present in an amount sufficient to provide a duration of local analgesia, local anesthesia or nerve blockade which lasts for at least about one day after administration.

Clause 1104. The drug delivery device according to any one of clauses 1099 to 1103, wherein the drug substance is selected from Bupivacaine, Levobupivacaine, Lidocaine, Mepivacaine, Prilocaine, Ropivacaine, Articaine, Trimecaine, and their salts and prodrugs.

Clause 1105. The drug delivery device according to any one of clauses 1099 to 1104, wherein the fibrillar collagen matrix is a Type I collagen matrix.

Clause 1106. The drug delivery device according to any one of clauses 1099 to 1105, wherein the fibrillar collagen matrix is a Type I collagen matrix and the at least one drug substance is an amino amide anesthetic selected from bupivacaine and salts and prodrugs thereof.

Clause 1107. The drug delivery device according to any one of clauses 1099 to 1106, wherein the drug delivery device comprises about 1.0 to about 30.0 mg/cm³ bupivacaine hydrochloride.

Clause 1108. The drug delivery device according to any one of clauses 1099 to 1106, wherein the drug delivery device comprises about 2.0 to about 22.0 mg/cm³ bupivacaine hydrochloride.

Clause 1109. The drug delivery device according to any one of clauses 1099 to 1106, wherein the drug delivery device comprises about 4.0 to about 15.0 mg/cm³ bupivacaine hydrochloride.

Clause 1110. The drug delivery device according to any one of clauses 1099 to 1106, wherein the drug delivery device comprises about 6.0 to about 12.0 mg/cm³ bupivacaine hydrochloride.

Clause 1111. The drug delivery device according to any one of clauses 1099 to 1106, wherein the drug delivery device comprises about 6.0 mg/cm³ bupivacaine hydrochloride, about 7.0 mg/cm³ bupivacaine hydrochloride, about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, about 15.0 mg/cm³ bupivacaine hydrochloride, about 16.0 mg/cm³ bupivacaine hydrochloride, about 17.0 mg/cm³ bupivacaine hydrochloride, about 18.0 mg/cm³ bupivacaine hydrochloride, about 19.0 mg/cm³ bupivacaine hydrochloride, about 20.0 mg/cm³ bupivacaine hydrochloride, about 21.0 mg/cm³ bupivacaine hydrochloride, or about 22.0 mg/cm³ bupivacaine hydrochloride.

Clause 1112. The drug delivery device according to any one of clauses 1099 to 1106, wherein the drug delivery device comprises about 8.0 mg/cm³ bupivacaine hydrochloride, about 9.0 mg/cm³ bupivacaine hydrochloride, about 10.0 mg/cm³ bupivacaine hydrochloride, about 11.0 mg/cm³ bupivacaine hydrochloride, about 12.0 mg/cm³ bupivacaine hydrochloride, about 13.0 mg/cm³ bupivacaine hydrochloride, about 14.0 mg/cm³ bupivacaine hydrochloride, or about 15.0 mg/cm³ bupivacaine hydrochloride.

Clause 1113. The drug delivery device according to any one of clauses 1099 to 1112, wherein the drug delivery device comprises about 1.0 to about 20.0 mg/cm³ type I collagen.

Clause 1114. The drug delivery device according to any one of clauses 1099 to 1106, wherein the drug delivery device comprises about 5.6 mg/cm³ type I collagen and between about 4.0 and about 22 mg/cm³ bupivacaine hydrochloride.

Clause 1115. The drug delivery device according to any one of clauses 1099 to 1114, wherein the drug substance release profile of the drug delivery device or a sum of segments thereof is within about 1% and about 20% at any point in time comparative to a control drug delivery device or a sum of segments thereof, wherein the total dose of drug substance in the drug delivery device or the sum segments thereof and the total dose of drug substance in the control drug delivery device or a sum of segments thereof is substantially similar.

Clause 1116. The drug delivery device according to any one of clauses 1099 to 1115, wherein the drug substance release profile of the drug delivery device or a sum of segments thereof is substantially similar at any point in time comparative to a control drug delivery device or a sum of segments thereof, wherein the total dose of drug substance in the drug delivery device or the sum segments thereof and the total dose of drug substance in the control drug delivery device or a sum of segments thereof is substantially similar.

Clause 1117. The drug delivery device according to any one of clauses 1099 to 1116, having a drug substance release of about 40-60% at about 30 minutes, about 65-85% at about 120 minutes, and at least 80% at about 360 minutes.

Clause 1118. The drug delivery device according to any one of clauses 1099 to 1115, wherein the total dose of drug substance in the drug delivery device or the sum of segments thereof is about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

Clause 1119. The drug delivery device according to any one of clauses 1099 to 1118, wherein the drug delivery device comprises one independent segment having dimensions of about 5 cm×5 cm×0.5 cm.

Clause 1120. The drug delivery device according to any one of clauses 1099 to 1118, wherein the drug delivery device comprises a compressed drug delivery device comprising one independent segment having dimensions of about 5 cm×5 cm×0.2 cm.

Clause 1121. The drug delivery device according to any one of clauses 1099 to 1118, wherein the drug delivery device comprises a compressed drug delivery device comprising one independent segment having dimensions of about 5 cm×5 cm×(about 0.2-0.4) cm.

Clause 1122. The drug delivery device according to any one of clauses 1099 to 1118, wherein the drug delivery device comprises a compressed drug delivery device cut into two independent segments having dimensions of about 5 cm×2.5 cm×0.2 cm.

Clause 1123. The drug delivery device according to any one of clauses 1099 to 1118, wherein the drug delivery device comprises a compressed drug delivery device cut into two independent segments having dimensions of about 5 cm×2.5 cm×(about 0.1-0.4) cm.

Clause 1124. The drug delivery device according to any one of clauses 1120 to 1123, wherein the compressed drug delivery device comprises about 2.5 mg/cm³ to about 7.5 mg/cm³ collagen.

Clause 1125. The drug delivery device according to any one of clauses 1120 to 1123, wherein the compressed drug delivery device comprises about 15 mg/cm³ to about 25 mg/cm³ bupivacaine, or a salt thereof.

Clause 1126. The drug delivery device according to any one of clauses 1120 to 1123, wherein the compressed drug delivery device comprises about 5 mg/cm³ collagen and about 20 mg/cm³ bupivacaine, or a salt thereof.

Clause 1127. The drug delivery device according to clause 1120 or 1121, wherein each independent segment is folded in half a first time such that a top side, comprising one or more beveled edges, is on the inside of the fold and a bottom side, lacking beveled edges, is on the outside of the fold.

Clause 1128. The drug delivery device according to clause 1127, wherein each folded segment has dimensions of about 2.5 cm×2.5 cm×0.4 cm.

Clause 1129. The drug delivery device according to clause 1127 or 1128, wherein the bottom side on the outside of the fold does not crack or break.

Clause 1130. The drug delivery device according to any one of clauses 1127 to 1129, wherein each folded segment is folded in half a second time, forming two twice folded segments each having dimensions of about 1.25 cm×2.5 cm×0.8 cm.

Clause 1131. The drug delivery device according to clause 1130, wherein the bottom side on the outside of the fold does not crack or break.

Clause 1132. The drug delivery device according to clause 1131, wherein one twice folded segment is placed into prongs of a grasper, wherein the prongs are configured for opening after insertion of the grasper through a trocar.

Clause 1133. The drug delivery device according to clause 1132, wherein the grasper is inserted through the trocar such that the prongs of the grasper extend beyond the end of the trocar.

Clause 1134. The drug delivery device according to clause 1122 or 1123, wherein each independent segment is rolled such that a top side, comprising one or more beveled edges, is exposed on the inside of the roll and a bottom side, lacking beveled edges, is on the inside of the roll.

Clause 1135. The drug delivery device according to clause 1134, wherein each rolled segment has a cylindrical shape with a length of about 2.5 mm and a radius of about 0.3 mm.

Clause 1136. The drug delivery device according to clause 1134 or 1135, wherein the bottom side exposed on the outside of the rolled segment does not crack or break.

Clause 1137. The drug delivery device according to clause 1136, wherein one rolled segment is placed into prongs of a grasper, wherein the prongs are configured for opening after insertion of the grasper through a trocar.

Clause 1138. The drug delivery device according to clause 1137, wherein the grasper is inserted through the trocar such that the prongs of the grasper extend beyond the end of the trocar.

Clause 1139. A method of compressing a drug delivery device, the method comprising: placing a drug delivery device on a bottom plate of a compression device; placing a top plate of the compression device directly on top of the bottom plate; firmly pressing on the top plate of the compression device for several seconds; and removing the top plate of the compression device to provide a compressed drug delivery device, wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 1140. The method according to clause 1139, wherein the bottom plate and the top plate each comprise a recessed portion such that when the top plate is placed on top of the bottom plate without the drug delivery device, a gap of about 0.75 mm to 1.25 mm is formed between the recessed portion of the top plate and the recessed portion of the bottom plate.

Clause 1141. The method according to clause 1140, wherein the gap is about 1.0 mm.

Clause 1142. The method according to any one of clauses 1139 to 1141, wherein the drug delivery device is placed into the compression device such that a top side, comprising one or more beveled edges, is facing upward and a bottom side, lacking beveled edges, is placed onto the bottom plate of the compression device.

Clause 1143. The method of clause 1142, wherein pressing on the top plate of the compression device for several seconds stamps an “UP” imprint and one or more lines to indicate cutting and rolling of the drug delivery device on the top side of the drug delivery device.

Clause 1144. The method according to any one of clauses 1139 to 1143, wherein the compressed drug delivery device has dimensions of about 5 cm×5 cm×0.2 cm.

Clause 1145. The method according to any one of clauses 1139 to 1143, wherein the compressed drug delivery device has dimensions of about 5 cm×5 cm×(about 0.1-0.4) cm.

Clause 1146. The method according to any one of clauses 1139 to 1145, wherein the compressed drug delivery device comprises about 2.5 mg/cm³ to about 7.5 mg/cm³ collagen.

Clause 1147. The method according to any one of clauses 1139 to 1145, wherein the compressed drug delivery device comprises about 15 mg/cm³ to about 25 mg/cm³ bupivacaine, or a salt thereof.

Clause 1148. The method according to any one of clauses 1139 to 1147, wherein the compressed drug delivery device comprises about 5 mg/cm³ collagen and about 20 mg/cm³ bupivacaine, or a salt thereof.

Clause 1149. The method according to any one of clauses 1139 to 1147, wherein the compressed drug delivery device comprises about 5 mg/cm³ collagen and about 20 mg/cm³ bupivacaine hydrochloride.

Clause 1150. A method of preparing a compressed drug delivery device for insertion into a trocar, the method comprising: cutting the compressed drug delivery device into two or more independent segments, each segment having a top side comprising one or more beveled edges and a bottom side lacking beveled edges; folding each segment in half a first time such that the top side is on the inside of the fold and the bottom side is on the outside of the fold; and folding each segment in half a second time; wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 1151. The method according to clause 1150, further comprising placing one twice folded segment into prongs of a grasper, wherein the prongs are configured for opening after insertion of the grasper through a trocar.

Clause 1152. The method according to clause 1151, further comprising inserting the grasper through the trocar such that the prongs of the grasper extend beyond the end of the trocar.

Clause 1153. The method according to any one of clauses 1150 to 1152, wherein the compressed drug delivery device has dimensions of about 5 cm×5 cm×0.2 cm.

Clause 1154. The method according to any one of clauses 1150 to 1152, wherein the compressed drug delivery device has dimensions of about 5 cm×5 cm×(about 0.1-0.4) cm.

Clause 1155. The method according to any one of clauses 1150 to 1154, wherein the compressed drug delivery device comprises about 2.5 mg/cm³ to about 7.5 mg/cm³ collagen.

Clause 1156. The method according to any one of clauses 1150 to 1154, wherein the compressed drug delivery device comprises about 15 mg/cm³ to about 25 mg/cm³ bupivacaine, or a salt thereof.

Clause 1157. The method according to any one of clauses 1150 to 1156, wherein the compressed drug delivery device comprises about 5 mg/cm³ collagen and about 20 mg/cm³ bupivacaine, or a salt thereof.

Clause 1158. The method according to any one of clauses 1150 to 1157, wherein the compressed drug delivery device is cut into two independent segments.

Clause 1159. The method according to clause 1158, wherein each segment has dimensions of about 5 cm×2.5 cm×0.2 cm.

Clause 1160. The method according to clause 1158, wherein each segment has dimensions of about 5 cm×2.5 cm×(about 0.1-0.4) cm

Clause 1161. The method according to clause 1159 or 1160, wherein each segment folded one time has dimensions of about 2.5 cm×2.5 cm×0.4 cm.

Clause 1162. The method according to clause 1159 or 1160, wherein each segment folded two times has dimensions of about 2.5 cm×1.25 cm×0.8 cm.

Clause 1163. The method according to any one of clauses 1150 to 1162, wherein the bottom side of the compressed drug delivery device does not crack or break when folded.

Clause 1164. A method of preparing a compressed drug delivery device for insertion into a trocar, the method comprising: cutting the compressed drug delivery device into two or more independent segments, each segment having a top side comprising one or more beveled edges and a bottom side lacking beveled edges; and rolling each segment such that the top side is exposed on the outside of the rolled segment and the bottom side is inside of the rolled segment; wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 1165. The method according to clause 1164, further comprising placing one rolled segment into prongs of a grasper, wherein the prongs are configured for opening after insertion of the grasper through a trocar.

Clause 1166. The method according to clause 1165, further comprising inserting the grasper through the trocar such that the prongs of the grasper extend beyond the end of the trocar.

Clause 1167. The method according to any one of clauses 1164 to 1166, wherein the compressed drug delivery device has dimensions of about 5 cm×5 cm×0.2 cm.

Clause 1168. The method according to any one of clauses 1164 to 1166, wherein the compressed drug delivery device has dimensions of about 5 cm×5 cm×(about 0.1-0.4) cm.

Clause 1169. The method according to any one of clauses 1164 to 1168, wherein the compressed drug delivery device comprises about 2.5 mg/cm³ to about 7.5 mg/cm³ collagen.

Clause 1170. The method according to any one of clauses 1164 to 1169, wherein the compressed drug delivery device comprises about 15 mg/cm³ to about 25 mg/cm³ bupivacaine, or a salt thereof.

Clause 1171. The method according to any one of clauses 1164 to 1170, wherein the compressed drug delivery device comprises about 5 mg/cm³ collagen and about 20 mg/cm³ bupivacaine, or a salt thereof.

Clause 1172. The method according to any one of clauses 1164 to 1171, wherein the compressed drug delivery device comprises about 5 mg/cm³ collagen and about 20 mg/cm³ bupivacaine hydrochloride.

Clause 1173. The method according to any one of clauses 1164 to 1172, wherein the compressed drug delivery device is cut into two independent segments.

Clause 1174. The method according to clause 1173, wherein each segment has dimensions of about 5 cm×2.5 cm×0.2 cm.

Clause 1175. The method according to clause 1173, wherein each segment has dimensions of about 5 cm×2.5 cm×(about 0.1-0.4) cm

Clause 1176. The method according to clause 1174 or 1175, wherein each rolled segment has a cylindrical shape with a length of about 2.5 cm and a radius of about 0.3 cm.

Clause 1177. The method according to any one of clauses 1164 to 1176, wherein the bottom side of the compressed drug delivery device does not crack or break when rolled.

Clause 1178. A kit for implanting a drug delivery device or a segment thereof during laparoscopic surgery, comprising: a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration; a compression device for compressing the drug delivery device; and instructions for a clinician for implanting the drug delivery device at an implantation site during a laparoscopic procedure.

Clause 1179. The kit of clause 1178, wherein the drug delivery device has marking thereon for indicating lines along which to cut the drug delivery device.

Clause 1180. The kit of clause 1178 or 1179, wherein the compression device comprises a top plate and a bottom plate.

Clause 1181. The kit of clause 1180, wherein the top plate of the compression device has an indent shaped to conform the same of the drug delivery device.

Clause 1182. The kit of clause of 1180, wherein one or both of the top plate and the bottom plate are sterilized prior to inclusion in the kit.

Clause 1183. The kit of clause 1180, wherein one or both of the top plate and the bottom plate comprise a ceramic.

Clause 1184. The kit of any one of clauses 1178 to 1183, further comprising a scissor.

Clause 1185. The kit of clause 1180, wherein the instructions include the steps for: placing a drug delivery device on a bottom plate of a compression device; placing a top plate of the compression device directly on top of the bottom plate; firmly pressing on the top plate of the compression device for several seconds; removing the top plate of the compression device to provide a compressed drug delivery device; cutting the compressed drug delivery device into a desired shape; and folding the cut compressed drug delivery device in a shape amenable to be inserted through the trocar.

Clause 1186. The kit of clause 1180, wherein the instructions include the steps for: placing a drug delivery device on a bottom plate of a compression device; placing a top plate of the compression device directly on top of the bottom plate; firmly pressing on the top plate of the compression device for several seconds; removing the top plate of the compression device to provide a compressed drug delivery device; cutting the compressed drug delivery device into a desired shape; and rolling the cut compressed drug delivery device into a rolled bundle to be inserted through the trocar.

Clause 1187. A kit for implanting a drug delivery device by laparoscopic surgery, comprising: a sterile trocar; a spray-bottle comprising a predetermined amount of sterile water or saline; a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a tamper device configured to compress the drug delivery device; a scissor for cutting the drug delivery device; and instructions for a clinician for implanting the drug delivery device at an implantation site during a laparoscopic procedure using the sterile trocar.

Clause 1188. The kit of clause 1187, wherein the instructions include the steps of: compressing the drug delivery device using the tamper; cutting the compressed drug delivery device into a desired shape; and folding the cut compressed drug delivery device into a shape amenable to be inserted through the trocar.

Clause 1189. A kit for implanting a drug delivery device by laparoscopic surgery, comprising: a sterile trocar; an enclosure device shaped and sized to enable a clinician to insert the enclosure device through a proximal end of the sterile trocar, the enclosure device enclosing a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration, wherein the drug delivery device has marking thereon for indicating lines along which to cut the drug delivery device; a scissor for cutting the drug delivery device; a vice configured to compress the drug delivery device; and instructions for a clinician for implanting the drug delivery device at an implantation site during a laparoscopic procedure using the sterile trocar.

Clause 1190. The kit of clause 1189, wherein the instructions include the steps of compressing the drug delivery device using the vice; cutting the compressed drug delivery device into a desired shape; and folding the cut compressed drug delivery device into a shape amenable to be inserted through the trocar.

Clause 1191. The method of clause 1037, wherein the step of the inserting the drug delivery device or a segment thereof into a trocar is preceded by the step of removing the drug delivery device or segment thereof from a blister pack enclosure, the drug delivery device comprising a blister side and a side opposite to the blister side, wherein the blister side has an increased elasticity compared to the side opposite to the blister side.

Clause 1192. The method of clause 1191, wherein the blister side is more stretchable compared to the side opposite to the blister side.

Clause 1193. The method of clause 1191 or 1192, wherein the blister side has one or more beveled surfaces.

Clause 1194. The method of any one of clauses 1191 to 1193, wherein the blister is convex, and the side opposite to the blister side is concave.

Clause 1195. The method of any one of clauses 1191 to 1194, wherein the blister side is more flexible compared to the side opposite to the blister side.

Clause 1196. The method of any one of clauses 1191 to 1195, wherein the step of placing the inserted delivery device or segment thereof at the implantation site is followed by unrolling the inserted delivery device or segment thereof.

Clause 1197. The use according to clause 1080, wherein the drug delivery device or segment thereof comprises a blister side and a side opposite to the blister side, wherein the compressed drug delivery device or segment thereof is rolled such that the blister side is exposed on the outside of the roll.

Clause 1198. The use according to clause 1197, wherein the blister side has an increased elasticity compared to the side opposite to the blister side.

Clause 1199. The use according to clause 1197 or 1198, wherein the blister side is more stretchable compared to the side opposite to the blister side.

Clause 1200. The use according to any one of clauses 1197 to 1199, wherein the blister side has one or more beveled surfaces.

Clause 1201. The use according to any one of clauses 1197 to 1200, wherein the blister is convex, and the side opposite to the blister side is concave.

Clause 1202. The use according to any one of clauses 1197 to 1201, wherein the blister side is more flexible compared to the side opposite to the blister side.

Clause 1203. The use according to any one of clauses 1197 or 1202, wherein the rolled compressed drug delivery device or segment thereof is unrolled at the implantation site.

Clause 1204. The drug delivery device according to clause 1122 or 1123, wherein each independent segment is rolled such that a blister side is exposed on the outside of the rolled segment and a side opposite to the blister side is on the inside of the rolled segment.

Clause 1205. The drug delivery device according to clause 1204, wherein each rolled segment has a cylindrical shape with a length of about 2.5 mm and a radius of about 0.3 mm.

Clause 1206. The drug delivery device according to clause 1204 or 1205, wherein the blister side has an increased elasticity compared to the side opposite to the blister side.

Clause 1207. The drug delivery device according to any one of clauses 1204 to 1206, wherein the blister side is more stretchable compared to the side opposite to the blister side.

Clause 1208. The drug delivery device according to any one of clauses 1204 to 1207, wherein the blister side has one or more beveled surfaces.

Clause 1209. The drug delivery device according to any one of clauses 1204 to 1208, wherein the blister is convex, and the side opposite to the blister side is concave.

Clause 1210. The drug delivery device according to any one of clauses 1204 to 1209, wherein the blister side is more flexible compared to the side opposite to the blister side.

Clause 1211. The drug delivery device according to any one of clauses 1204 to 1210, wherein the blister side exposed on the outside of the rolled segment does not crack or break.

Clause 1212. The drug delivery device according to any one of clauses 1204 to 1211, wherein one rolled segment is placed into prongs of a grasper, wherein the prongs are configured for opening after insertion of the grasper through a trocar.

Clause 1213. The drug delivery device according to clause 1212, wherein the grasper is inserted through the trocar such that the prongs of the grasper extend beyond the end of the trocar.

Clause 1214. A method of preparing a compressed drug delivery device for insertion into a trocar, the method comprising: cutting the compressed drug delivery device into two or more independent segments, each segment having a blister side and a side opposite to the blister side; and rolling each segment such that the blister side is exposed on the outside of the rolled segment and the side opposite the blister side is inside of the rolled segment; wherein the compressed drug delivery device comprises a fibrillar collagen matrix and at least one drug substance selected from the group consisting of amino amide anesthetics, amino ester anesthetics and mixtures thereof, the at least one drug substance being substantially homogeneously dispersed in the collagen matrix.

Clause 1215. The method according to clause 1214, further comprising placing one rolled segment into prongs of a grasper, wherein the prongs are configured for opening after insertion of the grasper through a trocar.

Clause 1216. The method according to clause 1215, further comprising inserting the grasper through the trocar such that the prongs of the grasper extend beyond the end of the trocar.

Clause 1217. The method according to any one of clauses 1214 to 1216, wherein the compressed drug delivery device has dimensions of about 5 cm×5 cm×0.2 cm.

Clause 1218. The method according to any one of clauses 1214 to 1216, wherein the compressed drug delivery device has dimensions of about 5 cm×5 cm×(about 0.1-0.4) cm.

Clause 1219. The method according to any one of clauses 1214 to 1218, wherein the compressed drug delivery device comprises about 2.5 mg/cm³ to about 7.5 mg/cm³ collagen.

Clause 1220. The method according to any one of clauses 1214 to 1219, wherein the compressed drug delivery device comprises about 15 mg/cm³ to about 25 mg/cm³ bupivacaine, or a salt thereof.

Clause 1221. The method according to any one of clauses 1214 to 1220, wherein the compressed drug delivery device comprises about 5 mg/cm³ collagen and about 20 mg/cm³ bupivacaine, or a salt thereof.

Clause 1222. The method according to any one of clauses 1214 to 1221, wherein the compressed drug delivery device comprises about 5 mg/cm³ collagen and about 20 mg/cm³ bupivacaine hydrochloride.

Clause 1223. The method according to any one of clauses 1214 to 1222, wherein the compressed drug delivery device is cut into two independent segments.

Clause 1224. The method according to clause 1223, wherein each segment has dimensions of about 5 cm×2.5 cm×0.2 cm.

Clause 1225. The method according to clause 1223, wherein each segment has dimensions of about 5 cm×2.5 cm×(about 0.1-0.4) cm

Clause 1226. The method according to clause 1224 or 1225, wherein each rolled segment has a cylindrical shape with a length of about 2.5 cm and a radius of about 0.3 cm.

Clause 1227. The method according to any one of clauses 1224 to 1226, wherein the bottom side of the compressed drug delivery device does not crack or break when rolled.

Clause 1228. The method according to any one of clauses 1224 to 1227, wherein the blister side has an increased elasticity compared to the side opposite to the blister side.

Clause 1229. The method according to any one of clauses 1224 to 1228, wherein the blister side is more stretchable compared to the side opposite to the blister side.

Clause 1230. The method according to any one of clauses 1224 to 1229, wherein the blister side has one or more beveled surfaces.

Clause 1231. The method according to any one of clauses 1224 to 1230, wherein the blister is convex, and the side opposite to the blister side is concave.

Clause 1232. The method according to any one of clauses 1224 to 1231, wherein the blister side is more flexible compared to the side opposite to the blister side.

### EXAMPLES

### Example 1: Feasibility Study for Investigating Laparoscopic Implantation of a Bupivacaine-Collagen Implant

The purpose of this study was to first determine whether it is feasible and potentially effective to treat laparoscopic patients with XaraColl as a precursor to conducting any randomized controlled trial. Ten patients to be adequate for this initial feasibility investigation. Two types of laparoscopic hernia repair, inguinal and umbilical were included. For the inguinal repair, both TAPP and TEP approaches were allowed, which are considered comparable. Surgeons reported that XaraColl was straightforward to implant in both the inguinal and umbilical repair procedures, thereby enabling the targeted release of bupivacaine at the major areas of tissue disruption and trauma.

Although the study was neither controlled nor powered to evaluate efficacy, pain scores were collected and analyzed, and patient use of opioid analgesia was recorded to compare with corresponding data obtained from earlier double-blind, randomized controlled trials that had demonstrated the efficacy of XaraColl for postoperative analgesia in men undergoing open hernioplasty. The findings suggest that laparoscopic patients experience similar levels of postoperative pain to open surgical patients over the first 24 hours, but probably somewhat less pain thereafter through 48 and 72 hours. This finding is not unexpected based on the literature and the extent of deep-tissue trauma caused by laparoscopic hernia repair. Most importantly, however, the study supports the view that immediate postoperative pain control is particularly important for ambulatory patients undergoing laparoscopic surgery who are scheduled for early hospital discharge.

When considered alongside the previous efficacy studies and the published literature in general, the feasibility study suggests that XaraColl may provide postoperative analgesia in patients undergoing laparoscopic surgery. However, randomized controlled trials are needed to confirm this hypothesis.

### Methods and Materials

A feasibility study was conducted to investigate the use of XaraColl in ten men undergoing laparoscopic inguinal or umbilical hernia repair (NCT 01224145). The study was performed at Kirby Surgical Center (Houston, TX, USA) in accordance with the Declaration of Helsinki and Good Clinical Practice guidelines following approval by an institutional review board.

Eligible patients included men at least 18 years of age who were generally healthy and scheduled for either a unilateral laparoscopic inguinal hernioplasty by the transabdominal preperitoneal (TAPP) or totally extraperitoneal (TEP) technique, or for a laparoscopic umbilical hernioplasty. Patients who were scheduled for a bilateral inguinal hernia repair, had already undergone the repair at the same site of the scheduled surgery, or who had any concomitant illness that would significantly increase their surgical risk or make it difficult to complete the required assessments were excluded. Patients who were being treated with agents that could affect their analgesic response, such as central alpha agents, neuroleptic agents, and other antipsychotic agents, monoamine oxidase inhibitors, or systemic corticosteroids, were also excluded. Patients who were considered suitable and provided written informed consent then underwent additional screening procedures, including a physical examination and routine laboratory tests, up to 42 days before surgery.

On the day of surgery, patients underwent confirmatory safety assessments, and those who continued to meet all study entry criteria were allocated to receive the study drug. Surgery was conducted under general anesthesia, allowing use of short-acting agents such as propofol, midazolam, and fentanyl. The use of epidural anesthesia or local anesthetic infiltrations was prohibited. All patients received a total of four XaraColl implants, each containing 50 mg of bupivacaine hydrochloride (i.e., 200 mg total dose), which were placed to achieve optimal coverage of the traumatized tissues and thereby target maximum postoperative analgesia. Time 0 was defined as the time of implantation of the first XaraColl implant.

For the laparoscopic inguinal hernia repair, the implants were positioned over the abdominal wall repair (the repair mesh was then placed on top) and along the area that was dissected through the laparoscope to get to the repair site. For the laparoscopic umbilical hernia repair, the implants were placed under the mesh or ventral patch prior to the mesh or patch being secured around the site of the repair. For both procedures, one of the four implants, or a portion of the implant, was also placed under the subcutaneous tissue on top of the closed fascia. The implants were allowed to be cut as needed to best cover the sites of hernia repair and fascial closure. After surgery, the surgeon completed a questionnaire to record the method and ease of implantation.

Patients were observed and evaluated by hospital staff for a minimum of 4 hours after their surgery. Immediate postoperative pain was treated with intravenous morphine at incremental doses of 1-2 mg, as needed to achieve pain control. Once patients could tolerate oral medication, they were provided with immediate-release oral morphine tablets and instructed to take only if necessary for breakthrough pain. Patients could be discharged following the observation period, but were required to return to the study site 72 hours after surgery for their final assessments and follow-up visit.

Study staff contacted patients by telephone approximately 1 week and 1 month after surgery to inquire about their surgical wound, use of concomitant medications, any adverse events, and their general well-being.

### Efficacy Assessments

Patients assessed their pain intensity after aggravated movement (defined as cough) by using a visual analog scale with the left anchor (0 mm) labeled “No pain” and the right anchor (100 mm) labeled “Worst pain imaginable”. These pain assessments were scheduled at approximately 1, 2, 4, 6, 8, 10, 12, 24, 48, and 72 hours after time 0 (provided the patient was awake). The summed pain intensity (SPI) was defined and calculated as the trapezoidal area under the visual analog scale curve from 1 hour through to 24, 48, or 72 hours after time 0. Missing assessments were imputed according to the following prospective rules:

(1) Any missing pain-intensity assessments before the first observed assessment were imputed with the patient's worst observation (i.e., their highest recorded visual analog scale score).

(2) All other missing pain-intensity assessments were imputed using the patient's last observation (i.e., the observation prior to that missing).

In addition, the total use of opioid analgesia (TOpA) administered from time 0 through 72 hours was recorded and converted to intravenous morphine equivalent using a published conversion table.

### Safety Assessments

Safety data, including physical findings, vital signs, and laboratory assessments, was collected at scheduled intervals and recorded all adverse events and serious adverse events throughout the study duration. An adverse event was defined as any clinically unfavorable and unintended sign (including abnormal laboratory findings), symptom, or disease, whether or not it was causally related to the treatment. A serious adverse event was defined as any adverse event that resulted in death, was life-threatening, required inpatient hospitalization or prolongation of existing hospitalization, resulted in permanent disability/incapacity, or was an important medical event. Each adverse event was designated based on clinical severity, defined as either “mild” (causes no limitation of usual activities), “moderate” (causes some limitation of usual activities), or “severe” (prevents or severely limits usual activities). Expectedness and relationship of the adverse events to treatment were assessed as either “definitely related,” “probably related,” “unlikely related,” or “not related.” Finally, the outcome of each adverse event at study completion was assessed and reported as either “recovered,” “resolved with sequelae,” “ongoing,” “death,” “other,” or “unknown.”

### Results

Ten patients were enrolled between March 2011 and May 2011, who all completed the study. Five patients had a laparoscopic inguinal hernia repair using the TAPP technique, and five had a laparoscopic umbilical hernia repair. Patient age ranged from 31 to 63 years (mean 48 years). All patients were white or Caucasian, four of whom were of Hispanic or Latino ethnicity.

### Feasibility of Implantation

In all cases, the surgeon reported that the four XaraColl implants were easily implanted and properly positioned. No difficulties were encountered passing the implants through the laparoscope ports or with the implantation procedure itself. In most cases (nine out of ten patients), the implants were cut before implantation and positioned according to the type of hernia repair performed to cover all disrupted tissue. In eight out of ten patients, implants were affixed to the mesh either using a vicryl suture or by being threaded through the mesh tags.

### Efficacy

The pain-intensity scores recorded at each assessment time are summarized in Table 1, and the calculated SPI and TOpA results are presented in Table 2. Since this was not a controlled study, values of TOpA and SPI were compared with previously reported pooled data from two studies of men undergoing open inguinal hernioplasty who also had the benefit of receiving XaraColl for postoperative analgesia. After 24 hours, the mean TOpA for this laparoscopic study (19.3 mg) was very similar to the value from the pooled open hernioplasty studies (19.5 mg). The same was true for the mean SPI (805 mm hour versus 883 mm hour for the laparoscopic and open procedures, respectively). However, after 48 and 72 hours, XaraColl-treated patients in this laparoscopic study had taken less opioid analgesia and recorded lower pain intensity than the XaraColl-treated patients in the open studies. The mean TOpA was 26% and 38% lower in this laparoscopic study after 48 and 72 hours, respectively, and the corresponding SPI values were 17% and 25% lower.

TABLE 1
Pain intensity over time

| | |
|---|---|
| Hours | Visual analog scale score (mm)* |

| post-time 0 | Mean | SD | Median | Range |
|---|---|---|---|---|
| 1 hour | 48.1 | 19.0 | 48 | 17-80 |
| 2 hours | 45.6 | 23.3 | 42 | 11-87 |
| 4 hours | 34.0 | 25.6 | 29 | 4-94 |
| 6 hours | 31.2 | 21.7 | 29 | 0-83 |
| 8 hours | 26.9 | 18.5 | 27 | 0-59 |
| 10 hours | 36.7 | 14.2 | 40.5 | 10-63 |
| 12 hours | 40.7 | 13.3 | 40 | 24-71 |
| 24 hours | 28.6 | 14.8 | 28 | 6-59 |
| 48 hours | 20.2 | 11.3 | 18 | 7-43 |
| 72 hours | 14.4 | 14.5 | 9 | 0-48 |

Note:
*Pain intensity assessed using a 0-100 mm visual analog scale after aggravated movement (defined as cough).
Abbreviation: SD, standard deviation.

### Safety

A total of 18 adverse events were reported by eight of the ten patients (Table 3). The most common events were itching, nausea, headache, and hypoxemia, with each being reported by two patients. All other adverse events were reported by only one patient. All adverse events were mild to moderate in severity, and all were assigned as either “unlikely related” or “not related” to study medication, as determined by the investigator. Only one serious adverse event was reported (sleep apnea), where the patient remained in the hospital overnight for monitoring. This condition was previously undiagnosed, but was not considered related to the study medication and resolved with sequelae. No deaths or discontinuations due to adverse events were reported.

TABLE 2
Results for SPI and TOpA through 24, 48, and 72 hours

| | Mean SPI | | | Mean TOpA | | |
|---|---|---|---|---|---|---|
| Time | (mm · hour) | | | (mg IV morphine eq) | | |
| period | Mean | SD | Range | Mean | SD | Range |
| 24 hours | 805 | 261 | 354-1233 | 19.3 | 15.4 | 2.0-58.0 |
| post-time 0 | | | | | | |
| 48 hours | 1390 | 503 | 519-2242 | 24.6 | 21.8 | 2.0-80.5 |
| post-time 0 | | | | | | |
| 72 hours | 1749 | 764 | 615-3124 | 26.6 | 23.2 | 2.0-85.5 |
| post-time 0 | | | | | | |

Abbreviations: IV, intravenous; eq, equivalents; SPI, summed pain intensity; TOpA, total use of opioid analgesia.

TABLE 3
All reported adverse events

| Adverse events (mapped to | Incidence |
|---|---|
| MedDRA system organ class) | n (%) |
| All adverse events | 8 (80) |
| Gastrointestinal disorders | 4 (40) |
| Nausea | 2 (20) |
| Constipation | 1 (10) |
| Diarrhea | 1 (10) |
| Vomiting | 1 (10) |
| General disorders and administration-site conditions | 1 (10) |
| Postoperative tremors | 1 (10) |
| Effusion at hernia site | 1 (10) |
| Nervous system disorders | 3 (30) |
| Headache | 2 (20) |
| Paresthesia | 1 (10) |
| Psychiatric disorders | 1 (10) |
| Anxiety | 1 (10) |
| Reproductive system and breast disorders | 1 (10) |
| Scrotal swelling | 1 (10) |
| Respiratory, thoracic, and mediastinal disorders | 3 (30) |
| Sleep apnea | 1 (10) |
| Hypoxemia | 2 (20) |
| Oropharyngeal pain | 1 (20) |
| Skin and subcutaneous tissue disorders | 2 (20) |
| Itching | 2 (20) |

Abbreviation:
MedDRA, Medical Dictionary for Regulatory Activities.

### Example 2: Initial Bench Test for Laparoscopic Implantation of XaraColl

Hands on testing was conducted in the lab to determine promising combinations of the following variables: implant shape, implant hold, trocar size, trocar manufacturer, trocars with and without valves, grasper type, grasper jaw length, and preformed implants. Some examples of trocar are described in US Patent Application Publication No. 2010/008199, which is incorporated herein by reference in its entirety for all purposes.

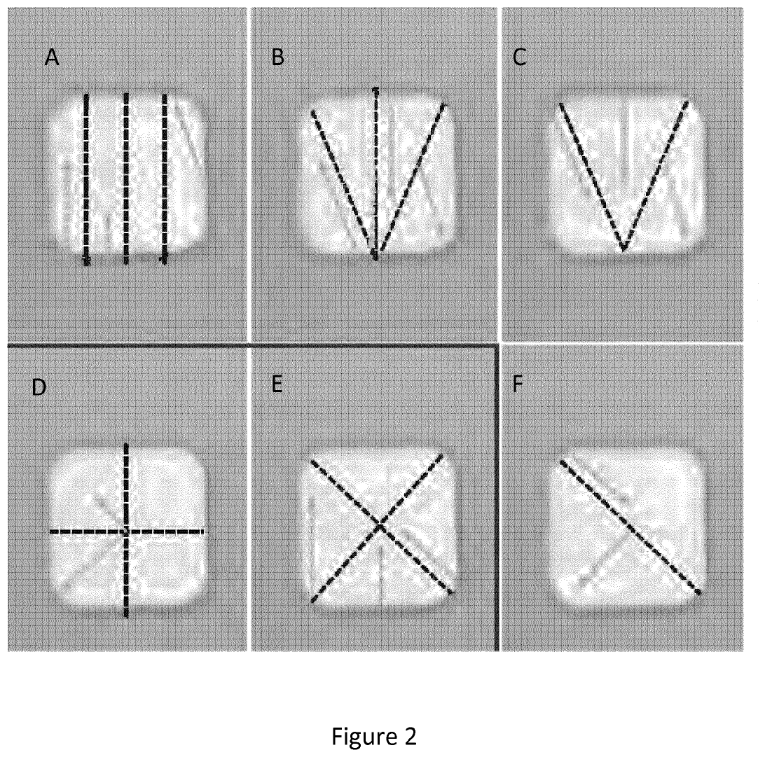

FIGS. 2A-2F schematically illustrate the different cuts and holds tested for a clinically approved XaraColl implant, for implantation during a laparoscopic procedure so that the implant (or a segment thereof) can be inserted through a trocar. The red dashed lines indicate the lines along which the clinically approved implant was cut, and the blue dashed lines indicate how the implant segments were held by a grasper, with arrows indicating the direction of insertion.

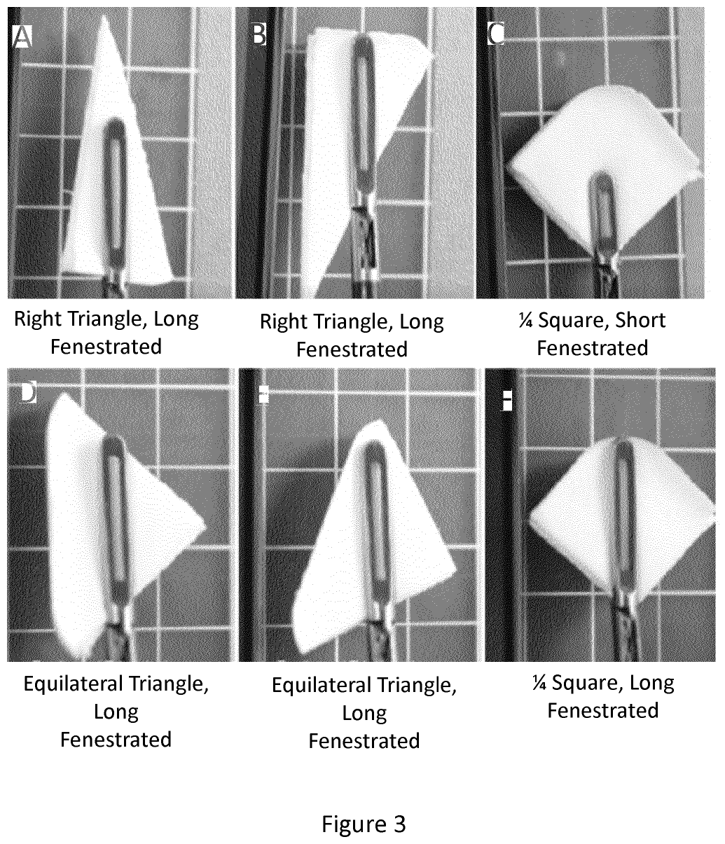

FIGS. 3A-3F are representative images of implant segments being held. The images illustrate long and short fenestrated graspers. For initial testing three types of graspers were tested: (1) Maryland; (2) Fenestrated; and (3) Babcock/Allis. For each type of grasper, graspers with three (long, short and medium) jaw-lengths were tested.

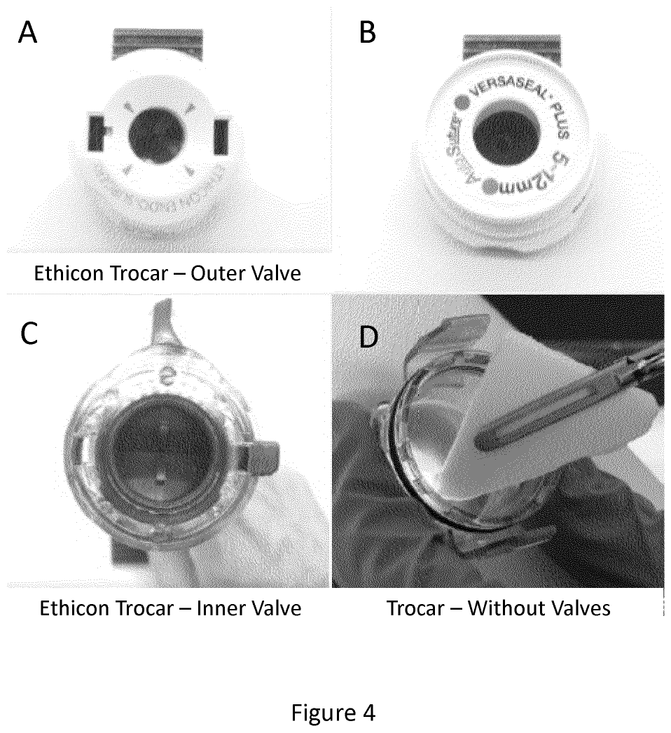

FIGS. 4A-4D are representative images of the trocars used for performing tests for laparoscopic implantation of implant segments. For initial bench testing, trocars with 10 mm and 12 mm diameter were evaluated. Trocars from various manufactures including Ethicon, Covidien, and secondary suppliers were evaluated. Many trocars can be opened to expose or remove their valves entirely. Trials were conducted with three varied states: (1) both inner and outer vales assembled; (2) outer valve removed; and (3) both inner and outer vales removed.

### Findings

Following the testing, it was found that longer, unsupported implant segments tend to tear when being passed through the trocar. It was further found that a wetted implant is relatively difficult to manipulate, it but becomes pliable, enabling it to pass through the trocar without tearing.

Not surprisingly, it was found to be difficult to pass implants through a 10 mm trocar with valve, and testing clinicians had little success in implanting the implant through such a trocar.

However, surprisingly, a 12 mm trocar with valve was found to provide substantial success. It is speculated that the outer valve of such a trocar may be aiding in gently rolling the implant and preventing pinching in the 2nd valve.

The testing also indicated that upon exiting the trocar, the implant is slightly compressed, likely because of travel through the trocar and from being held by the grasper. The amount of compression, however, was not found to affect the dissolution curves or the release profile of the drug from the implant.

The testing further indicated that generally longer tools that more completely support the implant (e.g., graspers with longer jaws), led to greater success rate in implantation. Additionally, wedge shaped implants that were broad but not long result in better success rates. For example, diagonally cut or right-angle square cut quarters resulted in greater implantation success. Finally, preforming the implant to have slightly rolled edges so that the implant conforms to the inner diameter of the trocar was found to provide greater success.

### Example 3: Statistical Bench Test for Laparoscopic Implantation of XaraColl

Based on the results of the initial bench testing, further statistical tests were conducted using a 12 mm trocar and a long grasper jaw length to further evaluate the following variables: Implant shape; Implant hold; 12 mm Trocars (with/without valves); Trocar manufacturer; Grasper type; Grasper technique; and Implant manufacturing (i.e., pre-forming).

The results of the statistical testing are detailed in Table 4. In brief, highest rates of success were observed with 12 mm Ethicon trocars, Long Fenestrated Graspers, Quarter square implants, and using a slow implantation technique. Although Configurations 17 & 20 represent the selection of these variables, their individual success rates varied from 50%-75%. Configurations 27 & 30 should be noted as they were identical to that of Configurations 17 & 20 except for using a new trocar and different Implant manufacturing lots and had success rates of 0% and 58% respectfully.

Generally, a slow technique was observed to increase success rates, but there was some nuance to the technique, that the clinicians could improve with practice. It was observed that new trocars that are recently opened (as they will be for surgeries) exhibit stiffer valves that reduce success rates. Further, there was some potentially implant variability from batch to batch which may affect the implant's structural properties.

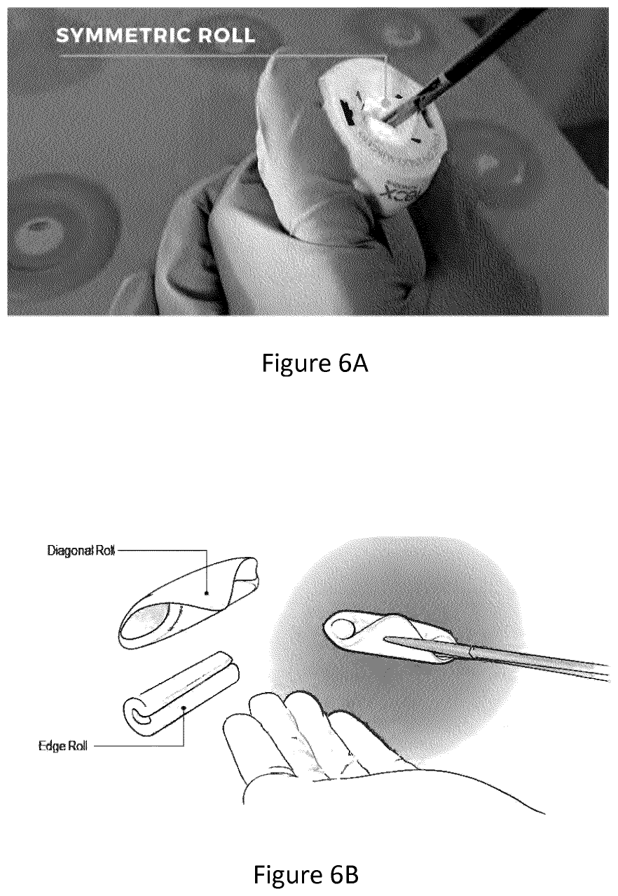

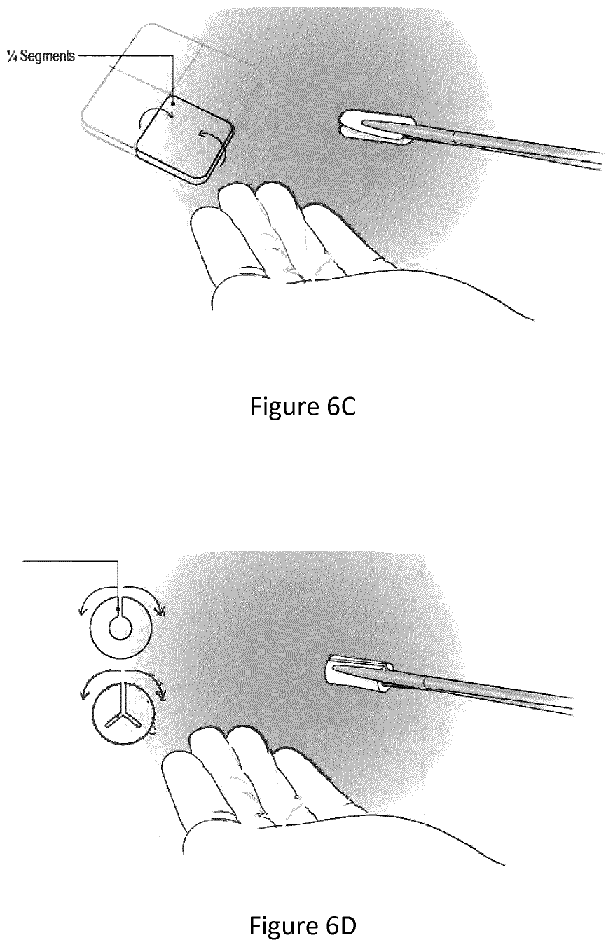

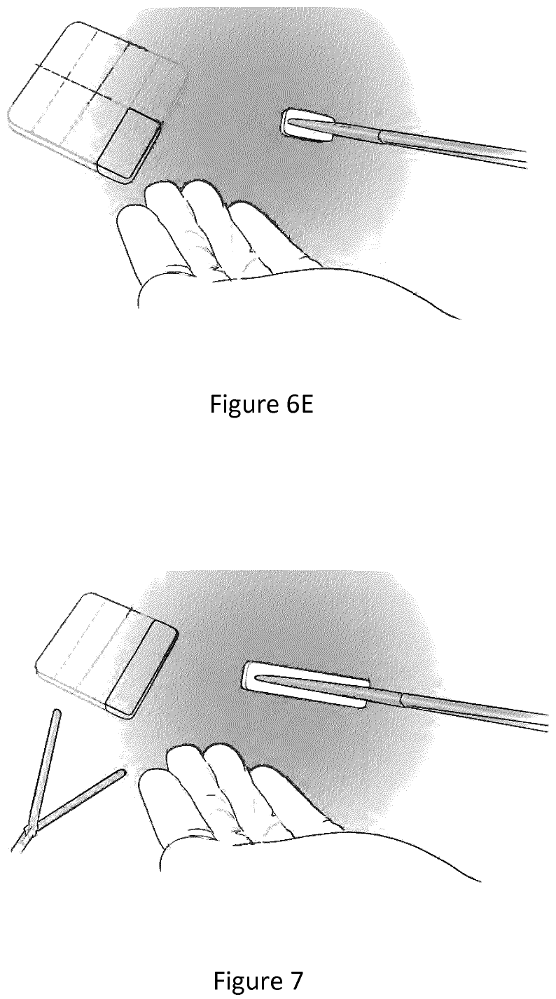

FIGS. 6A-6E are images and schematic representations of various cuts and molds of the implant segments that were tested.

In conclusion, it was found that in the statistical bench testing, the success rates varied from 0-75% based on configuration variables. Preferred configuration, that provided the highest reliable success rate, included 12 mm Ethicon trocar, Long Fenestrated Graspers, Quarter square implants, and using a slow implantation technique.

TABLE 4

| | | | | | Success |
|---|---|---|---|---|---|
| Configuration | Trocar Type | Implant Shape: | Tool: | Notes: | Rate |
| Configuration 1 | 12 mm Covidien | Right angle triangle | Long, Maryland | | 0% |
| Configuration 2 | 12 mm Covidien | Right angle triangle | Long, Fenestrated | | 0% |
| Configuration 3 | 12 mm Covidien | Right angle triangle | Long, Babcock | | skipped |
| Configuration 4 | 12 mm Covidien | Equilateral triangle | Long, Maryland | | 0% |
| Configuration 5 | 12 mm Covidien | Equilateral triangle | Long, Fenestrated | | skipped |
| Configuration 6 | 12 mm Covidien | Equilateral triangle | Long, Babcock | | skipped |
| Configuration 7 | 12 mm Covidien | Quarter Square | Long, Maryland | | 0% |
| Configuration 8 | 12 mm Covidien | Quarter Square | Long, Fenestrated | | skipped |
| Configuration 9 | 12 mm Covidien | Quarter Square | Long, Babcock | | skipped |
| Configuration 10 | 12 mm Ethicon | Right angle triangle | Long, Maryland | | 25% |
| Configuration 11 | 12 mm Ethicon | Right angle triangle | Long, Fenestrated | Molded edages at rear help | 33% |
| Configuration 12 | 12 mm Ethicon | Right angle triangle | Long, Babcock | Slow technique, Chipping | 17% |
| | | | | edges when cut | |
| Configuration 13 | 12 mm Ethicon | Equilateral triangle | Long, Maryland | | 13% |
| Configuration 14 | 12 mm Ethicon | Equilateral triangle | Long, Fenestrated | | 8% |
| Configuration 15 | 12 mm Ethicon | Equilateral triangle | Long, Babcock | Slow technique | 42% |
| Configuration 16 | 12 mm Ethicon | Quarter Square | Long, Maryland | | 0% |
| Configuration 17 | 12 mm Ethicon | Quarter Square | Long, Fenestrated | Slow technique | 50% |
| Configuration 18 | 12 mm Ethicon | Quarter Square | Long, Babcock | Slow technique | 42% |
| Configuration 19 | 12 mm Covidien | Quarter Square | Long, Fenestrated | Repeat of Config 7 using | 0% |
| | | | | Slow technique | |
| Configuration 20 | 12 mm Ethicon | Quarter Square | Long, Fenestrated | Slow technique, repeat of | 75% |
| | | | | Config 17 for more data | |
| Configuration 21 | OLD 12 mm Coviden | Quarter Square | Long, Fenestrated | Slow technique | 0% |
| Configuration 22 | OLD 12 mm Ethicon | Quarter Square | Long, Fenestrated | Slow technique | 92%* |
| Configuration 23 | 12 mm Ethicon | Formed ¼ Square | Long, Fenestrated | Slow technique, Preform = | 17% |
| | | | | stress, concentrations | |
| Configuration 27 | NEW 12 mm Ethicon | Quarter Square | Long, Fenestrated | New out of package trocar, | 0% |
| | | | | blade passed through | |
| | | | | trocar 10 times to simulate | |
| | | | | surical use (19002602) | |
| Configuration 28 | NEW 12 mm Ethicon | Right Triangle | Long, Fenestrated | New out of package trocar, | 0% |
| | | | | blade passed through | |
| | | | | trocar 10 times to simulate | |
| | | | | surical use (19002602) | |
| Configuration 30 | NEW 12 mm Ethicon | Quarter Square | Long, Fenestrated | Repeat of trial 27 with new | 58% |
| | | | | lot of implants (19001902) | |
| Configuration 31 | NEW 12 mm Ethicon | Quarter Square | Long, Fenestrated | Repeat of trial 27 with | 25% |
| | | | | same lot of implants | |
| | | | | (19002602) | |

### Example 4: Statistical Bench Test for Laparoscopic Implantation of XaraColl Using Sterilized Implants

Key configurations from Example 3 were repeated with sterilized implants to determine if sterilization influenced success rates. Following variables were tested in Example 4: Implant sterilization, implant shape, 12 mm trocars, slow technique, implant manufacturing batch.

It was observed that potentially implant variability from batch to batch could affect the implant's structural properties, and thus, success rates. Further, success rates achieved with sterilized implants (0%-67%) were similar or slightly worse than the bench testing conducted with implants that were not sterilized (0%-75%) in Example 3.

### Example 4: Wetted Implant Benchtop Testing

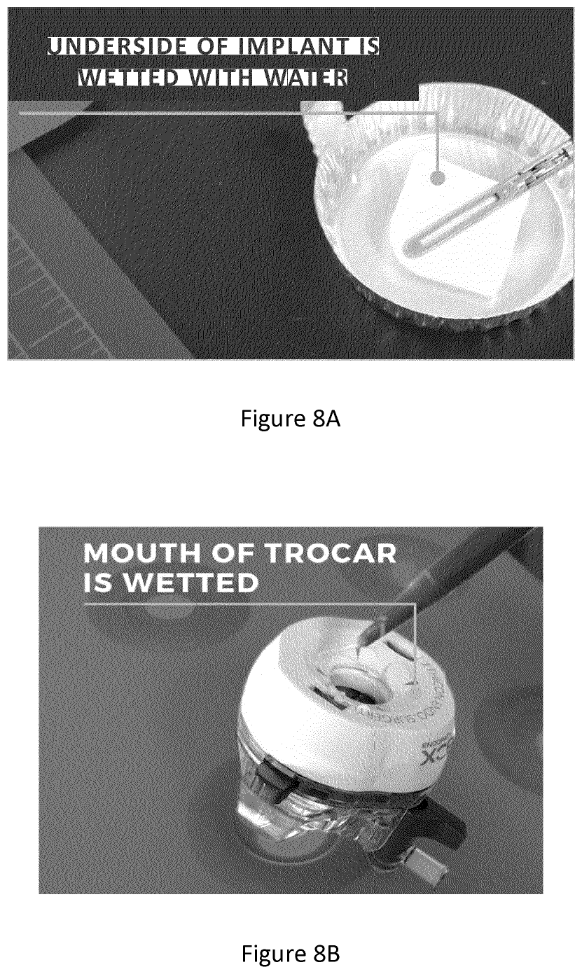

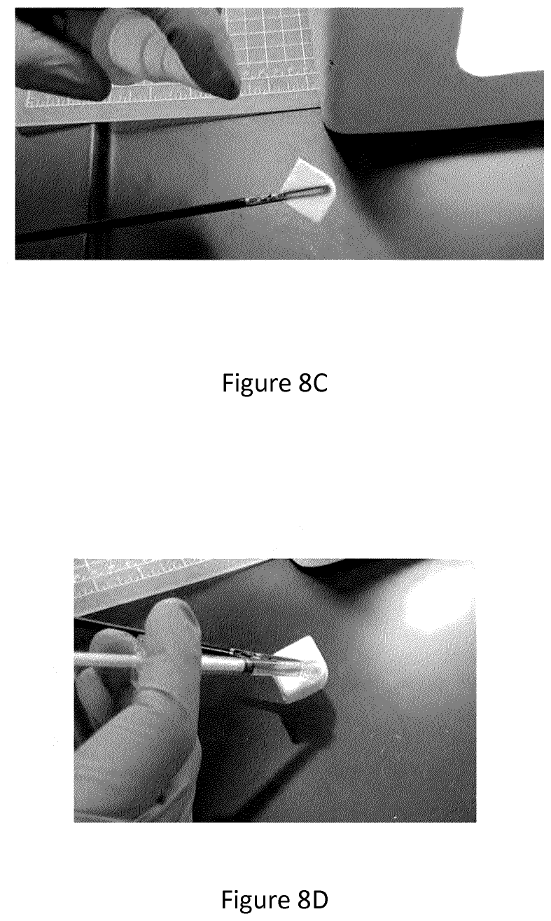

Given that none of the configurations provided a 100% success rate, alternate methods were explored in an effort to improve success rates. One of the concepts tested was wetting the implant or the trocar prior to insertion of the implant into the trocar. The implant may be wetted by: dipping the implant in water or saline (see FIG. 8A), spraying water or saline on the implant (see FIG. 8C), or applying water or saline on the implant with a syringe (see FIG. 8D). Similarly, the trocar may be wetted by applying water or saline to the mouth of the trocar by a syringe, a pipette, a dropper or a similar dispensing method (see FIG. 8B).

As a preliminary test, first, the amount of water/saline needed to allow bending into trocar widths/diameters was quantified with the goal of defining the minimum required amount of water/saline to make the implant pliable enough to pass through trocar diameters.

To quantify the amount of water, the test setup could consist of: (a) applying water/saline in a controlled and measured way (e.g., using a syringe) to an implant that is held in a grasper; (b) monitoring the time for the implant to absorb water/saline; and (c) passing the implant through a trocar of different sizes (12, 11, 5 mm). Ethicon trocars are used for this study.

An implant fracture is considered a failure in this study.

It was observed that upon wetting, the implant absorbs some amount of the water (or saline). Depending on the method of wetting, the rate of wetting (or time for which the implant is being wetted), and the size of the implant segment, the implant segments were observed to absorb between 0.1 mL-0.35 mL.

For example, when the implant segment is wetted by dispensing water or saline on the implant by a syringe, the implant was measured to absorb between 0.11 mL and 0.2 mL, and the amount of time needed for such application ranged from 5 seconds to 35 seconds. The amount of time needed for wetting by this method did not have any effect on the amount of water/saline absorbed by the implant segment.

When the implant segment was wetted by dipping the underside edges of implant segment into water (or saline), the implant was measured to absorb between 0.13 mL and 0.24 mL of water (or saline). It was also observed that dipping the implant for more than 1 minute weakens the implant.

When the implant is wetted by spraying the implant with, e.g., a mist bottle, the implant was observed to have a pot-marked surface with small craters, and the amount of water or saline absorbed is comparable to that by the syringe method.

Second, wetting implant and trocar is studied with focus on known configuration with the most past data and the goal of conducting a larger number of trials (˜24-48 for each wetted trocar and wetted implant) to better understand impact of a wetting. For this test, 12 mm Ethicon trocar, Long fenestrated grasper, ¼ square implant, and a slow insertion technique are used.

Images are captured before and after each trial similar to past testing, and absorption time of liquid is documented with video. Following this study, applying water by dipping the implant or with a syringe was determined to be preferred and more extensive trials confirmed increased success rates.

Once the preliminary test established that wetting the implant increased success rate, additional variables such as including trocar size and manufacturer, grasper type and length, implant size and shape were studied with the goal of testing the limits of a wetted implant or trocar to understand which tools, trocars, implant shapes, etc. are successful.

Following variables were tested: Grasper type (Maryland, Babcock, Fenestrated); Grasper length (Short (15 mm, Long 30 mm); Trocar size ( 10/11 mm, 12 mm); Trocar manufacturer (Ethicon, Covidien); Implant shape (¼ square, ¼ equilateral triangle, ¼ right triangle, ¼ rectangle, ½ triangle, whole implant); Wetting method (dip, syringe, spray).

Tests were conducted with the goal of isolating each of the following variables and determine their impact on success rates. This was achieved by comparing the impact of a variable to the established success rates of observed in the preliminary testing (Long, Fenestrated grasper, 11 mm Ethicon trocar, ¼ square implant segment, wetted implant by dipping). The following results were obtained:
Grasper type (long/short): Maryland (100%/67%), Babcock (100%/100%), Fenestrated (100%/67%).
Grasper Length: Short ˜78%, Long 100%
Trocar Size: 5 mm: 0%, 10/11 mm 100%, 12 mm 100%
Trocar Manufacturer: Ethicon 100%, Covidien 92%
Wetting Method: dip 100%, syringe 100%, spray 100%
Implant Shape: ¼ square 100%, ¼ equilateral triangle 100%, ¼ right triangle 58%, ¼ rectangle 50%, ½ triangle 83%, whole implant 0%

The testing result using long length (30 mm) grasper jaw resulted in 100% success rate for all three (Fenestrated, Maryland, and Babcock) jaw types. The high levels of success are attributed to the implant being well supported by the grasper jaws during insertion into the trocar. On the other hand, for short jaw length (15 mm) graspers, the Babcock type grasper provided a 100% result while the Fenestrated and Maryland type jaws resulted in 67% success rate. It was observed that when using a short grasper, it is more ideal to plunge the grasper vertically into the trocar as opposed to a rolling technique. This is due to the short grasper's inability to support the implant during rolling which leads to fracture. The Babcock gasper likely performs better due to its rounded tip which prevents stress concentration on the implant.

It was also observed that both (11 mm and 12 mm) Ethicon trocars resulted in a 100% success rate, likely because the mouth of the Ethicon trocars have angled lead-ins (chamfer) to the implant to more easily form to the diameter. The 10 mm Covidien trocars had a 92% success rate while the 12 mm Covidien trocars had a 100% success rate. The mouth of the Covidien trocars drops off steeply at a right angle into the diameter, this likely causes stress concentrations on the implants. It was observed that this geometry of the Covidien trocars requires more care to achieve high success compared to that of Ethicon trocars.

All three wetting methods (dipping, syringe and spray bottle) resulted in a 100% success rate. When testing these application methods, the target was to apply 0.2 mL to each implant segment. It was noted, however, that when dipping and spraying the implants, the least amount of effort to apply water is required. The syringe, on the other hand, required precision and care. One added benefit of the spray bottle is that the water dose can be controlled more easily.

With respect to implant shape, following success rates were observed: ¼ Square: 100%; ¼ Equilateral Triangle: 100%; ¼ Right Triangle: 58%; ¼ Rectangle: 50%; 12 Triangle: 83%.

Grasper holds on the right triangle and rectangle had significant impact on success. Failure typically occurred when the tail of the implant was unsupported when passing through the trocar valves. Choking up or holding the implant more toward the center, provided higher success.

Based on testing, the below variables are preferred because of their high success rates and ease of use: Long graspers; ¼ square or equilateral triangle; 10-12 mm Ethicon or Covidien trocars (but Ethicon is preferred); Wetting via dipping or spraying implant

### Example 5: Reformulate to Produce a Form/Size of Xaracoll which can be Directly Inserted Via a Trocar

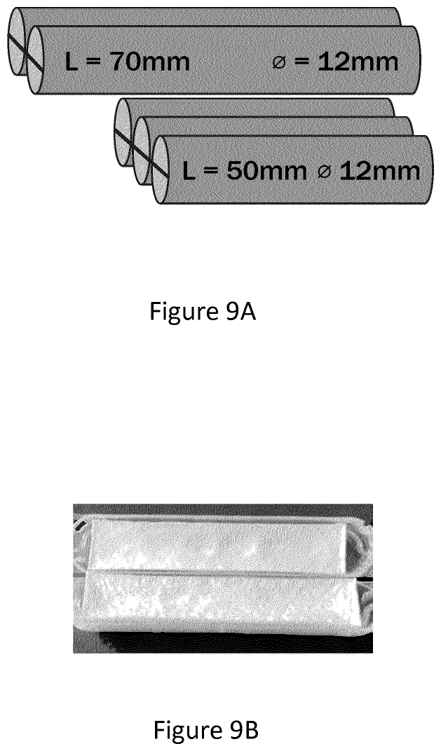

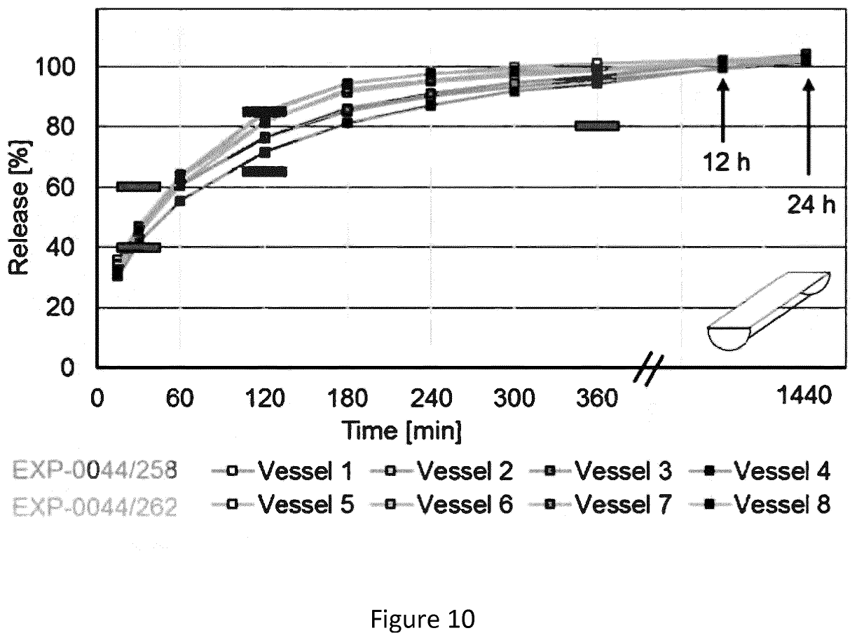

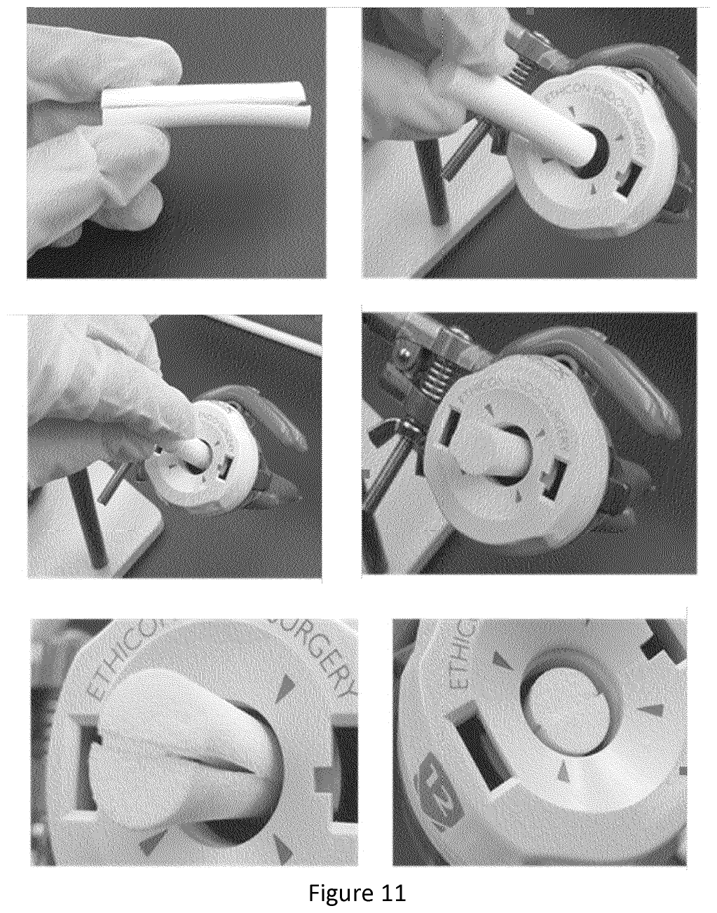

An alternate approach to wetting the implant to make it more amenable to insertion through a trocar is to reformulate the implant in a shape and size that will allow direct insertion through a trocar without having to cut or wet the implant. The dimensions of the implant in such formulation, are determined by trocar use. Without limitations, dimensions for the implant are as follows: Diameter ≤12 mm, or otherwise adapted to any specific trocar that can be used for a specific surgery procedure; Length ≤70 mm, or otherwise adapted to any specific trocar that can be used for a specific surgery procedure; 4 or 6 pieces (in halves). Various shapes, including, half-cylinders were tested. FIG. 9A shows embodiments for cylindrical/half-cylindrical drug delivery device implants, and FIG. 9B shows embodiments for cylindrical/half-cylindrical drug delivery device implants enclosed in a similarly shaped canister for delivery through a trocar. FIG. 10 shows the dissolution curves for different sizes and shapes of a square/prismatic implant (red bars) and reformulated implants. FIG. 11 shows representative images of the process of inserting cylindrical/half-cylindrical drug delivery device implants through a trocar.

The following table includes comparative properties of a square/prismatic implant (Xaracoll) and reformulated cylindrical/half-cylindrical implants (Case 1 and Case 2).

| | Xaracoll | Case 1 | Case 2 |
|---|---|---|---|
| How supplied | 3 matrices | 104 × 12 mmø | 70 × 12 mmø |
| | Cut into 6 pieces | 6 half cylinders | 6 half cylinders |
| | 50 × 25 × 5 mm | | |
| # of pieces/dose | 6 | 6 | 6 |
| Contact surface area (cm²) | 5 × 2.5 × 6 = 75 | 10.4 × 1.2 × 6 = 75 | 7 × 1.2 × 6 = 58.8 |
| Total Surface area (cm²) | 32.5 × 6 = 195 | 33.2 × 6 = 199 | 22.7 × 6 = 136.4 |
| API/piece (mg) | 50 | 50 | 50 |
| Vol./piece (cc) | 6.25 | 5.88 | 3.96 |
| Drug concentration (mg/cc) | 50/6.25 = 8.0 | 50/5.9 = 8.5 | 50/3.9 = 12.8 |

### Example 6: Use a Delivery Mechanism e.g. Canister to Aid the Insertion of Xaracoll

A further alternate approach that was tested was to insert the implant using a canister or enclosure device that can hold the implant. The canister or enclosure device is shaped and sized for easy insertion through a trocar. One example of such a canister or enclosure device is “StitchKit”, which is approved via 510k for sutures. The StitchKit canister is described in U.S. Pat. Nos. 8,418,851; and 6,986,780, both of which are incorporated herein by reference in their entireties for all purposes. Typical dimensions for a StitchKit canister are ˜50 mm in length, and 12 mm in diameter.

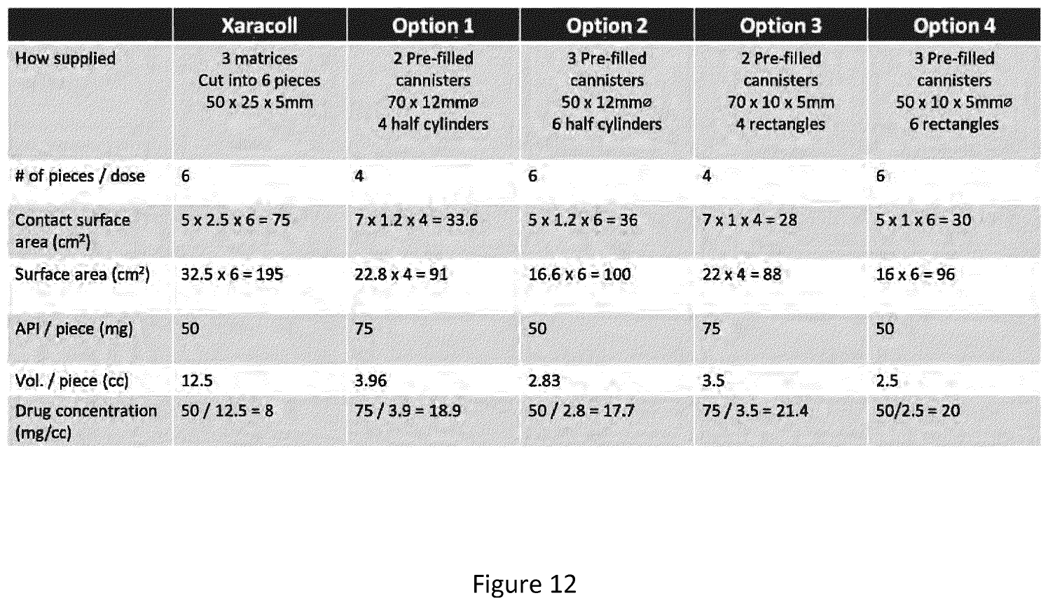

It was noted that if the clinically approved XaraColl implant was used as is, and inserted in the StitchKit canister by cutting the approved implant into appropriately sized pieces, several canisters would be required. On the other hand, the implant could be suitably reformulated to be appropriately shaped and sized to enable insertion in the StitchKit canister so as to require fewer canisters. FIG. 12 shows a chart including possible options that are being considered for reformulating the implant to allow insertion into an enclosure device or a canister for insertion through a trocar for implantation during a laparoscopic procedure.

### Example 7: Compressing, Cutting, and Folding XaraColl for Insertion into a Trocar

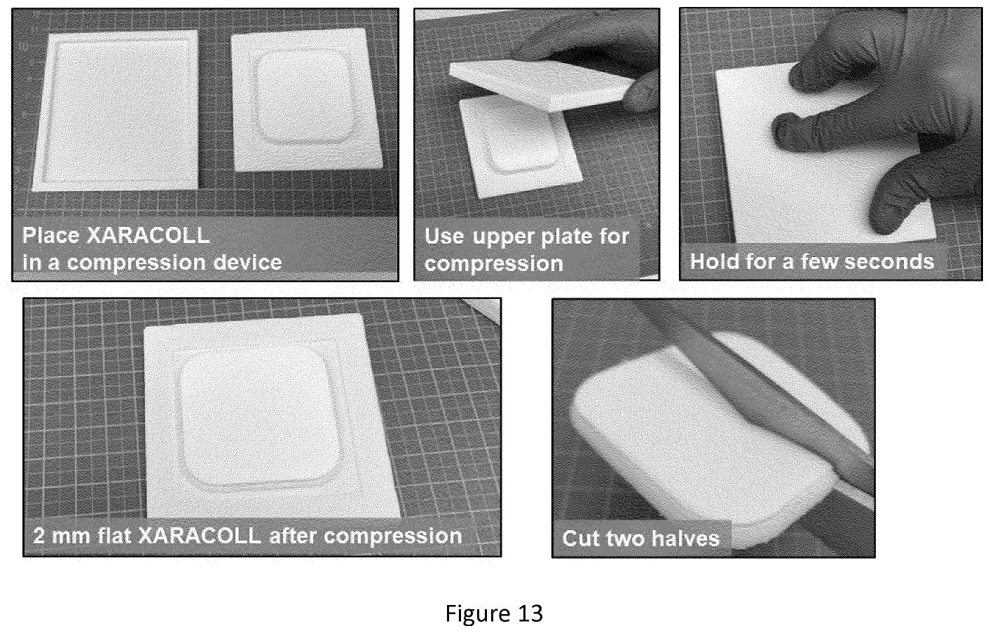

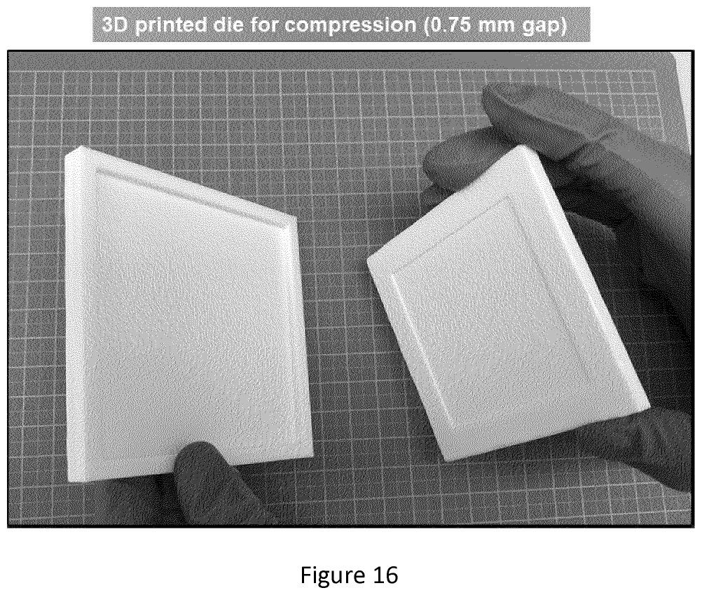

In one aspect, the present disclosure provides an approach to fit XaraColl into a trocar. XaraColl is a bupivacaine-collagen implant having dimensions of 5 cm×5 cm (×0.5 cm thick). First, a compression device having a bottom plate and a top plate is used to compress the 5 cm×5 cm (×0.5 cm thick) XaraColl product. XaraColl is placed on the bottom plate of the compression device, in the recessed portion of the bottom plate (FIG. 13). The top plate, also having a recessed portion, is placed directly on top of the bottom plate and pressed firmly for several seconds to compress XaraColl. The top plate is then removed, providing compressed XaraColl having a thickness of about 2 mm (0.2 cm), which is then cut in half (FIG. 13). The thickness of the recessed portion if the top plate is placed on top of the bottom plate of the compression device, in the absence of XaraColl, is about 0.75 cm (FIG. 16).

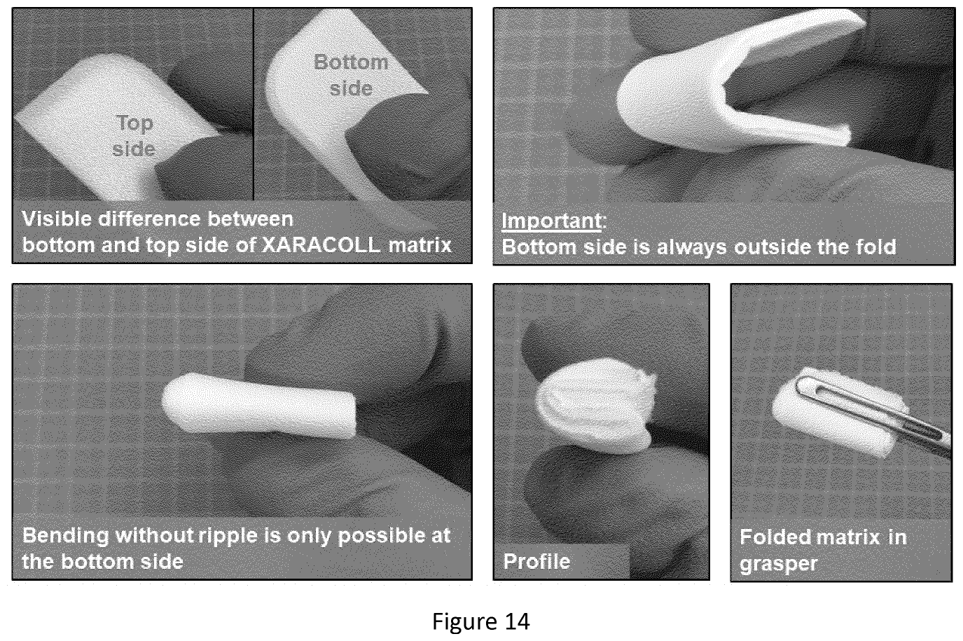

The cut XaraColl is inspected to determine the top side and the bottom side, wherein the top side has a beveled edge while the bottom side appears flat with no beveled edge (FIG. 14). The cut XaraColl is folded once along its long (5 cm) side such that the bottom side is on the outside of the folded XaraColl, forming a folded XaraColl product having dimensions of about 2.5 cm×2.5 cm (×0.4 cm thick). The folded XaraColl product is folded a second time, forming a folded XaraColl product having dimensions of about 1.25 cm×2.5 cm (×0.8 cm thick). The twice folded XaraColl product is placed into a grasper.

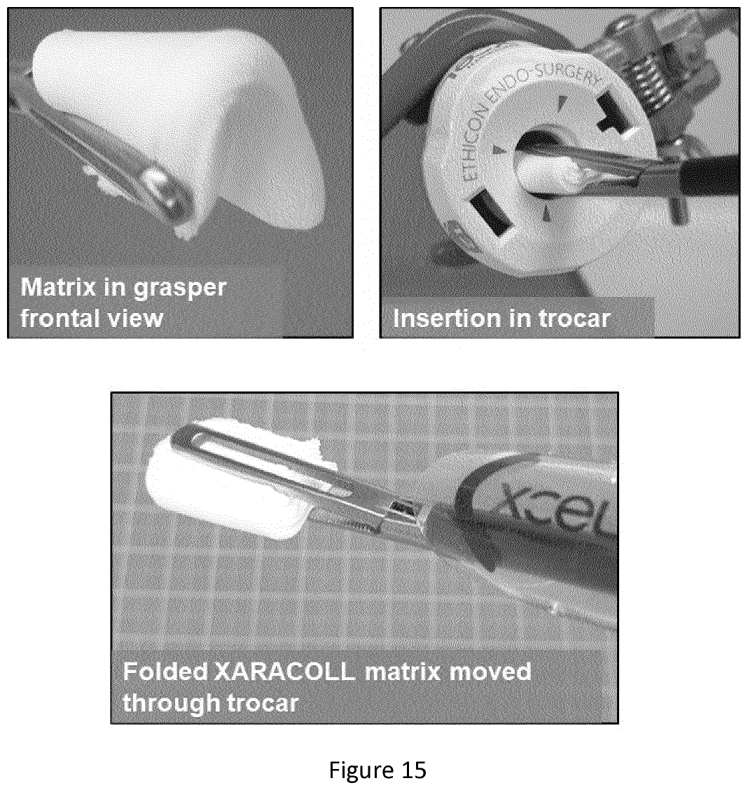

The grasper is placed into a trocar and moved through the trocar such that the prongs of the grasper holding the folded XaraColl product are outside of the trocar (FIG. 15).

### Example 8: Laparoscopic Application of XaraColl—Development of a Sample Preparation Technique (Folding Approach) Using Compression Tool Generation 1

### Introduction

For laparoscopic use, the XaraColl implant is moved through a trocar which functions as a portal for the placement of a XaraColl implant inside the body. The size of a trocar is limited to a maximum inner diameter between 8 mm and 12 mm. Due to this limited diameter, the XaraColl implant has to be compressed, folded and moved through the trocar by the use of a grasper. This process of sample preparation and passing through the trocar leads to physical stress (compression, folding, handling with a grasper and rubbing at the trocar seal), which could possibly damage the XaraColl matrix integrity and affect the drug release characteristics and profile.

In order to enable the laparoscopic use of XaraColl, an approach for the insertion of XaraColl through a trocar was investigated and the characteristics of the XaraColl implant were evaluated after this process. This procedure includes partial compression of the XaraColl implant to a thickness approximately 0.8 mm with a compression tool. This compressed matrix is then cut into two halves. Notably, the cutting of Xaracoll into two halves represents a standard process, which is already used for the application of XaraColl during traditional, open surgeries and thus no changes regarding the handling of XaraColl are introduced during this step. Nevertheless, studying the impact of cutting the XaraColl matrix on the dissolution properties suggested that the speed of API release differs for the respective XaraColl fragments, i.e. increases for smaller pieces. A f1/f2 factor analysis confirmed that cutting the XaraColl implant into two halves has only negligible impact on the dissolution behavior compared to uncut matrices. Subsequently, the two XaraColl halves are folded twice and moved through a trocar with a 12- or 8-mm diameter without major damage by using a grasper. However, this method exposes XaraColl to additional physical stress, i.e. folding, compression and rubbing, which might influence the drug release properties. To facilitate the movement of the XaraColl pieces through a trocar, moistening the XaraColl matrix was contemplated. While moistening of the XaraColl matrices resulted in an increased flexibility of the implants and thus facilitated the trocar insertion, studies indicated that the combination of cutting and moistening the XaraColl matrices followed by trocar insertion leads to accelerated API release. This effect was related to the moistening of the matrices rather than the trocar insertion. Moreover, by using this strategy for trocar insertion, it was not possible to push the XaraColl halves through the instrument.

In view of these findings, a handling technique was developed to minimize physical stress and maintain the XaraColl drug release characteristics as much as possible. This approach is based on partial compression of the XaraColl implant using a designated compression tool. After this slight compression, the flexibility of the collagen is increased and the matrix-implant gets foldable. The newly gained foldability is key for the laparoscopic use of XaraColl implants. The drug release characteristics, i.e. dissolution, of the XaraColl implants that have been moved through a trocar and prepared with the new “compress and fold” technique are evaluated in below.

This example focuses on three major topics:
1) The feasibility of the new “compress and fold” technique and evaluation of relevant XaraColl texture parameters. The evaluation is performed separately for each handling step (three steps: compression, cutting, folding).
2) The feasibility and handling of different medical grasper types that allow an improved handling of the folded XaraColl implant.
3) The evaluation of the impact that is caused by applying trocars of different diameters and from different manufacturers.

### Methods

Five different XaraColl batches were used for the evaluation: 21001005, 20000607, 20022906, 19002602, 18042611. The used equipment is listed in Table 5.

TABLE 5
Equipment used in Example 8

| | | Inv.- |
|---|---|---|
| Name | Manufacturer | No./SN |
| Compression frame 0.8 mm (3D print) | SYNTACOLL | N/A |
| Compression tool 0.8 mm (3D print), | SYNTACOLL | N/A |
| Generation 1.0 | | |
| Compression tool 0.8 mm (stainless | KFS | N/A |
| steel) Generation 1.1 | MASCHINENBAU | |
| | (Abensberg) | |
| Grasper; | N/A(*) | N/A |
| single action, intestinal grasping | | |
| forceps, 30 mm clamps | | |
| Grasper; | MÖLNLYCKE | N/A |
| double action, intestinal grasping | | |
| forceps, 30 mm clamps | | |
| Grasper; | MÖLNLYCKE | N/A |
| double action, Babcock | | |
| Grasper; | MÖLNLYCKE | N/A |
| double action, Maryland | | |
| Trocar; | ETHICON | N/A |
| tube length: 200 mm; ID: 12 mm | Endo-Surgery | |
| Trocar; | ETHICON | N/A |
| tube length: 200 mm; ID: 8 mm | Endo-Surgery | |
| Trocar; | MÖLNLYCKE | N/A |
| tube length: 200 mm; ID: 12 mm | Health Care | |
| Trocar; | AUTO SUTURE | N/A |
| tube length: 200 mm; ID: 12 mm | | |
| Universal testing machine Zwicki 500N | ZWICK/ROELL | 200134 |

### The Compression and Folding Technique

To prepare a XaraColl implant for laparoscopic use, three preparation steps are necessary: compression of XaraColl, cut compressed XaraColl into halves, and fold the compressed halved XaraColl.

### Compression:

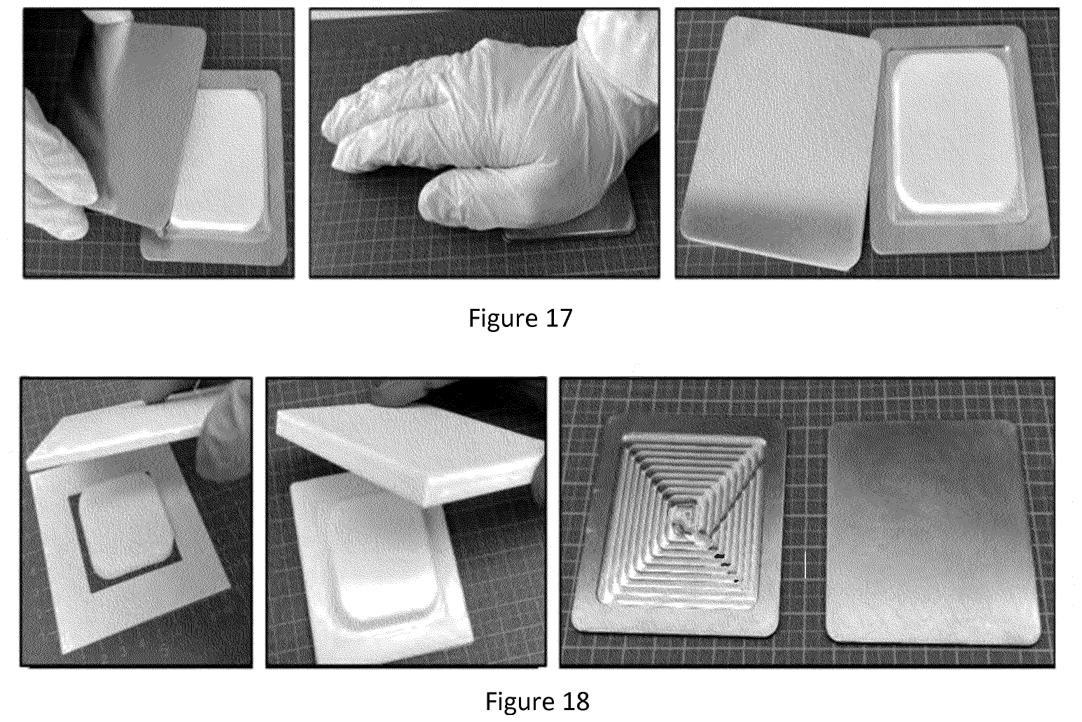

In order to increase the flexibility of the XaraColl implant to allow folding, a slight compression is necessary. For this purpose, a “compression tool” was developed. This compression tool is constituted of a bottom part, onto which the XaraColl implant is placed, and a top part, which is used as a lid and onto which the palms are placed for compression (FIG. 17). The matrices are compressed reliably to approximately 0.8 mm (as the height of the compression gap equals 0.08 mm) using this tool and re-expand to ˜2 mm upon release of the compression force. Within this study, three different compression tools were designed (FIG. 18, Table 5), which are identical regarding the relevant dimensions, i.e., their compression gap, and performance and differ only in their material.

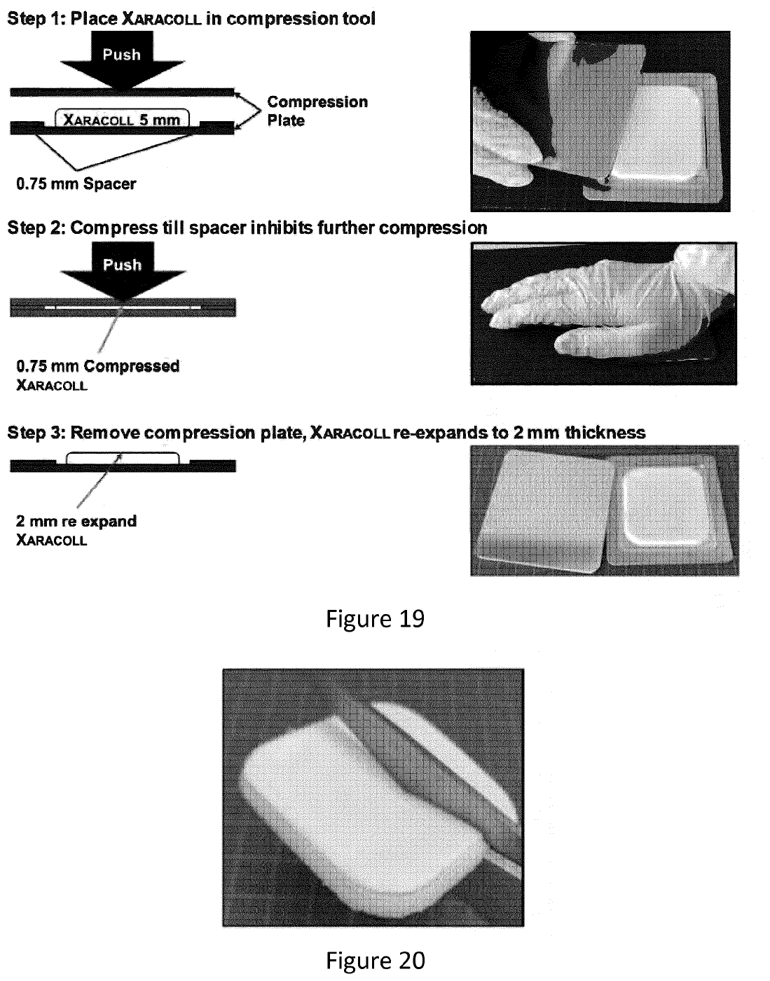

The sample compression is summarized in three steps in FIG. 19. In step 1, XaraColl is placed into the compression tool. In step 2, the compression plate is pushed until the spacer inhibits further compression. In step 3, the plate is removed and the XaraColl re-expands to ˜2 mm.

### Cutting:

In order to push the XaraColl implant through a trocar, it has to be cut into two halves by using scissors. This is necessary, since a complete XaraColl implant, even when it has been compressed and folded beforehand, is way too oversized for the limited diameter of a 12 mm or even 8 mm trocar. Moreover, cutting of XaraColl into two halves represents a standard process, which is already used for the application of XaraColl during traditional, open surgeries and thus no changes regarding the handling of XaraColl are introduced during this step. After compression XaraColl is cut into two halves. Cutting after compression is easily possible with scissors (FIG. 20).

### Folding:

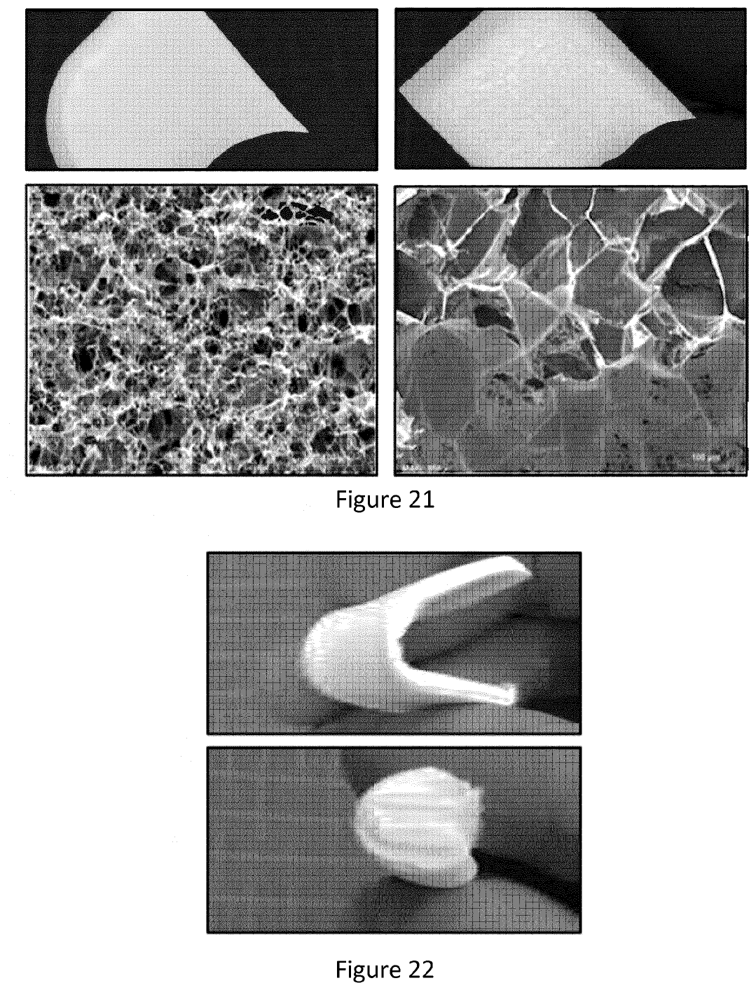

Due to the manufacturing process with freezing and lyophilization, the blister and the Tyvek side of XaraColl are not identical. The blister side has slightly rounded edges from the mold used for lyophilization matrix (FIG. 21, top left). In contrast, the Tyvek side has sharp edges and a concave (bowl) shape caused by capillary forces at the mold wall (FIG. 21, top right). Due to the nature of freeze-drying process, the micro-structure of XaraColl's Tyvek side and blister side are different. The blister side of the matrix (FIG. 21, bottom left) shows a very flexible fine collagen structure, whereas the Tyvek side (FIG. 21, bottom right) shows a more or less continuous closed epithelium-like surface with theoretically less flexibility. Hence, both sides of the XaraColl matrix are different and require distinction for optimal folding performance.

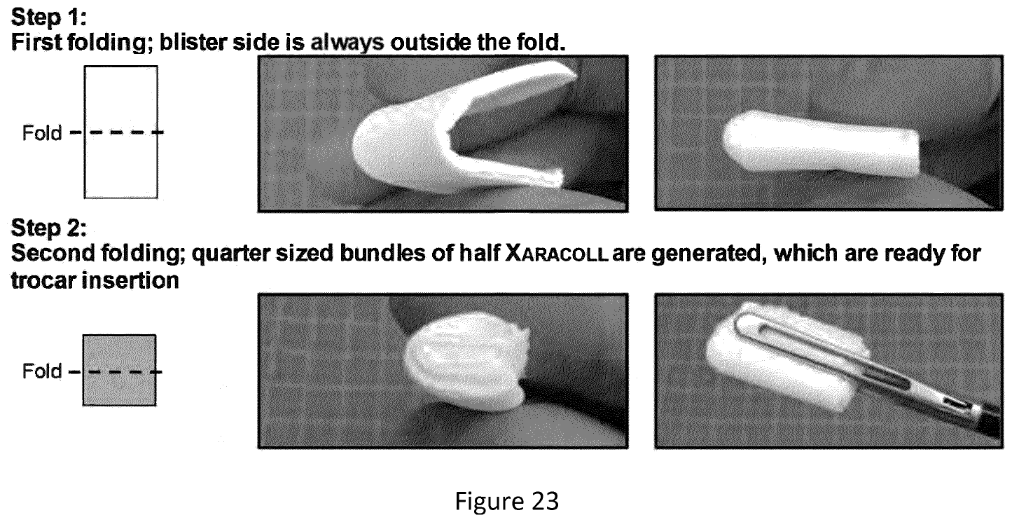

Due to these structural differences, the blister side of XaraColl is more elastic than the Tyvek side. This elastic side facilitates the folding process and reduces the chance of tearing the product during the folding step. During the folding process, the elastic blister side is stretched and no ripples or tears occur (FIG. 22). If the compressed half XaraColl is not folded correctly the surface could ripple or tear and the implant could potentially break in two parts. The folding process developed in this study is depicted in FIG. 23.

Handling of XaraColl with Medical Graspers

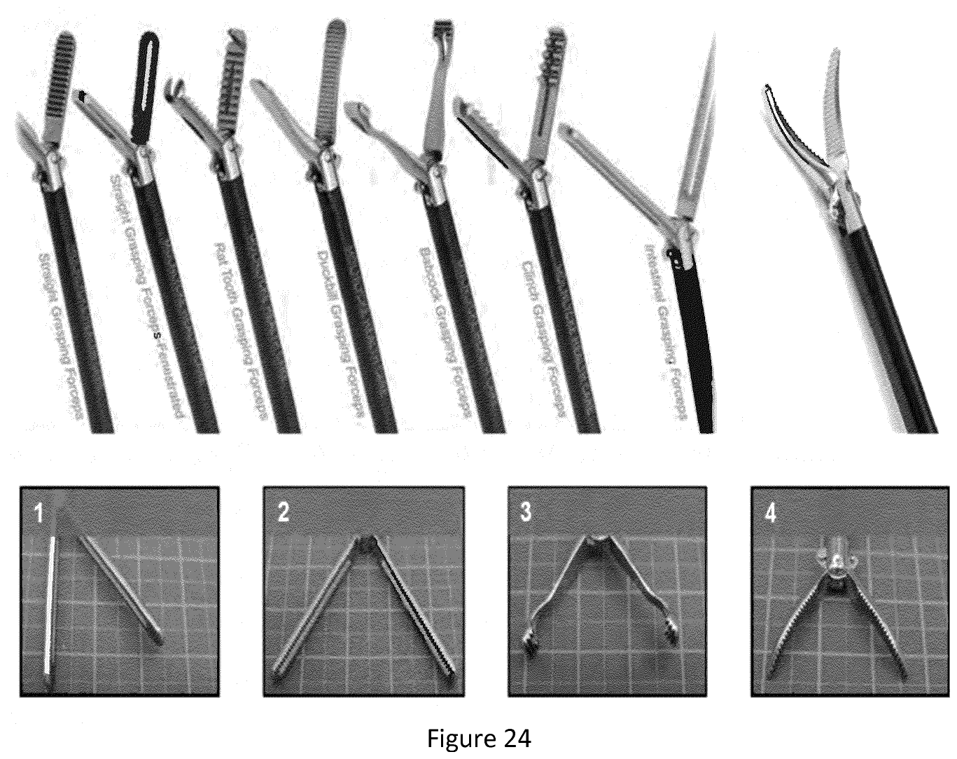

Medical graspers are surgical instruments and present in every operating room. The grasper is used to transfer the folded, compressed half XARACOLL implant through the trocar inside the body. Different types of graspers are available, which will be discussed and their performance regarding feasibility and handling evaluated (FIG. 24).

### XaraColl Insertion and Moving Through Trocar Tube

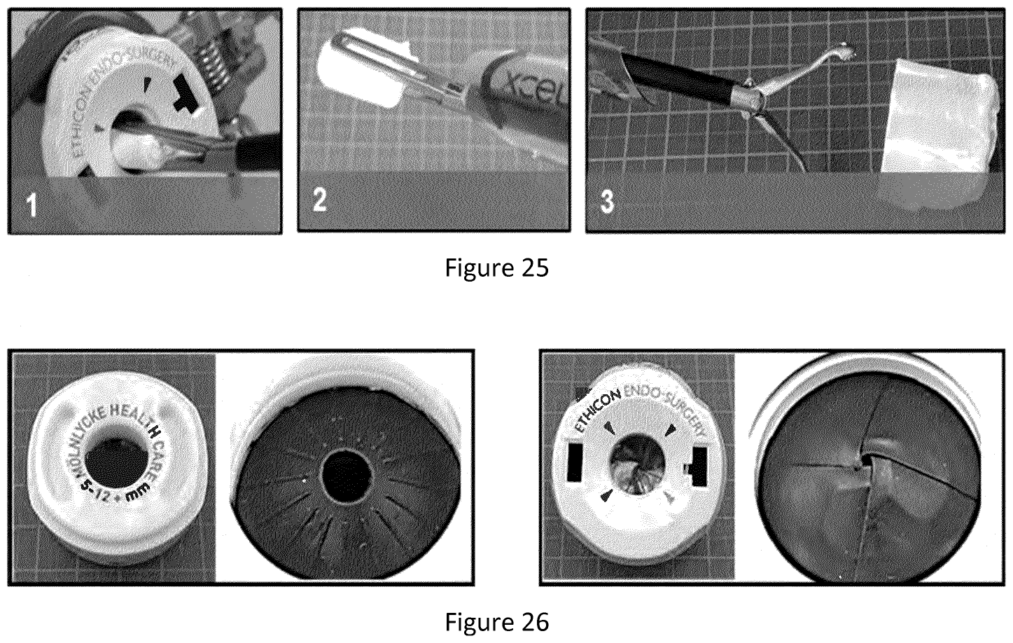

The folded implant is moved through the trocar, whereby it is held and supported by a suitable grasper. This process can be described in three steps (FIG. 25):
Step 1: Passing the folded implant through the seal.
Step 2: After passing the seal, the implant is moved down the trocar tube.
Step 3: Release of the implant from the grasper.

### Trocar Seals:

The trocar seal is the most important design element of the trocar as it represents the greatest resistance to passing XARACOLL into the abdomen. Additionally, the force required for moving the implant through the trocar tube strongly depends on the tube diameter while the trocar seal is identical for all tube sizes. Every trocar has a system of two seals. The first seal (called universal seal) can be a segmented seal or a simple elastic ring (FIG. 26). This seal is part of the trocar head and can be removed. The second seal, also called valve, is commonly a trap door design. The function of this valve is solely to inhibit the release of CO₂ gas.

### Tube Diameters:

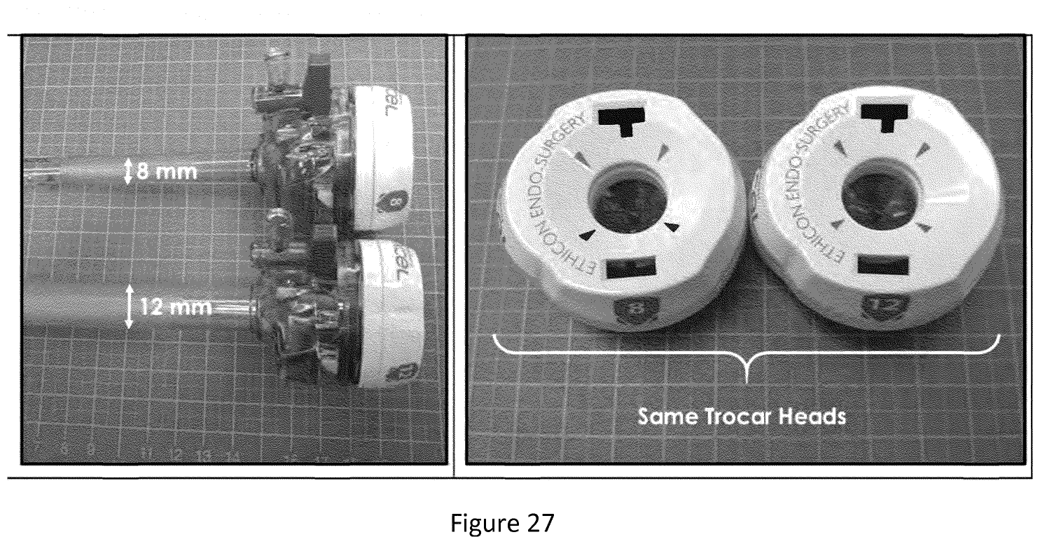

The tube diameter is independent from the seal/valve design and its resistance. That means that for different tube diameters the seal/valve is the same, see FIG. 27.

### Dissolution Testing

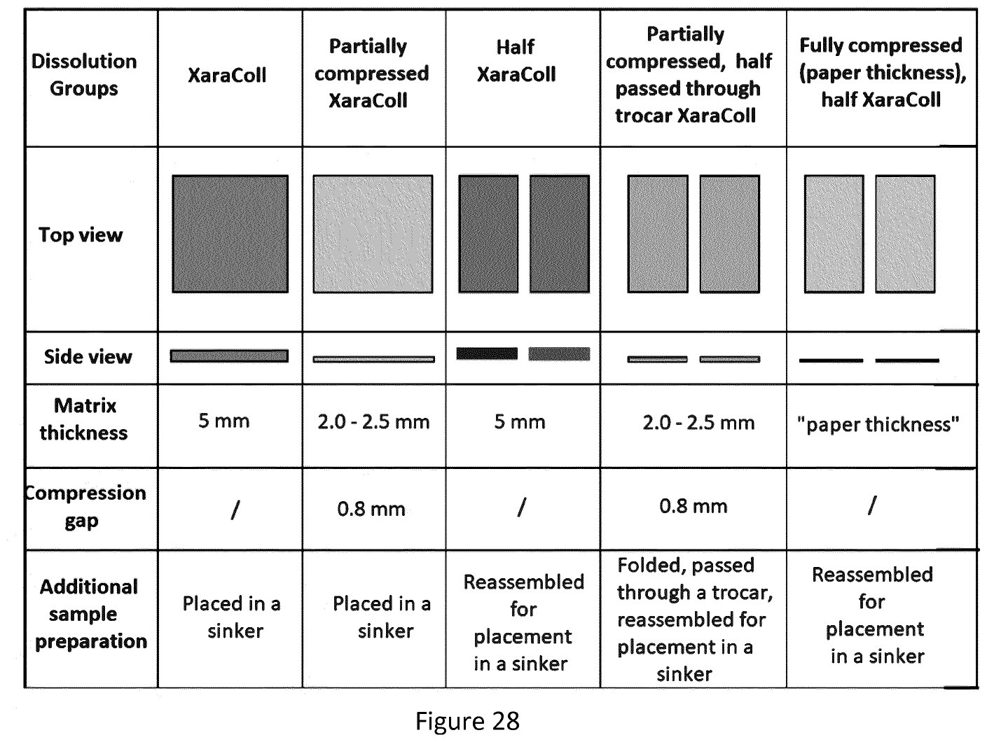

The impact of the trocar insertion process on the dissolution properties of XaraColl implants was studied. Thus, the XaraColl matrices were analyzed at different stages of this preparation process: the partially compressed matrices prior to cutting, the halved matrices, and the partially compressed and halved matrices which had been inserted through the trocar. Additionally, untreated XaraColl implants as well as half, completely pressed matrices, with paper thickness were characterized for comparison (FIG. 28). The half matrices were reassembled into a complete matrix and placed in a sinker prior to dissolution testing. In order to ensure comparability, all analyzed samples were derived from the identical XaraColl batch. Noteworthy, the “usual” release testing does not necessarily mirror the in vivo performance of XaraColl, as the routinely performed release test involves complete XaraColl matrices and not halved. Since the sample treatment described above is not expected to affect the performance of XaraColl in QC release tests, the latter are not subject of this study. For comparison, previous investigations of XaraColl's bupivacaine HCl content after trocar insertion showed no loss of bupivacaine during the procedure and further demonstrated that the swelling properties of untreated vs treated (compressed and inserted into trocar) matrices, are identical. Therefore, it can be assumed that the sample preparation technique applied in the present study generates equal results, especially since moistening of XaraColl is not necessary in this case and thus the probability of a loss of content is negligible.

### Calculation of f1 and f2 Factors and Interpretation:

The evaluation of f1 and f2 factors provides indications whether dissolution profiles are similar or dissimilar and is thus a potent tool to compare different data sets. The dissimilarity factor, f1, describes the difference between a reference and a test curve at each time point in percent and is a measurement of the relative error between the two curves wherein results with values in the range of 0-15 are entitled “not dissimilar.” The similarity factor, f2, is a measurement of the similarity on percent dissolution between the two profiles wherein dissolution curves with f2 values in between 50 to 100 are called “similar.” The two factors f1 and f2 are calculated according to Equation 1.

$$Calculation of f 1 and f 2 factors . \qquad Equation 1$$

$${f}_{1} = 100 ( \frac{{\sum }_{t = 1}^{k} \vert {R}_{t} - {T}_{t} \vert}{{\sum }_{t = 1}^{k} {R}_{t}} )$$

$${f}_{2} = 50 \cdot log \{ {[ ( 1 + ( \frac{1}{n} ) {\sum }_{t = 1}^{k} {( {R}_{t} - {T}_{t} )}^{2} ) ]}^{- 0.5} \cdot 100 \}$$

$$\begin{matrix}{R}_{t} & Average release [ \% ] of reference ( R ) at time point t \\ {T}_{t} & Average release [ \% ] of test ( T ) at time point t \\ k & Number of time points\end{matrix}$$

The calculation of f1 and f2 should obey the following requirements:

The calculation is most suitable when a minimum number of three to four dissolution time points are available.

The dissolution measurements of the test and the reference have to be conducted under exactly the same conditions, i.e. identical sampling time points.

Only one time point with a drug release of >85% may be taken into account.

The RSD of the average (n=12) release values should be less than 20% in case of time points ≤15 min and below 10% at any time point.

The test and the reference curve are considered similar if f1 is close to 0 (0-15) and f2 greater than 50 (Table 6).

TABLE 6
Criteria for dissimilarity (f1) and similarity (f2) of dissolution profiles.

| | | | Acceptance | Admissible |
|---|---|---|---|---|
| | | Mathematical dependence | criterion | deviation |
| f1 | Dissimilarity | Direct proportional to average profile | 0-15 | 15% |
| | (difference) | difference | | |
| f2 | Similarity | Inversely proportional to average | 50-100 | 15% |
| | (closeness) | squared profile difference | | |

Comparison with XaraColl Acceptance Criteria:

The acceptance criteria for the API release at 30, 120 and 360 mi are defined in Table 7. These specifications represent a quality test and are only valid for complete, untreated XaraColl matrices, which are represented in group 1. In case of the partially or fully compressed (groups 2, 4 and 5) and halved (group 3) matrices, these criteria are not applicable. These groups are compared using f1/f2 statistical analysis as described above.

TABLE 7
Specifications for dissolution testing.

| | | Acceptance criterion | | |
|---|---|---|---|---|
| | | 30 min | 120 min | 360 min |
| Level | | [%] | [%] | [%] |
| 1 (n = 6) | single values | 40-60 | 65-85 | NLT 80 |
| | average | 40-60 | 65-85 | NLT 80 |
| 2 (n = 12) | single values | 30-70 | 55-95 | NLT 70 |
| | average | 40-60 | 65-85 | NLT 80 |
| 3 (n = 24) | all single values | 20-80 | 45-105 | NLT 60 |
| | at least 22 single values | 30-70 | 55-95 | NLT 70 |
| | average | 40-60 | 65-85 | NLT 80 |

### Reagents and Instruments:

The reagents and instruments used for this study are listed in Table 8 and Table 9, respectively.

TABLE 8
Chemicals used for this study.

| | | Article | |
|---|---|---|---|
| Compound (Chemical Formula) | Supplier | number | Batch number |
| Potassium dihydrogen phosphate | Merck | 1.04873.1000 | AM0973973637 |
| p.a. (KH₂PO₄) | | | |
| Dipotassium hydrogen phosphate [symbol] | Merck | 1.05104.1000 | AM0840604638 |
| Sodium chloride (NaCl) | Merck | 1.06404.0500 | K51828804947 |
| Sodium hydroxide (NaOH), 1M | Merck | 1.091.411.000 | HC90868841 |
| Hydrochloric acid (HCl), 1M | Merck | 1.090.571.000 | HC03824657 |
| Triethylamine (NEt₃), ≥99.5% | Sigma Aldrich | 471283-500ML | STBH9365 |
| Trifluoroacetic acid | Sigma Aldrich | 153112E | QO638459 |
| (CF₃COOH), ≥99.9% | | | |
| Acetonitrile gradient grade | Merck | 1.00030 | I1083130 |
| for LC (MeCN) | | | |

| | | |
|---|---|---|
| Bupivacaine HCl, working | In house production from | Work-BUP- |
| standard (C₁₈H₂₈N₂O) | reference standard | 1119-01 |
| Purified Water for analytical | In-house production from | Day of use |
| applications (H₂O) | Elix ® device | |

[symbol]

indicates data missing or illegible when filed

TABLE 9
Instruments used for this study.

| | | Inv.-number/Serial |
|---|---|---|
| Instrument | Manufacturer | number |
| HPLC system 2695e with UV/VIS | Waters | 500302 |
| detector 2489 | | |
| pH-Meter 781 | Metrohm | 500240 |
| Analytical balance XP 205 DR | Mettler | INTECH 00006 |
| | Toledo | |
| Kinetex XB-C18 length 33 mm, ID | Phenomenex | H20-079366 |
| 3 mm, particle size 2.6 μm) | | |
| Trocar (tube length 200 mm, | Ethicon | N/A |
| diameter 12 mm) | | |
| Grasper (babcock, double-action) | Mölnlycke | N/A |

### Dissolution Testing and HPLC Analysis:

All characterized samples were derived from the identical XaraColl batch 20000607, which ensures comparability of the resulting dissolution data. Importantly, the impact on the API release of different stages of the preparation technique described above has been evaluated. For this purpose, complete, untreated XaraColl (group 1), as well as compressed, cut XaraColl, which was pushed through a trocar (group 4), has been investigated (Table 10). Additionally, compressed (group 2), half (group 3) and maximally compressed XaraColl (group 5) were characterized.

TABLE 10
Dissolution test groups investigated in this study.

| | Short | |
|---|---|---|
| Group | description | Preparation |
| 1 | XaraColl | Placed in a sinker |
| 2 | XaraColl, | Partially compressed with compression tool |
| | compressed | (0.8 mm), placed in a sinker |
| 3 | XaraColl, half | Cut into two halves, reassembled for |
| | | placement in a sinker |
| 4 | XaraColl, | Partially compressed with compression tool |
| | trocar | (0.8 mm), cut into two halves and pushed |
| | | through a 12 mm trocar; |
| | | reassembled and placed in a sinker |
| 5 | XaraColl, max. | Fully compressed (paper thickness) and placed |
| | compression, | in a sinker |
| | half | |

All dissolution experiments were performed using an Agilent dissolution system with dissolution workstation. Important system parameters applied for this study are summarized in Table 11. Samples were drawn at time points t=5, 10, 15, 30, 45, 60, 90, 120, 180, 240, 360 and 1080 minutes, which were collected in a MPLC vial tray and subsequently characterized via HPLC analyses. The settings for these analyses were according to those in Table 12. For data evaluation, an external Bupivacaine HCl standard was applied. All calculations were conducted using a reviewed Excel-sheet.

TABLE 11
Dissolution system configuration and method parameters.

| | Parameter | Setting value |
|---|---|---|
| System | System | R&D Fast Dissolution |
| Configuration | | CWe/R&D Fast Dissolution MLe |
| | System ID | 12.1 |

| | | |
|---|---|---|
| Plunger speed | 12 | ml/min |

| | | |
|---|---|---|
| Method | Method | R&D Dissolution all time |
| | | points (18) CWe |
| | Method ID | 19.5 |

| Sample volume | 1.5 | ml |
|---|---|---|
| Prime volume | 10 | ml |
| Purge volume | 10 | ml |
| Replacement volume | 4.5 | ml |
| Waste drop volume | 3.0 | ml |
| Bath temperature | 37.2° | C. |
| Vessel temperature | 37.0 ± 0.5° C. | |
| Paddle speed | 50 | rpm |

| | |
|---|---|
| Media volume | 500 mL ± 5 ml |

TABLE 12
HPLC settings used for the analysis of dissolution samples.

| Parameter | Setting value | |
|---|---|---|
| Column | Kinetex XB-C18 length 33 mm, | |
| | ID 3 mm, particle size 2.6 μm | |
| Mobile phase | TEA/TFA buffer (pH 3.4) and | |
| | MeCN in a ratio of 75:25 | |
| Flow rate | 0.8 | ml/min |
| Column temperature | 20° | C. |
| Sample temperature | 15° | C. |
| Injection volume | 5 | μl |
| Detection | 230 | nm |
| Run time | 2 | min |

### Results

Evaluation of Matrices after Compression, Cutting and Folding

### Texture Analysis:

In order to evaluate the feasibility of the compression technique, matrix thickness prior and after compression was measured. Additionally, different texture analyses, i.e. resistance to pressure and force to break, were conducted in order to unveil a possible correlation of the latter with the compressed thickness. For all analyses, the same batches (10 samples each) and the Zwicki 500N texture analyzer was used (Table 13) with ten samples each.

TABLE 13
List of texture analyses and samples.

| # | Texture analysis | Sample |
|---|---|---|
| 1 | Initial XaraColl thickness [mm] | 10 XaraColl matrix implants per batch |
| 2 | Re-expanded XaraColl | 10 compressed XaraColl matrix |
| | thickness [mm] | implants per batch |
| 3 | Resistance to pressure [N] | 10 XaraColl matrix implants per batch |
| 4 | Force to break [N] | 10 XaraColl matrix implants per batch |

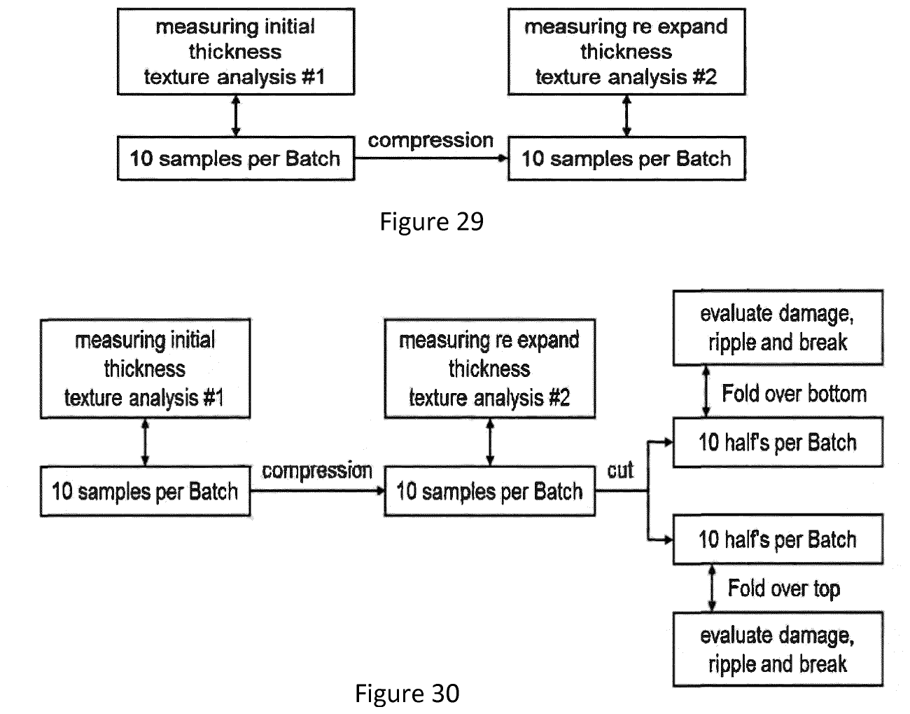

The texture analysis for thickness includes measurements prior to compression (#1) and post-compression (#2) were obtained with the same set of samples (FIG. 29). Moreover, the thickness after different compression times was measured.

### Folding of XaraColl

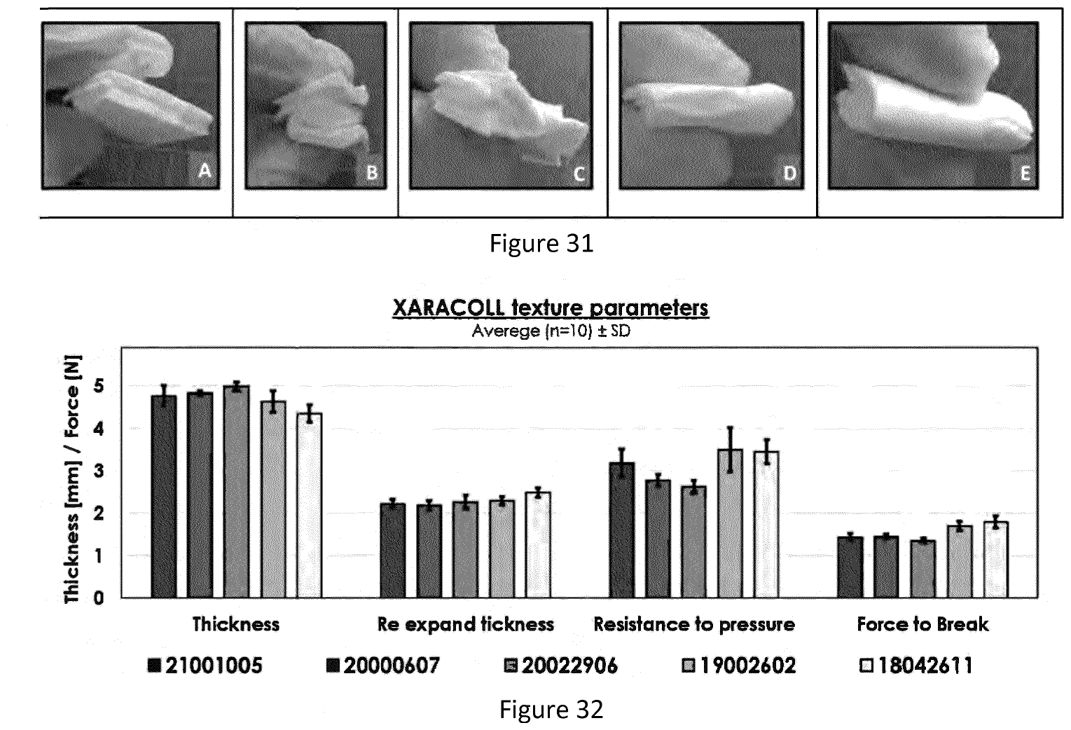

The requirement for folding is to minimize ripples or tears. This aspect was evaluated with five different XaraColl batches. Ten XaraColl implants per batch were compressed and cut into two halves, as described in above. One of these halves was folded in the correct orientation (bend with blister side out), the other half was folded with the wrong orientation (bend with Tyvek side out, FIG. 30). In order to achieve maximal comparability, the same samples that were already used for the thickness measurements were reused for the foldability evaluation. Results are evaluated in form of successful foldings. Non successful foldings are categorized and documented via photos as demonstrated in FIG. 31 and Table 14.

TABLE 14
Damage evaluation of folding.

| | Letter | | |
|---|---|---|---|
| Type of damage | (FIG. 31) | Description | Evaluation |
| First fold break | A | Separation into | Considered as FAIL |
| | | two parts | |
| Second fold break | B | Separation into | Considered as FAIL |
| | | two parts | |
| Strong rupture | C | Almost full range | Considered as FAIL |
| Moderate rupture | D | ~50% range | Considered as PASS |
| Light ripple or | E | Less than 50% | Considered as PASS |
| no rupture | | range | |

### Wetting and Swelling

The swelling (i.e. change in thickness of the implant due to water uptake) after wetting (i.e. placed on water) was evaluated. For every batch, a non-compressed half XaraColl matrix-implant was compared with a compressed half XaraColl matrix-implant. Both halves were placed on the water surface for 10 minutes. The thickness of the soaked pieces was visually inspected.

### Evaluation of Grasper Performance

For the successful insertion of the compressed, halved and folded XaraColl, three requirements for the grasper are important for the application which are:
Easy insertion: The grasper should hold and support the folded implant in form of a tight bundle during insertion. Support is required in order to move the soft implant through the trocar seals.
Minimum damage: The grasper should not damage the folded implant with rough teeth or spikes.
Release: After the folded implant was moved through the trocar tube, the grasper should release the implant by opening the clamps.
These requirements were evaluated with five different XaraColl batches and all four shown grasper models and a 12 mm trocar. The results are given in form of a detailed description of advantages and disadvantages in the Results and Discussion section.

### Evaluation of Trocar Performance

As discussed above, the successful movement of XaraColl through the trocar depends on the quality and nature of the trocar seal as well as on the trocar tube diameter. These criteria, including the seal resistance of different trocars, were tested to identify possibly important design elements.

### Results and Discussion

### Compression Relevant Texture Parameters

### Texture Parameter Test Condition 1:

5 different batches, compression time 3 seconds, compression tool 0.8 mm gap.

Compression relevant texture parameters were tested for five different XaraColl batches with n=10 samples. The re-expanded thickness was measured after a compression time of 3 seconds (Table 15, FIG. 32). The batches with lower initial thickness show a higher resistance to pressure (batches 19002602 and 18042611) wherein batch 18042611 particularly tends to larger re-expansion. The texture parameters, resistance to pressure, and breaking force, show typical values for XaraColl and there is no obvious correlation with the re-expanded thickness.

TABLE 15
Texture parameters of 5 batches, 3 sec compression.

| | | | Average | |
|---|---|---|---|---|
| | | Average | resistance | Average force |
| | Average initial | re-expanded | to pressure | to break |
| | thickness ± SD | thickness ± SD | (F2mm) ± SD | (Fmax) ± SD |
| Batch # | [mm] | [mm] | [N] | [N] |
| 21001005 | 4.8 ± 0.24 | 2.2 ± 0.11 | 3.2 ± 0.33 | 1.4 ± 0.09 |
| 20000607 | 4.8 ± 0.05 | 2.2 ± 0.11 | 2.8 ± 0.14 | 1.5 ± 0.07 |
| 20022906 | 5.0 ± 0.10 | 2.3 ± 0.16 | 2.6 ± 0.14 | 1.4 ± 0.07 |
| 19002602 | 4.6 ± 0.25 | 2.3 ± 0.10 | 3.5 ± 0.52 | 1.7 ± 0.11 |
| 18042611 | 4.4 ± 0.21 | 2.5 ± 0.12 | 3.5 ± 0.28 | 1.8 ± 0.14 |

### Texture Parameter Test Condition 2:

2 different batches, 3 different compression times, compression tool 0.8 mm gap

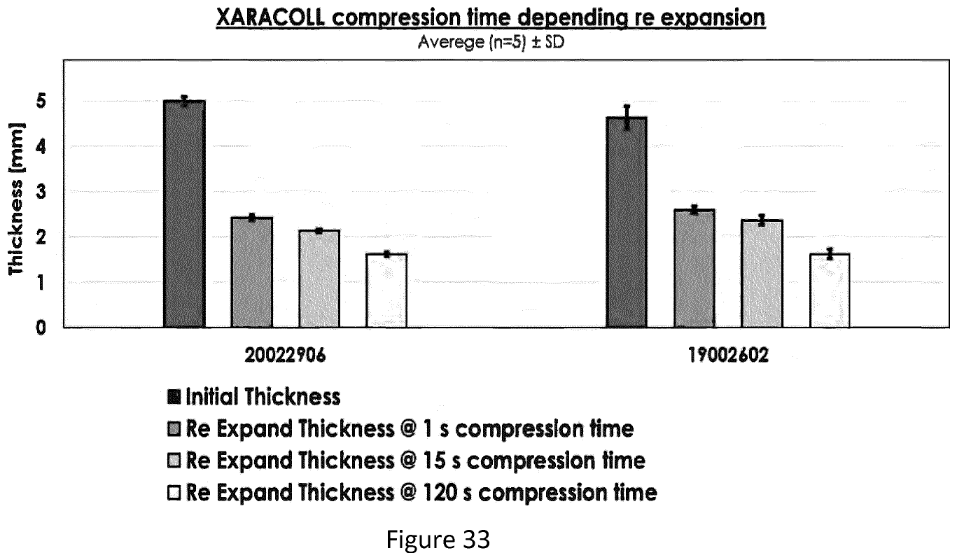

The texture parameters initial thickness and re-expanded thickness were tested for two different XaraColl batches (batch 20022906 with lowest resistance to pressure and batch 19002602 with highest resistance to pressure) with n=5 samples. The re-expanded thickness was measured after three different compression times: 1, 15 and 120 seconds (Table 16, FIG. 33).

TABLE 16
Re-expansion after 1, 15 and 120 seconds of compression.

| | | Re-expanded | Re-expanded | Re-expanded |
|---|---|---|---|---|
| | Initial | thickness 1 s | thickness 15 s | thickness 120 s |
| | thickness ± SD | compression ± SD | compression ± SD | compression ± SD |
| Batch # | [mm] | [mm] | [mm] | [mm] |
| 20022906 | 5.0 ± 0.10 | 2.4 ± 0.06 | 2.1 ± 0.04 | 1.6 ± 0.05 |
| 19002602 | 4.6 ± 0.25 | 2.6 ± 0.07 | 2.4 ± 0.10 | 1.6 ± 0.11 |

The re-expanded thicknesses clearly depend on the compression time. For the matrix, which was compressed for 120 s, the smallest re-expanded thickness was measured, while the matrix, which was compressed the shortest time (1 s) shows the highest re-expanded thickness. Considering that the matrix thickness is a crucial parameter for the folding process, a short compression time of only 1 s can be defined as worst case and was used throughout all further evaluations.

### Folding

5 different batches, compression time 3 seconds, 0.8 mm compression gap: Five different XaraColl batches with n=20 samples (halves) were tested. Ten halves were folded correctly (blister side out) and ten were folded incorrectly (Tyvek side out). The gained results are summarized in Table 17.

TABLE 17
Folding results

| | | Break at | | | Light |
|---|---|---|---|---|---|
| Batch | Folding | 1. or 2. | Strong | Moderate | ripple or |
| # | side | Fold | ripple | ripple | no ripple |
| 21001005 | Correctly | 0/10 | 0/10 | 0/10 | 10/10 |
| | (Blister side) | | | | |
| | Incorrectly | 10/10 | N/A | N/A | N/A |
| | (Tyvek side) | | | | |
| 20000607 | Correctly | 0/10 | 0/10 | 0/10 | 10/10 |
| | (Blister side) | | | | |
| | Incorrectly | 10/10 | N/A | N/A | N/A |
| | (Tyvek side) | | | | |
| 20022906 | Correctly | 0/10 | 0/10 | 0/10 | 10/10 |
| | (Blister side) | | | | |
| | Incorrectly | 10/10 | N/A | N/A | N/A |
| | (Tyvek side) | | | | |
| 19002602 | Correctly | 0/10 | 0/10 | 0/10 | 10/10 |
| | (Blister side) | | | | |
| | Incorrectly | 10/10 | N/A | N/A | N/A |
| | (Tyvek side) | | | | |
| 18042611 | Correctly | 0/10 | 0/10 | 0/10 | 10/10 |
| | (Blister side) | | | | |
| | Incorrectly | 10/10 | N/A | N/A | N/A |
| | (Tyvek side) | | | | |

Clearly, the folding orientation (blister side vs Tyvek side) is crucial in order to achieve the best possible results. In cases where XaraColl is folded correctly (blister side on the outside of the fold), the matrix breaks in none of the samples. If the matrix is folded incorrectly (Tyvek side is outside), however, the matrix ruptured in all attempts. Thus, it can be concluded that the folding process is only successful when the correct folding orientation is adhered and fails if the matrix is folded incorrectly. Additionally, if the correct technique was used, the folding process was successful independent of which XaraColl batch was used.

### Wet Swelling

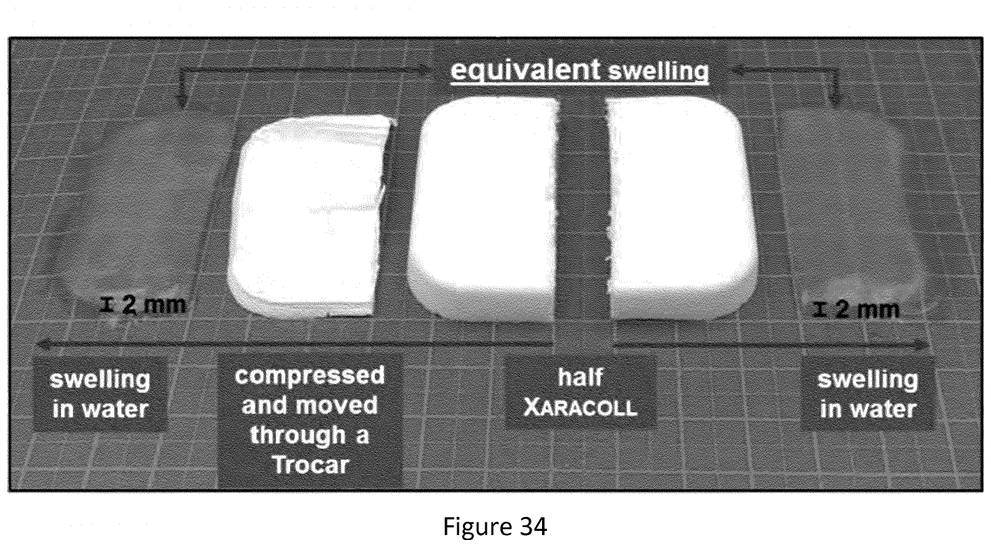

For every batch, a non-compressed half XaraColl implant was compared with a compressed half XaraColl implant (FIG. 34). The swelling for compressed and non-compressed is equivalent for all batches, meaning that the sample preparation process developed on this study has no effect on the wet swelling properties of XaraColl.

### Grasper Performance

The performance of four different graspers was evaluated. As stated elsewhere herein, the utilized graspers need to fulfill certain requirements in order to ensure minimum damage of the XaraColl implant during trocar insertion which are:

Graspers with rough teeth or spikes are not suitable due to potential damage of the folded matrix.

The grasping forceps needs to be sufficiently long so that it encloses the XaraColl implant as complete as possible and provides optimum support of the matrix and diminishes potential damage during the trocar insertion step.

Hence, a pre-selection of graspers (FIG. 24) was conducted and 4 out of 8 graspers were found as potentially suitable and were selected for further tests. Due to the criteria stated above, solely the single action intestinal grasper, the double action intestinal grasper, the double action Babcock grasper, and the double action Maryland grasper were tested in detail.

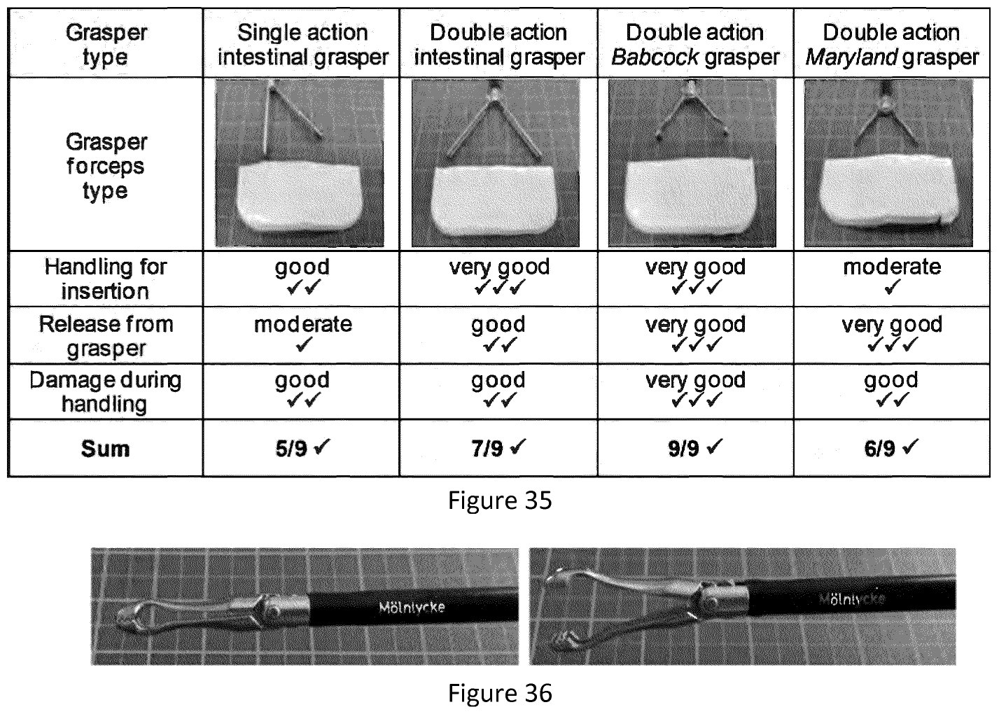

Based on the results of these detailed investigations (FIG. 35), the following aspects can be concluded. The performance of the single action intestinal grasper is not optimal. The asymmetric opening and closing is a disadvantage for the insertion as the opening angle impedes the release of the XaraColl implant from the grasper. The structure of the forceps is suitable and damage to the matrix is limited. The overall performance of the double action intestinal grasper is good and overcomes all disadvantages from the prior discussed single action grasper. The symmetric opening is more suitable for matrix insertion and the larger opening angle is essential for a good release from the grasper. The overall performance of the Babcock grasper is very good. The grasper has all the above-mentioned advantages from a double action grasper. The Babcock forceps design reduces the surface contact to the matrix to a minimum and thus likewise reduces surface damage. Due to the reduced contact, the release from the grasper is improved as well. The Maryland forceps design has some disadvantages. The forceps is short and does not support the insertion well.

In summary, four out of eight graspers are suitable for the laparoscopic application of XaraColl. Out of the four suitable graspers tested in more detail, the performance of the Babcock grasper outweighed the performances of the others. The advantages of the Babcock grasper (FIG. 36) compared to the others are as follows. The Babcock grasper type is a double action grasper with a rather large opening angle, facilitating the release of XaraColl. The Babcock forceps solely has teeth at the outer rim of the forceps. This reduces the surface contact with the XaraColl implant and thus surface damage. The reduced contact further facilitates the release of XaraColl from the grasper. The opening angle of the Babcock forceps is not too sharp (v-shaped), thus preventing additional squeezing and thus damage of the matrix. Moreover, this type of opening angle further eases the release.

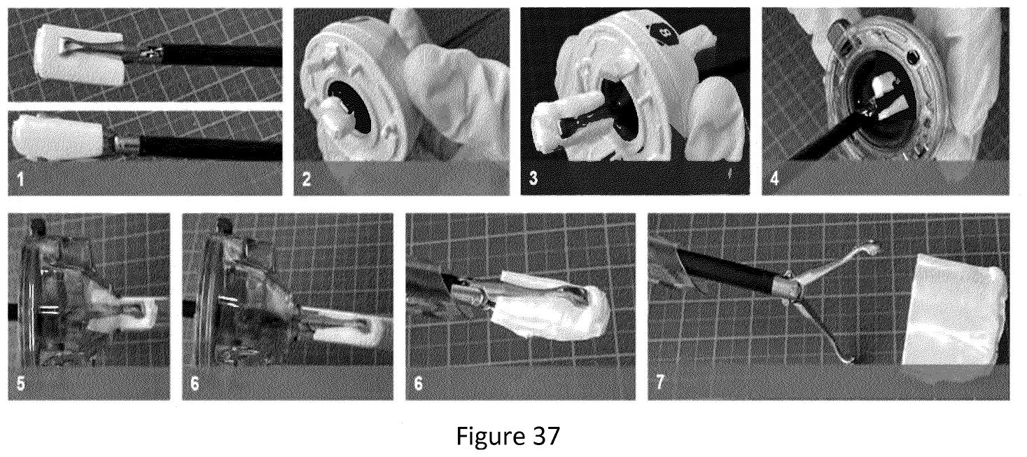

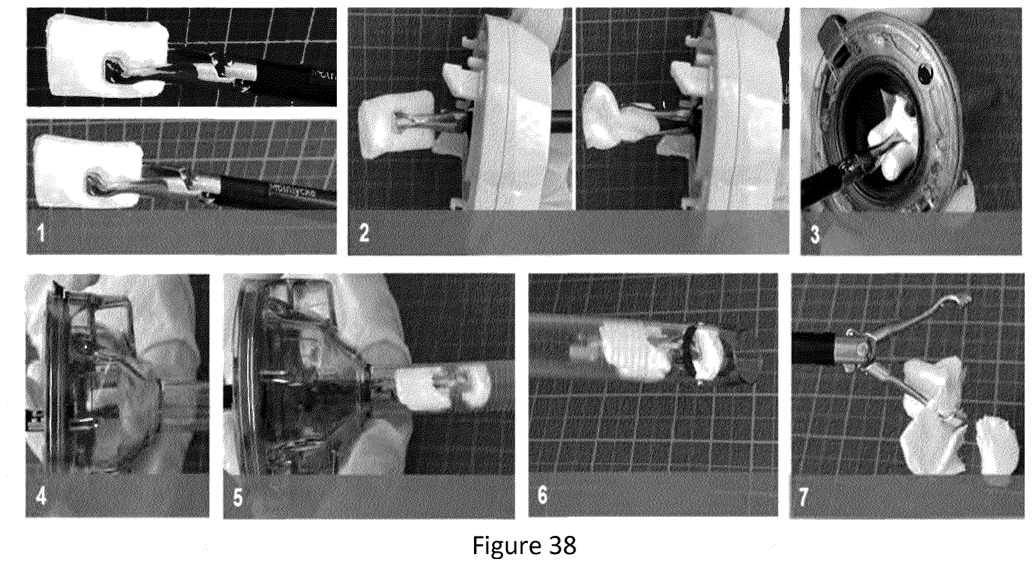

Apart from the grasper type, the correct handling of XaraColl with the grasper is likewise important. In cases wherein the grasper encloses large parts of the matrix (“long grip”), the XaraColl implant is adequately protected and the trocar insertion is successful. The XaraColl implant can be released without damage (FIG. 37). If the grasper does not enclose the matrix sufficiently enough (“short grip”), the XaraColl implant is more prone to damage during the insertion process (FIG. 38).

### Trocar Performance

### Trocar Seal:

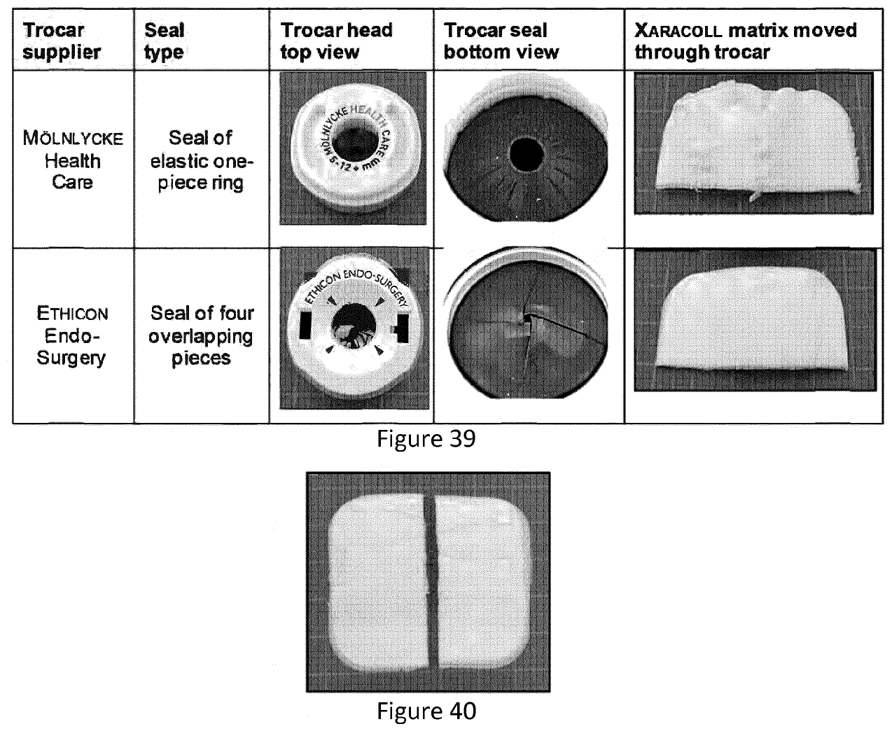

Within this study, the performances of trocars purchased from MOLNLYCKE Health Care or ETHICON Endo-Surgery with respect to the trocar seal were evaluated (FIG. 39). The resistance of a trocar seal has a strong impact of the degree of physical stress for the matrix. This stress can result in damage to the matrix surface. All tests of this evaluation were performed using a Babcock grasper.

The ETHICON four-piece seal gives only light resistance and the folded half XaraColl implant can be easily moved through with a grasper. The matrix shows no damage due to the insertion process step.

The resistance of the elastic one-piece ring seal by the MOLNLYCKE trocar is significantly higher. It is clearly more force required to move a folded, halved XaraColl implant through this type of seal by using a grasper. Additionally, some sort of surface damage is visible.

### Trocar Tube Diameter:

The diameter of a trocar tube can have a strong impact on the force that is required to move the folded half matrix through the tube with the grasper. However, the force that is required for an 8 mm trocar tube is still acceptable and easy to handle. Noteworthy, the XaraColl implant is more compressed in an 8 mm tube compared to a 12 mm tube, however, this increased compression did not show any visible additional damage. A side-by-side comparison of two XaraColl implant halves indicates that the tube diameters of 12 mm and 8 mm do not affect the visual appearance of the matrix (FIG. 40).

Concluding, all tested trocars are principally suitable. Clearly preferred is the ETHICON trocar due its patented seal construction. 12 mm and 8 mm trocar tubes are both suitable.

### Dissolution Testing

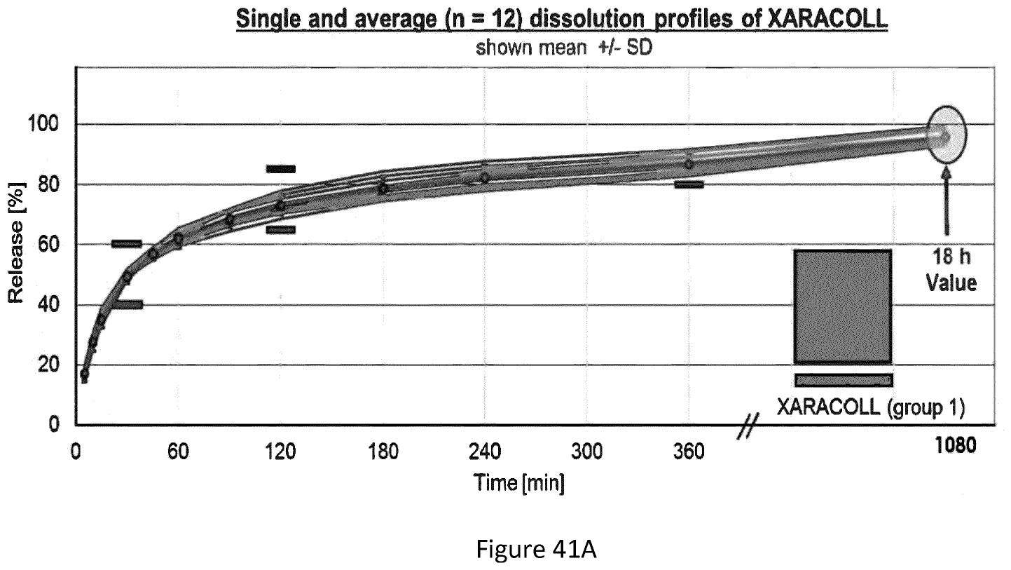

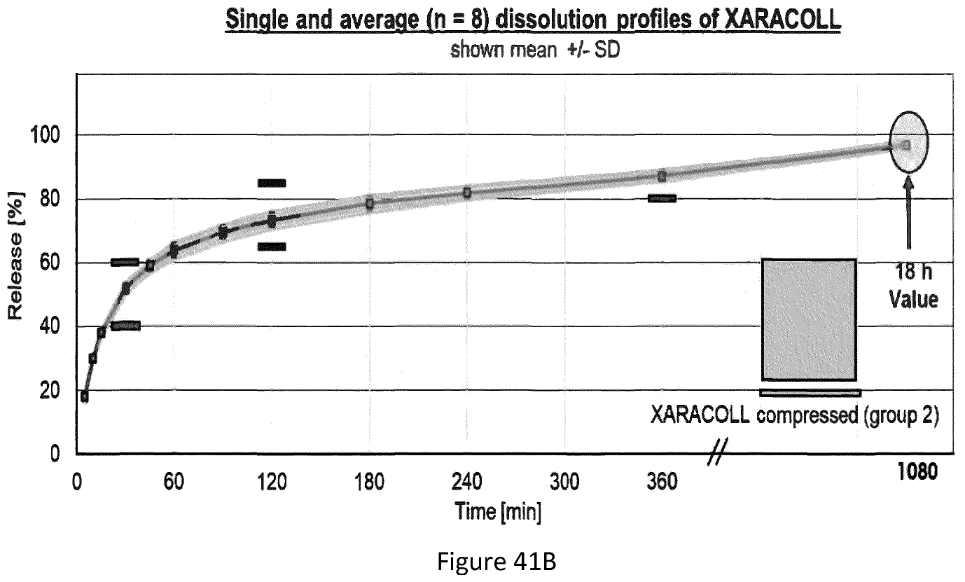

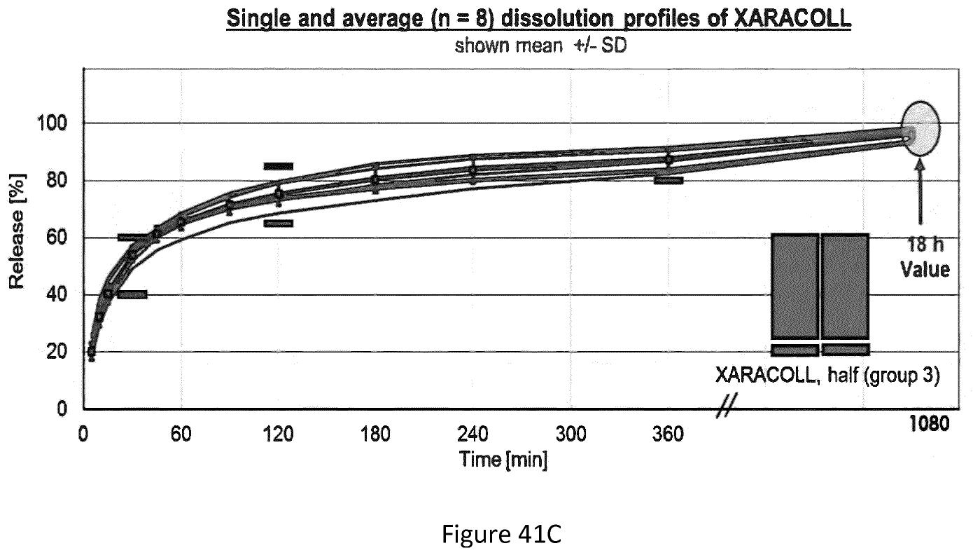

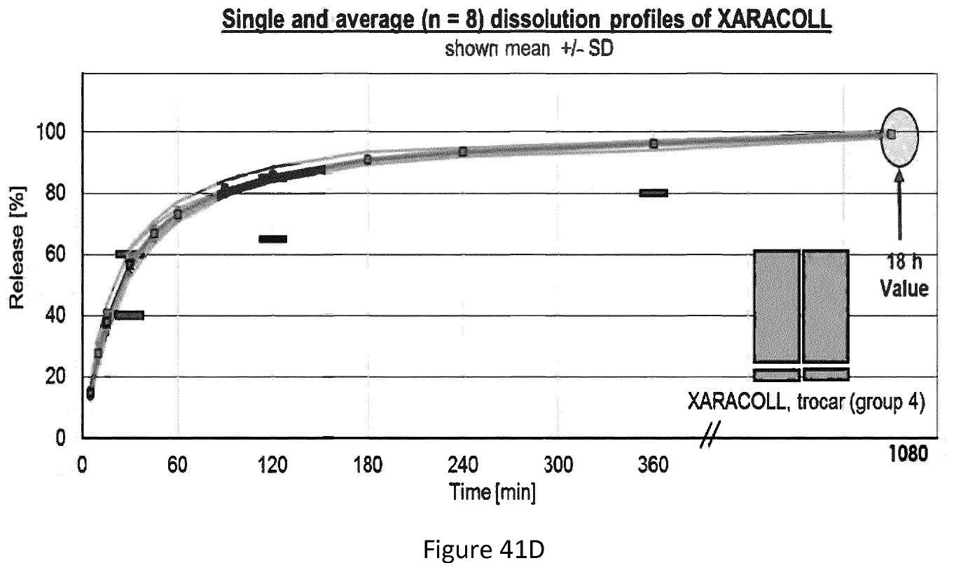

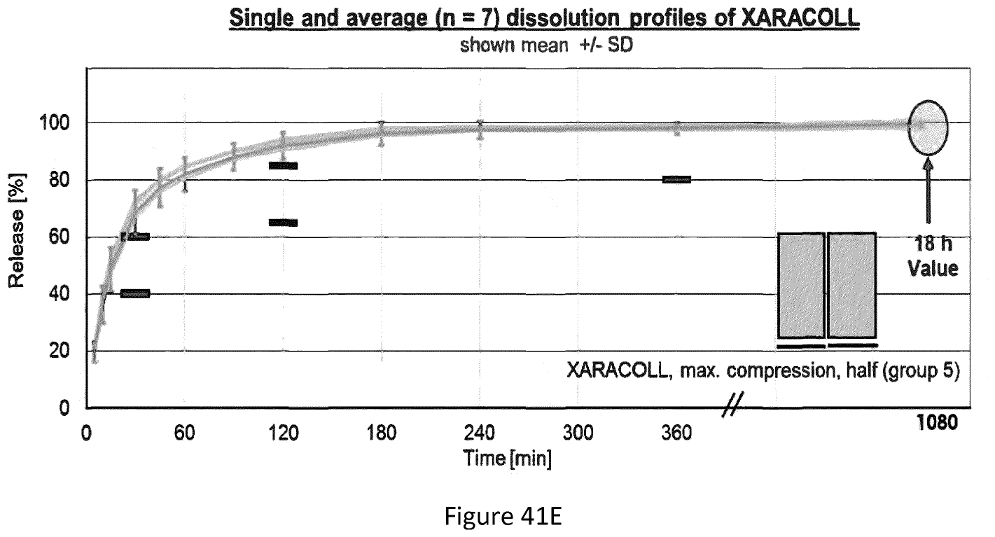

The results obtained from dissolution testing, followed by HPLC analysis, are presented in FIGS. 41A-41E and Tables 18-22. All test groups 1-5 resulted in single dissolution profiles with negligible scattering. The single dissolution profiles of XaraColl (group 1), XaraColl and partially compressed XaraColl (group 2) matched the acceptance criteria stated in Table 7 at each time point. In case of the halved XaraColl (group 3) and XaraColl, trocar (group 4) these acceptance criteria are not applicable as they are only valid for complete, uncut matrices and the results cannot be compared with group 1 and 2. The dissolution profiles of XaraColl, group 3 (i.e. cut matrices) were shown to be statistically equal as confirmed by f1/f2 statistical analysis. Likewise, f1/f2 statistical analysis confirmed that the results of XaraColl, group 4 (i.e. full procedure, compressed, cut, through trocar) can be considered similar/not dissimilar (Table 14). In contrast, the maximum compression of XaraColl (group 5) had a bigger impact on the dissolution properties compared to group 1-4, as a greatly accelerated API release was observed. Accordingly, the f1/f2 statistical evaluation proved the dissimilarity of this group from the XaraColl reference group 1, as the obtained values lie exterior the acceptance criteria.

TABLE 18
Dissolution data of XaraColl (group 1) from FIG. 41A.
XaraColl (n = 12)

| Time | Average | SD | RSD | a1 | a2 | a3 | a4 | a5 | a6 | a7 | a8 | a9 | a10 | a11 | a12 |
|---|---|---|---|---|---|---|---|---|---|---|---|---|---|---|---|
| [min] | [%] | [%] | [%] | [%] | [%] | [%] | [ %] | [ %] | [%] | [%] | [ %] | [%] | [%] | [%] | [%] |
| 5 | 17 | 1.5 | 9.0 | 16 | 17 | 16 | 15 | 18 | 18 | 16 | 16 | 20 | 19 | 17 | 15 |
| 10 | 28 | 1.7 | 6.2 | 26 | 27 | 27 | 25 | 28 | 28 | 27 | 27 | 31 | 30 | 29 | 26 |
| 15 | 35 | 1.7 | 4.8 | 33 | 33 | 35 | 33 | 35 | 36 | 35 | 34 | 39 | 37 | 36 | 34 |
| 30 | 49 | 1.1 | 2.2 | 48 | 48 | 49 | 48 | 49 | 50 | 49 | 49 | 52 | 51 | 49 | 50 |
| 45 | 57 | 1.2 | 2.2 | 57 | 57 | 56 | 56 | 55 | 57 | 56 | 56 | 59 | 58 | 56 | 59 |
| 60 | 62 | 1.7 | 2.7 | 61 | 63 | 60 | 61 | 59 | 62 | 61 | 61 | 64 | 62 | 61 | 65 |
| 90 | 68 | 2.2 | 3.3 | 68 | 71 | 66 | 68 | 64 | 69 | 67 | 67 | 71 | 69 | 66 | 72 |
| 120 | 73 | 2.7 | 3.7 | 73 | 76 | 71 | 73 | 69 | 73 | 71 | 71 | 75 | 74 | 70 | 78 |
| 180 | 79 | 3.0 | 3.9 | 79 | 83 | 76 | 79 | 74 | 79 | 76 | 77 | 81 | 80 | 75 | 84 |
| 240 | 82 | 3.1 | 3.7 | 83 | 86 | 79 | 82 | 78 | 82 | 80 | 81 | 85 | 79 | 79 | 88 |
| 360 | 87 | 2.9 | 3.3 | 87 | 89 | 85 | 87 | 83 | 86 | 84 | 86 | 91 | 84 | 84 | 92 |
| 1080 | 96 | 2.0 | 3.1 | 96 | 96 | 94 | 95 | 93 | 95 | 94 | 95 | 99 | 95 | 95 | 99 |

All single values comply with level 1 testing.

TABLE 19
Dissolution data of XaraColl compressed (group 2) from FIG. 41B.
XaraColl, compressed (n = 12)

| Time | Average | SD | RSD | b1 | b2 | b3 | b4 | b5 | b6 | b7 | b8 |
|---|---|---|---|---|---|---|---|---|---|---|---|
| [min] | [%] | [%] | [%] | [%] | [%] | [%] | [%] | [%] | [%] | [%] | [%] |
| 5 | 18 | 1.3 | 7.4 | 18 | 16 | 20 | 19 | 19 | 19 | 17 | 18 |
| 10 | 29 | 1.1 | 3.9 | 30 | 28 | 31 | 31 | 31 | 31 | 28 | 29 |
| 15 | 38 | 0.9 | 2.5 | 38 | 37 | 38 | 39 | 39 | 39 | 36 | 37 |
| 30 | 52 | 1.2 | 2.4 | 52 | 52 | 50 | 52 | 53 | 53 | 50 | 51 |
| 45 | 59 | 1.7 | 2.8 | 59 | 60 | 57 | 59 | 61 | 61 | 58 | 58 |
| 60 | 64 | 2.1 | 3.2 | 63 | 65 | 61 | 64 | 66 | 66 | 62 | 63 |
| 90 | 70 | 2.5 | 3.5 | 69 | 71 | 66 | 70 | 72 | 72 | 68 | 69 |
| 120 | 74 | 2.9 | 3.9 | 72 | 75 | 70 | 73 | 76 | 76 | 72 | 73 |
| 180 | 80 | 3.2 | 4.0 | 77 | 80 | 76 | 78 | 81 | 81 | 77 | 78 |
| 240 | 83 | 3.1 | 3.7 | 80 | 83 | 79 | 82 | 84 | 84 | 81 | 81 |
| 360 | 88 | 2.7 | 3.1 | 86 | 88 | 85 | 87 | 89 | 89 | 85 | 87 |
| 1080 | 97 | 1.5 | 1.6 | 96 | 98 | 96 | 97 | 97 | 97 | 95 | 96 |

All single values theoretically comply with level 1 testing (only used as an approximate reference point, since in principle the acceptance criteria stated in section 2.4 are only valid for complete, untreated matrices).

TABLE 20
Dissolution data of XaraColl, half (group 3) from FIG. 41C.
XaraColl, half (n = 8)

| Time | Average | SD | RSD | c1 | c2 | c3 | c4 | c5 | c6 | c7 | c8 |
|---|---|---|---|---|---|---|---|---|---|---|---|
| [min] | [%] | [%] | [%] | [%] | [%] | [%] | [%] | [%] | [%] | [%] | [%] |
| 5 | 20 | 2.9 | 14.6 | 19 | 18 | 23 | 25 | 19 | 17 | 22 | 18 |
| 10 | 32 | 3.5 | 10.9 | 30 | 29 | 36 | 39 | 31 | 30 | 34 | 30 |
| 15 | 40 | 3.4 | 8.4 | 37 | 37 | 44 | 46 | 39 | 39 | 42 | 39 |
| 30 | 54 | 2.6 | 4.8 | 49 | 52 | 56 | 57 | 55 | 55 | 54 | 55 |
| 45 | 61 | 2.6 | 4.3 | 56 | 60 | 62 | 63 | 63 | 63 | 61 | 63 |
| 60 | 66 | 2.9 | 4.4 | 59 | 65 | 66 | 66 | 67 | 68 | 65 | 69 |
| 90 | 72 | 3.3 | 4.6 | 65 | 72 | 71 | 71 | 74 | 74 | 70 | 75 |
| 120 | 75 | 3.9 | 5.2 | 69 | 76 | 74 | 73 | 79 | 79 | 73 | 80 |
| 180 | 80 | 4.6 | 5.7 | 73 | 81 | 78 | 77 | 86 | 84 | 78 | 86 |
| 240 | 84 | 4.5 | 5.4 | 77 | 85 | 80 | 80 | 89 | 88 | 82 | 89 |
| 360 | 87 | 3.7 | 4.3 | 82 | 88 | 84 | 83 | 92 | 90 | 87 | 91 |
| 1080 | 96 | 2.2 | 2.3 | 93 | 97 | 94 | 94 | 98 | 97 | 96 | 99 |

All single values theoretically comply with level 1 testing (only used as an approximate reference point, since in principle the acceptance criteria stated in section 2.4 are only valid for complete, untreated matrices).

TABLE 21
Dissolution data of XaraColl, trocar (group 4) from FIG. 41D.
XaraColl, trocar (n = 8)

| Time | Average | SD | RSD | d1 | d2 | d3 | d4 | d5 | d6 | d7 | d8 |
|---|---|---|---|---|---|---|---|---|---|---|---|
| [min] | [%] | [%] | [%] | [%] | [%] | [%] | [%] | [%] | [%] | [%] | [%] |
| 5 | 15 | 1.7 | 11.8 | 15 | 16 | 17 | 16 | 12 | 13 | 14 | 15 |
| 10 | 28 | 3.3 | 11.8 | 27 | 29 | 33 | 32 | 23 | 25 | 26 | 28 |
| 15 | 38 | 3.8 | 10.0 | 36 | 38 | 44 | 43 | 33 | 35 | 36 | 37 |
| 30 | 57 | 3.3 | 5.9 | 55 | 57 | 61 | 62 | 52 | 55 | 55 | 56 |
| 45 | 67 | 2.5 | 3.7 | 65 | 68 | 70 | 71 | 63 | 66 | 66 | 66 |
| 60 | 73 | 2.0 | 2.8 | 71 | 74 | 75 | 77 | 71 | 72 | 73 | 72 |
| 90 | 81 | 1.6 | 2.0 | 79 | 81 | 81 | 84 | 79 | 80 | 81 | 80 |
| 120 | 86 | 1.4 | 1.7 | 84 | 86 | 85 | 89 | 85 | 86 | 86 | 85 |
| 180 | 91 | 1.3 | 1.5 | 89 | 91 | 89 | 94 | 90 | 91 | 91 | 90 |
| 240 | 93 | 1.1 | 1.2 | 92 | 93 | 92 | 95 | 94 | 94 | 94 | 94 |
| 360 | 96 | 1.1 | 1.1 | 96 | 96 | 94 | 97 | 97 | 97 | 97 | 96 |
| 1080 | 99 | 0.9 | 0.9 | 99 | 98 | 98 | 99 | 100 | 100 | 100 | 100 |

*All single values theoretically comply with acceptance level 2 (only used as an approximate reference point, since in principle the acceptance criteria stated in section 2.4 are only valid for complete, untreated matrices)

TABLE 22
Dissolution data of XaraColl, max. compression, half (group 5) from FIG. 41E.
XaraColl, max. compression, half (n = 7)

| Time | Average | SD | RSD | d1 | d2 | d3 | d4 | d5 | d6 | d7 |
|---|---|---|---|---|---|---|---|---|---|---|
| [min] | [%] | [%] | [%] | [%] | [%] | [%] | [%] | [%] | [%] | [%] |
| 5 | 21 | 1.2 | 6.0 | 20 | 21 | 20 | 21 | 20 | 23 | 22 |
| 10 | 38 | 1.9 | 5.0 | 35 | 39 | 37 | 39 | 37 | 41 | 40 |
| 15 | 51 | 2.2 | 4.3 | 47 | 52 | 50 | 52 | 50 | 54 | 53 |
| 30 | 71 | 2.2 | 3.1 | 67 | 72 | 71 | 72 | 71 | 73 | 72 |
| 45 | 79 | 1.7 | 2.1 | 76 | 80 | 80 | 80 | 80 | 80 | 80 |
| 60 | 84 | 1.6 | 1.9 | 80 | 85 | 84 | 85 | 84 | 85 | 85 |
| 90 | 90 | 1.2 | 1.3 | 87 | 90 | 90 | 90 | 90 | 90 | 90 |
| 120 | 93 | 1.5 | 1.7 | 90 | 95 | 94 | 95 | 94 | 93 | 93 |
| 180 | 97 | 1.0 | 1.1 | 95 | 98 | 98 | 98 | 98 | 97 | 97 |
| 240 | 98 | 0.6 | 0.6 | 97 | 99 | 99 | 99 | 99 | 98 | 99 |
| 360 | 99 | 0.6 | 0.6 | 97 | 99 | 99 | 99 | 99 | 99 | 99 |
| 1080 | 100 | 1.0 | 1.0 | 98 | 100 | 99 | 100 | 99 | 101 | 101 |

All single values theoretically comply with level 3 (only used as an approximate reference point, since in principle the acceptance criteria stated in section 2.4 are only valid for complete, untreated matrices).

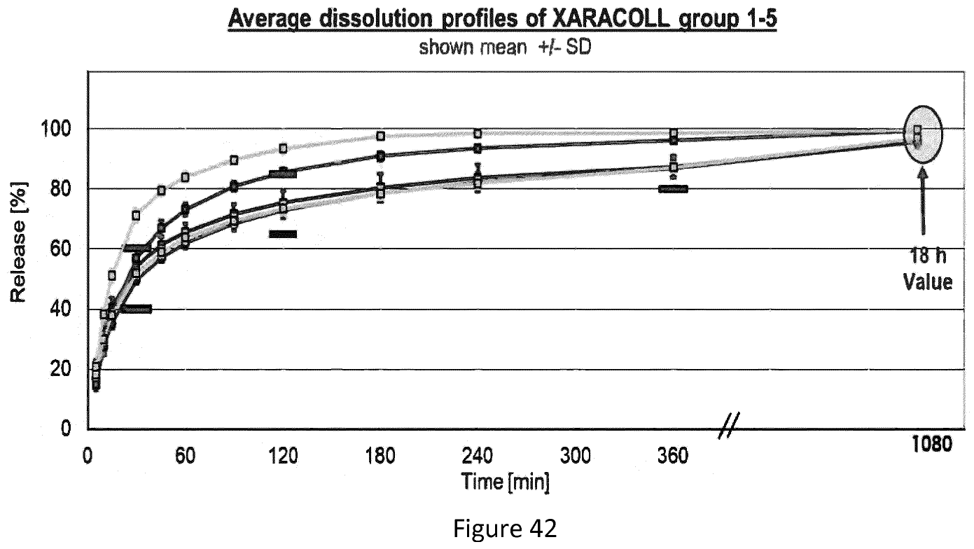

The average dissolution profiles of test groups 1-4 (FIG. 42) resembled each other. Even though the acceptance criteria stated in Table 7 are only valid for complete, untreated matrices (only group 1), the compressed and halved (groups 2 and 3, respectively) implants as well as those, that have been passed through the trocar (group 4) theoretically complied with test levels 1 and 2. Moreover, these groups were similar/not dissimilar according to f1/f2 statistical analysis. In contrast, group 5, i.e. the completely and controlled compressed was different compared to the complete implants in group 1, as revealed by f1/f2 statistical test. This difference is most likely due to a reduction in diffusion path lengths caused by the compression of the implants. However, this effect was only visible for the completely compressed XaraColl implants (group 5) (which persistently remained at highly reduced thickness), whereas the partial (partial reversible) compression (group 2 and 4) which was followed by largely rebounding of thickness did not trigger this effect. Therefore, when XaraColl was only partially compressed (group 2 and 4), the difference in drug release was negligible with the f1/f2 statistical analysis indicating similar/not dissimilar curves. In case XaraColl was fully compressed (group 5), the impact on the drug release was pronounced and the latter was accelerated due to tremendously decreased diffusion path lengths. Accordingly, the f1/f2 statistical analysis revealed dissimilarity/non similarity compared to XaraColl group 1.

The detailed results of the f1/f2 statistical analysis are listed in Table 23 wherein groups 2-5 were compared with the regular XaraColl dissolution profile (group 1). The f1/f2 statistical analysis comparing group 2, 3, and 4 with the complete and untreated XaraColl (group 1) confirmed that the dissolution profiles of the respective test groups were “similar” (f2) as well as “not dissimilar” (f1). That means that, even though the XaraColl matrices of test groups 2-4 (including those moved through the trocar) experience a non-negligible amount of physical stress, the resulting dissolution properties were comparable and thus indicates that the drug release in vitro will stay unchanged. In contrast, the positive control group 5 (XaraColl, max. compression, half) indicated dissimilarity, as both factors, f1 and f2, mismatched the acceptance criteria.

TABLE 23
f1/f2 statistical analysis comparing groups 2-5 with group
1 with dissimilar results in bold text.

| | | | | Acceptance | |
|---|---|---|---|---|---|
| | | | | criterion | |
| Compared groups | | f1 | f2 | f1 | f2 |
| XaraColl | XaraColl, compressed | 3.2 | 82.9 | 0-15 | 50-100 |
| | XaraColl, half | 5.6 | 71.6 | | |
| | XaraColl, trocar | 15.0 | 51.2 | | |
| | XaraColl, max. compression, | 37.3 | 37.1 | | |
| | half | | | | |

### Result Summary

Batches: All used batches were suitable for the laparoscopic application of XaraColl.

Process of compression and folding: The process of compression and folding allows the laparoscopic application of XaraColl without moistening pieces or dividing the implant into smaller pieces than halves. Additional improvements of the compression tool could improve the compression and fold process.

Grasper: All pre-selected graspers of this evaluation (4 out of 8 grasper types) are suitable. Most suitable is the Babcock grasper.

Trocar: Trocars similar to the ETHICON trocar patented seal construction result in minimal damage. 12 mm and 8 mm trocar tubes are both suitable. A summary of the results from the batches, folding/compression, grasper, and trocar studies is found in Table 24.

Dissolution: Statistical analysis confirmed that partial controlled compression (group 4) with the compression tool and a defined compression gap did not significantly influence XaraColl's drug release. In contrast to that, uncontrolled, complete compression of XaraColl (group 5) should be avoided, as significant differences compared to untreated XaraColl (group 1) were found. The result further underlined the suitability of the sample preparation technique of XaraColl for laparoscopic application as described in this Example.

TABLE 24
Result summary (excluding dissolution).

| Object of | Evaluation | | | |
|---|---|---|---|---|
| evaluation | Parameter | Values | Evaluation | Result |
| 5 XaraColl | Initial thickness | 4.4 to 5.0 mm | Suitability | Folding with |
| Batches | Re-expanded | 2.2 to 2.5 mm | for folding | correct side |
| | thickness | | | up results in |
| | Resistance to | 2.6 to 3.5N | | minimal |
| | pressure | | | damage |
| | Force to break | 1.4 to 1.8N | | |
| Compression | Re-expanded | 2.4 to 2.6 mm | Suitability | |
| process, | thickness | | for folding | |
| Compression time | worst case @ | | | |
| (1, 15, 120 s) | 1 s | | | |
| Single action | Different | Quality of | Over all | Good suitable |
| Intestinal grasper | performance | handling and | suitability | |
| Double action | criteria | performance | for XaraColl | Good suitable |
| Intestinal grasper | | | insertion | |
| Double action | | | | Best suitable |
| Babcock grasper | | | | |
| Double action | | | | Good suitable |
| Maryland grasper | | | | |
| Grasper with | | | | Can be used |
| straight grasping | | | | (not optimal) |
| forceps-fenestrated | | | | |
| Grasper with rat | | | | Can be used |
| tooth grasping | | | | (not optimal) |
| forceps | | | | |
| Grasper with | | | | Can be used |
| duckbill grasping | | | | (not optimal) |
| forceps | | | | |
| Grasper with clinch | | | | Can be used |
| grasping forceps | | | | (not optimal) |
| ETHICON 4 piece | | No damage | Suitability | Best suitable |
| trocar seal | | minimum force | for | |
| MÖLNLYCKE | Damage at | light damage | laparoscopic | Suitable |
| single piece | matrix and | moderate force | application | |
| trocar seal | required force | | for | |
| Trocar tube | to move | No damage | XaraColl | Best suitable |
| diameter 12 mm | through | minimum force | | |
| Trocar tube | | No damage | | Good suitable |
| diameter 8 mm | | moderate force | | |

### Conclusions

Within this study, a sample preparation technique for the laparoscopic application of XaraColl was developed. This preparation technique is based on three key steps. First, the XaraColl implant is compressed using a compression tool featuring a defined compression gap of approximately 0.8 mm height. After release of the compression force, the XaraColl implant re-expands to a thickness between 2 and 2.5 mm. Subsequently, the matrix can be easily cut with scissors and the halved matrices are folded twice. These compressed, halved, and folded matrices can then be inserted into a trocar using a grasper. During this investigation, some important points were determined:

For the sufficient compression of XaraColl, it is preferable to use the palms to put sufficient pressure on the lid. For complete compression, at least 30 kg of force are necessary.

The choice of grasper is likewise important. Suitable graspers are double action graspers with no or small tooth and not too sharp opening angles. The Babcock type grasper fulfills all of these requirements and was found to be very suitable for the handling of XaraColl.

The seal of the utilized trocar is important as well. The performance of the ETHICON seal with four overlapping pieces is preferred and outweighed the performance of the elastic one-piece MOLNLYCKE seal.

In summary, this study presents a successful sample preparation technique for the laparoscopic application of XaraColl which avoids further cutting or moistening of XaraColl.

Dissolution testing of five different test groups (untreated XaraColl matrices (group 1), compressed matrices (0.8 mm, group 2) and halved matrices (group 3) as well as compressed, halved XaraColl matrices, which were inserted into a trocar (group 4)) was performed. Additionally, a completely compressed (“paper thickness”) and halved XaraColl matrix (group 5) was characterized. All test groups 1-5 resulted in homogeneous dissolution profiles with negligible scattering. Moreover, the study confirmed the suitability of the new handling procedure as f1/f2 statistical analysis revealed similar/not dissimilar dissolution profiles of XaraColl, group 4 (trocar) compared to untreated XaraColl (group 1) which is an improvement on previous sample preparation techniques wherein XaraColl matrices were cut into quarters and moistened. In case of the completely compressed XaraColl (group 5, “paper thickness”), dissolution properties clearly different from the untreated XaraColl were observed as proven by f1/f2 analysis. Thus, it can be summarized, that complete and uncontrolled compression of XaraColl (group 5) should be avoided, whereas compression of XaraColl with the developed compression technique and a defined compression gap of approximately 0.8 mm (partial compression of XaraColl, group 4) does not change the drug release properties of XaraColl. Thus, the XaraColl preparation technique described above is suitable for laparoscopic application of XaraColl and maintains XaraColl's unique drug release characteristics.

### Example 9: Laparoscopic Application of XaraColl—Sample Preparation and Performance Evaluation of XaraColl Treated with 1st to 5th Generation Compression Tool

### Introduction

In order to enable the laparoscopic use of XaraColl, it is vital to investigate an approach for the insertion of XaraColl through a trocar as well as to evaluate the characteristics of the XaraColl implant after this process. Example 8 describes a straightforward strategy for the insertion of XaraColl through a trocar where this procedure includes pressing of the XaraColl implant to a thickness of 0.75 mm with a compression tool. The compressed matrix is then cut into two halves, which are folded twice. Subsequently, XaraColl can be moved through a trocar with 8- or 12-mm diameter without major damage by using a grasper. However, this method exposes XaraColl to physical stress, i.e. folding, cutting, compression, and rubbing, which might influence the drug release properties. Therefore, Example 8 also includes an investigation of the influence of this manipulation on the dissolution profiles. The study suggested that the drug release characteristics of the samples, which were treated as described above, are maintained, as determined by f1/f2 statistical analysis.

Encouraged by these promising results, this Example focuses on the further development and fine-tuning of the compression tool. More precisely, a compression tool prototype series is introduced and the performance of these tools are investigated, including the handling, the necessary compression force, and the compression timing. Additionally, the effects of these on the dissolution properties of XaraColl are investigated.

### Methods

### Sample Preparation Technique

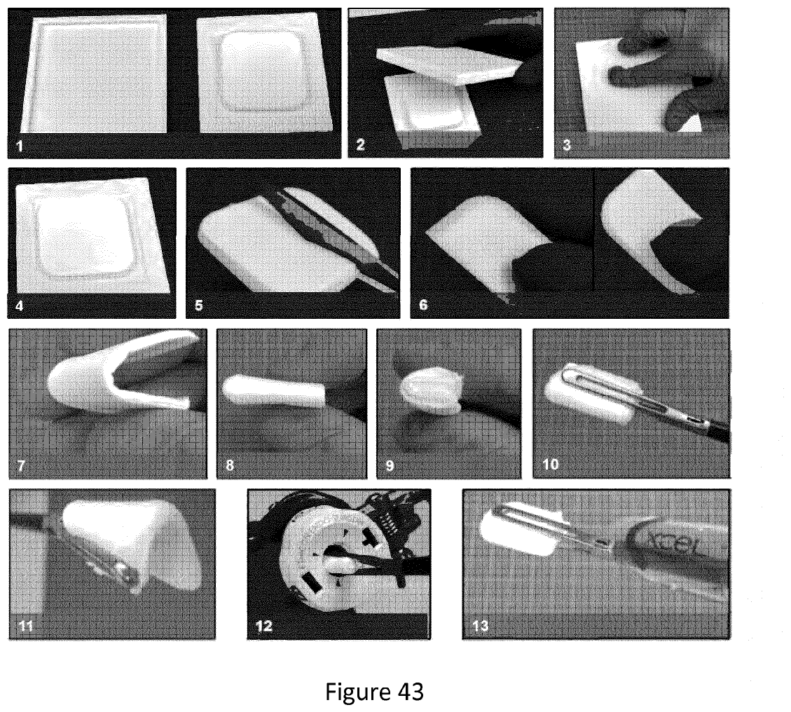

The sample preparation technique applied for this study is described in detail in Example 8. In order to insert XaraColl into a trocar, the fragility and roughness of the implant have to be reduced to a certain extent, so that the XaraColl matrix can be folded easily and without rupturing. This can be achieved through the compression of XaraColl with a so-called compression tool with a defined compression gap. Upon release of the compression force, the XaraColl implant re-expands up to a thickness of 2.0 to 2.5 mm. Subsequently, the compressed XaraColl implant can be easily cut into two halves by using scissors. The two halves are folded twice and the folded bundle can be finally inserted through the trocar by using a grasper. Noteworthy, it is crucial that the bottom side of the XaraColl implant is on the outside of the folding as the top side and the bottom side of the XaraColl implant are not identical. In detail, the single steps of this preparation procedure include (illustrated in FIG. 43).
1) XaraColl is placed on the bottom part of a compression tool.
2) The top part of the compression tool serves as a lid onto which the palms are placed for compression.
3) The compression force is maintained for a few seconds.
4) After compression, the XaraColl implant re-expands to a certain extent (2.0-2.5 mm)
5) The XaraColl implant is cut into two halves.
6) The top side (left) and the bottom side of the XaraColl implant are not identical.
7) It is crucial that the bottom side of the XaraColl implant is located outside the folding.
8) The two XaraColl halves are folded separately.
9) Second folding of the XaraColl implant.
10) The halved, folded XaraColl implants are held with a grasper.
11) Side view of the halved, folded XaraColl implant held with the grasper.
12) The XaraColl implant is inserted into the trocar with the help of the grasper.
13) The XaraColl implant is released from the grasper. Upon release, the XaraColl implant unfolds.

### Compression Tool Prototypes

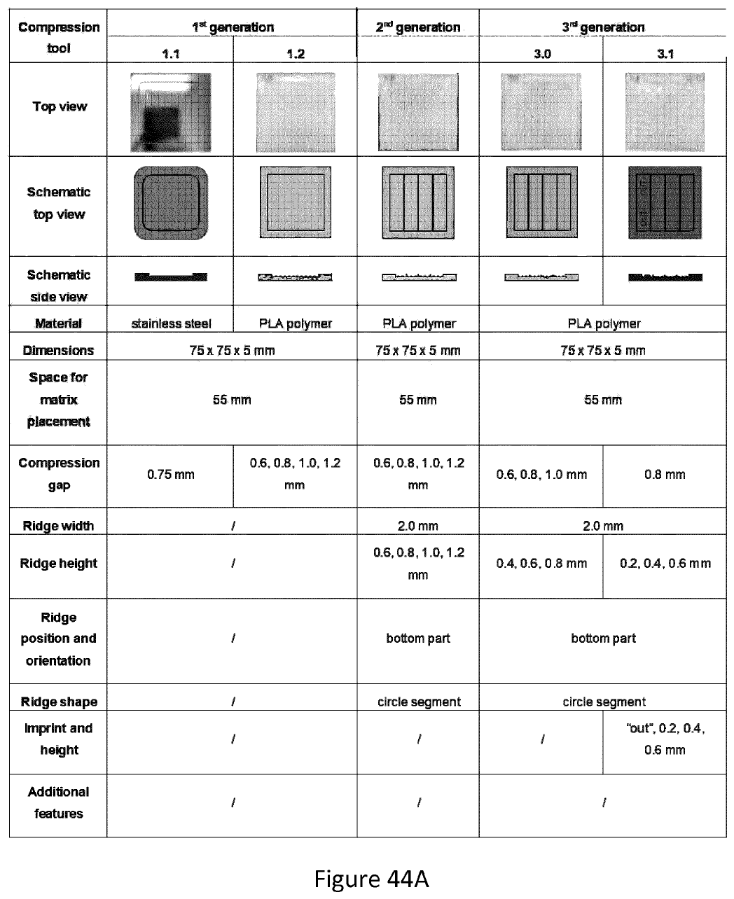

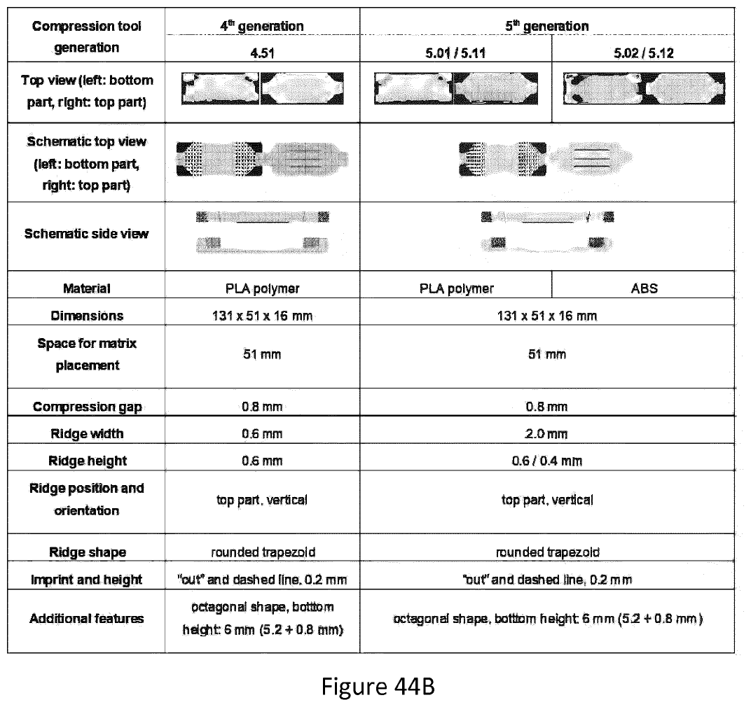

A prototype series of compression tools was designed (FIGS. 44A and 44B). Each compression tool is constituted of a bottom part, onto which the implant is placed for compression, and a corresponding top part. This top part serves as a lid, onto which the palms are placed for compression. The respective tools were categorized into generations (generation 1-4), which differ in the material as well as in the height of the compression gap and the general design. Some of the tools additionally feature ridges (generation 2 to 4), which should facilitate the folding process described in Example 8.

The performance of each compression tool is evaluated by analyzing the foldability of the XaraColl implant after compression. Thereby, the following parameters are taken into account:
Necessary force required for the compression of XaraColl
Impact of the compression gap size on the foldability of XaraColl
Impact of the compression time on the foldability of XaraColl
Impact of matrix equilibration at high and low relative humidity on the foldability of XaraColl

Since the drug release properties of XaraColl represent a highly important parameter, which might be influenced by physical stress, dissolution studies were conducted as well. Therefore, XaraColl implants, which were treated as described in Example 8 (compressed, cut, folded and pushed through a trocar), were characterized.

### Reagents and Instruments

The following XaraColl batches are used for this study: 18042611, 21001406, 20000607, 19002602, 21032106, 21031406, 21011207.

A universal tester Zwicki 500 N (Zwicki/Roll, serial number 200134) and a climatic chamber KBF 240 (Binder, serial number 400692) were used. All further reagents and instruments used are listed in Table 8 and Table 9 of Example 8, respectively.

The impact of the sample preparation described in Example 8, using the above described compression tools, on the API release was evaluated. For this purpose, dissolution test groups 1-5 were be characterized, which differ in the utilized compression tool, the trocar size as well as the matrix equilibration conditions (Table 25).

### Dissolution Studies

TABLE 25
Dissolution test groups investigated in this study.

| | | Com- | | Matrix | |
|---|---|---|---|---|---|
| | Compression | pression | Trocar | equilibration | XaraColl |
| Group | tool | gap | size | conditions | batch |
| 0 | / | / | / | / | 20000607/ |
| | | | | | 21032106/ |
| | | | | | 21031406/ |
| | | | | | 21011207 |
| 1 | 1^{st} generation 1.2 | 0.75 mm | 12 mm | / | 20000607 |
| 2a | 2^{nd} generation | 0.80 mm | 8 mm | / | 20000607 |
| 2b | 2^{nd} generation | 0.80 mm | 12 mm | / | 20000607 |
| 2c | 2^{nd} generation | 0.80 mm | 8 mm | 20% r.h. | 21032106 |
| 2d | 2^{nd} generation | 0.80 mm | 12 mm | 20% r.h. | 21032106 |
| 3a | 3^{rd} generation 3.0 | 0.80 mm | 8 mm | 20% r.h. | 21032106 |
| 3b | 3^{rd} generation 3.0 | 0.80 mm | 12 mm | 20% r.h. | 21032106 |
| 3c | 3^{rd} generation 3.0 | 0.80 mm | 8 mm | 80% r.h. | 21032106 |
| 3d | 3^{rd} generation 3.0 | 0.80 mm | 12 mm | 80% r.h. | 21032106 |
| 5 | 5^{th} generation | 0.80 mm | 8 mm | / | 21011207 (5a) |
| | 5.02 | | | | 21032106 (5b) |
| | | | | | 21031406 (5c) |
| | | | | | 20000607 (5d) |

All dissolution experiments were performed using an Agilent dissolution system with dissolution workstation. Important system parameters applied for this study are summarized in Table 11 of Example 8. Samples were drawn at time points t=5, 10, 15, 30, 45, 60, 90, 120, 180, 240, 360, and 1080 minutes, which were collected in a HPLC vial tray and subsequently characterized via HPLC analyses. The settings for these analyses are according Table 12 of Example 8. For data evaluation, an external Bupivacaine HCl standard was applied. All calculations were conducted using a reviewed Excel-sheet.

### Calculation of f1 and f2 Factors and Interpretation

The f1 and f2 factors were determined and interpreted as outline in Example 8, using Equation 1 and Table 8.

### Results and Discussion

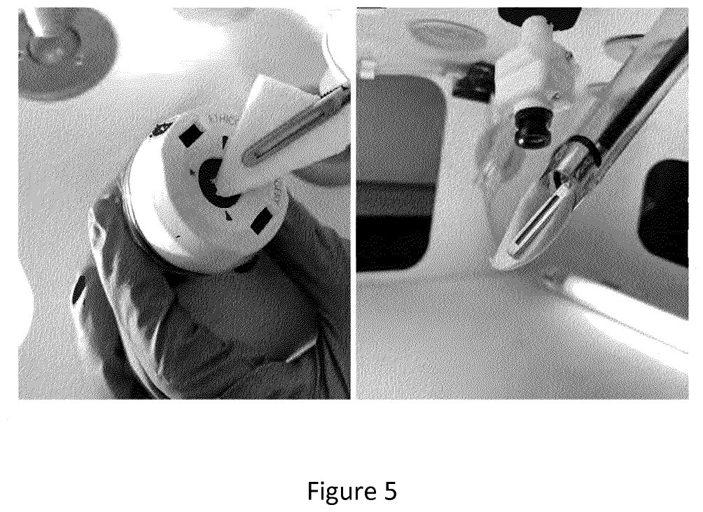

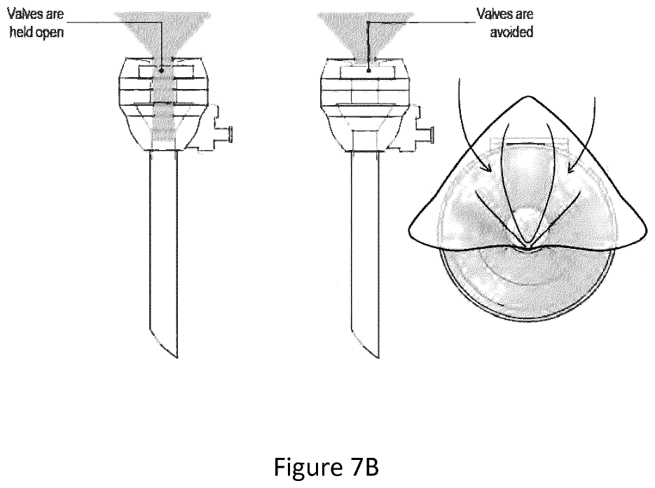

The results obtained from dissolution testing, followed by HPLC analysis, are presented in FIGS. 3-19 and Tables (10-26). All test groups 1-5 result in single dissolution profiles with negligible scattering. The single dissolution profiles of XaraColl batches 20000607, 21032106, 21031406y, and 21011207 (group 0, FIGS. 3-7, tables 10-14) match the acceptance criteria stated in Table 7. On the contrary, XaraColl, trocar (group 4) results in a slightly accelerated drug release. This means that the single dissolution results of XaraColl, trocar (group 4) do not match and exceed the acceptance criterion at time point 30 min in two out of eight cases and in four out of eight cases at t=120 min. At t=360 min, group 4 fulfills the acceptance criterion. The maximum compression of XaraColl (group 5) had an even bigger impact on the dissolution properties compared to group 1-4, as a greatly accelerated API release was observed. Thus, the single dissolution results of group 5 do not match the acceptance criteria at t=30 and t=120 min in each case (seven out of seven). Importantly, these results are not OS, since the acceptance criteria are only valid for complete XaraColl matrices. At t=360 min, all single curves match the specification.

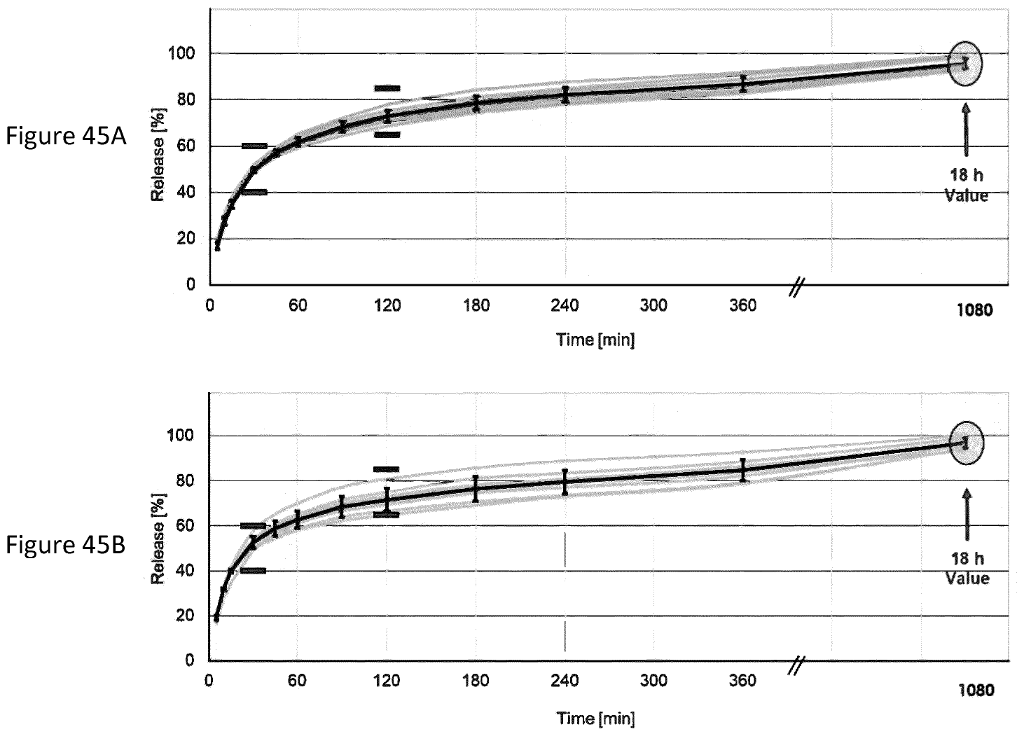

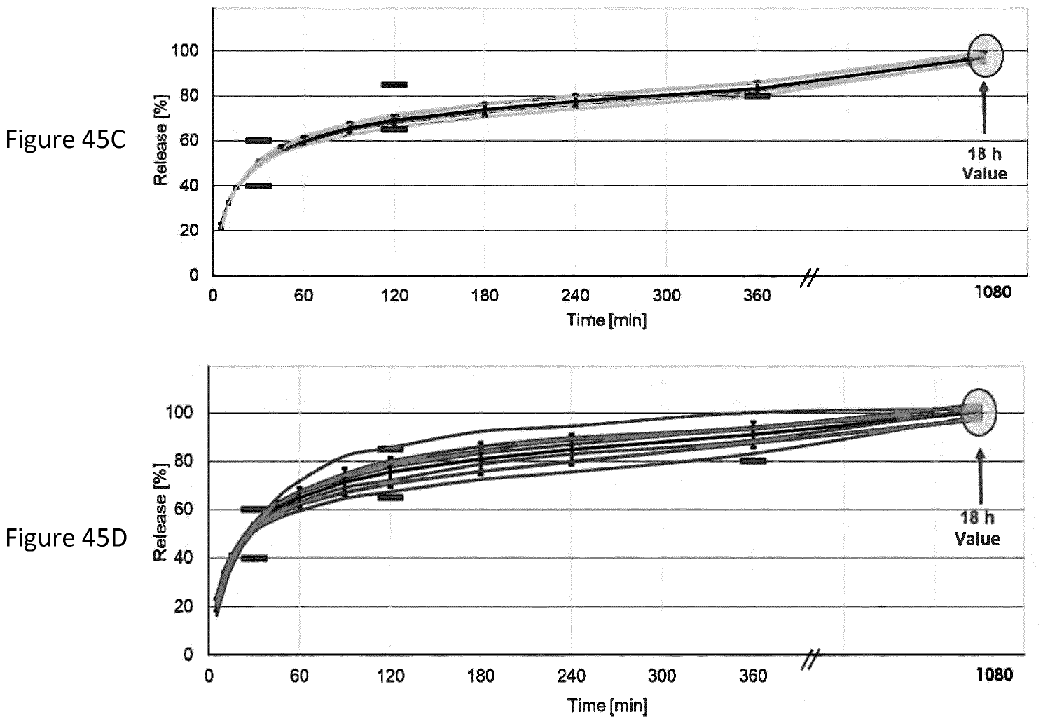

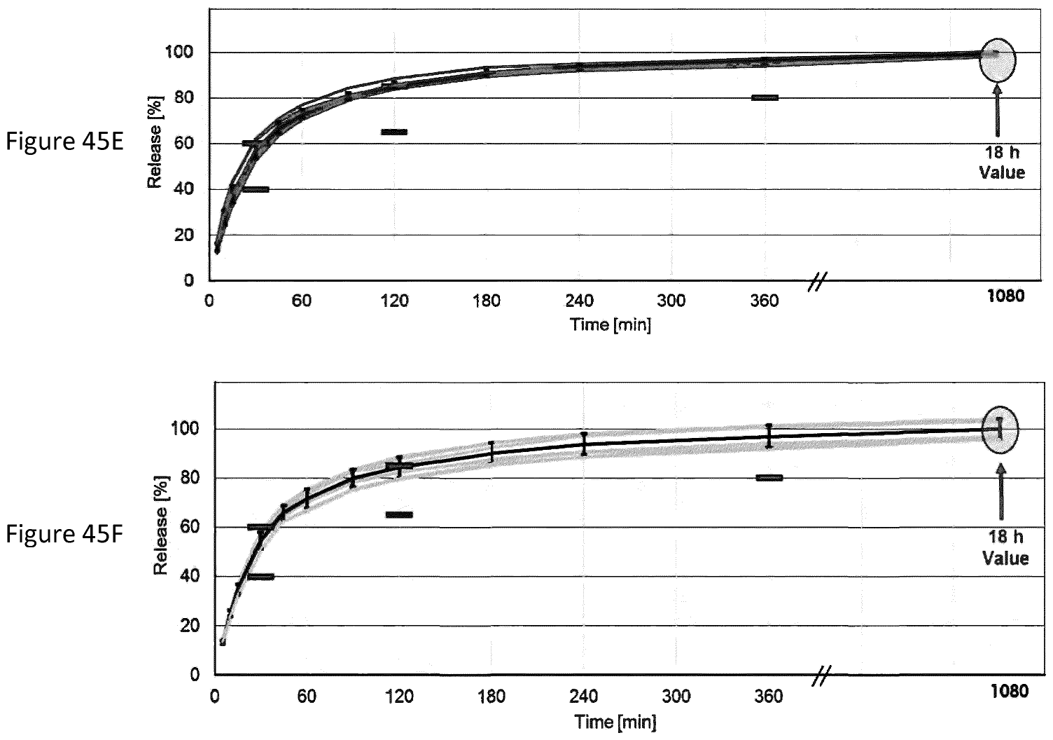

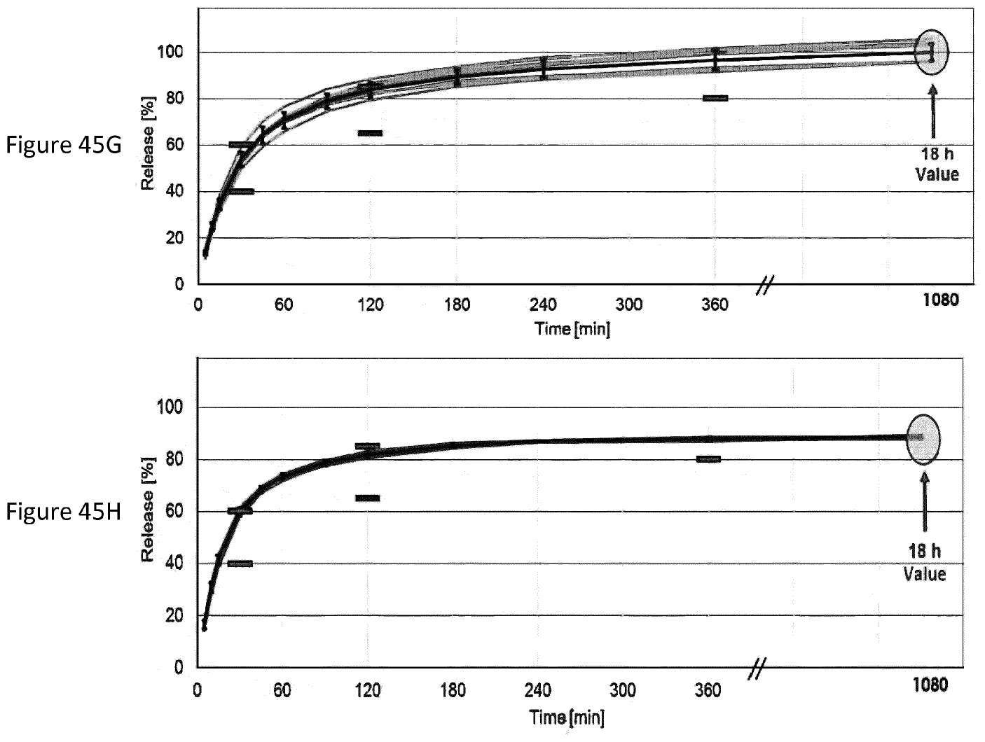

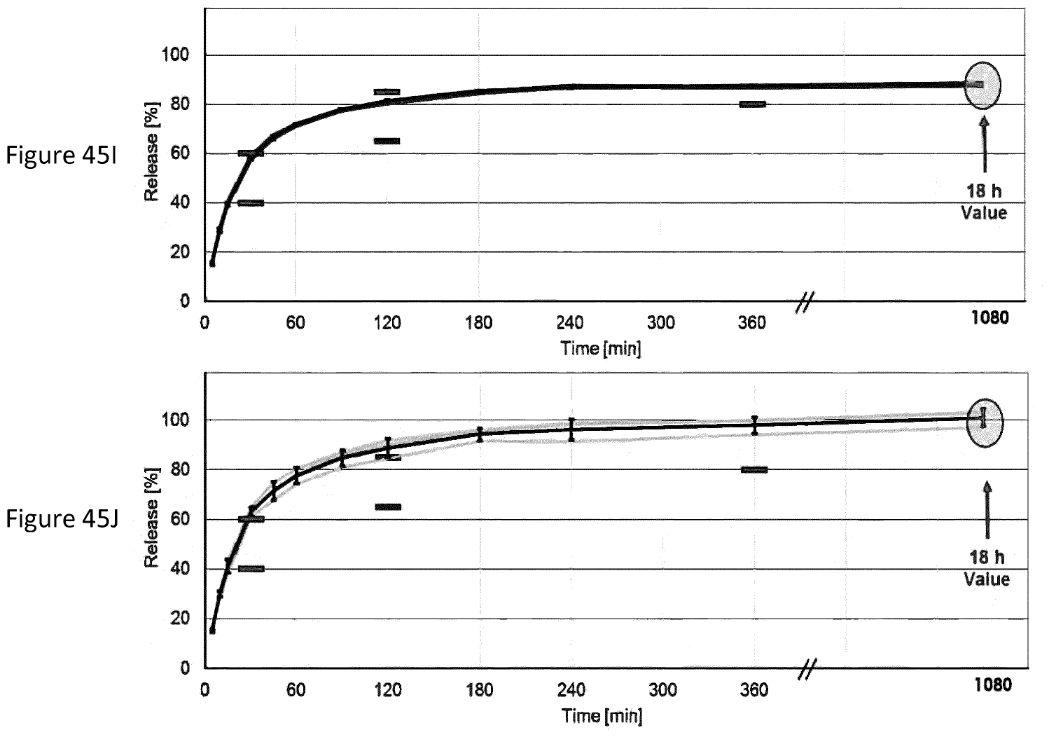

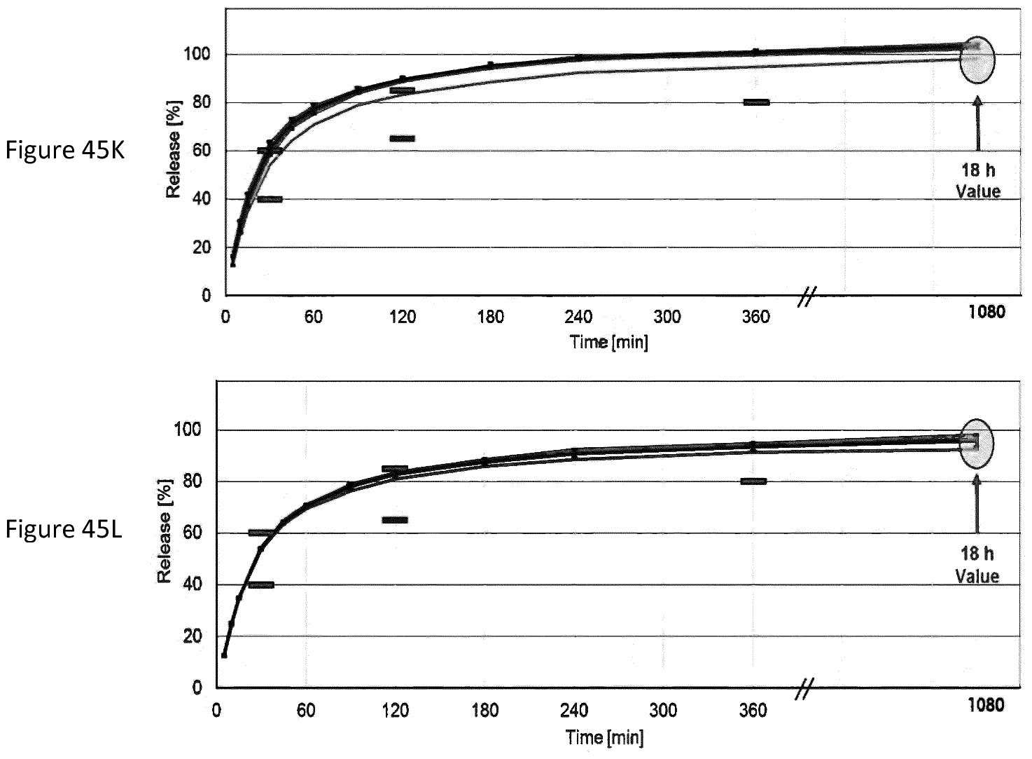

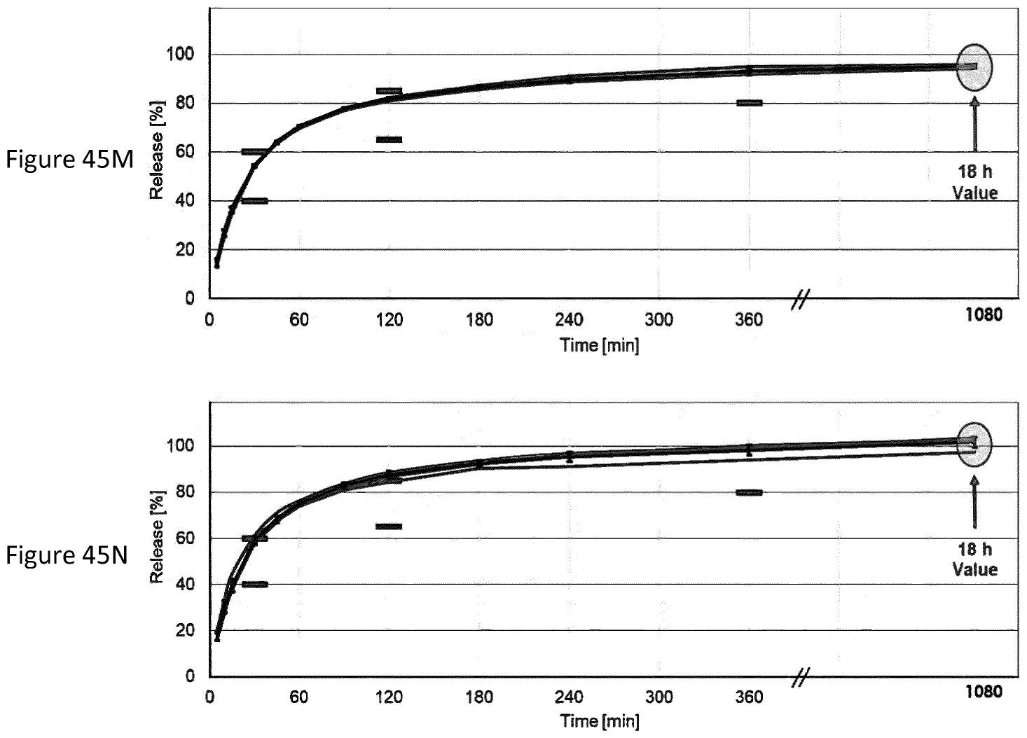

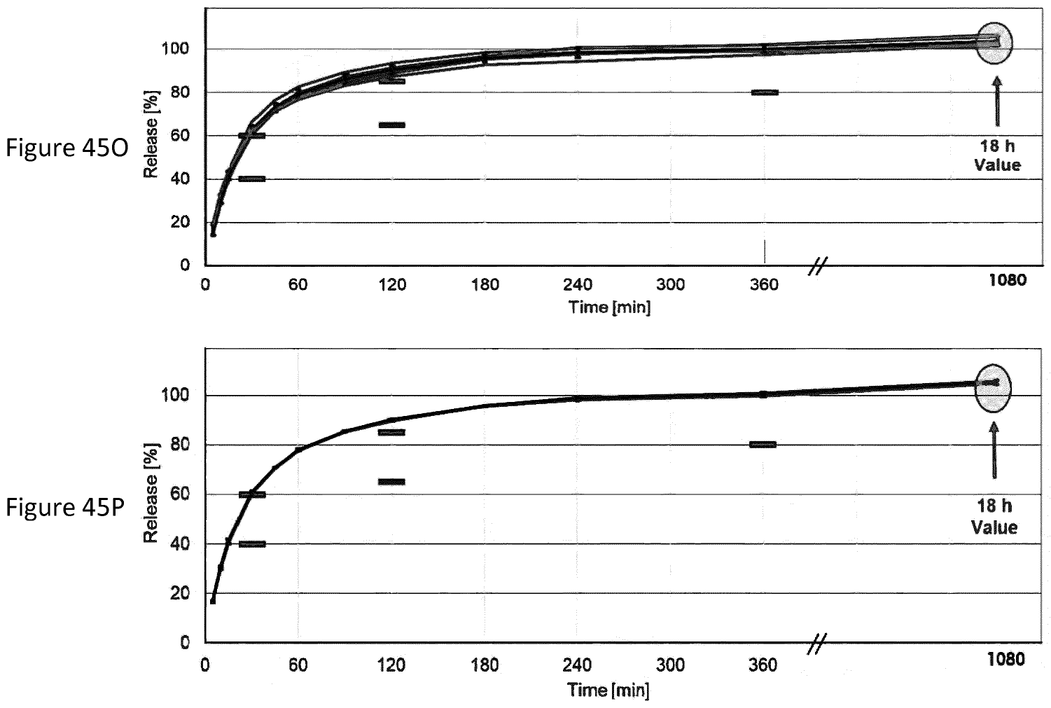

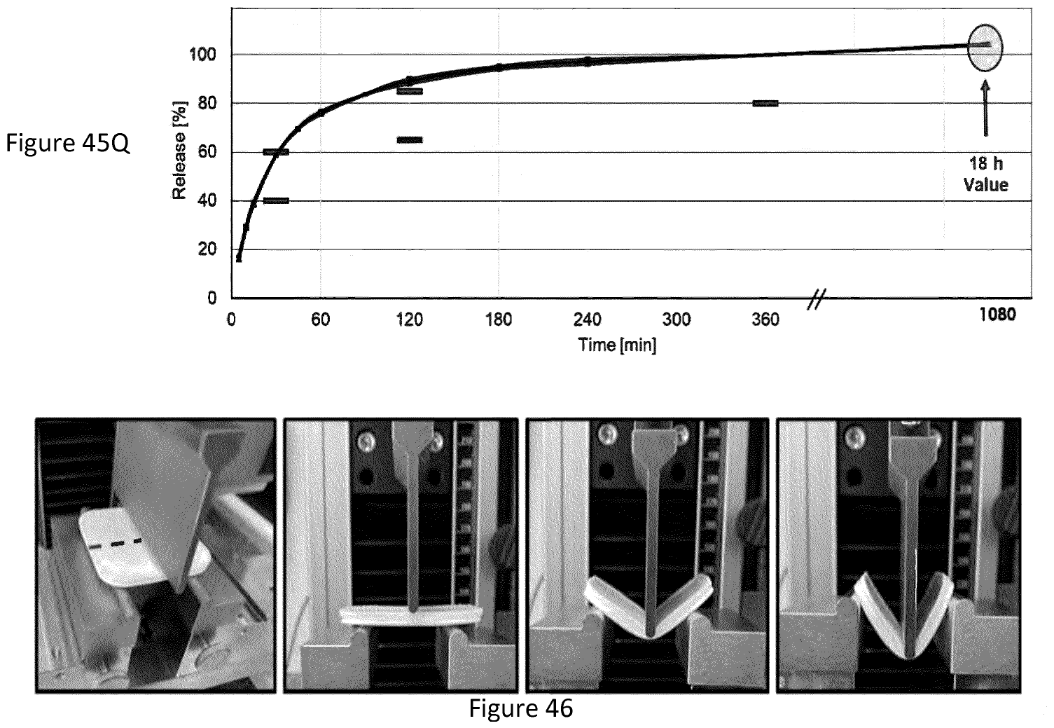

TABLE 26
Dissolution data of XaraColl (group 0, XaraColl batch 20000607) from FIG. 45A.
Group 0, XaraColl batch 20000607

| Time | Average | SD | RSD | a1 | a2 | a3 | a4 | a5 | a6 | a7 | a8 | a9 | a10 | a11 | a12 |
|---|---|---|---|---|---|---|---|---|---|---|---|---|---|---|---|
| [min] | [%] | [%] | [%] | [%] | [%] | [%] | [%] | [%] | [%] | [%] | [%] | [%] | [%] | [%] | [%] |
| 5 | 17 | 1.5 | 9.0 | 16 | 17 | 16 | 15 | 18 | 18 | 16 | 16 | 20 | 19 | 17 | 15 |
| 10 | 28 | 1.7 | 6.2 | 26 | 27 | 27 | 25 | 28 | 28 | 27 | 27 | 31 | 30 | 29 | 26 |
| 15 | 35 | 1.7 | 4.8 | 33 | 33 | 35 | 33 | 35 | 36 | 35 | 34 | 39 | 37 | 36 | 34 |
| 30 | 49 | 1.1 | 2.2 | 48 | 48 | 49 | 48 | 49 | 50 | 49 | 49 | 52 | 51 | 49 | 50 |
| 45 | 57 | 1.2 | 2.2 | 57 | 57 | 56 | 56 | 55 | 57 | 56 | 56 | 59 | 58 | 56 | 59 |
| 60 | 62 | 1.7 | 2.7 | 61 | 63 | 60 | 61 | 59 | 62 | 61 | 61 | 64 | 62 | 61 | 65 |
| 90 | 68 | 2.2 | 3.3 | 68 | 71 | 66 | 68 | 64 | 69 | 67 | 67 | 71 | 69 | 66 | 72 |
| 120 | 73 | 2.7 | 3.7 | 73 | 76 | 71 | 73 | 69 | 73 | 71 | 71 | 75 | 74 | 70 | 78 |
| 180 | 79 | 3.0 | 3.9 | 79 | 83 | 76 | 79 | 74 | 79 | 76 | 77 | 81 | 80 | 75 | 84 |
| 240 | 82 | 3.1 | 3.7 | 83 | 86 | 79 | 82 | 78 | 82 | 80 | 81 | 85 | 79 | 79 | 88 |
| 360 | 87 | 2.9 | 3.3 | 87 | 89 | 85 | 87 | 83 | 86 | 84 | 86 | 91 | 84 | 84 | 92 |
| 1080 | 96 | 2.0 | 3.1 | 96 | 96 | 94 | 95 | 93 | 95 | 94 | 95 | 99 | 95 | 95 | 99 |

TABLE 27
Dissolution data of XaraColl (group 0, XaraColl batch 21032106) from FIG. 45B.
Group 0, XaraColl batch 21032106

| Time | Average | SD | RSD | b1 | b2 | b3 | b4 | b5 | b6 | b7 | b8 |
|---|---|---|---|---|---|---|---|---|---|---|---|
| [min] | [%] | [%] | [%] | [%] | [%] | [%] | [%] | [%] | [%] | [%] | [%] |
| 5 | 19 | 1.1 | 5.6 | 18 | 19 | 20 | 21 | 20 | 19 | 19 | 18 |
| 10 | 32 | 0.6 | 1.7 | 31 | 32 | 32 | 33 | 32 | 32 | 31 | 32 |
| 15 | 40 | 0.9 | 2.1 | 40 | 39 | 39 | 40 | 40 | 40 | 40 | 42 |
| 30 | 53 | 2.6 | 4.9 | 53 | 52 | 49 | 50 | 52 | 53 | 54 | 58 |
| 45 | 59 | 3.4 | 5.7 | 60 | 57 | 54 | 56 | 58 | 60 | 61 | 65 |
| 60 | 63 | 3.8 | 6.0 | 64 | 62 | 58 | 59 | 62 | 63 | 65 | 70 |
| 90 | 68 | 4.7 | 6.9 | 70 | 67 | 62 | 64 | 67 | 69 | 71 | 77 |
| 120 | 72 | 5.1 | 7.1 | 73 | 69 | 65 | 66 | 70 | 72 | 75 | 81 |
| 180 | 76 | 5.4 | 7.1 | 78 | 75 | 69 | 70 | 75 | 77 | 81 | 86 |
| 240 | 80 | 5.2 | 6.6 | 81 | 77 | 73 | 73 | 79 | 81 | 83 | 89 |
| 360 | 85 | 4.8 | 5.6 | 87 | 82 | 79 | 79 | 84 | 86 | 89 | 93 |
| 1080 | 98 | 2.2 | 2.2 | 96 | 96 | 94 | 94 | 97 | 97 | 99 | 100 |

TABLE 28
Dissolution data of XaraColl (group 0, XaraColl batch 21031406) from FIG. 45C.
Group 0, XaraColl batch 21031406

| Time | Average | SD | RSD | c1 | c2 | c3 | c4 | c5 | c6 | c7 | c8 |
|---|---|---|---|---|---|---|---|---|---|---|---|
| [min] | [%] | [%] | [%] | [%] | [%] | [%] | [%] | [%] | [%] | [%] | [%] |
| 5 | 21 | 1.6 | 7.6 | 22 | 21 | 22 | 24 | 19 | 21 | 19 | 20 |
| 10 | 32 | 0.4 | 1.2 | 32 | 32 | 32 | 33 | 32 | 32 | 32 | 32 |
| 15 | 40 | 0.9 | 2.4 | 39 | 39 | 39 | 40 | 41 | 41 | 40 | 39 |
| 30 | 53 | 2.8 | 5.3 | 49 | 50 | 51 | 50 | 55 | 53 | 55 | 56 |
| 45 | 60 | 4.1 | 6.9 | 55 | 56 | 58 | 55 | 64 | 59 | 63 | 65 |
| 60 | 63 | 3.8 | 6.0 | 58 | 61 | 62 | 59 | 68 | 63 | 66 | 68 |
| 90 | 68 | 4.2 | 6.2 | 62 | 68 | 68 | 65 | 73 | 65 | 72 | 74 |
| 120 | 72 | 4.5 | 6.2 | 66 | 70 | 72 | 68 | 77 | 68 | 76 | 77 |
| 180 | 77 | 5.2 | 6.7 | 70 | 76 | 76 | 73 | 84 | 73 | 80 | 85 |
| 240 | 80 | 6.0 | 7.5 | 74 | 80 | 80 | 76 | 86 | 72 | 83 | 90 |
| 360 | 88 | 6.0 | 6.8 | 80 | 86 | 86 | 82 | 93 | 84 | 93 | 96 |
| 1080 | 99 | 3.4 | 3.4 | 96 | 98 | 99 | 95 | 105 | 99 | 102 | 102 |

TABLE 29
Dissolution data of XaraColl (group 0, XaraColl batch 21011207) from FIG. 45D.
Group 0, XaraColl batch 21011207

| Time | Average | SD | RSD | d1 | d2 | d3 | d4 | d5 | d6 | d7 | d8 |
|---|---|---|---|---|---|---|---|---|---|---|---|
| [min] | [%] | [%] | [%] | [%] | [%] | [%] | [%] | [%] | [%] | [%] | [%] |
| 5 | 21 | 2.5 | 11.9 | 19 | 21 | 20 | 16 | 24 | 22 | 20 | 23 |
| 10 | 32 | 2.3 | 7.2 | 31 | 32 | 32 | 27 | 35 | 33 | 32 | 34 |
| 15 | 40 | 1.7 | 4.4 | 39 | 40 | 39 | 36 | 41 | 42 | 40 | 41 |
| 30 | 53 | 1.5 | 2.8 | 53 | 51 | 51 | 54 | 51 | 55 | 55 | 53 |
| 45 | 60 | 2.9 | 4.9 | 62 | 58 | 58 | 65 | 56 | 63 | 62 | 59 |
| 60 | 65 | 4.0 | 6.1 | 67 | 62 | 62 | 72 | 60 | 67 | 67 | 63 |
| 90 | 72 | 5.6 | 7.8 | 73 | 67 | 67 | 82 | 65 | 74 | 75 | 69 |
| 120 | 75 | 6.1 | 8.1 | 78 | 71 | 71 | 86 | 67 | 79 | 80 | 72 |
| 180 | 81 | 6.6 | 8.1 | 83 | 76 | 76 | 92 | 73 | 84 | 86 | 79 |
| 240 | 85 | 6.3 | 7.4 | 87 | 80 | 80 | 95 | 76 | 89 | 90 | 83 |
| 360 | 91 | 5.3 | 5.8 | 94 | 88 | 87 | 100 | 83 | 93 | 94 | 89 |
| 1080 | 101 | 2.3 | 2.3 | 100 | 98 | 97 | 102 | 99 | 103 | 103 | 102 |

TABLE 30
Dissolution data of XaraColl (group 1, compression tool 1st
generation, 12 mm trocar, XaraColl batch 20000607) from FIG. 45E.
Group 1, 1^{st} generation, 12 mm trocar, batch 20000607

| Time | Average | SD | RSD | d1 | d2 | d3 | d4 | d5 | d6 | d7 | d8 |
|---|---|---|---|---|---|---|---|---|---|---|---|
| [min] | [%] | [%] | [%] | [%] | [%] | [%] | [%] | [%] | [%] | [%] | [%] |
| 5 | 20 | 1.7 | 11.8 | 15 | 16 | 17 | 16 | 12 | 13 | 14 | 15 |
| 10 | 32 | 3.3 | 11.8 | 27 | 29 | 33 | 32 | 23 | 25 | 26 | 28 |
| 15 | 40 | 3.8 | 10.0 | 36 | 38 | 44 | 43 | 33 | 35 | 6 | 37 |
| 30 | 54 | 3.3 | 5.9 | 55 | 57 | 61 | 62 | 52 | 55 | 55 | 56 |
| 45 | 61 | 2.5 | 3.7 | 65 | 68 | 70 | 71 | 63 | 66 | 66 | 66 |
| 60 | 66 | 2.0 | 2.8 | 71 | 74 | 75 | 77 | 71 | 72 | 73 | 72 |
| 90 | 72 | 1.6 | 2.0 | 79 | 81 | 81 | 84 | 79 | 80 | 81 | 80 |
| 120 | 75 | 1.4 | 1.7 | 84 | 86 | 85 | 89 | 85 | 86 | 86 | 85 |
| 180 | 80 | 1.3 | 1.5 | 89 | 91 | 89 | 94 | 90 | 9 | 91 | 90 |
| 240 | 84 | 1.1 | 1.2 | 92 | 93 | 92 | 94 | 94 | 94 | 94 | 94 |
| 360 | 87 | 1.1 | 1.1 | 96 | 96 | 94 | 97 | 97 | 97 | 97 | 96 |
| 1080 | 96 | 0.9 | 0.9 | 99 | 98 | 98 | 99 | 100 | 100 | 100 | 100 |

TABLE 31
Dissolution data of XaraColl (group 2a, compression tool 2nd
generation, 8 mm trocar, XaraColl batch 20000607) from FIG. 45F.
Group 2a, 2^{nd} generation, 8 mm trocar, batch 20000607

| Time | Average | SD | RSD | e1 | e2 | e3 | e4 | e5 | e6 | e7 | e8 |
|---|---|---|---|---|---|---|---|---|---|---|---|
| [min] | [%] | [%] | [%] | [%] | [%] | [%] | [%] | [%] | [%] | [%] | [%] |
| 5 | 13 | 0.6 | 4.6 | 13 | 13 | 13 | 14 | 13 | 13 | 13 | 13 |
| 10 | 25 | 1.3 | 5.2 | 23 | 24 | 24 | 26 | 26 | 26 | 25 | 25 |
| 15 | 35 | 2.1 | 6.1 | 32 | 33 | 34 | 36 | 37 | 37 | 35 | 36 |
| 30 | 55 | 3.4 | 6.2 | 50 | 51 | 54 | 55 | 58 | 58 | 55 | 56 |
| 45 | 66 | 3.0 | 4.5 | 62 | 63 | 64 | 65 | 69 | 69 | 67 | 68 |
| 60 | 72 | 3.9 | 5.4 | 67 | 67 | 71 | 70 | 76 | 76 | 73 | 74 |
| 90 | 80 | 3.7 | 4.6 | 75 | 76 | 79 | 78 | 83 | 83 | 82 | 83 |
| 120 | 84 | 4.1 | 4.9 | 79 | 80 | 83 | 82 | 89 | 89 | 86 | 88 |
| 180 | 90 | 4.2 | 4.7 | 85 | 86 | 87 | 88 | 95 | 95 | 92 | 94 |
| 240 | 94 | 4.5 | 4.8 | 89 | 88 | 90 | 91 | 98 | 98 | 97 | 98 |
| 360 | 97 | 4.5 | 4.6 | 93 | 92 | 92 | 94 | 101 | 101 | 101 | 101 |
| 1080 | 100 | 4.1 | 4.1 | 96 | 96 | 96 | 97 | 104 | 104 | 103 | 103 |

TABLE 32
Dissolution data of XaraColl (group 2b, compression tool 2nd
generation, 12 mm trocar XaraColl batch 20000607) from FIG. 45G.
Group 2b, 2^{nd} generation, 12 mm trocar, batch 20000607

| Time | Average | SD | RSD | f1 | f2 | f3 | f4 | f5 | f6 | f7 | f8 |
|---|---|---|---|---|---|---|---|---|---|---|---|
| [min] | [%] | [%] | [%] | [%] | [%] | [%] | [%] | [%] | [%] | [%] | [%] |
| 5 | 14 | 0.7 | 5.4 | 14 | 15 | 14 | 13 | 14 | 14 | 13 | 11 |
| 10 | 25 | 1.6 | 6.6 | 25 | 27 | 26 | 23 | 27 | 25 | 24 | 23 |
| 15 | 34 | 2.2 | 6.5 | 35 | 36 | 35 | 32 | 38 | 34 | 33 | 33 |
| 30 | 54 | 3.1 | 5.9 | 54 | 55 | 55 | 50 | 59 | 52 | 52 | 53 |
| 45 | 64 | 3.5 | 5.5 | 64 | 65 | 64 | 59 | 70 | 63 | 64 | 65 |
| 60 | 71 | 3.4 | 4.8 | 70 | 70 | 70 | 66 | 76 | 70 | 71 | 72 |
| 90 | 79 | 3.2 | 4.0 | 77 | 78 | 78 | 74 | 84 | 79 | 80 | 81 |
| 120 | 84 | 3.1 | 3.7 | 82 | 82 | 82 | 79 | 89 | 84 | 86 | 87 |
| 180 | 89 | 3.2 | 3.6 | 88 | 87 | 86 | 85 | 94 | 91 | 92 | 93 |
| 240 | 93 | 3.8 | 4.0 | 90 | 90 | 90 | 88 | 98 | 94 | 95 | 97 |
| 360 | 96 | 4.1 | 4.2 | 94 | 92 | 92 | 92 | 101 | 99 | 101 | 102 |
| 1080 | 100 | 3.7 | 3.7 | 96 | 96 | 96 | 96 | 103 | 103 | 104 | 106 |

TABLE 33
Dissolution data of XaraColl (group 2c, compression tool 2nd generation,
8 mm trocar, 20% r.h., XaraColl batch 21032106) from FIG. 45H.
Group 2c, 2^{nd} generation, 8 mm trocar, 20% r.h., batch 21032106

| Time | Average | SD | RSD | g1 | g2 | g3 | g4 |
|---|---|---|---|---|---|---|---|
| [min] | [%] | [%] | [%] | [%] | [%] | [%] | [%] |
| 5 | 16 | 1.7 | 10.1 | 16 | 18 | 14 | 18 |
| 10 | 31 | 1.9 | 6.0 | 31 | 32 | 28 | 32 |
| 15 | 41 | 1.8 | 4.4 | 42 | 41 | 39 | 43 |
| 30 | 60 | 1.5 | 2.5 | 61 | 59 | 58 | 61 |
| 45 | 68 | 1.1 | 1.7 | 69 | 68 | 67 | 69 |
| 60 | 73 | 1.1 | 1.5 | 74 | 73 | 72 | 74 |
| 90 | 79 | 1.0 | 1.3 | 79 | 78 | 78 | 80 |
| 120 | 82 | 1.2 | 1.4 | 83 | 81 | 81 | 82 |
| 180 | 85 | 0.7 | 0.9 | 86 | 84 | 85 | 85 |
| 240 | 87 | 0.2 | 0.2 | 87 | 87 | 87 | 87 |
| 360 | 88 | 0.6 | 0.7 | 89 | 87 | 88 | 87 |
| 1080 | 89 | 0.6 | 0.7 | 88 | 88 | 88 | 89 |

TABLE 34
Dissolution data of XaraColl (group 2d, compression tool 2nd generation,
12 mm trocar, 20% r.h., XaraColl batch 21032106) from FIG. 45I.
Group 2d, 2^{nd} generation, 12 mm trocar, 20% r.h., batch 21032106

| Time | Average | SD | RSD | h1 | h2 | h3 | h4 |
|---|---|---|---|---|---|---|---|
| [min] | [%] | [%] | [%] | [ %] | [%] | [%] | [%] |
| 5 | 15 | 0.7 | 4.3 | 15 | 16 | 15 | 15 |
| 10 | 29 | 0.7 | 2.3 | 29 | 30 | 28 | 29 |
| 15 | 39 | 0.7 | 1.8 | 39 | 40 | 39 | 39 |
| 30 | 58 | 0.8 | 1.3 | 58 | 59 | 58 | 57 |
| 45 | 66 | 0.7 | 1.1 | 66 | 67 | 66 | 66 |
| 60 | 72 | 0.5 | 0.7 | 71 | 72 | 72 | 72 |
| 90 | 78 | 0.4 | 0.5 | 77 | 78 | 78 | 78 |
| 120 | 81 | 0.6 | 0.7 | 80 | 81 | 82 | 82 |
| 180 | 85 | 0.5 | 0.6 | 84 | 85 | 85 | 86 |
| 240 | 87 | 0.5 | 0.6 | 87 | 87 | 88 | 87 |
| 360 | 87 | 0.4 | 0.5 | 87 | 87 | 87 | 88 |
| 1080 | 88 | 0.7 | 0.8 | 87 | 88 | 88 | 89 |

TABLE 35
Dissolution data of XaraColl (group 3a, compression tool 3rd generation,
8 mm trocar, 20% r.h., XaraColl batch 21032106) from FIG. 45J.
Group 3a, 3^{rd} generation, 8 mm trocar, 20% r.h., batch 21032106

| Time | Average | SD | RSD | i1 | i2 | i3 |
|---|---|---|---|---|---|---|
| [min] | [%] | [%] | [%] | [%] | [%] | [%] |
| 5 | 15 | 0.7 | 4.4 | 14 | 16 | 15 |
| 10 | 30 | 1.1 | 3.7 | 29 | 30 | 31 |
| 15 | 41 | 2.8 | 6.8 | 38 | 42 | 43 |
| 30 | 63 | 1.8 | 2.8 | 61 | 63 | 65 |
| 45 | 71 | 3.7 | 5.1 | 68 | 72 | 75 |
| 60 | 78 | 3.2 | 4.1 | 74 | 78 | 80 |
| 90 | 85 | 3.3 | 3.9 | 81 | 86 | 87 |
| 120 | 89 | 3.8 | 4.3 | 84 | 90 | 92 |
| 180 | 94 | 2.5 | 2.6 | 91 | 95 | 96 |
| 240 | 96 | 4.2 | 4.4 | 91 | 98 | 99 |
| 360 | 98 | 3.3 | 3.4 | 94 | 100 | 100 |
| 1080 | 101 | 3.5 | 3.4 | 97 | 103 | 103 |

TABLE 36
Dissolution data of XaraColl (group 3b, compression tool 3rd generation,
12 mm trocar, 20% r.h., XaraColl batch 21032106) from FIG. 45K.
Group 3b, 3^{rd} generation, 12 mm trocar, 20% r.h., batch 21032106

| Time | Average | SD | RSD | j1 | j2 | j3 | j4 |
|---|---|---|---|---|---|---|---|
| [min] | [ %] | [%] | [%] | [%] | [%] | [%] | [%] |
| 5 | 14 | 2.0 | 13.7 | 17 | 13 | 13 | 14 |
| 10 | 29 | 2.5 | 8.9 | 32 | 27 | 26 | 29 |
| 15 | 40 | 2.7 | 6.9 | 43 | 37 | 38 | 41 |
| 30 | 61 | 2.9 | 4.7 | 63 | 58 | 59 | 64 |
| 45 | 71 | 2.1 | 3.0 | 73 | 69 | 70 | 73 |
| 60 | 77 | 2.1 | 2.7 | 79 | 76 | 75 | 80 |
| 90 | 85 | 1.2 | 1.5 | 85 | 84 | 84 | 87 |
| 120 | 90 | 1.0 | 1.1 | 90 | 89 | 89 | 91 |
| 180 | 95 | 1.0 | 1.1 | 95 | 95 | 94 | 97 |
| 240 | 98 | 0.7 | 0.7 | 98 | 99 | 98 | 99 |
| 360 | 101 | 0.9 | 0.9 | 100 | 101 | 101 | 102 |
| 1080 | 103 | 0.9 | 0.9 | 102 | 105 | 103 | 103 |

TABLE 37
Dissolution data of XaraColl (group 3c, compression tool 3rd generation,
8 mm trocar, 80% r.h., XaraColl batch 21032106) from FIG. 45L.
Group 3c, 3^{rd} generation, 8 mm trocar, 80% r.h., batch 21032106

| Time | Average | SD | RSD | k1 | k2 | k3 | k4 |
|---|---|---|---|---|---|---|---|
| [min] | [%] | [%] | [%] | [%] | [%] | [%] | [%] |
| 5 | 13 | 0.2 | 1.8 | 13 | 12 | 13 | 13 |
| 10 | 25 | 0.4 | 1.5 | 25 | 25 | 24 | 25 |
| 15 | 35 | 0.2 | 0.6 | 35 | 35 | 35 | 35 |
| 30 | 54 | 0.4 | 0.8 | 54 | 53 | 54 | 54 |
| 45 | 64 | 0.5 | 0.7 | 63 | 64 | 65 | 64 |
| 60 | 70 | 0.7 | 1.0 | 70 | 70 | 71 | 71 |
| 90 | 78 | 1.2 | 1.5 | 76 | 79 | 78 | 79 |
| 120 | 83 | 1.2 | 1.5 | 81 | 83 | 84 | 83 |
| 180 | 88 | 1.1 | 1.3 | 86 | 88 | 88 | 89 |
| 240 | 91 | 1.6 | 1.8 | 89 | 91 | 92 | 92 |
| 360 | 93 | 1.5 | 1.6 | 91 | 94 | 94 | 95 |
| 1080 | 96 | 2.4 | 2.6 | 92 | 96 | 97 | 98 |

TABLE 38
Dissolution data of XaraColl (group 3d, compression tool 3rd generation,
12 mm trocar, 80% r.h., XaraColl batch 21032106) from FIG. 45M.
Group 3d, 3^{rd} generation, 12 mm trocar, 80% r.h., batch 21032106

| Time | Average | SD | RSD | l1 | l2 | l3 | l4 |
|---|---|---|---|---|---|---|---|
| [min] | [%] | [%] | [%] | [%] | [%] | [%] | [%] |
| 5 | 15 | 1.5 | 10.5 | 16 | 16 | 13 | 14 |
| 10 | 27 | 1.3 | 4.7 | 27 | 28 | 25 | 27 |
| 15 | 36 | 1.2 | 3.4 | 37 | 37 | 35 | 36 |
| 30 | 54 | 0.5 | 0.9 | 54 | 54 | 54 | 55 |
| 45 | 64 | 0.5 | 0.8 | 64 | 64 | 64 | 63 |
| 60 | 70 | 0.5 | 0.6 | 70 | 71 | 71 | 70 |
| 90 | 78 | 0.5 | 0.7 | 77 | 78 | 78 | 77 |
| 120 | 82 | 0.5 | 0.6 | 81 | 82 | 82 | 82 |
| 180 | 86 | 0.7 | 0.8 | 86 | 87 | 87 | 86 |
| 240 | 90 | 1.0 | 1.2 | 89 | 90 | 91 | 89 |
| 360 | 93 | 1.3 | 1.4 | 92 | 93 | 95 | 93 |
| 1080 | 95 | 0.7 | 0.7 | 94 | 95 | 96 | 96 |

TABLE 39
Dissolution data of XaraColl (group 5a, compression tool 5th generation,
8 mm trocar, XaraColl batch 21011207) from FIG. 45N.
Group 5a, 5th generation, 8 mm trocar, batch 21011207

| Time | Average | SD | RSD | m1 | m2 | m3 | m4 | m5 | m6 |
|---|---|---|---|---|---|---|---|---|---|
| [min] | [%] | [%] | [%] | [%] | [%] | [%] | [%] | [%] | [%] |
| 5 | 17 | 1.0 | 6.0 | 18 | 17 | 16 | 16 | 16 | 20 |
| 10 | 30 | 1.6 | 5.4 | 33 | 30 | 29 | 30 | 27 | 33 |
| 15 | 39 | 0.9 | 2.4 | 38 | 40 | 38 | 40 | 37 | 45 |
| 30 | 59 | 1.4 | 2.4 | 61 | 58 | 58 | 59 | 57 | 61 |
| 45 | 68 | 0.6 | 0.9 | 68 | 68 | 67 | 69 | 68 | 71 |
| 60 | 75 | 0.6 | 0.9 | 74 | 74 | 75 | 75 | 76 | 76 |
| 90 | 82 | 1.2 | 1.5 | 81 | 82 | 84 | 83 | 84 | 84 |
| 120 | 87 | 1.9 | 2.2 | 84 | 87 | 88 | 89 | 89 | 88 |
| 180 | 92 | 1.3 | 1.4 | 90 | 92 | 93 | 93 | 94 | 93 |
| 240 | 95 | 2.3 | 2.4 | 91 | 95 | 96 | 96 | 97 | 96 |
| 360 | 98 | 2.8 | 2.8 | 94 | 99 | 100 | 99 | 100 | 98 |
| 1080 | 102 | 2.8 | 2.7 | 97 | 104 | 103 | 102 | 102 | 102 |

TABLE 40
Dissolution data of XaraColl (group 5b, compression tool 5th generation,
8 mm trocar, XaraColl batch 21032106) from FIG. 45O.
Group 5b, 5th generation, 8 mm trocar, batch 21032106

| Time | Average | SD | RSD | m1 | m2 | m3 | m4 | m5 | m6 |
|---|---|---|---|---|---|---|---|---|---|
| [min] | [%] | [%] | [%] | [%] | [%] | [%] | [%] | [%] | [%] |
| 5 | 16 | 2.3 | 14.2 | 16 | 15 | 15 | 15 | 20 | 19 |
| 10 | 31 | 1.9 | 6.2 | 31 | 29 | 29 | 29 | 30 | 34 |
| 15 | 42 | 2.0 | 4.7 | 44 | 41 | 41 | 40 | 40 | 44 |
| 30 | 63 | 1.9 | 3.1 | 66 | 62 | 60 | 62 | 63 | 62 |
| 45 | 73 | 1.0 | 2.7 | 77 | 72 | 72 | 73 | 73 | 71 |
| 60 | 79 | 2.2 | 2.8 | 83 | 78 | 77 | 79 | 80 | 77 |
| 90 | 86 | 2.1 | 2.5 | 89 | 86 | 85 | 87 | 87 | 83 |
| 120 | 90 | 2.1 | 2.3 | 93 | 90 | 89 | 91 | 91 | 87 |
| 180 | 96 | 1.9 | 2.0 | 98 | 95 | 95 | 97 | 97 | 93 |
| 240 | 98 | 2.3 | 2.3 | 100 | 98 | 98 | 101 | 98 | 94 |
| 360 | 100 | 1.8 | 1.8 | 102 | 100 | 100 | 102 | 99 | 97 |
| 1080 | 104 | 2.1 | 20 | 107 | 104 | 103 | 106 | 101 | 102 |

TABLE 41
Dissolution data of XaraColl (group 5c, compression
tool 5th generation, 8 mm trocar, XaraColl batch
21031406) from FIG. 45P.
Group 5c, 5th generation, 8 mm
trocar, batch 21031406

| Time | Average | SD | RSD | m1 | m2 |
|---|---|---|---|---|---|
| [min] | [%] | [%] | [%] | [%] | [%] |
| 5 | 17 | 0.2 | 1.3 | 17 | 17 |
| 10 | 30 | 0.7 | 2.3 | 31 | 30 |
| 15 | 41 | 0.8 | 1.9 | 42 | 40 |
| 30 | 61 | 0.6 | 1.0 | 61 | 60 |
| 45 | 71 | 0.0 | 0.0 | 71 | 71 |
| 60 | 78 | 0.3 | 0.4 | 78 | 78 |
| 90 | 85 | 0.2 | 0.3 | 85 | 86 |
| 120 | 90 | 0.4 | 0.4 | 90 | 90 |
| 180 | 96 | 0.2 | 0.2 | 96 | 96 |
| 240 | 99 | 0.6 | 0.6 | 99 | 98 |
| 360 | 100 | 0.8 | 0.8 | 101 | 100 |
| 1080 | 105 | 1.0 | 0.9 | 106 | 105 |

TABLE 42
Dissolution data of XaraColl (group 5d, compression
tool 5^{th} generation, 8 mm trocar,
XaraColl batch 20000607) from FIG. 45Q.
Group 5d, 5th generation, 8 mm
trocar, batch 20000607

| Time | Average | SD | RSD | m1 | m2 |
|---|---|---|---|---|---|
| [min] | [%] | [%] | [%] | [%] | [%] |
| 5 | 16 | 0.8 | 5.1 | 17 | 16 |
| 10 | 29 | 0.8 | 2.8 | 30 | 28 |
| 15 | 39 | 0.9 | 2.2 | 39 | 38 |
| 30 | 58 | 0.0 | 0.1 | 58 | 58 |
| 45 | 70 | 0.5 | 0.8 | 69 | 70 |
| 60 | 76 | 0.9 | 1.1 | 76 | 77 |
| 90 | 84 | 0.3 | 0.4 | 83 | 84 |
| 120 | 89 | 1.4 | 1.6 | 88 | 90 |
| 180 | 95 | 1.0 | 1.1 | 94 | 95 |
| 240 | 97 | 1.5 | 1.5 | 96 | 98 |
| 360 | 100 | 0.0 | 0.0 | 100 | 100 |
| 1080 | 104 | 0.4 | 0.4 | 104 | 104 |

TABLE 43
f1/f2 statistical analysis with dissimilar results in bold text.

| | | | | Acceptance | |
|---|---|---|---|---|---|
| | | | | criterion | |
| Compared groups | | f1 | f2 | f1 | f2 |
| XaraColl group 1 | [symbol] | 3.1 | 82.8 | 0-15 | 50-100 |
| XaraColl group 2a | 20000607, group 0 | 10.4 | 53.8 | | |
| XaraColl group 2b | | 12.9 | 55.9 | | |
| XaraColl group 5d | | 18.8 | 47.9 | | |
| XaraColl group 2c | XaraColl batch | 20.4 | 55.5 | | |
| XaraColl group 2d | 21032106, group 0 | 19.2 | 57.5 | | |
| XaraColl group 3a | | 9.4 | 46.8 | | |
| XaraColl group 3b | | 13.7 | 47.0 | | |
| XaraColl group 3c | | 12.3 | 55.2 | | |
| XaraColl group 3d | | 11.6 | 57.7 | | |
| XaraColl group 5b | | 17.1 | 47.4 | | |
| XaraColl group 5a | XaraColl batch | 11.7 | 55.2 | | |
| | 21011207, group 0 | | | | |
| XaraColl group 5c | XaraColl batch | 18.9 | 46.8 | | |
| | 21031406, group 0 | | | | |

### Example 10: Laparoscopic Application of XaraColl—Development of an Improved Sample Preparation Technique (Rolling Approach) Using Compression Tool Generation 6

### Introduction

Example 8 provided a new sample preparation strategy which avoided moistening of the XaraColl samples and uses XaraColl halves instead of quarters. This preparation strategy was found to have a negligible impact on the dissolution behavior compared to uncut matrices, as confirmed by f1/f2 factor analysis. In order to grab the implant with a grasper and pass the implant through a trocar, the implant's compactness needed to be increased. Therefore, a folding approach was developed, whereby the implant was folded twice along its short axis. Subsequently, the folded XaraColl implant halves could be inserted into a 12 mm trocar easily. Example 8 summarizes the drug release characteristics of XaraColl implants which were treated with this approach. It was found that the handling procedure involving the compression tool is suitable, since f1/f2 statistical analysis revealed similar/not dissimilar dissolution profiles compared to complete, untreated XaraColl implants. In contrast, the dissolution properties of a completely compressed (“paper thickness”) XaraColl implant revealed dissolution properties clearly different from the untreated implant, as proven by f1/f2 statistical test. The compression of XaraColl with the developed compression technique and a defined compression gap (partial compression of XaraColl) does not change the drug release properties of XaraColl.

In order to fine-tune this approach, which includes compression, cutting, and folding of the implants, the compression tool was improved with regard to its handling properties and general design. Thereby, the design has been updated starting from compression tool generation 1, which was used for the studies described in Example 8, up to generation 5. Example 9 summarizes this development and includes all relevant handling as well as performance data.

Although the folding approach was generally suitable for the laparoscopic placement of XaraColl implants, it was not unrestrictedly combinable with all trocar types. Therefore, a method that works independently from the applied trocar and grasper type is desired. In addition to that, most surgeons are used to rolling materials before passing them through a trocar, e.g., the mesh used for hernia repair, rather than folding. Furthermore, rolled XaraColl implants are expected to unravel more easily compared to the folded ones. Thus, the sample preparation technique was modified to include loose rolling of the XaraColl implant instead of folding. This approach was found to optimally function even for the insertion of implants into a trocar with a small diameter of 8 mm lumen. The optimum gap size for the compression tool was also investigated in the course of this study, whereby gaps ranging from 0.6- and 1.0-mm function equally well.

Even though the rolling approach was already working reliably for a certain type of trocar, namely the J&J Ethicon trocar series, so far no testing had been performed using different trocar types, e.g. the Applied trocar series. Thus, the present report refines this improved approach and aims for a strategy that works independently of the utilized grasper or trocar types. In addition to that, it includes the development of the final compression tool generation 6.6 and the laparoscopic handling of XaraColl implants using the latter.

The present example provides a new (generation 6) compression tool. This compression tool features a defined compression gap of 1.0 mm, resulting in a defined and controlled compression of XaraColl. Moreover, the compression tool imprints marks into the XaraColl implant, which serve as orientation for the correct handling and thus support the laparoscopic placement of the implants.

With this compression tool in hand, the rolling technique was conducted and the suitability of the compression tool for the intended laparoscopic use was evaluated. For this purpose, the compressed implants were compared with an uncompressed control group. These two groups have been characterized using texture analyses, determining important structural parameters which include resistance to pressure and resistance to bending measurements as well as the implant deformation upon bending. Moreover, the implant thickness in dry and in wet state was determined while SEM imaging of compressed and uncompressed implants was conducted.

In a second part, the applicability of the overall handling technique for the intended laparoscopic use, including partial compression, cutting, and rolling of the implant, has been investigated. On that account, distinct criteria for the evaluation of the implants condition during this process were determined. Since it is envisaged that the handling approach functions independently of the manufacturer and type of the used instruments (manufacturer and type), not only different XaraColl batches, but also different grasper and trocar models have been evaluated.

Lastly, the capability of the implants to unroll after trocar passage have been investigated, using a wet dishcloth as model system mimicking the abdominal wall.

### Methods

### Reagents and Instruments

Within this study, all texture and compression and rolling experiments are performed using the identical XaraColl batches 21011207 and 21010507. For the unrolling experiments XaraColl batch 19011203 was used. The instruments used for this study are listed in Table 44.

TABLE 44
Instruments used for this study.

| | | Inv.-number/ |
|---|---|---|
| Instrument | Manufacturer | serial number |
| Compression tool generation 6.6 | Syntacoll | N/A |
| Grasper (35 mm jaws, double action | New Med | N/A |
| atraumatic fenestrated bowel) | | |
| Grasper (40 mm jaws, double action | Mediflex | N/A |
| atraumatic fenestrated bowel) | | |
| Grasper (30 mm jaws, double action | Mediflex | N/A |
| Babcock) | | |
| Trocar (tube length 100 mm, | Ethicon | N/A |
| diameter 8 mm) | | |
| Trocar (tube length 100 mm, | Applied | N/A |
| diameter 8 mm) | | |
| Climatic chamber KBF 240 | Binder | 400692 |
| Universal tester Zwicki 500N | Zwick/Röll | 200134 |
| 3D Printer Pro 2 | Raise 3D | 700152 |

### Texture Analyses

The three key steps for the preparation of a XaraColl implant for laparoscopic placement steps are compression, cutting, and rolling of the implant. In order to facilitate this preparation, the flexibility of the implant needs to be improved so that the XaraColl implant can be rolled easily and without rupturing. This increase in flexibility can be achieved through the controlled compression of the implant with a so-called compression tool featuring a defined compression gap. Importantly, this controlled and partial compression has no impact on the implants characteristic drug release properties, whereas uncontrolled and complete compression should be avoided. In order to evaluate the impact of the compression technique on the implants and to characterize the properties of the latter after this process, various measurements have been conducted. This includes SEM measurements as well as other relevant analyses of texture parameters (i.e., resistance to pressure, resistance to bending, deformation at break, thickness, thickness wet, and tensile strength wet), which were analyzed using the universal testing equipment Zwicki 500 N. For all measurements, dry and uncompressed implants were used as removed from the packaging, compressed implants were treated with compression tool generation 6.6 prior to the analysis.

Resistance to pressure and dry thickness measurements: A spherical test body with a one-inch diameter is pressed into the implant and the resulting force for 1 mm intrusion is determined. Additionally, the matrix thickness at first contact (F=0.01 N) of the test body is recorded.

Resistance to bending analysis: The analysis is conducted using a three-point bending setup, whereby a rounded tool with 1.5 mm radius is lowered down, thus bending the implant, which is placed on a flat table 90° to the dotted line (FIG. 46). Thereby, the maximum force prior to breaking and the deformation of the implant at break are recorded.

Thickness measurements of wet implants: The implants were placed in a water bath with the Tyvek side facing up for 1 h. Upon removal of the implants from the water bath, they were placed on an even surface and positioned in the Zwicki 500 N apparatus. A four-square centimeter sized test body was lowered down towards the implant and upon the first contact between the square test body and the implant (F=0.005 N) the distance was recorded.

Tensile strength of wet implants: Dry compressed and uncompressed implants were cut into dumbbell shaped test specimens. The dumbbell shaped samples were clamped at their wider section and are expected to break in their narrow section (8 mm width). The clamped sections are pulled apart and the force prior to breaking is recorded.

### Sample Preparation: Compression, Rolling and Trocar Insertion

The sample preparation was investigated involving two different XaraColl batches, 21011207 and 21010507, which were compressed, rolled, and passed through the trocar. Prior to this study, the implants were removed from their outer pouch and conditioned in the climatic chamber KBF 240 at 30% r.h. and 22° C. overnight. As the implants are in equilibrium with their environment, the moistness of the implants decreases and adjusts to the dry conditions and thus their brittleness increases to a maximum. These conditions represent an artificial worst case, as the humidity in surgery rooms lies between 35% and 65% and the pouch, which the implants are usually stored inside, would normally hamper this equilibration under usual conditions. After compression of the implant using the compression tool, the compressed implant can be easily cut into two halves by using scissors. The two halves are tightly rolled and the obtained bundle can be finally inserted through the trocar by using a grasper. Noteworthy, it is advisable that the bottom side of the XaraColl implant is on the outside of the rolled bundle as the top side and the bottom side of the XaraColl implant are not identical regarding withstanding physical stress. These differences arise from the nature of the freeze-drying process. The blister side has slightly rounded edges from the mold used for lyophilization (FIG. 21, top left). In contrast, the Tyvek side has sharp edges and a concave (bowl) shape caused by capillary forces at the mold wall (FIG. 21, top right). Further, the micro-structure of XaraColl's Tyvek side and blister side likewise differ. The blister side of the matrix (FIG. 21, bottom left) shows a very flexible fine collagen structure, whereas the Tyvek side (FIG. 21, bottom right) shows a more or less continuous closed epithelium-like surface, morphologically indicating less flexibility. Hence, both sides of the XaraColl implant are different and require distinction for optimal rolling performance. Noteworthy, mixing up these two different sides is excluded by distinct marks, which are imprinted into the implant upon compression and serve as a guide for the surgeon.

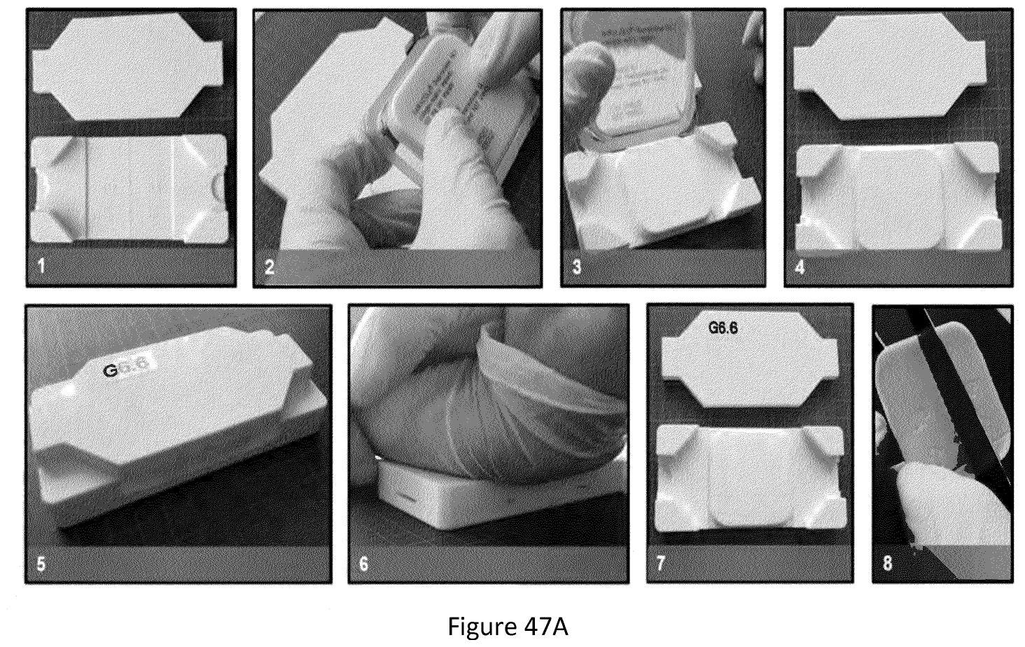

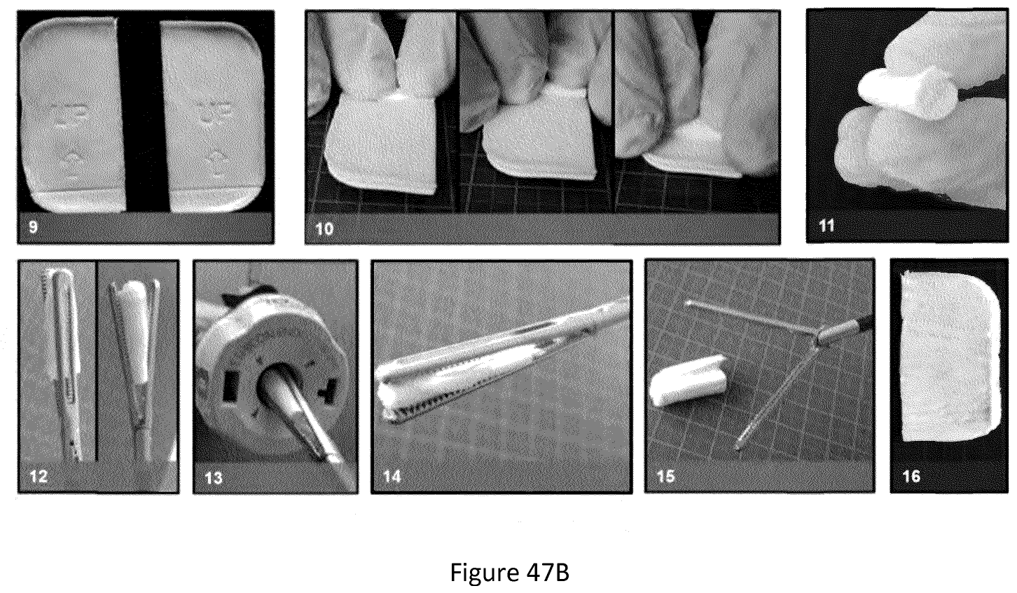

In detail, the single steps of this preparation procedure include (illustrated in FIGS. 47A-47B)
1) The compression tool consists of a top part (above) and a bottom part (below).
2) The seal from the XaraColl blister container is peeled off and the blister turned.
3) Enough pressure is applied to the back of the blister container to carefully drop the implant onto the base of the compression tool.
4) The implant is positioned in the middle of the base centered within the square.
5) The top of the compression tool is placed onto the base.
6) The top is pressed down evenly until the top sits flush with the base. The compression is held for two seconds.
7) The top part of the compression tool is removed. After removal, the implant re-expands to a certain extent.
8) The implant is removed from the compression tool and cut into two halves along the dotted line.
9) The implant halves are placed on a surface with the “UP” marking facing up and the arrow pointing away from the operator.
10) The implant halves are rolled tightly. The rolling is initiated by folding at the highlighted line.
11) The rolled bundle should be as compact as possible. Importantly, steady downward pressure against the table surface should be applied whilst rolling.
12) The rolled bundle is placed into a dry laparoscopic atraumatic grasper with a minimum jaw length of 28 mm. The implant is centered in the grasper, so that there is space both at the tip and crotch end of the grasper.
13) The laparoscopic grasper is inserted together with the implant through a surgical trocar with at least 8 mm diameter.
14) Due to the tight bundle, the grasper completely encloses the implant and thus the implant is intact after trocar passage.
15) The XaraColl implant is released from the grasper.
16) Upon release, the XaraColl implant can be unrolled and placed.

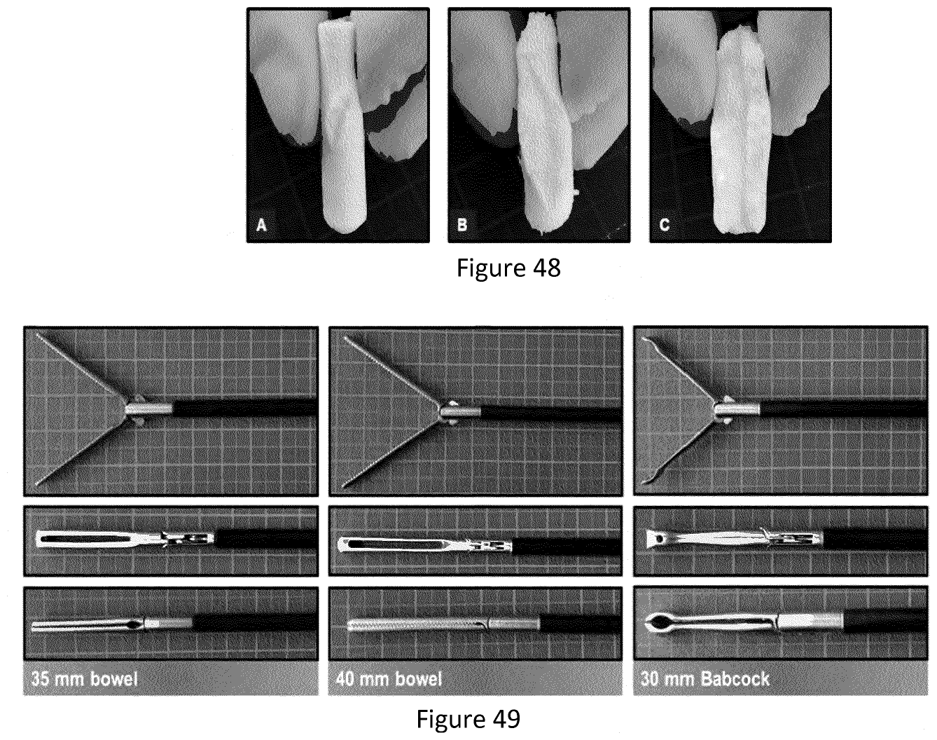

For the evaluation of the successful insertion of the implants, definite criteria were determined. First, the impact of the compression on the implant was assessed by texture analyses. Second, the rolling process was evaluated by grading the condition of the implant afterwards. Thereby, the implants were categorized according to their damage, namely minor damage (<50% of the implant is torn), major damage (>50% of the implant is torn) or complete break (FIG. 48). To ensure that the approach described above functions for several trocar and grasper models, as well as all possible combinations of these instruments, this study includes different types from different manufacturers, i.e. an Ethicon and an Applied trocar, each with 8 mm diameter, a smaller fenestrated bowel grasper with approximately 35 mm jaw length and a somewhat larger bowel grasper with 40 mm jaw length (FIG. 49). In addition to that, the performance of a 30 mm Babcock grasper was evaluated.

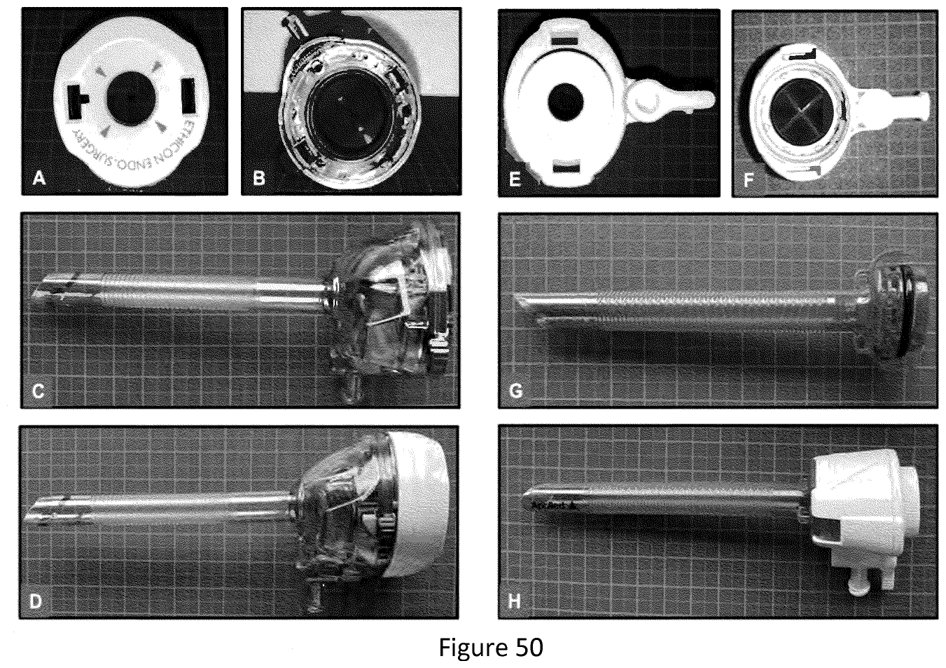

Regarding the trocars, different models from various manufacturers are available. All models with a true inner diameter of at least 8 mm should be suitable in combination with the presented handling approach. However, different trocar types feature different trocar heads with different seals and their performance needs to be evaluated, since the seal offers the most resistance while passing the implant into the abdomen. The J&J Ethicon as well as the Applied trocar series hold a substantial share of the market (i.e. together approximately 95%) and thus they were chosen for this evaluation (FIG. 50).

In addition to the experiments described above, also the unrolling of the implants after trocar passage has been evaluated. For this purpose, XaraColl batch 19011203 was used, which was stored under approximately 30% RH. The unrolling of three samples, that means six halves, was tested, as this dosage corresponds to the number of implants inserted during open inguinal hernia repair surgeries. For this purpose, the handling procedure was conducted as described above, i.e. the implants were compressed, cut, and rolled into a tight bundle. The bundles were held with a 30 mm Babcock grasper and passed through an 8 mm Ethicon trocar. Noteworthy, these instrument combinations represent an exemplary set up and all other possible grasper-trocar combinations are expected to perform identical. Subsequently, the implants were ejected from the grasper on the table. The grasper was then used to grab the implant on one of the endings and placed on a wet sponge. The wet sponge thereby mimics the abdominal wall, onto which the implants usually are attached during surgery. The implant was then grabbed with a second grasper and the implants were unrolled.

### Dissolution Testing

### Reagents and Equipment:

The XaraColl batches and instruments used for this study besides the instruments referred to in the respective method SOPs (for reference see Table 44 above) are listed in Tables 45 and 46, respectively.

TABLE 45
XaraColl batches used for this study.

| | Product | Batch |
|---|---|---|
| Product | code | number |
| XaraColl ® (bupivacaine HCl) | 102040 | 21011207 |
| | | 21020507 |
| | | 21041406 |

TABLE 46
Instruments used for dissolution experiments.

| | | Inv.- | Used for |
|---|---|---|---|
| | | number/ | experiments |
| | Manu- | serial | in |
| Instrument | facturer | number | section |
| Kinetex XB-C18 length 33 mm, | Phenomenex | H20- | 4.1/5.1 |
| ID 3 mm, particle size 2.6 μm | | 079366 | |
| Compression tool generation 6.6 | Syntacoll | N/A | 4.1/5.1 |
| Compression tool generation | Syntacoll | N/A | 4.2/5.2 |
| Trocar (tube length 100 mm, | Ethicon | N/A | 4.1/5.1 |
| diameter 8 mm) | | | |
| Grasper (35 mm jaws, double | New Med | N/A | 4.1/5.1 |
| action atraumatic fenestrated | | | |
| bowel) | | | |

### Dissolution Testing:

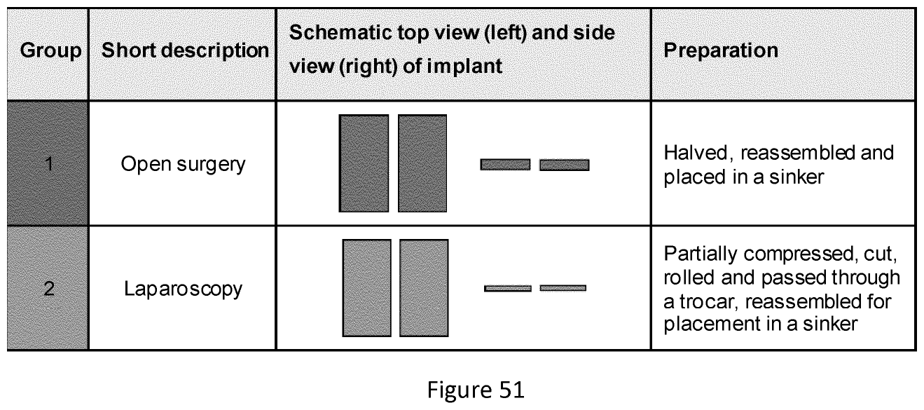

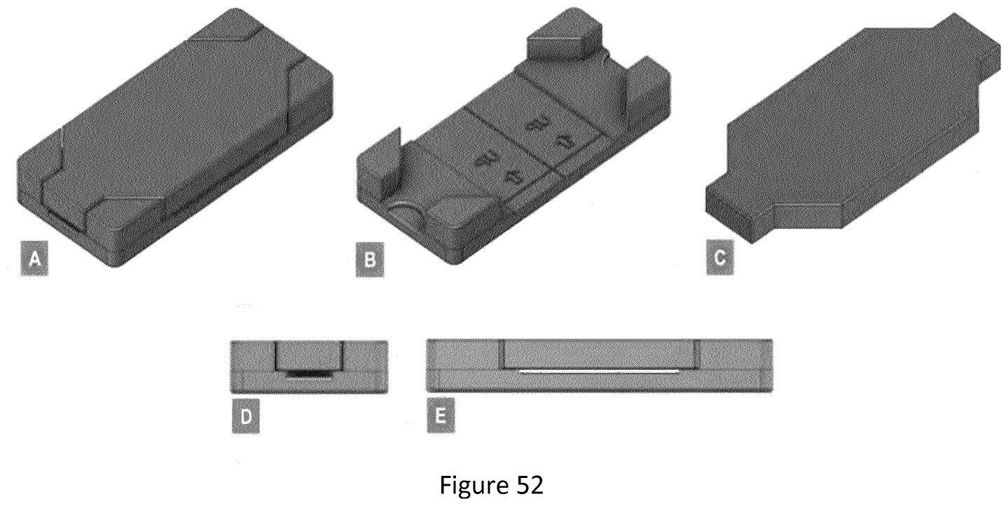

The impact of the sample preparation for laparoscopy on the API release was compared with respect to the drug release of samples prepared for open surgery. For this purpose, halved XaraColl (open surgery, group 1) and compressed, halved and rolled XaraColl, which was pushed through a trocar (laparoscopy, group 2), were investigated (FIG. 51). The analyzed samples are derived from XaraColl batches 21011207 and 21010507. All dissolution experiments were performed using an Agilent dissolution system with dissolution workstation. Samples were collected in a HPLC vial tray and subsequently characterized by HPLC analyses.

### Calculation of f1 and f2 Factors and Interpretation:

The f1 and f2 factors were determined and interpreted as outline in Example 8, using Equation 1 and Table 8.

### QC Release Tests:

To assure that partial compression with the compression tool has no impact on important QC release parameters, all relevant analyses were performed, including content uniformity, related substances, the DSC peak denaturation temperature, identity and content of bupivacaine HCl, SDS-page and dry weight. Prior to these measurements, the implants were compressed using compression tool generation 5.12. Although this was not the final R&D prototype, the results should be equivalent when using the final compression tool generation 6.6, as only negligible changes in the design (without affecting the essential physical parameters) were implemented within the further evolution of the tool.

### Results and Discussion

### Development and Design of the Compression Tool

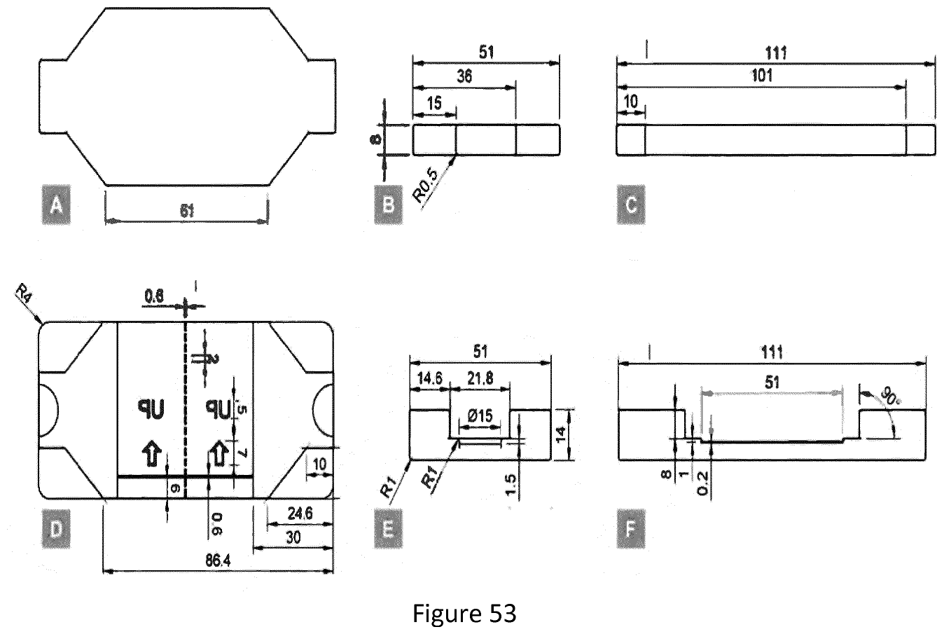

Within the scope of this study, the compression tool has been modified and improved to obtain the final design. In order to exclude misuse of the compression tool, a design which guarantees an intuitive usage within the target group of surgeons and surgery personnel has been developed. The compression tool features distinct details (FIGS. 52 and 53).

The compression tool is composed of a top part and a base, building an assembly with the size of 111×51×14 mm. The implants are centered on the bottom whereas the top part serves as a lid, onto which the palms are placed for compression. The overall assembly is designed with rounded edges and corners to exclude damage of the compression tool packaging, thus maintaining sterility. Compared to previous generations, the angles between the top and the base part were adjusted to a square angle, which allows usage of the top part in either orientation. Between the base and the top part, a compression gap with a defined size of 1 mm is located, which allows controlled compression of the implants. The base part of the compression tool features a defined space of 51×51 mm onto which the implant should be placed. This is only 1 mm larger compared to the implants, which have a size of 50×50 mm, thus assuring correct positioning of the implant. Further, the base part includes several stamps, which are imprinted into the implant whilst compressing it and guide the correct handling. Thereby, “UP” imprint specifies the orientation of the implant, as the instructions which will be provided to the surgeon indicate that the UP imprint should be on the upside of the implant, facing towards the operator. A dotted line in the middle of the implant indicates the cutline. Additionally, the rolling orientation is predetermined by the two arrows, hence serving as further orientation for the scrub tech or surgeon. As the developed handling approach recommends tight rolling of the implants, a small line with 0.6 mm width and 0.2 mm height is imprinted into the bottom end of the implants, which indicates the first fold of the implant prior to rolling, thus defining the size of the rolled bundle.

### Texture Analyses

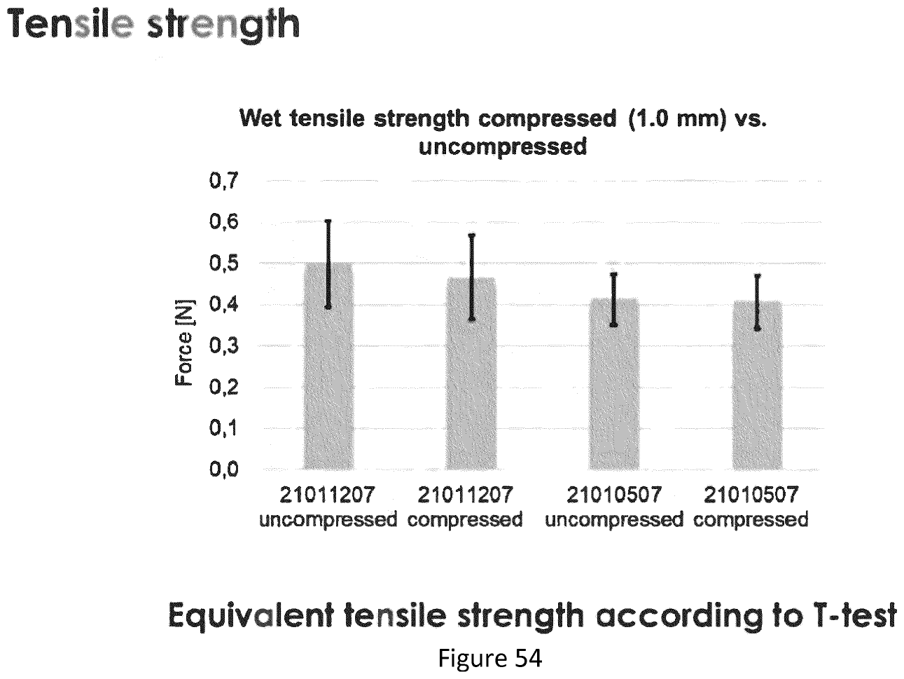

The compression of the XaraColl implants is the key step for the increase in flexibility and thus for the successful trocar passage. In order to estimate the impact of the compression step, various texture measurements have been conducted, including resistance to pressure and resistance to bending measurements as well as the deformation as a consequence thereof (Table 47). In addition to that, the thickness of dry implants as well as measurement of wet implants, including the thickness and tensile strength (FIG. 54), have been determined. Noteworthy, the experiments involving wet implants mirror the behavior and the required space (implant thickness) in vivo.

TABLE 47
Texture data for XaraColl batches 21010507 and
21011207, uncompressed vs compressed implants.

| | | Resistance | Resistance | Deformation | Tensile | | |
|---|---|---|---|---|---|---|---|
| | | to | to | at | strength | Thickness | Thickness |
| | | pressure | bending | break | wet | dry | wet |
| Batch | Group | [N] | [N] | [mm] | [N] | [mm] | [mm] |
| 21010507 | uncompressed | 1.39 | 1.68 | 6.53 | 0.41 | 4.88 | N/A |
| | | (±0.09) | (±0.07) | (±0.71) | (±0.06) | (±0.10) | |
| | partially | 0.38 | 0.59 | 25 | 0.41 | 3.04 | N/A |
| | compressed | (±0.04) | (±0.06) | (no break) | (±0.07) | (±0.10) | |
| 21011207 | uncompressed | 1.53 | 1.87 | 6.56 | 0.50 | 4.81 | 3.10 |
| | | (±0.14) | (±0.09) | (±0.63) | (±0.11) | (±0.17) | (±0.19) |
| | partially | 0.40 | 0.57 | 25 | 0.47 | 3.16 | 2.88 |
| | compressed | (±0.04) | (±0.05) | (no break) | (±0.10) | (±0.17) | (±0.27) |

After compression, the implants were considerably more flexible, which is an important characteristic enabling the trocar insertion. While the uncompressed implants resulted in resistance to pressure values of 1.39 N (±0.09, batch 21010507) and 1.53 N (+0.14, batch 21011207), the values of the compressed samples are decreased to 0.38 N (+0.04, batch 21010507) and 0.40 N (+0.04, 21011207), respectively.

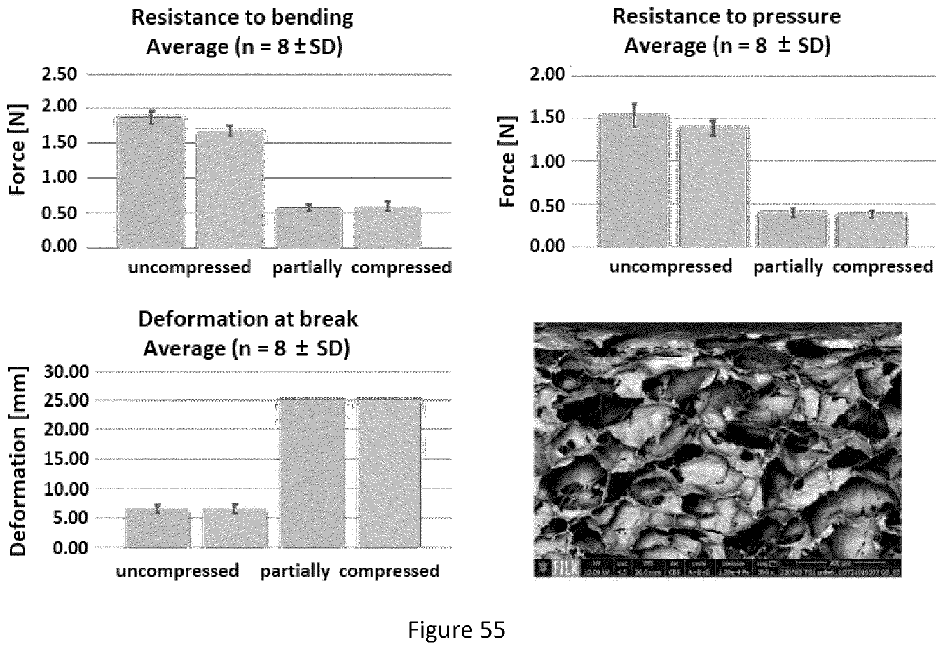

The compression was likewise important for the bending properties of the XaraColl implants, which facilitated rolling the implants without rupturing. Prior to compression, resistance to bending values of 1.68 N (+0.07) and 1.87 N (+0.09) were determined for batch 21010507 and 21011207, respectively, which was accompanied by a deformation at break of 6.53 mm (+0.71, batch 21010507) and 6.56 mm (+0.63, 21011207) (FIG. 55). After exceeding this maximum force, the implants broke. Upon compression, these properties were optimized, as resistance to bending of 0.59 N (+0.06, batch 21010507) and 0.57 N (+0.05, batch 21011207) with corresponding deformation of 25 mm (without breakage) for both batches were measured. Importantly, not only the maximum force was decreased considerably, but also no breaks of the implants were observed after compression.

Noteworthy, even though the implants are partially compressed to a thickness of 1.0 mm using the compression tool, the resulting thickness after release of the compression force amounts to 3.04 mm (±0.10, batch 21010507) and 3.16 mm (+0.17, batch 21011207), due to a considerable re-expansion of the implants. Thus, although the implants are compressed by 79% using the compression tool, the implants are solely 36% reduced in thickness after re-expansion compared to the uncompressed samples, for which heights of 4.88 (±0.10) mm and 4.81 (+0.17) mm were measured for batch 21010507 and 21011207, respectively. Hence, the compression process using the compression tool can be considered as partially reversible.

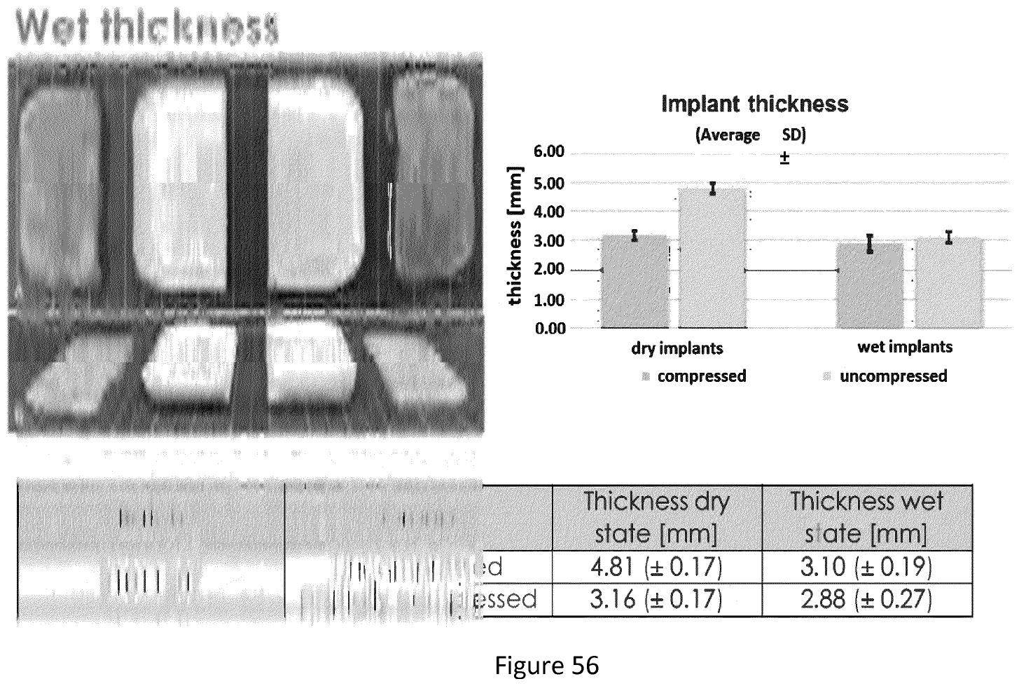

Once the implants were soaked in water, the differences in height adjusted even more. The compressed implants (XaraColl batch 21011207) resulted in a wet thickness of 2.88 (+0.27) mm, whereas for the uncompressed implants a height of 3.10 (+0.19) mm was measured averagely (FIG. 56). This corresponds to a height reduction of only 7% after compression. A statistical T-test confirmed that these height differences are insignificant and thus the wet thickness of compressed and uncompressed implants can be considered equivalent (Table 48).

TABLE 48
Independent 2-tailed T-test results.

| | | | Tensile | Thickness |
|---|---|---|---|---|
| Batch | Group | Parameter | strength | wet |
| 21010507 | uncompressed | t-statistic | 0.42 | N/A |
| | vs partially | tₖᵣᵢₜ | 2.11 | N/A |
| | compressed | P | 0.68 | N/A |
| 21011207 | uncompressed | t-statistic | 0.24 | 1.45 |
| | vs partially | tₖᵣᵢₜ | 2.10 | 2.31 |
| | compressed | P | 0.81 | 0.18 |

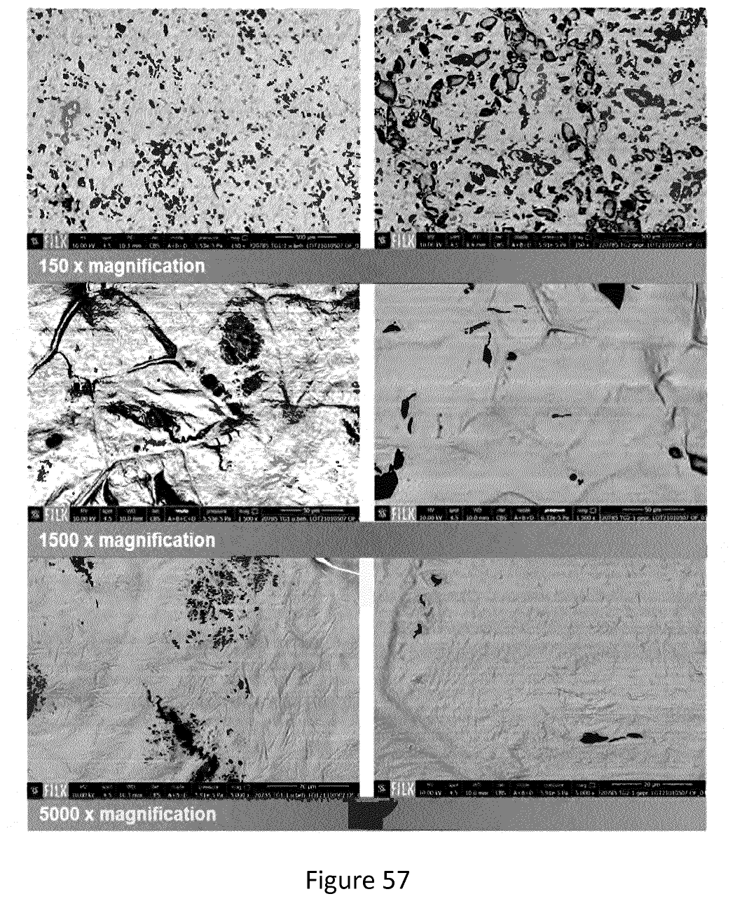

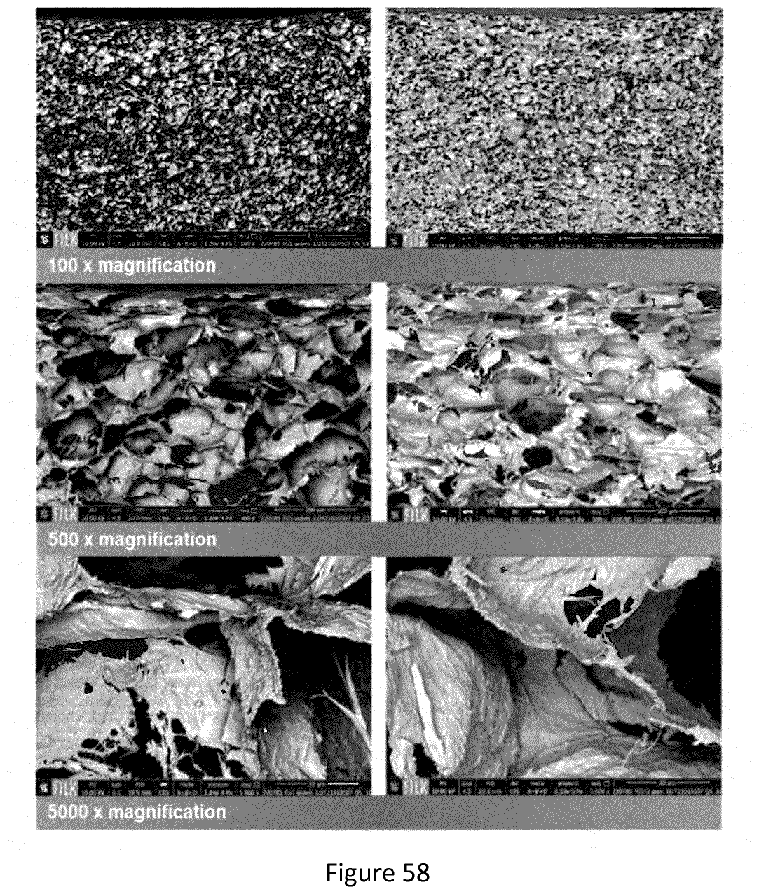

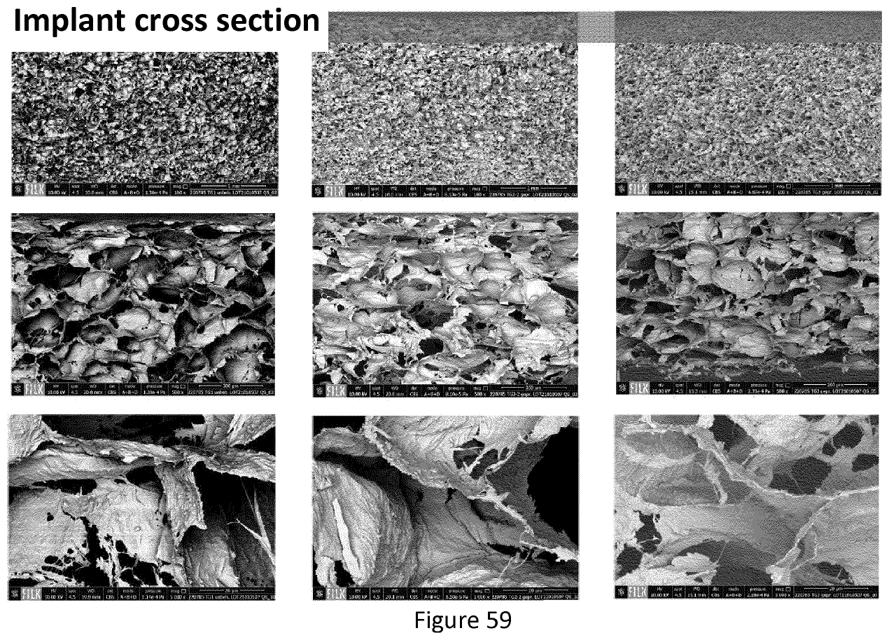

The same equivalence for uncompressed and compressed implants was also observed for tensile strength measurements of wet implants, whereby average values of 0.4 N and 0.5 N were determined for XaraColl batch 21010507 and 21011207, respectively. Again, these values were similar as proven by a statistical T-test. Moreover, SEM measurements were conducted, which demonstrated that the collagen structure remains unaffected by the compression process, revealing the same porous honeycomb structure in both the compressed and the uncompressed implants (FIGS. 57-59).

### Sample Preparation: Compression, Rolling and Trocar Insertion

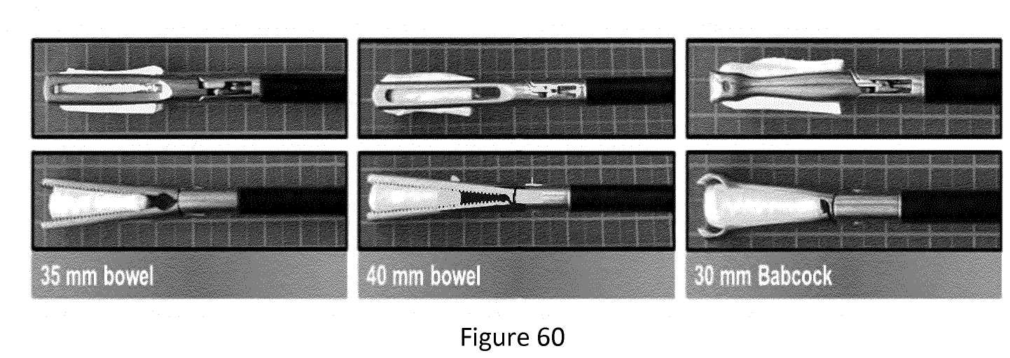

The compression tool handling was investigated involving two different XaraColl batches 21011207 and 21010507, which were rolled and passed through the trocar. Prior to this study, the implants were conditioned at 30% RH. Again, these conditions represent an artificial worst case and are naturally not reached. In order to ensure that the technique works independently from the used trocar or grasper, different types were used, including an Ethicon and an Applied trocar with 8 mm diameter, a 35 and a 40 mm fenestrated bowel grasper with small, atraumatic teeth, as well as a 30 mm Babcock grasper. All graspers were found to be sufficiently long to completely enclose the rolled implant, thus protecting it from surface damage as well as facilitating trocar insertion (FIG. 60).

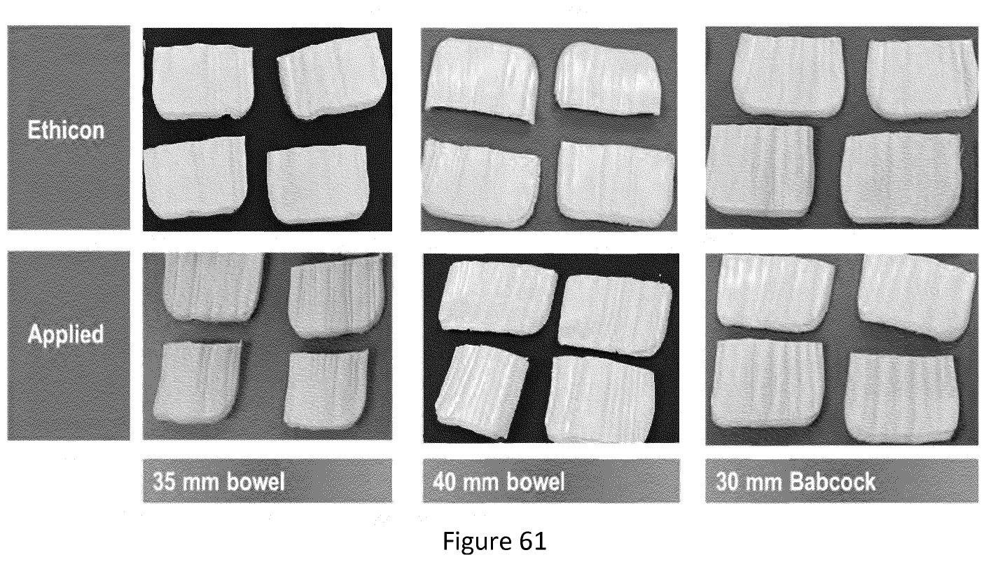

Due to the optimal enclosure of the implant by the different graspers, the implant could be inserted into any 8 mm trocar without serious damage, regardless of the trocar opening architecture (FIG. 61). All implants passed through the trocars using different instrument combinations were in equal and sufficiently good condition and thus all possible trocar-(atraumatic) grasper combinations are suitable for the laparoscopic application of XaraColl. Among a total number of 240 implant halves, only two breaks have been observed, which is an exceptionally low number, given it equates to 0.8% (Table 49). That means that the remaining 99.2% implants passed the trocar in one piece without breakage. Among these 240 samples, nine minor and four major implant damages were found. Again, this is a considerably low number and equals to 3.75% (minor) and 1.67% (major). Noteworthy, even the samples with the recorded damages still allow the usage of the implant for laparoscopic placement and do not exclude the implant from its intended use. These criteria were solely used as a reference point for the evaluation of the process.

TABLE 49
Handling results for two different XaraColl batches, including different grasper and trocar types.

| | | | | | | | Mean | Implant |
|---|---|---|---|---|---|---|---|---|
| | | | | | | No | (no | condition |
| | | | | | | ripping | ripping, | after trocar |
| Batch | Trocar | Grasper | Minor | Major | Break | (%) | %) | passage |
| 21010507 | Ethicon | 35 mm bowel | 0 | 0 | 1 | 98 | ≥95 | Slight surface |
| | 8 mm | (n = 40) | | | | | | damage |
| | | 30 mm Babcock | 2 | 0 | 0 | 90 | | No surface |
| | | (n = 20) | | | | | | damage |
| | Applied | 35 mm bowel | 3 | 1 | 0 | 95 | | Slight to medium |
| | 8 mm | (n = 40) | | | | | | surface damage |
| | | 40 mm bowel | 1 | 1 | 0 | 90 | | No surface |
| | | (n = 20) | | | | | | damage, teeth |
| | | | | | | | | imprints |
| 21011207 | Ethicon | 35 mm bowel | 1 | 0 | 1 | 95 | | Slight surface |
| | 8 mm | (n = 40) | | | | | | damage |
| | | 40 mm bowel | 0 | 0 | 0 | 100 | | No surface |
| | | (n = 20) | | | | | | damage, teeth |
| | | | | | | | | imprints |
| | Applied | 35 mm bowel | 2 | 2 | 0 | 95 | | Slight to medium |
| | 8 mm | (n = 40) | | | | | | surface damage |
| | | 30 mm Babcock | 0 | 1 | 0 | 95 | | No surface |
| | | (n = 20) | | | | | | damage |

Unrolling of the Implants after Trocar Passage

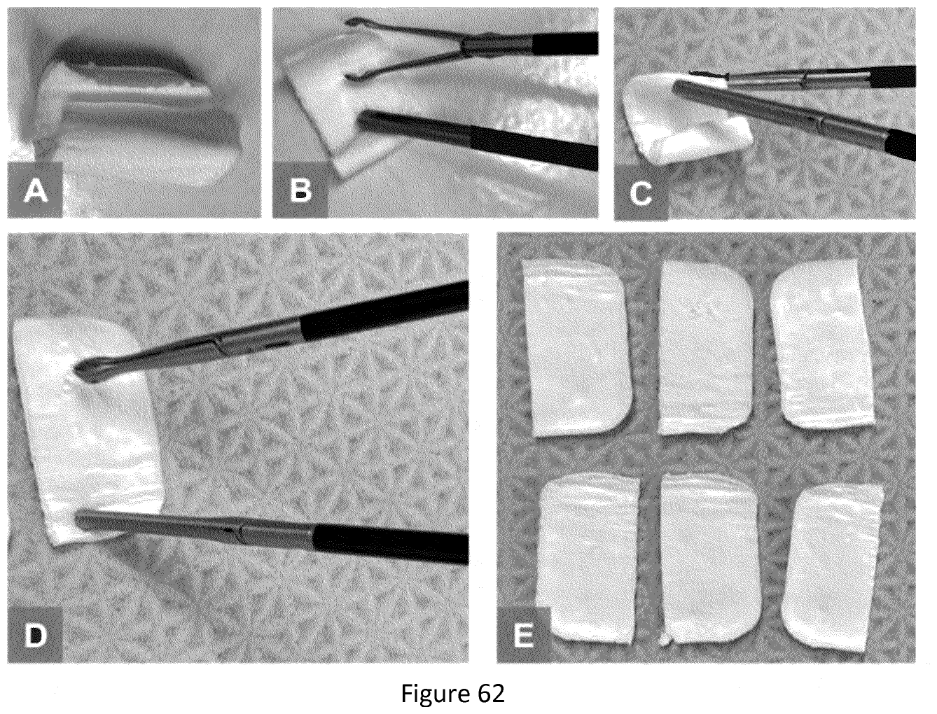

Unrolling of the implants was evaluated using a wet sponge to mimic the abdominal wall, onto which the implants are usually placed during surgery. For this experiment, a 30 mm Babcock grasper was used to pass the implants through an 8 mm Ethicon trocar. Noteworthy, these instrument combinations represent an exemplary set up and all other possible grasper-trocar combinations are expected to perform identical. In total three implants, i.e. six implant halves, were tested, which corresponds to the XaraColl dosage. More precise, the complete handling procedure described above was conducted. Subsequently, the implants were ejected from the grasper on the table after trocar passage, whereupon the bundle re-expanded and partly unrolled autonomously (FIG. 62, A). The grasper was then used to grab the implant on one of the endings (FIG. 62, B) and placed on a wet sponge, which is attached to an inclined plane (FIG. 62, C). Thereupon, the implant slightly sticked to the wet sponge and was unrolled with the help of a second grasper (FIG. 62, D). By proceeding as described above, all six implant halves were placed on the wet sponge successfully and without damage (FIG. 62, E). The successful unrolling was thereby due to the implant's exceptional properties, such as their flexibility gained through the compression process and thus the handling without breakage, their natural and autonomous re-expansion, which allowed easy grabbing of the implant endings with graspers, as well as their ability to stick to moistened surfaces.

### Dissolution Testing

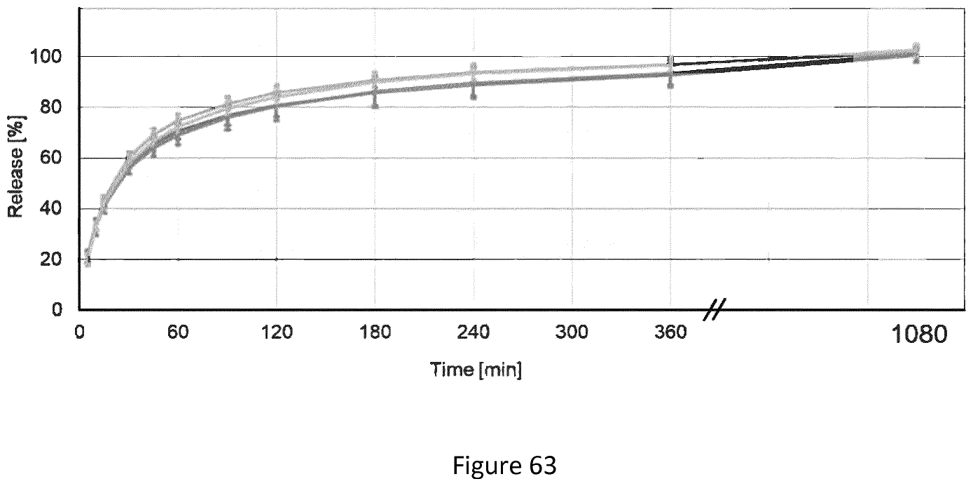

The results obtained from dissolution testing, followed by HPLC analysis, are presented in the following tables (Tables 50-53) and FIG. 63. All samples of test groups 1 and 2 resulted in highly reproducible dissolution profiles with low standard deviations. Furthermore, the standard deviations of the dissolution profiles overlap indicating no statistically significant differences.

TABLE 50
Dissolution data of XaraColl group 1, batch 21010507.
XaraColl batch 21010507, open surgery (n = 17)

| Time | Mean | SD | RSD | c1 | c2 | c3 | c4 | c5 | c6 | c7 | c8 | c9 | c10 | c11 | c12 | c13 | c14 | c15 | c16 | c17 |
|---|---|---|---|---|---|---|---|---|---|---|---|---|---|---|---|---|---|---|---|---|
| [min] | [%] | [%] | [%] | [%] | [%] | [%] | [%] | [%] | [%] | [%] | [%] | [%] | [%] | [%] | [%] | [%] | [%] | [%] | [%] | [%] |
| 5 | 21 | 2.3 | 10.8 | 18 | 20 | 23 | 22 | 17 | 18 | 24 | 21 | 20 | 22 | 22 | 24 | 22 | 19 | 21 | 21 | 21 |
| 10 | 34 | 2.4 | 7.1 | 32 | 32 | 36 | 34 | 29 | 32 | 36 | 34 | 30 | 33 | 35 | 37 | 35 | 32 | 35 | 34 | 34 |
| 15 | 43 | 2.0 | 4.6 | 40 | 41 | 44 | 42 | 39 | 43 | 45 | 44 | 42 | 43 | 45 | 45 | 43 | 41 | 43 | 43 | 43 |
| 30 | 58 | 2.0 | 3.4 | 57 | 55 | 58 | 56 | 56 | 61 | 58 | 60 | 59 | 60 | 60 | 59 | 56 | 56 | 57 | 58 | 58 |
| 45 | 66 | 2.6 | 3.9 | 65 | 61 | 65 | 63 | 65 | 70 | 65 | 68 | 68 | 68 | 68 | 66 | 62 | 64 | 64 | 66 | 66 |
| 60 | 71 | 3.1 | 4.4 | 70 | 66 | 69 | 68 | 71 | 76 | 70 | 74 | 74 | 73 | 73 | 70 | 66 | 69 | 69 | 71 | 71 |
| 90 | 77 | 4.0 | 5.2 | 77 | 72 | 75 | 73 | 78 | 82 | 76 | 81 | 83 | 81 | 80 | 75 | 70 | 76 | 75 | 77 | 76 |
| 120 | 81 | 4.4 | 5.4 | 82 | 76 | 78 | 77 | 83 | 87 | 78 | 84 | 87 | 85 | 83 | 78 | 73 | 79 | 79 | 81 | 80 |
| 180 | 86 | 5.1 | 5.9 | 86 | 81 | 83 | 82 | 89 | 92 | 82 | 89 | 93 | 93 | 88 | 83 | 77 | 84 | 84 | 86 | 86 |
| 240 | 89 | 4.6 | 5.2 | 90 | 84 | 86 | 85 | 94 | 95 | 86 | 92 | 96 | 94 | 92 | 87 | 81 | 87 | 87 | 88 | 89 |
| 360 | 93 | 4.2 | 4.5 | 94 | 89 | 91 | 90 | 97 | 98 | 90 | 96 | 98 | 98 | 95 | 92 | 84 | 91 | 92 | 92 | 92 |
| 1080 | 101 | 2.2 | 2.2 | 101 | 98 | 101 | 102 | 103 | 103 | 98 | 101 | 102 | 103 | 103 | 103 | 97 | 98 | 99 | 99 | 101 |

TABLE 51
Dissolution data of XaraColl group 1, batch 21011207.
XaraColl batch 21011207, open surgery (n = 16)

| Time | Mean | SD | RSD | c1 | c2 | c3 | c4 | c5 | c6 | c7 | c8 | c9 | c10 | c11 | c12 | c13 | c14 | c15 | c16 |
|---|---|---|---|---|---|---|---|---|---|---|---|---|---|---|---|---|---|---|---|
| [min] | [%] | [%] | [%] | [%] | [%] | [%] | [%] | [%] | [%] | [%] | [%] | [%] | [%] | [%] | [%] | [%] | [%] | [%] | [%] |
| 5 | 21 | 2.8 | 13.3 | 23 | 25 | 25 | 24 | 21 | 22 | 18 | 16 | 19 | 22 | 19 | 19 | 20 | 21 | 20 | 21 |
| 10 | 33 | 3.4 | 10.3 | 36 | 37 | 39 | 37 | 32 | 35 | 29 | 27 | 30 | 33 | 31 | 31 | 30 | 33 | 32 | 33 |
| 15 | 41 | 2.7 | 6.7 | 44 | 45 | 43 | 45 | 41 | 43 | 38 | 37 | 39 | 42 | 41 | 40 | 37 | 40 | 40 | 41 |
| 30 | 56 | 2.5 | 4.5 | 58 | 59 | 55 | 59 | 54 | 56 | 55 | 56 | 58 | 60 | 57 | 56 | 51 | 53 | 56 | 55 |
| 45 | 64 | 3.4 | 5.4 | 65 | 65 | 61 | 65 | 62 | 63 | 64 | 67 | 67 | 69 | 68 | 67 | 57 | 60 | 63 | 62 |
| 60 | 69 | 4.1 | 5.9 | 69 | 69 | 65 | 70 | 67 | 67 | 71 | 75 | 74 | 75 | 73 | 71 | 61 | 64 | 68 | 67 |
| 90 | 76 | 4.9 | 6.5 | 75 | 75 | 71 | 76 | 73 | 73 | 79 | 83 | 82 | 82 | 81 | 79 | 67 | 71 | 76 | 73 |
| 120 | 80 | 5.7 | 7.1 | 78 | 78 | 75 | 80 | 77 | 77 | 85 | 88 | 87 | 87 | 86 | 85 | 71 | 73 | 81 | 77 |
| 180 | 86 | 6.2 | 7.2 | 84 | 83 | 80 | 85 | 83 | 83 | 91 | 94 | 93 | 94 | 92 | 91 | 75 | 78 | 87 | 83 |
| 240 | 90 | 6.1 | 6.8 | 88 | 87 | 83 | 88 | 87 | 85 | 94 | 97 | 96 | 97 | 96 | 95 | 79 | 82 | 91 | 86 |
| 360 | 93 | 5.5 | 5.9 | 92 | 91 | 89 | 93 | 91 | 90 | 98 | 101 | 100 | 99 | 99 | 97 | 83 | 87 | 95 | 90 |
| 1080 | 101 | 3.2 | 3.2 | 100 | 99 | 100 | 101 | 101 | 101 | 102 | 105 | 105 | 104 | 104 | 101 | 93 | 97 | 103 | 100 |

TABLE 52
Dissolution data of XARAOLL group 2, batch 21010507.
XaraColl batch 21010507, laparoscopy (n = 12)

| Time | Mean | SD | RSD | a1 | a2 | a3 | a4 | a5 | a6 | a7 | a8 | a9 | a10 | a11 | a12 |
|---|---|---|---|---|---|---|---|---|---|---|---|---|---|---|---|
| [min] | [%] | [%] | [%] | [%] | [%] | [%] | [%] | [%] | [%] | [%] | [%] | [%] | [%] | [%] | [%] |
| 5 | 20 | 2.1 | 10.9 | 20 | 22 | 19 | 18 | 23 | 24 | 19 | 19 | 20 | 17 | 17 | 19 |
| 10 | 33 | 1.6 | 4.8 | 34 | 35 | 34 | 31 | 35 | 35 | 32 | 34 | 33 | 31 | 31 | 33 |
| 15 | 44 | 1.7 | 3.9 | 43 | 45 | 45 | 41 | 46 | 46 | 42 | 44 | 43 | 42 | 41 | 44 |
| 30 | 60 | 1.9 | 3.2 | 61 | 61 | 62 | 58 | 63 | 62 | 58 | 62 | 58 | 61 | 58 | 61 |
| 45 | 69 | 2.2 | 3.2 | 68 | 70 | 72 | 68 | 72 | 71 | 68 | 71 | 65 | 71 | 67 | 70 |
| 60 | 75 | 2.5 | 3.4 | 74 | 76 | 78 | 72 | 77 | 76 | 74 | 76 | 70 | 77 | 73 | 76 |
| 90 | 81 | 2.7 | 3.3 | 80 | 82 | 85 | 80 | 83 | 83 | 80 | 83 | 76 | 85 | 80 | 82 |
| 120 | 86 | 2.8 | 3.3 | 86 | 87 | 89 | 85 | 87 | 87 | 85 | 86 | 79 | 88 | 84 | 87 |
| 180 | 91 | 2.9 | 3.2 | 90 | 92 | 94 | 90 | 93 | 92 | 89 | 91 | 84 | 93 | 90 | 92 |
| 240 | 94 | 2.9 | 3.1 | 92 | 95 | 98 | 93 | 96 | 95 | 93 | 94 | 87 | 96 | 93 | 93 |
| 360 | 97 | 2.7 | 2.8 | 95 | 99 | 100 | 96 | 100 | 98 | 96 | 98 | 91 | 99 | 96 | 96 |
| 1080 | 102 | 1.5 | 1.5 | 100 | 101 | 103 | 103 | 103 | 102 | 103 | 103 | 98 | 102 | 103 | 102 |

TABLE 53
Dissolution data of XaraColl group 2, batch 21011207.
XaraColl batch 21011207, laparoscopy (n = 12)

| Time | Mean | SD | RSD | b1 | b2 | b3 | b4 | b5 | b6 | b7 | b8 | b9 | b10 | b11 | b12 |
|---|---|---|---|---|---|---|---|---|---|---|---|---|---|---|---|
| [min] | [%] | [%] | [%] | [%] | [%] | [%] | [%] | [%] | [%] | [%] | [%] | [%] | [%] | [%] | [%] |
| 5 | 20 | 2.1 | 10.4 | 21 | 20 | 22 | 21 | 22 | 23 | 19 | 17 | 20 | 18 | 19 | 19 |
| 10 | 33 | 1.7 | 5.3 | 34 | 33 | 33 | 34 | 34 | 36 | 33 | 30 | 32 | 31 | 31 | 32 |
| 15 | 42 | 2.2 | 5.2 | 44 | 42 | 41 | 43 | 43 | 46 | 43 | 40 | 40 | 38 | 40 | 41 |
| 30 | 58 | 2.2 | 3.8 | 59 | 59 | 59 | 58 | 60 | 62 | 62 | 59 | 55 | 55 | 55 | 58 |
| 45 | 67 | 2.8 | 4.1 | 67 | 64 | 67 | 66 | 68 | 69 | 72 | 70 | 63 | 65 | 63 | 67 |
| 60 | 72 | 2.9 | 4.1 | 72 | 71 | 73 | 71 | 74 | 73 | 78 | 76 | 68 | 71 | 69 | 74 |
| 90 | 80 | 3.4 | 4.2 | 79 | 78 | 81 | 78 | 81 | 80 | 85 | 85 | 74 | 78 | 76 | 81 |
| 120 | 84 | 3.8 | 4.6 | 83 | 81 | 86 | 82 | 86 | 84 | 90 | 90 | 78 | 82 | 81 | 85 |
| 180 | 90 | 3.8 | 4.2 | 88 | 87 | 91 | 87 | 93 | 90 | 96 | 95 | 84 | 89 | 87 | 91 |
| 240 | 93 | 3.8 | 4.0 | 92 | 91 | 97 | 91 | 95 | 93 | 99 | 99 | 88 | 92 | 90 | 95 |
| 360 | 97 | 3.3 | 3.4 | 96 | 94 | 99 | 94 | 98 | 96 | 102 | 102 | 92 | 95 | 94 | 98 |
| 1080 | 103 | 2.4 | 2.4 | 102 | 101 | 105 | 102 | 103 | 103 | 107 | 105 | 99 | 101 | 102 | 102 |

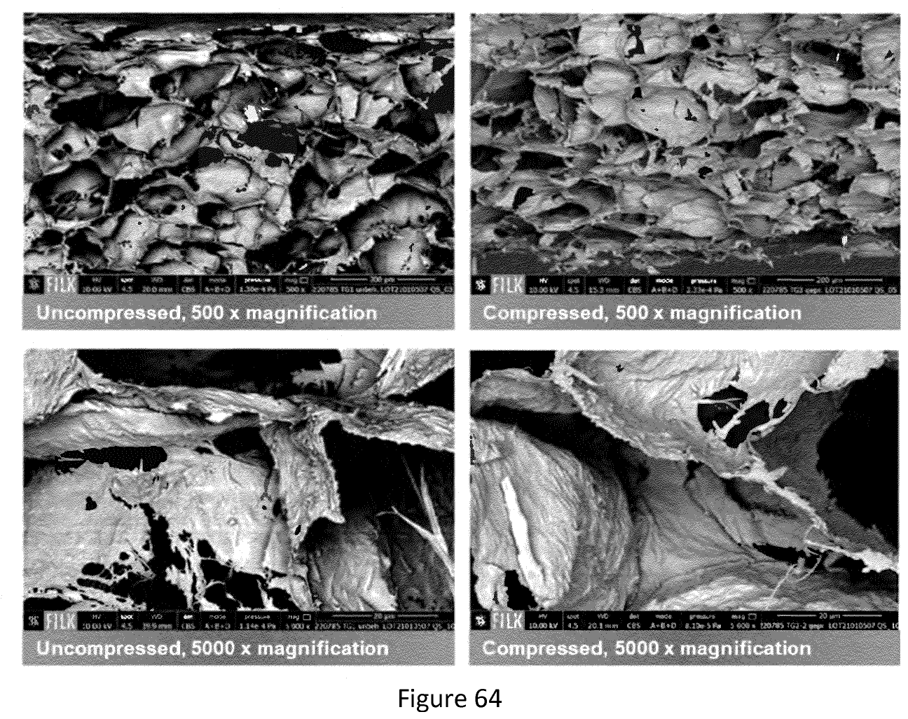

Moreover, f1/f2 statistical analysis confirmed that the results of XaraColl, group 1 can be considered similar/not dissimilar compared to XaraColl group 2 (Table 54). These data indicate that the sample preparation technique introduced above in this Example does not change the drug release characteristics of XaraColl. These observations were in alignment with the SEM cross section images of compressed and uncompressed XaraColl implants (FIG. 64). Upon compression, the typical porous honeycomb-structure of the implants is maintained. In addition to that, the bupivacaine HCl crystals that are adsorbed onto the porous walls were visible and intact in both cases, for the uncompressed as well as for the compressed implants. Thus, the overall structure of XaraColl is unchanged, supporting further that the drug release mechanism is similar for both, uncompressed and compressed implants.

TABLE 54
f1/f2 statistical analysis comparing groups
1 and 2 (open surgery and laparoscopy).

| | | | | | Acceptance | |
|---|---|---|---|---|---|---|
| XaraColl | | | | | criterion | |
| batch | Compared groups | | f1 | f2 | f1 | f2 |
| 21010507 | Open surgery | Laparoscopy | 5.1 | 72.8 | 0-15 | 50-100 |
| 21011207 | Open surgery | Laparoscopy | 3.9 | 78.2 | | |

### QC Release Tests

To assure that partial compression using the compression tool has no impact on important QC release parameters, all relevant analyses were performed, including content uniformity, related substances, the DSC peak denaturation temperature, identity and content of bupivacaine HC), SDS-page and dry weight (Tables 55 and 56). Prior to these measurements, the implants were compressed using compression tool generation 5.12.

TABLE 55
Testing summary for XaraColl batch
21011207 after partial compression.

| | | | Complies |
|---|---|---|---|
| Analysis | Result | Specification | yes/no |
| Dry weight | 179.89 | 157-193 mg | yes |
| Identification | complies | Identification of | yes |
| collagen (SDS-page) | | collagen alpha bands | |
| Identity bupivacaine | complies | Sample RT ± 2% of | yes |
| (HPLC) | | ref. std RT | |
| Assay bupivacaine | 99.23 mg | 90-110 mg | yes |
| (HPLC) | | | |
| Content uniformity | 3.2 | Level 1: AV ≤15 | yes |
| (HPLC) | | | |
| Related substance | <0.01% | ≤0.01% | yes |
| 2,6-DMA (HPLC) | | | |
| Related substance | <0.1% | Any single | yes |
| (GC) | | impurity ≤0.2% | |
| | <0.1% | Total impurities | |
| | | ≤1.0% | |
| Peak denaturation | 43.62° C. | 42° C.-52° C. | yes |
| temperature (DSC) | | | |

TABLE 56
Testing summary for XaraColl batch
21031406 after partial compression.

| | | | Complies |
|---|---|---|---|
| Analysis | Result | Specification | yes/no |
| Dry weight | 170.61 | 157-193 mg | yes |
| Identification | complies | Identification of | yes |
| collagen (SDS-page) | | collagen alpha bands | |
| Identity bupivacaine | complies | Sample RT ± 2% of | yes |
| (HPLC) | | ref. std RT | |
| Assay bupivacaine | 101.63 | 90-110 mg | yes |
| (HPLC) | | | |
| Content uniformity | 3.1 | Level 1: AV ≤15 | yes |
| (HPLC) | | | |
| Related substance | <0.01% | ≤0.01% | yes |
| 2,6-DMA (HPLC) | | | |
| Related substance | <0.1% | Any single impurity ≤0.2% | yes |
| (GC) | <0.1% | Total impurities ≤1.0% | |
| Peak denaturation | 43.18° C. | 42° C.-52° C. | yes |
| temperature (DSC) | | | |

Notably, all tested parameters complied with the specifications for uncompressed XaraColl implants. This fact further emphasizes the equivalence of compressed and uncompressed implants and thus the suitability of the handling technique described above in this Example.

### Conclusions

This study focused on the refinement of the handling technique for the laparoscopic placement of XaraColl and the characterization of the design and handling of the final compression tool 6.6. This handling technique included three steps, i.e. compression of the implants with a designated compression tool, cutting, and rolling. Notably, cutting of the implants is not a newly introduced step for the laparoscopic placement of XaraColl, but is a standard process, which is likewise used for the application of XaraColl during traditional, open surgeries. Hence, compression and rolling can be considered as the key steps for the laparoscopic placement of XaraColl. In order to control these processes to the maximal extent, distinct procedures were developed within this study.

First, the compression tool already introduced and further developed Examples 8 and 9, was refined, yielding the final tool generation 6.6. Tool 6.6 was presented as an assembly of a base and a top part with the size of 111×51×14. Onto the base part, the implants could be centered, whereas the top part served as a lid, onto which the palms are placed for compression. By ingenious design, this compression tool guaranteed correct and intuitive usage by the target groups of surgeons and surgery personnel, as confirmed by the formative study. This included not only the option to use the top part on the compression tool in either way, but also certain stamps onto the base part, which were imprinted into the implant upon compression. These imprints assured the correct orientation of the implant as well as defined the thickness of the rolled bundle by specifying the position for the initiation of the rolling process. Besides, the compression tool featured a defined compression gap of 1 mm, which allowed the controlled and partial compression of the implants. This is of major importance, since controlled and partial compression of the implants was confirmed to be unproblematic, whereas complete and uncontrolled compression should be avoided. Mentionable, the compression with the compression tool did not permanently decrease the thickness of the implants to 1 mm, but the implants re-expanded to a certain extent upon release of the compression force, so that the re-expanded implants had a thickness of approximately 3.1 mm, which is only 36% decreased compared to uncompressed implants. Texture analyses of the compressed implants demonstrated the increased flexibility and bending properties, enabling rolling of the implants without breakage. Remarkably, during these bending experiments the compressed implants were not breaking at all. Noteworthy, SEM images confirmed that while the flexibility and bending properties of the implants are improved, the collagen structure does not change and the typical porous honeycomb network is maintained.

In a second step, the rolling process was intensively screened. Therefore, a total number of 240 implants were rolled and the condition of the implants after trocar passage was evaluated, resulting in a high number of 99.2% successful attempts. In addition to that, the suitability of this process involving different grasper and trocar types was investigated. This included 8 mm (Ethicon and Applied) trocars, a 35 mm and a 40 mm atraumatic bowel grasper as well as a 30 mm Babcock grasper. Additionally, the handling approach was found to function independently of the applied grasper or trocar type.

Lastly, the unrolling of the implants after trocar passage was investigated by using a wet sponge, mimicking the peritoneum or the inner abdominal wall. Successful unrolling of the implant halves was possible by grabbing the ending of the implants with the help of a grasper and attaching them to the moistened sponge with the help of a second grasper. By using this approach, three implants, i.e., six halves, were attached and successfully unrolled without any damage, which corresponds to the dosage used for inguinal hernia repair. The successful unrolling of the implants is due to the implants exceptional properties, such as their natural and autonomous re-expansion, which allowed easy grabbing of the implant endings with graspers, as well as their ability to stick to moistened surfaces.

Summarizing, the herein presented handling technique including controlled, partial compression, cutting, and rolling prior to trocar passage is suitable for the laparoscopic placement of XaraColl. By rolling the implants, their natural tendency for re-expansion and sticking to wet surfaces could be exploited and a further improvement was achieved, namely the efficient unrolling of the implants on a wet surface, which mimics the abdominal wall. Thus, this study covers all preparation steps necessary for laparoscopic procedures and the resounding success of these experiments constitutes a milestone for the laparoscopic application of XaraColl.

Additionally, the sample preparation of XaraColl matrices for laparoscopy described in this Example was evaluated to determine its impact on the performance characteristics. All relevant performance parameters were evaluated within this study, which confirmed the structural and functional equivalence of untreated XaraColl implants and implants that were prepared for laparoscopic placement. These parameters most importantly included the dissolution properties, but also other analyses, such as content uniformity, related substances, the DSC peak denaturation temperature, identity and content of bupivacaine HCl, SDS-page, and dry weight.

f1/f2 statistical analysis confirmed that the drug release of these two groups is similar/not dissimilar, further underlining that the sample preparation technique introduced above in this Example (and mimicking the treatment during an open surgery) does not change the drug release characteristics of XaraColl. QC release tests were performed, including content uniformity, related substances, the DSC peak denaturation temperature, identity and content of bupivacaine HCl, SDS-page, and dry weight. Even though the release criteria are formally only valid for untreated XaraColl implants, the compressed implants comply with all specifications, which again highlights the equivalence of compressed and uncompressed implants. Summarizing, the technique described above in this Example does not change the performance characteristics of XaraColl implants and is therefore suitable for laparoscopic placement of XaraColl.

### Example 11: Laparoscopic Application of XaraColl—Generation 1 Compression Tool Studies with Varying Compression Gaps

### Introduction

The development of a modified sample preparation technique for the laparoscopic application of XaraColl is reported. This sample preparation approach includes three key steps, which are compressing the XaraColl implant with a designated compression tool, cutting the implant into two halves, and rolling the implant, so that a small bundle is formed. This bundle can then be inserted into a trocar by using a grasper. Moreover, the design and 3D-print of a compression tool prototype series with varying compression gaps allowed for the evaluation of the optimum compression gap for this process, which lies in the range between 0.6 and 1.0 mm.

In order to enable the laparoscopic use of XaraColl, it is vital to investigate an approach for the insertion of XaraColl through a trocar as well as to evaluate the characteristics of the XaraColl implant after this process. A first strategy for the trocar insertion of XaraColl has already been described in Example 8. This procedure includes pressing of the XaraColl implant to a thickness of 0.75 mm with a compression tool. The compressed implant is then cut into two halves, which are folded twice. Subsequently, XaraColl can be moved through a trocar with 8 or 12 mm diameter without major damage by using a grasper. However, most surgeons are used to rolling materials before passing them through a trocar rather than folding and, in addition to that, the rolled XaraColl implants are expected to unravel more easily compared to the folded ones.

Hence, this report focuses on the further development of a handling technique of XaraColl which can be applied during laparoscopic surgeries. More precisely, a compression tool prototype series with different compression gaps ranging from 0.6-2.0 mm was manufactured and their handling performance, involving two different XaraColl batches, was evaluated. Moreover, important XaraColl texture parameters, such as resistance to bending and resistance to pressure, were investigated in order to possibly draw performance-texture relationships.

### Methods

### Compression Tool Prototypes

The optimum compression gap for the rolling approach was evaluated. For this purpose, a 1st generation compression tool prototype series was designed and 3D-printed. For an overview of this compression tool series, see FIG. 44A, 1^{st} Generation 1.2 tool, noting that the compression gaps in this prototype series included 0.6 mm, 0.8 mm, 1.0 mm, and 1.2 mm as shown in FIG. 44A in addition to gaps of 1.4 mm, 1.6 mm, 1.8 mm, and 2.0 mm. Each compression tool is constituted of a bottom part, onto which the matrix is placed for compression and which has a defined compression gap. The size of the latter was varied between 0.6 mm and 2.0 mm. A top part of the compression tool serves as a lid, onto which the palms are placed for compression.

The performance of each compression tool is evaluated by analyzing the successful trocar passing attempts of XaraColl after compressing, cutting, and rolling the implants. Additionally, texture parameters, such as resistance to bending and resistance to pressure, were investigated in order to possibly draw performance-texture relationships.

### Interpretation of Results

In order to evaluate the quality and performance of the compression tools, the following parameters are considered:

Rupturing of the XaraColl implant upon rolling. Thereby, the damages are categorized as minor ripping, i.e. <50% of the implant is ruptured, and major ripping, i.e. >50% of the implant is ruptured, and break, i.e. complete breakage of the implant into two parts.

Visual observation of the performance during rolling, e.g. size of the bundle.

Observations and condition of the XaraColl matrix after the trocar insertion process.

### Reagents and Instruments

The following XaraColl batches are used for this study: 21011207, 21010507. All further instruments used are listed in Table 57.

TABLE 57
Instruments used for this study.

| | Manu- | Inv.-number/ |
|---|---|---|
| Instrument | facturer | Serial number |
| Climatic chamber KBF 240 | Binder | 400692 |
| Universal tester Zwicki 500N | Zwicki/Röll | 200134 |
| Trocar (8 mm diameter) | Ethicon | N/A |
| Grasper (double action, 30 mm) | Molnlycke | N/A |
| 3D Printer Pro 2 | Raise 3D | 700152 |

### Sample Preparation: Compression, Rolling and Trocar Insertion

In order to insert a XaraColl implant into a trocar, the fragility and roughness of the matrix have to be reduced to a certain extent, so that the XaraColl implant can be rolled easily and without rupturing. This can be achieved through the compression of XaraColl with a so-called compression tool with a defined compression gap. Upon release of the compression force, the XaraColl implant re-expands to a certain extent. Subsequently, the compressed XaraColl implant can be easily cut into two halves by using scissors. The two halves are rolled and the obtained bundle can be finally inserted through the trocar by using a grasper. Noteworthy, it is crucial that the bottom side of the XaraColl implant is on the outside of the folding as the top side and the bottom side of the XaraColl implant are not identical regarding withstanding physical stress.

### Texture Measurements: Resistance to Bending and Resistance to Pressure

In order to draw conclusions and to unveil important texture-performance relationships, the following texture analyses were conducted:

For resistance to pressure measurements, a one-inch diameter round ball is pressed into the matrix 2 mm deep and the resulting force for 2 mm intrusion is determined. The resistance to bending is measured with a three-point bending set up, whereby the maximum force prior to breaking is determined. Both parameters were quantified by using the universal testing equipment Zwicki 500 N (Table 58). During these measurements a load cell with a maximum capacity of up to 50 N was used.

TABLE 58
Parameters quantified with Zwicki 500N
with corresponding method references.

| Parameter | Load cell |
|---|---|
| Resistance to pressure (F 2 mm) [N] | 50N |
| Resistance to bending (F max) [N] | 50N |

### Sample Preparation: Compression, Rolling and Trocar Insertion

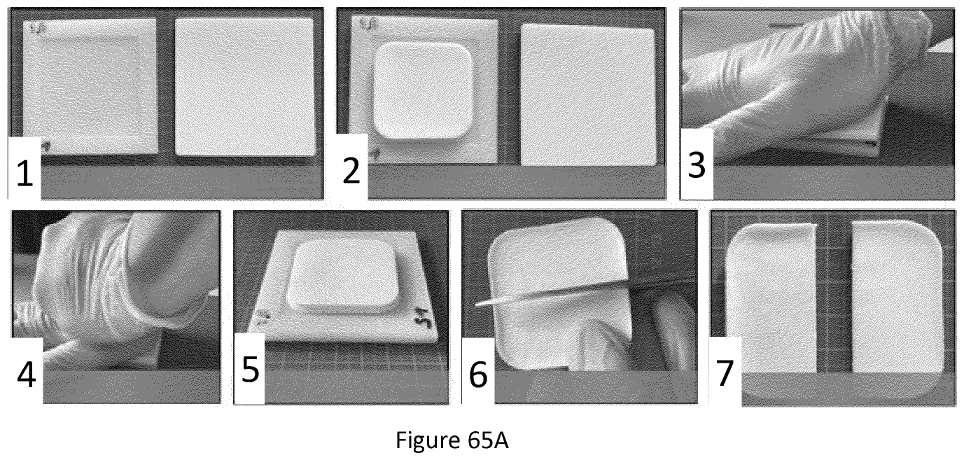

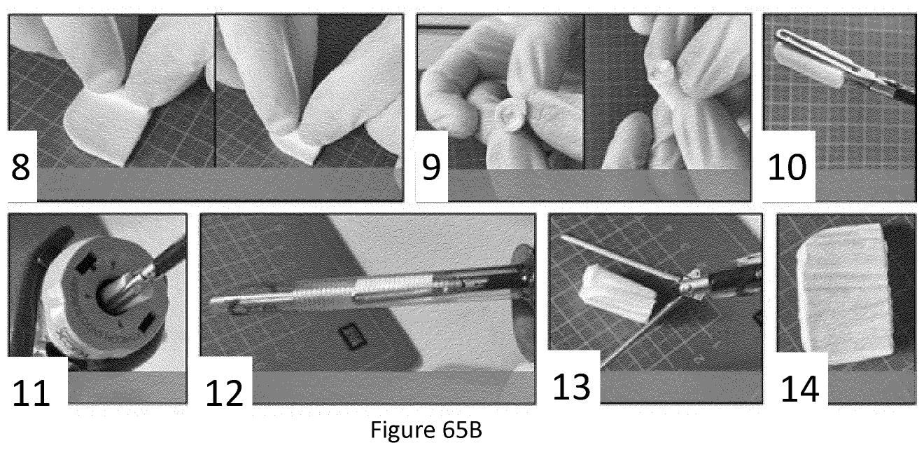

In order to insert a XaraColl implant into a trocar, the fragility and roughness of the matrix have to be reduced to a certain extent, so that the XaraColl implant can be rolled easily and without rupturing. This can be achieved through the compression of XaraColl with a so-called compression tool with a defined compression gap. Upon release of the compression force, the XaraColl implant re-expands to a certain extent. Subsequently, the compressed XaraColl implant can be easily cut into two halves by using scissors. The two halves are rolled and the obtained bundle can be finally inserted through the trocar by using a grasper. Noteworthy, it is crucial that the bottom side of the XaraColl implant is on the outside of the folding as the top side and the bottom side of the XaraColl implant are not identical regarding withstanding physical stress. In detail, the single steps of this preparation procedure include (illustrated in FIGS. 65A-65B):
1) The compression tool consists of a bottom part (left) and a top part (right)
2) XaraColl is placed on the bottom part of a compression tool.
3) The top part of the compression tool serves as a lid onto which the palms are placed for compression.
4) The compression force is maintained for a few seconds.
5) After compression, the XaraColl implant re-expands to a certain extent
6) The XaraColl implant is cut into two halves.
7) The XaraColl halves are placed with the blister side facing towards the table and the Tyvek side facing towards the operator.
8) The XaraColl halves are rolled loosely, resulting in a small bundle
9) Front view and side view of the rolled XaraColl bundle
10) The halved, rolled XaraColl implants are held with a grasper.
11) The XaraColl implant is inserted into the trocar with the help of the grasper.
12) The XaraColl implant is moved through the 8 mm trocar tube
13) The XaraColl implant is released from the grasper.
14) Upon release, the XaraColl implant unfolds.

### Results and Discussion

The compression tool handling was investigated involving two different XaraColl batches with 20 matrices each, i.e. 40 halves, which were rolled and passed through the trocar and three operators (Table 59). In order to assure that the applied technique also functions at worst case conditions, the XaraColl samples were conditioned at 22° C. and 30% r.h. prior to these experiments, since at these conditions the fragility and brittleness of the implants is expected to be at a maximum.

TABLE 59
Handling results for two different XaraColl batches, 40 halves each.

| | | Minor | | | | Major | | | | | | | | No | | |
|---|---|---|---|---|---|---|---|---|---|---|---|---|---|---|---|---|
| | | ripping n/40 | | | | ripping n/40 | | | | Break n/40 | | | | ripping | | |
| Batch | Compression | Operator | | | | Operator | | | | Operator | | | | n/40 | | Observations |
| # | gap size | 1 | 2 | 3 | Σ | 1 | 2 | 3 | Σ | 1 | 2 | 3 | Σ | (%) | | (trocar) |
| 21010507 | 0.6 | 0 | / | 0 | 0 | 0 | 0 | 0 | 0 | 0 | 0 | 0 | 0 | 40 | (100) | Medium force, slight |
| | | | | | | | | | | | | | | | | abrasion |
| 21010507 | 0.8 | / | 0 | 1 | 1 | / | 0 | 2 | 2 | 0 | 0 | 0 | 0 | 37 | (93) | Medium force, slight |
| | | | | | | | | | | | | | | | | abrasion |
| 21010507 | 1.0 | / | 0 | 5 | 5 | / | 0 | 2 | 2 | 0 | 0 | 0 | 0 | 33 | (83) | Medium force, slight |
| | | | | | | | | | | | | | | | | abrasion |
| 21010507 | 1.2 | / | 1 | 0 | 1 | / | 1 | 5 | 6 | / | 1 | 1 | 2 | 31 | (78) | Medium force, slight |
| | | | | | | | | | | | | | | | | abrasion |
| 21010507 | 1.4 | 0 | / | 4 | 4 | 0 | / | 2 | 2 | 0 | / | 2 | 2 | 32 | (80) | Medium force, slight |
| | | | | | | | | | | | | | | | | abrasion |
| 21010507 | 1.6 | 0 | / | 2 | 2 | 0 | / | 1 | 1 | 0 | / | 2 | 2 | 35 | (88) | Medium force, slight |
| | | | | | | | | | | | | | | | | abrasion |
| 21010507 | 1.8 | 0 | / | 0 | 0 | 0 | / | 7 | 7 | 2 | / | 8 | 10 | 23 | (58) | Medium force, slight |
| | | | | | | | | | | | | | | | | abrasion |
| 21010507 | 2.0 | / | 0 | 0 | 0 | / | 0 | 5 | 5 | / | 6 | 8 | 14 | 21 | (53) | Medium to high force, |
| | | | | | | | | | | | | | | | | slight abrasion |
| 21011207 | 0.6 | 0 | / | 1 | 1 | 0 | / | 1 | 1 | 0 | / | 0 | 0 | 38 | (95) | Medium force, slight |
| | | | | | | | | | | | | | | | | abrasion |
| 21011207 | 0.8 | 0 | / | 3 | 3 | 0 | / | 0 | 0 | 0 | / | 0 | 0 | 37 | (93) | Medium force, slight |
| | | | | | | | | | | | | | | | | abrasion |
| 21011207 | 1.0 | 0 | / | 4 | 4 | 0 | / | 4 | 4 | 0 | / | 0 | 0 | 32 | (80) | Medium force, slight |
| | | | | | | | | | | | | | | | | abrasion |
| 21011207 | 1.2 | / | 0 | 4 | 4 | / | 0 | 0 | 0 | / | 0 | 0 | 0 | 36 | (90) | Medium force, slight |
| | | | | | | | | | | | | | | | | abrasion |
| 21011207 | 1.4 | 1 | / | 6 | 7 | 0 | / | 4 | 4 | 1 | / | 0 | 1 | 28 | (70) | Medium force, slight |
| | | | | | | | | | | | | | | | | abrasion |
| 21011207 | 1.6 | / | 0 | 5 | 5 | / | 2 | 4 | 6 | / | 4 | 3 | 7 | 22 | (55) | Medium force, slight |
| | | | | | | | | | | | | | | | | abrasion |
| 21011207 | 1.8 | / | 0 | 4 | 4 | / | 2 | 5 | 7 | / | 6 | 4 | 10 | 19 | (48) | Medium force, slight |
| | | | | | | | | | | | | | | | | abrasion |
| 21011207 | 2.0 | / | 1 | 5 | 6 | / | 1 | 4 | 5 | / | 6 | 5 | 11 | 18 | (45) | Medium force, slight |
| | | | | | | | | | | | | | | | | abrasion |

Clearly, the compression gap has a major impact on the successful laparoscopic handling of XaraColl. In cases where XaraColl is compressed with a compression tool with a smaller gap, i.e. between 0.6 and 1.0 mm, the best results are obtained and no complete breaks are observed. In case of XaraColl batch 21010507, complete breaks are observed starting from a compression gap size of equal or greater 1.2 mm, whereas in case of batch 21011207 the first breakages are occurring for compression gap sizes starting from 1.4 mm. When further increasing the compression gap to up to 2 mm, the number of minor/major tears as well as breaks likewise increases, so that with a 2 mm gap only 53% and 45% of the XaraColl implants can be successfully inserted into the trocar for batches 21010507 and 21011207, respectively. Overall, the handling performance with XaraColl batch 21011207 produces slightly better results compared to batch 21010507, however, these differences are minimal and thus it can be stated that similar results independent from the respective XaraColl batch are produced.

Notably, it was found that the insertion attempts were more likely to be successful if the XaraColl implants were rolled rather loosely than tight, as tight rolling increases the risk of ripping and of breaks. In addition to that, the successful insertion attempts depended on the experience of the respective operators. Operator 1 and 2, that are more experienced with the product as well as the sample preparation technique, produced only three minor and six major tears and 25 breaks in total, whereas the less experienced operator 3 caused 44 minor and 46 major damages and 33 breaks out of a total number of 160 samples.

Notably, the choice of a suitable grasper as well as the correct grasper is likewise important for the successful trocar insertion (for more details refer to Example 8).

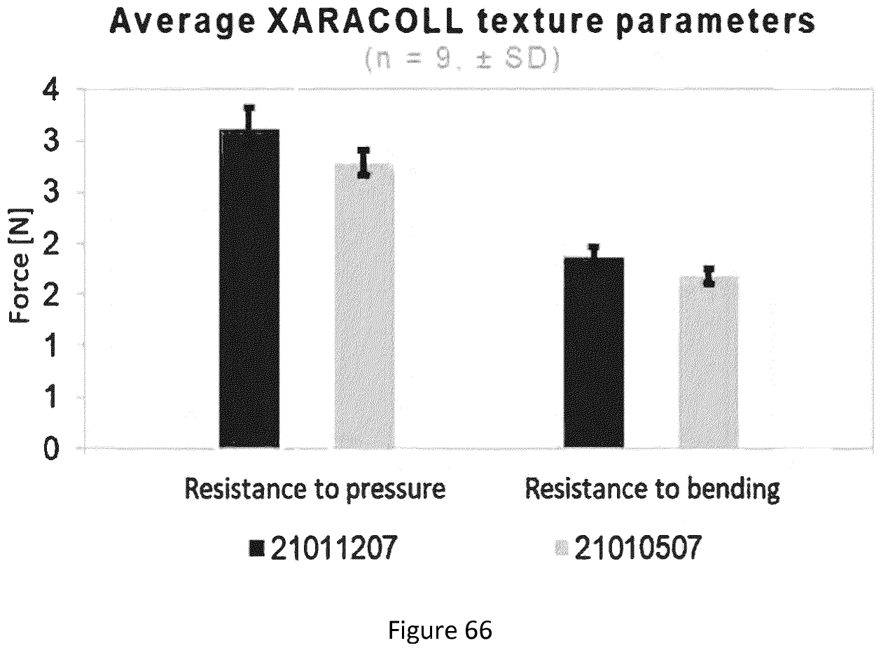

The texture parameters of resistance to pressure and resistance to bending reveal similar and typical values for XaraColl, that means there is not obvious correlation between these parameters and the handling performance (FIG. 66).

### Conclusions

Within this study, a modified sample preparation technique for the laparoscopic application of XaraColl was developed. Different from the process described in Example 8, which involves folding of the XaraColl implant, the present approach includes rolling of the implant. In detail, the key steps of this process are: First, the XaraColl implant is compressed using the designated compression tool. Upon release of the compression force, the implant re-expands up to a certain extent and is subsequently cut into two halves. The compressed, halved implants are loosely rolled and are then eventually inserted through a trocar by using a grasper. In addition to that, the optimum compression gap size for this process was investigated. For this purpose, a compression tool prototype series with compression gaps ranging from 0.6 mm to 2.0 mm was designed and 3D printed and their performance was evaluated. Thereby, the best results were obtained for the smaller compression gaps of up to 1 mm.

In summary, this study presents a successful sample preparation technique for the laparoscopic application of XaraColl. In comparison with prior studies of Example 8, this approach additionally targets different compression gap sizes, whereby an optimum compression gap size between 0.6 m and 1 mm was determined.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

1. A method of implanting a drug delivery device for controlled, sustained release of a drug substance at an implantation site, the method comprising:
inserting the drug delivery device or a segment thereof into a trocar;
moving the drug delivery device or segment thereof through the trocar; and
placing the drug delivery device or segment thereof at the implantation site.

2. The method of claim 1, wherein the drug delivery device comprises a fibrillar collagen matrix and at least one drug substance selected from an amino amide anesthetic, an amino ester anesthetic, and mixtures thereof.

3. The method of claim 2, wherein the drug substance is selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix.

4. The method of any one of claims 1 to 3, wherein the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm.

5. The method of any one of claims 1 to 4, further comprising removing the drug delivery device from a blister pack enclosure, the drug delivery device comprising a first side which is the blister side, and a second side which is the side opposite to the blister side, wherein the blister side has an increased elasticity compared to the side opposite to the blister side.

6. The method of any one of claims 1 to 4, wherein the drug delivery device comprises a first side and a second side, wherein the first side has an increased elasticity compared to the second side.

7. The method of any one of claims 1 to 4, wherein the drug delivery device comprises a first side and a second side, wherein the first side is more stretchable compared to the second side.

8. The method of any one of claims 1 to 4, wherein the drug delivery device comprises a first side and a second side, wherein the first side comprises one or more beveled surfaces or edges.

9. The method of any one of claims 1 to 4, wherein the drug delivery device comprises a first side and a second side, wherein the first side is convex, and the second side is concave.

10. The method of any one of claims 1 to 5, wherein the drug delivery device comprises a first side and a second side, wherein the first side is more flexible compared to the second side.

11. The method of any one of claims 1 to 10, further comprising compressing the drug delivery device prior to inserting into a trocar.

12. The method of claim 11, wherein the compressing comprises compressing the drug delivery device between two substantially parallel compression surfaces, wherein when fully closed the compression surfaces define a gap of about 0.5 mm to about 1.6 mm, or about 0.6 mm to about 1.2 mm, or about 0.8 mm to about 1 mm.

13. The method of claim 11 or 12, wherein the compressing comprises compressing the drug delivery device to a thickness of between about 10% to about 35% of the uncompressed drug delivery device.

14. The method of any one of claims 11 to 13, wherein after compressing, the drug delivery device re-expands to a thickness of between about 30% to about 70% of an uncompressed drug delivery device.

15. The method of any one of claims 11 to 14, wherein after compressing, the drug delivery device re-expands to a thickness of between about 2 mm to about 3 mm.

16. The method of any one of claims 1 to 15, further comprising partitioning the drug delivery device into two or more segments, wherein each segment is placed at the implantation site independently.

17. The method of any one of claims 1 to 16, further comprising folding or rolling the drug delivery device or segment thereof prior to insertion into the trocar.

18. The method of any one of claims 5 to 16, further comprising folding or rolling the drug delivery device or drug delivery device segment prior to insertion into the trocar, wherein the first side is substantially at the exterior of the folded or rolled drug delivery device or drug delivery device segment.

19. The method of any one of claims 1 to 18, wherein the trocar has an internal diameter in a range from about 5 mm to about 16 mm.

20. The method of any one of claims 1 to 18, wherein the trocar has an internal diameter of about 5 mm, about 8 mm, about 10 mm, or about 12 mm.

21. The method of any one of claims 1 to 20, wherein the inserting of the drug delivery device or a segment thereof into a trocar is performed with a grasper.

22. The method of any one of claims 1 to 21, wherein the moving of the drug delivery device or segment thereof through the trocar is performed with a grasper.

23. The method of any one of claims 1 to 22, wherein the placing of the drug delivery device or segment thereof at the implantation site is performed with a grasper.

24. The method of claim 23, further comprises unrolling or unfolding the drug delivery device or segment thereof after placement at the implantation site.

25. The method of any one of claims 1 to 24, wherein the release dissolution profile of a sum of drug delivery device segments is substantially similar to the release dissolution profile of the unpartitioned drug delivery device, wherein the total dose of drug substance in the sum of drug delivery device segments and total dose of drug substance in the unpartitioned drug delivery device is substantially similar.

26. The method of any one of claims 1 to 24, wherein the release dissolution profile of the sum of drug delivery device segments is within about 1% and about 20% at any point in time substantially similar to the release dissolution profile of the unpartitioned drug delivery device, wherein the total dose of drug substance in the sum of drug delivery device segments and total dose of drug substance in the unpartitioned drug delivery device is substantially similar.

27. The method of claim 25 or 26, wherein the total dose is about 100 mg, about 200 mg, or about 300 mg.

28. Use of a drug delivery device or segments thereof for controlled, sustained release of a drug substance at an implantation site, wherein the drug delivery device or segments thereof are configured for inserting into a trocar, moving through the trocar, and placing at the implantation site.

29. The use of claim 28, wherein the drug delivery device comprises a fibrillar collagen matrix and at least one drug substance selected from an amino amide anesthetic, an amino ester anesthetic, and mixtures thereof, or wherein the drug substance is selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix.

30. The use of claim 28 or 29, wherein the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm.

31. The use of any one of claims 28 to 30, the drug delivery device being removable from a blister pack enclosure, the drug delivery device comprising a first side which is the blister side, and a second side which is the side opposite to the blister side, wherein the blister side has an increased elasticity compared to the side opposite to the blister side.

32. The use of any one of claims 28 to 30, wherein the drug delivery device or segment thereof comprises a first side and a second side, wherein the first side has an increased elasticity compared to the second side, and/or wherein the first side is more stretchable compared to the second side, and/or wherein the first side comprises one or more beveled surfaces or edges, and/or wherein the first side is convex, and the second side is concave, and/or wherein the first side is more flexible compared to the second side.

33. The use of any one of claims 28 to 32, wherein the drug delivery device or segment thereof is further configured to be compressed between two substantially parallel compression surfaces, wherein when fully closed the compression surfaces define a gap of about 0.5 mm to about 1.6 mm, or about 0.6 mm to about 1.2 mm, or about 0.8 mm to about 1 mm, and/or wherein the drug delivery device is configured to be compressed to a thickness of between about 10% to about 35% of the uncompressed drug delivery device.

34. The use of claim 33, wherein the drug delivery device is configured to re-expand after compressing to a thickness of between about 30% to about 70% of an uncompressed drug delivery device, and/or to a thickness of between about 2 mm to about 3 mm.

35. The use of any one of claims 28 to 34, wherein the drug delivery device is configured to be partitioned into two or more segments, wherein each segment is configured to be placed at the implantation site independently.

36. The use of any one of claims 28 to 35, wherein the drug delivery device or segment thereof is configured to be folded or rolled.

37. The use of any one of claims 31 to 35, wherein the drug delivery device or segment thereof is configured to be folded or rolled, wherein the first side is substantially at the exterior of the folded or rolled drug delivery device or drug delivery device segment.

38. The use of any one of claims 28 to 37, wherein the drug delivery device or segment thereof is configured for delivery through a trocar having an internal diameter in a range from about 5 mm to about 16 mm, and/or an internal diameter of about 5 mm, about 8 mm, about 10 mm, or about 12 mm.

39. The use of any one of claims 31 to 38, wherein the drug delivery device or segment thereof is configured to be inserted into a trocar with a grasper, and/or to be moved through the trocar with a grasper, and/or placed at the implantation site with a grasper.

40. The use of claim 39, wherein the drug delivery device or segment thereof is configured to be unfolded or unrolled after placement at the implantation site.

41. The use of any one of claims 28 to 40, wherein the release dissolution profile of a sum of drug delivery device segments is substantially similar to the release dissolution profile of the unpartitioned drug delivery device, wherein the total dose of drug substance in the sum of drug delivery device segments and total dose of drug substance in the unpartitioned drug delivery device is substantially similar, or wherein the release dissolution profile of the sum of drug delivery device segments is within about 1% and about 20% at any point in time substantially similar to the release dissolution profile of the unpartitioned drug delivery device, wherein the total dose of drug substance in the sum of drug delivery device segments and total dose of drug substance in the unpartitioned drug delivery device is substantially similar.

42. The use of claim 41, wherein the total dose is about 100 mg, about 200 mg, or about 300 mg.

43. A kit for implanting a drug delivery device or a segment thereof during laparoscopic surgery, the kit comprising:
a drug delivery device for controlled, sustained release of a drug substance at an implantation site, and
a compression device for compressing the drug delivery device.

44. The kit of claim 43, further comprising instructions for a clinician for implanting the drug delivery device at an implantation site during a laparoscopic procedure.

45. The kit of claim 43 or 44, wherein the compression device comprises a top plate and a bottom plate.

46. The kit of any one of claims 43 to 46, wherein the instructions comprise the steps for:
placing a drug delivery device between the plates of the compression device;
compressing the drug delivery device between the plates of the compression device;
optionally cutting the compressed drug delivery device into a desired shape; and
folding or rolling the cut compressed drug delivery device in a shape amenable to be inserted through a trocar.

47. The kit of any one of claims 43 to 46, wherein the drug delivery device comprises a fibrillar collagen matrix and at least one drug substance selected from bupivacaine, levobupivacaine, lidocaine, mepivacaine, prilocaine, ropivacaine, articaine, trimecaine, and their salts and prodrugs, and wherein the fibrillar collagen matrix comprises a Type I collagen matrix.

48. The kit of any one of claims 43 to 46, wherein the drug delivery device comprises one or more collagen sponges, wherein a collagen sponge comprises about 4.0 to about 15.0 mg/cm³ bupivacaine hydrochloride, and has a length of about 50 mm, a width of about 50 mm, and a thickness of about 5 mm.

49. The kit of any one of claims 43 to 46, wherein the drug delivery device is removable from a blister pack enclosure, the drug delivery device comprising a first side which is the blister side, and a second side which is the side opposite to the blister side, wherein the blister side has an increased elasticity compared to the side opposite to the blister side, and/or wherein the blister side is more stretchable compared to the side opposite to the blister side, and/or wherein the blister side comprises one or more beveled surfaces or edges, and/or wherein the blister side is more stretchable compared to the side opposite to the blister side, and/or wherein the blister side is convex, and the side opposite to the blister side is concave, and/or wherein the blister side is more flexible compared to the side opposite to the blister side.

50. The kit of any one of claims 43 to 46, wherein the folding or rolling the cut compressed drug delivery device step further comprises folding or rolling the drug delivery device or segment thereof with the first side substantially at the exterior of the folded or rolled drug delivery device or drug delivery device segment, and wherein the instructions further comprise the step of unrolling or unfolding the drug delivery device or segment thereof after placement at the implantation site.
